SEQUENCE LISTING (209 sequences)

SEQ ID NO: 1 - DNA, 1490 bp, Pseudomonas fluorescens
  source 1..1490: mol_type = genomic DNA
agtcagacatgcaagtcgagcggtagagagaagcttgcttctcttgagagcggcggacgggtgagtaaagcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 2 - DNA, 1017 bp, Hanseniaspora opuntiae
  source 1..1017: mol_type = genomic DNA
ttgttgctcgagttcttgtttagatcttttacaataatgtgtatctttaatgaagatgngngcttaattgcgctgctttattagagtgtcgcagtagaagtagtcttgcttgaatctcagtcaacgtttacacacattggagtttttttactttaatttaattctttctgctttgaatcgaaaggttcaaggcaaaaaacaaacacaaacaattttattttattataattttttaaactaaaccaaaattcctaacggaaattttaaaataatttaaaactttcaacaacggatctcttggttctcgcatcgatgaaaaacgtaccgaattgcgataagtaatgtgaattgcaaatactcgtgaatcattgaatttttgaacgcacattgcgcccttgagcattctcaagggcatgcctgtttgagcgtcatttccttctcaaaaaataattttttattttttggttgtgggcgatactcagggttagcttgaaattggagactgtttcagtcttttttaattcaacacttancttctttggagacgctgttctcgctgtgatgtatttatggatttattcgttttactttacaagggaaatggtaatgtaccttaggcaaagggttgcttttaatattcatcaagtttgacctcaaatcaggtaggattacccgctgaacttaagcatatcaataagcggaggaaaagaaaccaactgggattaccttagtaacggcgagtgaagcggtaaaagctcaaatttgaaatctggtactttcagtgcccgagttgtaatttgtagaatttgtctttgattaggtccttgtctatgttccttggaacaggacgtcatagagggtgagantcccgtttgnngaggataccttttctctgtannactttttcnaagagtcgagttgnttgggaatgcagctcaaanngggtngnaaattccatctaaagctaaatattngncnagagaccganagcgacantacagngatggaaagangaaa

SEQ ID NO: 3 - DNA, 1548 bp, Leuconostoc mesenteroides
  source 1..1548: mol_type = genomic DNA
attgagagtttgatcctggctcaggatgaacgctggcggcgtgcctaatacatgcaagtcgaacgcacagcgaaaggtgcttgcacctttcaagtgagtggcgaacgggtgagtaacacgtggacaacctgcctcaaggctggggataacatttggaaacagatgctaataccgaataaaactcagtgtcgcatgacacaaagttaaaaggcgctttggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtacgggaagaacagctagaatagggaatgattttagtttgacggtaccataccagaaagggacggctaaatacgtgccagcagccgcggtaatacgtatgtcccgagcgttatccggatttattgggcgtaaagcgagcgcagacggttgattaagtctgatgtgaaagcccggagctcaactccggaatggcattggaaactggttaacttgagtgcagtagaggtaagtggaactccatgtgtagcggtggaatgcgtagatatatggaagaacaccagtggcgaaggcggcttactggactgtaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggattgtagtctgcaactcgactacatgaagtcggaatcgctagtaatcgcggatcagcacgccgcggtgaatacgttcccgggtcttgtacacaccgcccgtcacaccatgggagtttgtaatgcccaaagccggtggcctaaccttttaggaaggagccgtctaaggcaggacagatgactggggtgaagtcgtaacaaggtagccgtaggagaacctgcggctggatcacctccttt

SEQ ID NO: 4 - DNA, 916 bp, Aureobasidium pullulans
  source 1..916: mol_type = genomic DNA
ctttgttgttaaaactaccttgttgctttggcgggaccgctcggtctcgagccgctggggattcgtcccaggcgagcgcccgccagagttaaaccaaactcttgttatttaaccggtcgtctgagttaaaattttgaataaatcaaaactttcaacaacggatctcttggttctcgcatcgatgaagaacgcagcgaaatgcgataagtaatgtgaattgcagaattcagtgaatcatcgaatctttgaacgcacattgcgccccttggtattccgaggggcatgcctgttcgagcgtcattacaccactcaagctatgcttggtattgggcgtcgtccttagttgggcgcgccttaaagacctcggcgaggccactccggctttaggcgtagtagaatttattcgaacgtctgtcaaaggagaggaactctgccgactgaaacctttatttttctaggttgacctcggatcaggtagggatacccgctgaacttaagcatatcaataagcggaggaaaagaaaccaacagggattgccctagtaacggcgagtgaagcggcaacagctcaaatttgaaagctagccttcgggttcgcattgtaatttgtagaggatgatttggggaagccgcctgtctaagttccttggaacaggacgtcatagagggtgagaatcccgtatgtgacaggaaatggcaccctatgtaaatctccttcgacgagtcgagttgtttgggaatgcagctctaaatgggaggtaaatttcttctaaagctaaatattggcgagagaccgatagcgcacaagtagagtgatcgaaagatgaaaagcactttggaaagagagttaaaaagcacgtgaaattgttgaaagggaagcgcttgcaatcagacttgtttaaactgttcggccggt

SEQ ID NO: 5 - DNA, 1042 bp, Debaromyces hansenii
  source 1..1042: mol_type = genomic DNA
gcgcttattgcgcggcgaaaaaaccttacacacagtgttttttgttattacannaacttttgctttggtctggactagaaatagtttgggccagaggttactaaactaaacttcaatatttatattgaattgttatttatttaattgtcaatttgttgattaaattcaaaaaatcttcaaaactttcaacaacggatctcttggttctcgcatcgatgaagaacgcagcgaaatgcgataagtaatatgaattgcagattttcgtgaatcatcgaatctttgaacgcacattgcgccctctggtattccagagggcatgcctgtttgagcgtcatttctctctcaaaccttcgggtttggtattgagtgatactcttagtcgaactaggcgtttgcttgaaatgtattggcatgagtggtactggatagtgctatatgactttcaatgtattaggtttatccaactcgttgaatagtttaatggtatatttctcggtattctaggctcggccttacaatataacaaacaagtttgacctcaaatcaggtaggattacccgctgaacttaagcatatcaataagcggaggaaaagaaaccaacagggattgccttagtaacggcgagtgaagcggcaaaagctcaaatttgaaatctggcaccttcggtgtccgagttgtaatttgaagaaggtaactttggagttggctcttgtctatgttccttggaacaggacgtcacagagggtgagaatcccgtgcgatgagatgcccaattctatgtaaagtgctttcgaagagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaaagggcttgagatcagacttggtattttgcgatcctttccttcttggttgggttcctcgcagcttactgggncagcatcggtttggatgg

SEQ ID NO: 6 - DNA, 1165 bp, Bacillus cereus
  source 1..1165: mol_type = genomic DNA
gaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctgcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcat

SEQ ID NO: 7 - DNA, 871 bp, Pichia fermentans
  source 1..871: mol_type = genomic DNA
ccacnctgcgtgggcgacacgaaacaccgaaaccgaacgcacgccgtcaagcaagaaatccacaaaactttcaacaacggatctcttggttctcgcatcgatgaagagcgcagcgaaatgcgatacctagtgtgaattgcagccatcgtgaatcatcgagttcttgaacgcacattgcgcccgctggtattccggcgggcatgcctgtctgagcgtcgtttccttcttggagcggagcttcagacctggcgggctgtctttcgggacggcgcgcccaaagcgaggggccttctgcgcgaactagactgtgcgcgcggggcggccggcgaacttataccaagctcgacctcagatcaggcaggagtacccgctgaacttaagcatatcaataagcggaggaaaagaaaccaacagggattgccccagtagcggcgagtgaagcggcaaaagctcagatttggaatcgcttcggcgagttgtgaattgcaggttggcgcctctgcggcggcggcggtccaagtcccttggaacagggcgccattgagggtgagagccccgtgggaccgtttgcctatgctctgaggcccttctgacgagtcgagttgtttgggaatgcagctctaagcgggtggtaaattccatctaaggctaaatactggcgagagaccgatagcgaacaagtactgtgaaggaaagatgaaaagcactttgaaaagagagtgaaacagcacgtgaaattgttgaaagggaagggtattgcgcccgacatggagcgtgcgcaccgctgcccctcgtgggcggcgctctgggcgtgctctgggccagcatcggtttttgccgcgggagaagggcggcgggcatgtagctcttc

SEQ ID NO: 8 - DNA, 942 bp, Hanseniaspora opuntiae
  source 1..942: mol_type = genomic DNA
gttgctcgagttcttgtttagatcttttacnataatgtgtatctttaatgaagatgtgcgcttaattgcgctgctttattagagtgtcgcagtagaagtagtcttgcttgaatctcagtcaacgtttacacacattggagtttttttactttaatttaattctttctgctttgaatcgaaaggttcaaggcaaaaaacaaacacaaacaattttattttattataattttttaaactaaaccaaaattcctaacggaaattttaaaataatttaaaactttcaacaacggatctcttggttctcgcatcgatgaaaaacgtagcgaattgcgataagtaatgtgaattgcaaatactcgtgaatcattgaatttttgaacgcacattgcgcccttgagcattctcaagggcatgcctgtttgagcgtcatttccttctcaaaagataattttttattttttggttgtgggcgatactcagggttagcttgaaattggagactgtttcagtcttttttaattcaacacttancttctttggagacgctgttctcgctgtgatgtatttatggatttattcgttttactttacaagggaaatggtaatgtaccttaggcaaagggttgcttttaatattcatcaagtttgacctcaaatcaggtaggattacccgctgaacttaagcatatcaataagcggaggaaaagaaaccaactgggattaccttagtaacggcgagtgaagcggtaaaagctcaaatttgaaatctggtactttcanngcccgagttgtaatttgtagaatttgtctttgattaggtccttgtctatgttccttggancaggacgtcatanagggtgantcccntttggcganganaccttttctctgtanactttttcnanagtcgagttgtttnggatgcagctcnaagtggggngg

SEQ ID NO: 9 - DNA, 1572 bp, Pediococcus pentosaceus
  source 1..1572: mol_type = genomic DNA
atgagagtttgatcttggctcaggatgaacgctggcggcgtgcctaatacatgcaagtcgaacgaacttccgttaattgattatgacgtacttgtactgattgagattttaacacgaagtgagtggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggcaaaggctcaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgagaccgcgaggttaagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagtcgtaacaaggtagccgtaggagaacctgcggctggatcacctcctt

SEQ ID NO: 10 - DNA, 1300 bp, Bacillus velezensis
  source 1..1300: mol_type = genomic DNA
cagatagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 11 - DNA, 1419 bp, Pseudomonas putida
  source 1..1419: mol_type = genomic DNA
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggttcgttaagttggatgtgaaagccccgggctcaacctgggaactgcattcaaaactgacgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggatgggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacatcccacacgaattgcttg

SEQ ID NO: 12 - DNA, 1520 bp, Microbacterium sp.
  source 1..1520: mol_type = genomic DNA
tacggagagtttgatcctggctcaggatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagccttcatcgcatggtgggggttggaaagattttttggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggaccattccacggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatacaccagaacgggccagaaatggtcaactctttggacactggtgaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcgtaacaaggtagccgtaccggaaggtgcggctggatcacctccttt

SEQ ID NO: 13 - DNA, 1457 bp, Bacillus mycoides
  source 1..1457: mol_type = genomic DNA
agttagcggcggacgggtgagtaacacgtgggtaacctgcctataagactgggataactccgggaaaccggggctaataccggataacattttgcaccgcatggtgcgaaattgaaaggcggcttcggctgtcacttatagatggacctgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgatgaaggctttcgggtcgtaaagttctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgcaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttccccttcgggggcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagtcgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaaccttttggagccagccgcctaaggtgggacagatgattggggtgaagtcgtaacaaggtagccgtatcggaaggtgcggctggatcacctccttt

SEQ ID NO: 14 - DNA, 1526 bp, Arthrobacter luteolus
  source 1..1526: mol_type = genomic DNA
tacggagagtttgatcctggctcaggatgaacgctggcggcgtgcttaacacatgcaagtcgaacgatgacttctgtgcttgcacagaatgattagtggcgaacgggtgagtaacacgtgagtaacctgcccttaacttcgggataagcctgggaaaccgggtctaataccggatacgacctcctggcgcatgccatggtggtggaaagctttagcggttttggatggactcgcggcctatcagcttgttggttggggtaatggcccaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttatccggaattattgggcgtaaagagctcgtaggcggtttgtcgcgtctgctgtgaaagcccggggctcaaccccgggtctgcagtgggtacgggcagactagagtgcagtaggggagactggaattcctggtgtagcggtgaaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaagacctggaaacaggtcccccacttgtggccggtttacaggtggtgcatggttgtcgtcagctcgtgtcgtgagatgttgggttaagtcccgcaacgagcgcaaccctcgttctatgttgccagcgggttatgccggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattgaggtctgcaactcgacctcatgaagttggagtcgctagtaatcgcagatcagcaacgctgcggtgaatacgttcccgggccttgtacacaccgcccgtcaagtcacgaaagttggtaacacccgaagccggtggcctaaccccttgtgggagggagccgtcgaaggtgggaccggcgattgggactaagtcgtaacaaggtagccgtaccggaaggtgcggctggatcacctccttt

SEQ ID NO: 15 - DNA, 1520 bp, Curtobacterium sp.
  source 1..1520: mol_type = genomic DNA
tacggagagtttgatcctggctcaggacgaacgctggcggcgtgcttaacacatgcaagtcgaacgatgatcaggagcttgctcctgtgattagtggcgaacgggtgagtaacacgtgagtaacctgcccctgactctgggataagcgttggaaacgacgtctaatactggatatgatcactggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcgtggggagcgaacaggattagataccctggtagtccacgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtcgaaggtgggatcggtgattaggactaagtcgtaacaaggtagccgtaccggaaggtgcggctggatcacctccttt

SEQ ID NO: 16 - skipped

SEQ ID NO: 17 - DNA, 898 bp, Rahnella aquatilis
  source 1..898: mol_type = genomic DNA
gtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaattcgccagagatggcttagtgccttcgggaaccgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacgg

SEQ ID NO: 18 - DNA, 1532 bp, Pseudomonas sp.
  source 1..1532: mol_type = genomic DNA
tgaagagtttgatcatggctcagattgaacgctggcggcaggcctaacacatgcaagtcgagcggatgaaaggagcttgctcctggattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaaattaatactttgctgttttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 19 - DNA, 1526 bp, Curtobacterium pusillum
  source 1..1526: mol_type = genomic DNA
tacggagagtttgatcctggctcaggacgaacgctggcggcgtgcttaacacatgcaagtcgaacgatgatgcccagcttgctgggtggattagtggcgaacgggtgagtaacacgtgagtaacctgcccctgactctgggataagcgttggaaacgacgtctaatactggatatgactgccggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagggagcttgctcttgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcatggggagcgaacaggattagataccctggtagtccatgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtcgaaggtgggatcggtgattaggactaagtcgtaacaaggtagccgtaccggaaggtgcggctggatcacctccttt

SEQ ID NO: 20 - DNA, 1543 bp, Stenotrophomonas rhizophila
  source 1..1543: mol_type = genomic DNA
tgaagagtttgatcctggctcagagtgaacgctggcggtaggcctaacacatgcaagtcgaacggcagcacagtaagagcttgctcttatgggtggcgagtggcggacgggtgaggaatacatcggaatctaccttttcgtgggggataacgtagggaaacttacgctaataccgcatacgaccttcgggtgaaagcaggggaccttcgggccttgcgcggatagatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccataccgcgtgggtgaagaaggccttcgggttgtaaagcccttttgttgggaaagaaaagcagtcggctaatacccggttgttctgacggtacccaaagaataagcaccggctaacttcgtgccagcagccgcggtaatacgaagggtgcaagcgttactcggaattactgggcgtaaagcgtgcgtaggtggttgtttaagtctgttgtgaaagccctgggctcaacctgggaattgcagtggatactgggcgactagagtgtggtagagggtagtggaattcccggtgtagcagtgaaatgcgtagagatcgggaggaacatccatggcgaaggcagctacctggaccaacactgacactgaggcacgaaagcgtggggagcaaacaggattagataccctggtagtccacgccctaaacgatgcgaactggatgttgggtgcaatttggcacgcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggtcttgacatgtcgagaactttccagagatggattggtgccttcgggaactcgaacacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacgaccagggctacacacgtactacaatggtagggacagagggctgcaaacccgcgagggcaagccaatcccagaaaccctatctcagtccggattggagtctgcaactcgactccatgaagtcggaatcgctagtaatcgcagatcagcattgctgcggtgaatacgttcccgggccttgtacacaccgcccgtcacaccatgggagtttgttgcaccagaagcaggtagcttaaccttcgggagggcgcttgccacggtgtggccgatgactggggtgaagtcgtaacaaggtagccgtatcggaaggtgcggctggatcacctccttt

SEQ ID NO: 21 - skipped

SEQ ID NO: 22 - DNA, 1539 bp, Rahnella sp.
  source 1..1539: mol_type = genomic DNA
ttgaagagtttgatcatggctcagattgaacgctggcggcaggcctaacacatgcaagtcgagcggtagcacaggagagcttgctctccgggtgacgagcggcggacgggtgagtaatgtctgggaaactgcctgatggagggggataactactggaaacggtagctaataccgcatgacgtcgcaagaccaaagtgggggaccttcgggcctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcgttgcagttaatagctgcagcgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 23 - DNA, 1536 bp, Erwinia billingiae
  source 1..1536: mol_type = genomic DNA
ttgaagagtttgatcatggctcagattgaacgctggcggcaggcctaacacatgcaagtcgaacggtagcacagagagcttgctcttgggtgacgagtggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatacggttaataaccgtgtcgattgacgttacccgcagaagaagcaccggctaactccgtgccagcagccgcggtaatacggagggtgcaagcgttaatcggaattactgggcgtaaagcgcacgcaggcggtctgtcaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccacagaattcggcagagatgccttagtgccttcgggaactgtgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 24 - DNA, 1441 bp, Filobasidium globisporum
  source 1..1441: mol_type = genomic DNA
cggggaattagggttcgattccggagagggagcctgagaaacggctaccacatccaaggaaggcagcaggcgcgcaaattacccaatcccgacacggggaggtagtgacaataaataacaatacagggccctttgggtcttgtaattggaatgagtacaatttaaatcccttaacgaggaacaattggagggcaagtctggtgccagcagccgcggtaattccagctccaatagcgtatattaaagttgttgcagttaaaaagctcgtagttgaacttcaggcttggcggggtggtctgcctcacggtatgtactatccggctgagccttacctcctggtgagcctgcatgtcgtttattcggtgtgtaggggaaccaggaattttactttgaaaaaattagagtgttcaaagcaggcatatgcccgaatacattagcatggaataatagaataggacgtgcggttctattttgttggtttctaggatcgccgtaatgattaatagggacggttgggggcattagtattcagttgctagaggtgaaattcttagatttactgaagactaactactgcgaaagcatttgccaaggacgttttcattaatcaagaacgaaggttaggggatcaaaaacgattagataccgttgtagtcttaacagtaaactatgccgactagggatcgggccacgttcatcttttgactggctcggcaccttacgagaaatcaaagtctttgggttctggggggagtatggtcgcaaggctgaaacttaaaggaattgacggaagggcaccaccaggcgtggagcctgcggcttaatttgactcaacacggggaaactcaccaggtccagacatagtaaggattgacagattgatagctctttcttgattctatgggtggtggtgcatggccgttcttagttggtggagtgatttgtctggttaattccgataacgaacgagaccttaacctgctaaatagtccggccggcttcggctggtcgctgacttcttagagggactaacagcgtttagctgttggaagtttgaggcaataacaggtctgtgatgcccttagatgttctgggccgcacgcgcgctacactgactgagccagcgagtttataaccttggccgaaaggtctgggtaatcttgtgaaactcagtcgtgctggggatagagcattgcaattattgctcttcaacgaggaatgcctagtaagcgtgagtcatcagctcacgttgattacgtccctgccctttgtacacaccgcccgtcgctactaccgattgaatggcttagtgagatctccggattggctttgggaagctggcaacggctacctattgctgaaaagctgatcaaacttggtcatttagaggaagtaaaagtcgtaacaaggtttccgtaggtgaacctgcggaaggatcatt

SEQ ID NO: 25 - skipped

SEQ ID NO: 26 - DNA, 1482 bp, Methylobacterium sp.
  source 1..1482: mol_type = genomic DNA
cttgagagtttgatcctggctcagagcgaacgctggcggcaggcttaacacatgcaagtcgagcgggcatcttcggatgtcagcggcagacgggtgagtaacacgtgggaacgtacccttcggttcggaataacgctgggaaactagcgctaataccggatacgcccttttggggaaaggtttactgccgaaggatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccattcaagtcgggggtgaaagcctgtggctcaaccacagaattgccttcgatactgtttggcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcacctcagtagcgcagctaacgctttaagcattccgcctggggagtacggtcgcaagattaaaactcaaaggaattgacgggggcccgcacaagcggtggagcatgtggtttaattcgaagcaacgcgcagaaccttaccatcccttgacatggcatgttacccggagagattcggggtccacttcggtggcgtgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaaaccgtctcagttcagattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacctccttt

SEQ ID NO: 27 - DNA, 935 bp, Sphingomonas sp.
  source 1..935: mol_type = genomic DNA
cttgagagtttgatcctggctcagaacgaacgctggcggcatgcctaacacatgcaagtcgaacgatgctttcgggcatagtggcgcacgggtgcgtaacgcgtgggaatctgccctcaggttcggaataacagctggaaacggctgctaataccggatgatatcgcaagatcaaagatttatcgcctgaggatgagcccgcgttggattaggtagttggtggggtaaaggcctaccaagccgacgatccatagctggtctgagaggatgatcagccacactgggactgagacacggcccagactcctacgggaggcagcagtggggaatattggacaatgggcgcaagcctgatccagcaatgccgcgtgagtgatgaaggccctagggttgtaaagctcttttacccgggaagataatgactgtaccgggagaataagccccggctaactccgtgccagcagccgcggtaatacggagggggctagcgttgttcggaattactgggcgtaaagcgcacgtaggcggctttgtaagtcagaggtgaaagcctggagctcaactccagaactgcctttgagactgcatcgcttgaatccaggagaggtcagtggaattccgagtgtagaggtgaaattcgtagatattcggaagaacaccagtggcgaaggcggctgactggactggtattgacgctgaggtgcgaaagcgtggggagcaaacaggattagataccctggtagtccacgccgtaaacgatgataactagctgtccgggcacttggtgcttgggtggcgcagctaacgcattaagttatccgcctggggagtacggccgcaaggttaaaactcaaaggaattgacgggggcctgcacaagcggtggagcatgtggtttaattcgaagcaacgcgcagaaccttaccagcgtttgac

SEQ ID NO: 28 - DNA, 931 bp, Aureobasidium pullulans
  source 1..931: mol_type = genomic DNA
atagtcgggggcatcagtattcaattgtcagaggtgaaattcttggatttattgaagactaactactgcgaaagcatttgccaaggatgttttcattaatcagtgaacgaaagttaggggatcgaagacgatcagataccgtcgtagtcttaaccataaactatgccgactagggatcgggcgatgttatcattttgactcgctcggcaccttacgagaaatcaaagtctttgggttctggggggagtatggtcgcaaggctgaaacttaaagaaattgacggaagggcaccaccaggcgtggagcctgcggcttaatttgactcaacacggggaaactcaccaggtccagacacaataaggattgacagattgagagctctttcttgattttgtgggtggtggtgcatggccgttcttagttggtggagtgatttgtctgcttaattgcgataacgaacgagaccttaacctgctaaatagcccggcccgctttggcgggtcgccggcttcttagagggactatcggctcaagccgatggaagtttgaggcaataacaggtctgtgatgcccttagatgttctgggccgcacgcgcgctacactgacagagccaacgagttcatttccttgcccggaagggttgggtaatcttgttaaactctgtcgtgctggggatagagcattgcaattattgctcttcaacgaggaatgcctagtaagcgtacgtcatcagcgtgcgttgattacgtccctgccctttgtacacaccgcccgtcgctactaccgattgaatggctgagtgaggccttcggactggcccagggaggtcggcaacgaccacccagggccggaaagttggtcaaactccgtcatttagaggaagtaaaagtcgtaacaaggtttccgtaggtgaacctgcggaaggatca

SEQ ID NO: 29 - DNA, 1002 bp, Pseudoclavibacter helvolus
  source 1..1002: mol_type = genomic DNA
tacggagagtttgatcctggctcaggacgaacgctggcggcgtgcttaacacatgcaagtcgaacgatgaagcccagcttgctgggttgattagtggcgaacgggtgagtaacacgtgagcaacgtgcccataactctgggataacctccggaaacggtggctaatactggatatctaacacgatcgcatggtctgtgtttggaaagattttttggttatggatcggctcacggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggcattcgggttgtaaacctcttttagtagggaagaagcgaaagtgacggtacctgcagaaaaagcaccggctaactacgtgccagcagccgctgtaatacgtagggtgcaagcgttgtccggaattattgggcgtaaagagctcgtaggcggtttgtcgcgtctgctgtgaaatcccgaggctcaacctcgggtctgcagtgggtacgggcagactagagtgtggtaggggagattggaattcctggtgtagcggtggaatgcgcagatatcaggaggaacaccgatggcgaaggcagatctctgggccattactgacgctgaggagcgaaagcatggggagcgaacaggattagataccctggtagtccatgccgtaaacgttgggcgctagatgtggggaccattccacggtttccgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatataccggaaacgttcagaaatgttcgcc

SEQ ID NO: 30 - DNA, 1520 bp, Microbacterium testaceum
  source 1..1520: mol_type = genomic DNA
tacggagagtttgatcctggctcaggatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagacgtgatcgcatggtcgtgtttggaaagatttttcggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggaccattccacggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcgtaacaaggtagccgtaccggaaggtgcggctggatcacctccttt

SEQ ID NO: 31 - DNA, 941 bp, Sporisorium reilianum
  source 1..941: mol_type = genomic DNA
cagccgggggcattagtatttgcacgctagaggtgaaattcttggattgtgcaaagacttcctactgcgaaagcatttgccaagaatgttttcattaatcaagaacgaaggttagggtatcgaaaacgattagataccgttgtagtcttaacagtaaactatgccgactccgaatcggtcgatgctcatttcactggctcgatcggcgcggtacgagaaatcaaagtttttgggttctggggggagtatggtcgcaaggctgaaacttaaagaaattgacggaagggcaccaccaggagtggagcctgcggcttaatttgactcaacacgggaaaactcaccgggtccggacatagtaaggattgacagattgatggcgctttcatgattctatgggtggtggtgcatggccgttcttagttggtggagtgatttgtctggttaattccgataacgaacgagaccttgacctgctaaatagacgggttgacattttgttggccccttatgtcttcttagagggacaatcgaccgtctaggtgatggaggcaaaaggcaataacaggtctgtgatgcccttagatgttccgggctgcacgcgcgctacactgacagagacaacgagtggggccccttgtccgaaatgactgggtaaacttgtgaaactttgtcgtgctggggatggagctttgtaatttttgctcttcaacgaggaattcctagtaagcgcaagtcatcagcttgcgttgactacgtccctgccctttgtacacaccgcccgtcgctactaccgattgaatggcttagtgaggacttgggagagtacatcggggagccagcaatggcaccctgacggctcaaactcttacaaacttggtcatttagaggaagtaaaagtcgtaacaaggtatctgtaggtgaacctgcagatggatcatttc

SEQ ID NO: 32 - DNA, 1072 bp, Hafnia paralvei
  source 1..1072: mol_type = genomic DNA
actgagcattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 33 - DNA, 853 bp, Erwinia persicinus
  source 1..853: mol_type = genomic DNA
ggaggaaggcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccacggaattcggcagagatgccttagtgccttcgggaaccgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 34 - DNA, 1519 bp, Plantibacter flavus
  source 1..1519: mol_type = genomic DNA
tacggagagtttgatcctggctcaggacgaacgctggcggcgtgcttaacacatgcaagtcgaacgatgaagcccagcttgctgggtggattagtggcgaacgggtgagtaacacgtgagtaacctgcccttgactctgggataagcgttggaaacgacgtctaataccggatacgagcttccaccgcatggtgagttgctggaaagaattttggtcaaggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggacgacggccttcgggttgtaaacctcttttagcagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggtctgcagtgggtacgggcagactagagtgcggtaggggagattggaattcctggtgtagcggtggaatgcgcagatatcaggaggaacaccgatggcgaaggcagatctctgggccgctactgacgctgaggagcgaaagggtggggagcaaacaggcttagataccctggtagtccaccccgtaaacgttgggcgctagatgtggggaccattccacggtttccgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggacgacgtcaaatcatcatgccccttatgtcttgggcttcacgcatgctacaatggccagtacaaagggctgcaataccgtaaggtggagcgaatcccaaaaagctggtcccagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccagtggcctaaccgcaaggatggagctgtctaaggtgggatcggtaattaggactaagtcgtaacaaggtagccgtaccggaaggtgcggctggatcacctccttt

SEQ ID NO: 35 - DNA, 1536 bp, Pantoea ananatis
  source 1..1536: mol_type = genomic DNA
ttgaagagtttgatcatggctcagattgaacgctggcggcaggcctaacacatgcaagtcggacggtagcacagagagcttgctcttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatgaggttaataaccgcgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagcgaacttagcagagatgctttggtgccttcgggaacgctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcattactggggtgaagtcgtaacaaggtaaccgtaggggaacctgcggttggatcacctcctt

SEQ ID NO: 36 - DNA, 993 bp, Pantoea vagans
  source 1..993: mol_type = genomic DNA
ttgaagagtttgatcatggctcagattgaacgctggcggcaggcctaacacatgcaagtcggacggtagcacagagagcttgctcttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatgcggttaataaccgcgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatc

SEQ ID NO: 37 - DNA, 1532 bp, Pseudomonas rhodesiae
  source 1..1532: mol_type = genomic DNA
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacgaacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggccattacctaatacgtgatggttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggacggttaccacggtgtgattcatgactggggtgaagtcgtaacaaggtagccgtaggggaacctgcggctggatcacctcctt

SEQ ID NO: 38 - DNA, 1517 bp, Rhodococcus sp.
  source 1..1517: mol_type = genomic DNA
tacggagagtttgatcctggctcaggacgaacgctggcggcgtgcttaacacatgcaagtcgagcggtaaggcctttcggggtacacgagcggcgaacgggtgagtaacacgtgggtgatctgccctgcactctgggataagcttgggaaactgggtctaataccggatatgaccacagcatgcatgtgttgtggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcggaagcctgatgcagcgacgccgcgtgagggatgaaggccttcgggttgtaaacctctttcagcagggacgaagcgtgagtgacggtacctgcagaagaagcaccggctaactacgtgccagcagccgcggtaatacgtagggtgcgagcgttgtccggaattactgggcgtaaagagttcgtaggcggtttgtcgcgtcgtttgtgaaaacccggggctcaacttcgggcttgcaggcgatacgggcagacttgagtgtttcaggggagactggaattcctggtgtagcggtgaaatgcgcagatatcaggaggaacaccggtggcgaaggcgggtctctgggaaacaactgacgctgaggaacgaaagcgtgggtagcaaacaggattagataccctggtagtccacgccgtaaacggtgggcgctaggtgtgggttccttccacgggatctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatacaccggaaaaccgtagagatacggtcccccttgtggtcggtgtacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttacggggagggagccgtcgaaggtgggatcggcgattgggacgaagtcgtaacaaggtagccgtaccggaaggtgcggctggatcacctccttt

SEQ ID NO: 39 - DNA, 1482 bp, Agrobacterium tumefaciens
  source 1..1482: mol_type = genomic DNA
cttgagagtttgatcctggctcagaacgaacgctggcggcaggcttaacacatgcaagtcgaacgccccgcaaggggagtggcagacgggtgagtaacgcgtgggaatctaccgtgccctgcggaatagctccgggaaactggaattaataccgcatacgccctacgggggaaagatttatcggggtatgatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccttagggttgtaaagctctttcaccggagaagataatgacggtatccggagaagaagccccggctaacttcgtgccagcagccgcggtaatacgaagggggctagcgttgttcggaattactgggcgtaaagcgcacgtaggcggatatttaagtcaggggtgaaatcccagagctcaactctggaactgcctttgatactgggtatcttgagtatggaagaggtaagtggaattccgagtgtagaggtgaaattcgtagatattcggaggaacaccagtggcgaaggcggcttactggtccattactgacgctgaggtgcgaaagcgtggggagcaaacaggattagataccctggtagtccacgccgtaaacgatgaatgttagccgtcgggcagtatactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagctcttgacattcggggtttgggcagtggagacattgtccttcagttaggctggccccagaacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgagacagcgatgtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttttacccgaaggtagtgcgctaaccgcaaggaggcagctaaccacggtagggtcagcgactggggtgaagtcgtaacaaggtagccgtaggggaacctgcggctggatcacctccttt

SEQ ID NO: 40 - DNA, 903 bp, Pantoea sp.
  source 1..903: mol_type = genomic DNA
ttgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcgcgtgatggcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgt

SEQ ID NO: 41 - DNA, 1457 bp, Corynebacterium mucifaciens
  source 1..1457: mol_type = genomic DNA
gtggagagtttgatcctggctcaggatgaacgctggcggcgtgcttaacacatgcaagtcgaacggaaaggcccaagcttgcttgggtactcgagtggcgaacgggtgagtaacacgtgggtgatctgccctgcacttcgggataagcctgggaaactgggtctaataccggataggacgatggtttggatgccattgtggaaagttttttcggtgtgggatgagctcgcggcctatcagcttgttggtggggtaatggcctaccaaggcgtcgacgggtagccggcctgagagggtgtacggccacattgggactgagatacggcccagactcctacgggaggcagcagtggggaatattgcacaatgggcgcaagcctgatgcagcgacgccgcgtgggggatgacggccttcgggttgtaaactcctttcgctagggacgaagcgttttgtgacggtacctggagaagaagcaccggctaactacgtgccagcagccgcggtaatacgtagggtgcgagcgttgtccggaattactgggcgtaaagagctcgtaggtggtttgtcgcgtcgtttgtgtaagcccgcagcttaactgcgggactgcaggcgatacgggcataacttgagtgctgtaggggagactggaattcctggtgtagcggtggaatgcgcagatatcaggaggaacaccgatggcgaaggcaggtctctgggcagtaactgacgctgaggagcgaaagcatgggtagcgaacaggattagataccctggtagtccatgccgtaaacggtgggcgctaggtgtgagtcccttccacggggttcgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatacaccagatcgccgtagagatacggtttccctttgtggttggtgtacaggtggtgcatggttgtcgtcagctcgtgtcgtgagatgttgggttaagtcccgcaacgagcgcaacccttgtcttatgttgccagcacgtgatggtggggactcgtgagagactgccggggttaactcggaggaaggtggggatgacgtcaaatcatcatgccccttatgtccagggcttcacacatgctacaatggtcggtacaacgcgcatgcgagcctgtgagggtgagcgaatcgctgtgaaagccggtcgtagttcggattggggtctgcaactcgaccccatgaagtcggagtcgctagtaatcgcagatcagcaacgctgcggtgaatacgttcccgggccttgtacacaccgcccgtcacaccatgggagtgggttgcaaaagaagtaggtagcttaaccttcgggagggcgcttaccactttgtgat

SEQ ID NO: 42 - DNA, 1532 bp, Pseudomonas lundensis
  source 1..1532: mol_type = genomic DNA
tgaagagtttgatcatggctcagattgaacgctggcggcaggcctaacacatgcaagtcgagcggtagagaggtgcttgcacctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggaaccttgagttcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccctcgggaggacggttaccacggtgtgattcatgactggggtgaagtcgtaacaaggtagccgtaggggaacctgcggctggatcacctcctt

SEQ ID NO: 43 - DNA, 1525 bp, Janthinobacterium sp.
  source 1..1525: mol_type = genomic DNA
ctgagtttgatcctggctcagattgaacgctggcggcatgccttacacatgcaagtcgaacggcagcacggagcttgctctggtggcgagtggcgaacgggtgagtaatatatcggaacgtaccctggagtgggggataacgtagcgaaagttacgctaataccgcatacgatctaaggatgaaagtgggggatcgcaagacctcatgctcgtggagcggccgatatctgattagctagttggtagggtaaaagcctaccaaggcatcgatcagtagctggtctgagaggacgaccagccacactggaactgagacacggtccagactcctacgggaggcagcagtggggaattttggacaatgggcgaaagcctgatccagcaatgccgcgtgagtgaagaaggccttcgggttgtaaagctcttttgtcagggaagaaacggtgagagctaatatctcttgctaatgacggtacctgaagaataagcaccggctaactacgtgccagcagccgcggtaatacgtagggtgcaagcgttaatcggaattactgggcgtaaagcgtgcgcaggcggttttgtaagtctgatgtgaaatccccgggctcaacctgggaattgcattggagactgcaaggctagaatctggcagaggggggtagaattccacgtgtagcagtgaaatgcgtagatatgtggaggaacaccgatggcgaaggcagccccctgggtcaagattgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatggctggaatccttgagagatcagggagtgctcgaaagagaaccagtacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagcgccgccaacccgcgagggggagctaatcgcagaaagtgtatcgtagtccggattgtagtctgcaactcgactgcatgaagttggaatcgctagtaatcgcggatcagcatgtcgcggtgaatacgttcccgggtcttgtacacaccgcccgtcacaccatgggagcgggttttaccagaagtaggtagcttaaccgtaaggagggcgcttaccacggtaggattcgtgactggggtgaagtcgtaacaaggtagccgtatcggaaggtgcggctggatcacctccttt

SEQ ID NO: 44 - DNA, 1497 bp, Herbaspirillum sp.
  source 1..1497: mol_type = genomic DNA
tggcggcatgccttacacatgcaagtcgaacggcagcataggagcttgctcctgatggcgagtggcgaacgggtgagtaatatatcggaacgtgccctagagtgggggataactagtcgaaagactagctaataccgcatacgatctacggatgaaagtgggggatcgcaagacctcatgctcctggagcggccgatatctgattagctagttggtggggtaaaagctcaccaaggcgacgatcagtagctggtctgagaggacgaccagccacactgggactgagacacggcccagactcctacgggaggcagcagtggggaattttggacaatgggggcaaccctgatccagcaatgccgcgtgagtgaagaaggccttcgggttgtaaagctcttttgtcagggaagaaacggttctggataatacctaggactaatgacggtacctgaagaataagcaccggctaactacgtgccagcagccgcggtaatacgtagggtgcaagcgttaatcggaattactgggcgtaaagcgtgcgcaggcggttgtgtaagtcagatgtgaaatccccgggctcaacctgggaattgcatttgagactgcacggctagagtgtgtcagaggggggtagaattccacgtgtagcagtgaaatgcgtagatatgtggaggaataccgatggcgaaggcagccccctgggataacactgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatggatggaatcccgaagagatttgggagtgctcgaaagagaaccatcacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggattggagtctgcaactcgactccatgaagttggaatcgctagtaatcgcggatcagcatgtcgcggtgaatacgttcccgggtcttgtacacaccgcccgtcacaccatgggagcgggttttaccagaagtgggtagcctaaccgcaaggagggcgctcaccacggtaggattcgtgactggggtgaagtcgtaacaaggtagccgtatcggaaggtgcggctggatcacctccttt

SEQ ID NO: 45 - DNA, 1522 bp, Sanguibacter keddieii
  source 1..1522: mol_type = genomic DNA
tacggagagtttgatcctggctcaggacgaacgctggcggcgtgcttaacacatgcaagtcgaacggtgacgctagagcttgctctggttgatcagtggcgaacgggtgagtaacacgtgagtaacctgcccttgactctgggataactccgggaaaccggggctaataccggatacgagacgcgaccgcatggtcggcgtctggaaagtttttcggtcaaggatggactcgcggcctatcagcttgttggtgaggtaatggctcaccaaggcgtcgacgggtagccggcctgagagggcgaccggccacactgggactgagacacggcccagactcctacgggaggcagcagtggggaatattgcacaatgggcgaaagcctgatgcagcgacgccgcgtgagggatgaaggccttcgggttgtaaacctctttcagtagggaagaagcgaaagtgacggtacctgcagaagaagcgccggctaactacgtgccagcagccgcggtaatacgtagggcgcaagcgttgtccggaattattgggcgtaaagagctcgtaggcggtttgtcgcgtctggtgtgaaaactcaaggctcaaccttgagcttgcatcgggtacgggcagactagagtgtggtaggggtgactggaattcctggtgtagcggtggaatgcgcagatatcaggaggaacaccgatggcgaaggcaggtcactgggccactactgacgctgaggagcgaaagcatggggagcgaacaggattagataccctggtagtccatgccgtaaacgttgggcactaggtgtggggctcattccacgagttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatacaccggaatcatgcagagatgtgtgcgtcttcggactggtgtacaggtggtgcatggttgtcgtcagctcgtgtcgtgagatgttgggttaagtcccgcaacgagcgcaaccctcgtcctatgttgccagcacgttatggtggggactcataggagactgccggggtcaactcggaggaaggtggggatgacgtcaaatcatcatgccccttatgtcttgggcttcacgcatgctacaatggccggtacaaagggctgcgataccgcgaggtggagcgaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaaccccttgtgggatggagccgtcgaaggtgggattggcgattgggactaagtcgtaacaaggtagccgtaccggaaggtgcggctggatcacctccttt

SEQ ID NO: 46 - DNA, 831 bp, Pantoea agglomerans
  source 1..831: mol_type = genomic DNA
ttgaagagtttgatcatggctcagattgaacgctggcggcaggcctaacacatgcaagtcggacggtagcacagaggagcttgctccttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgacagggttaataaccctgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggctttagtcttgtagagtggggtagaattccaggtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgtt

SEQ ID NO: 47 - DNA, 960 bp, Cronobacter dublinensis
  source 1..960: mol_type = genomic DNA
agggtgcaagcgttaatcggaattactgggcgtaaagcgcgcgtaggtggtttgttaagttgaatgtgaaatccccgggctcaacctgggaactgcatttgaaactggcaagctagagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgcaacgagcgcaacccttgtcctgtgttgccagcgcgtaatggcggggactcgcaggagactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagt

SEQ ID NO: 48 - DNA, 1548 bp, Bacillus paralicheniformis
  source 1..1548: mol_type = genomic DNA
catggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgcttgattgaaccgcatggttcaattataaaaggtggcttttagctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacctccttt

SEQ ID NO: 49 - DNA, 1551 bp, Bacillus gibsonii
  source 1..1551: mol_type = genomic DNA
tatggagagtttgatcctggctcaggacgaacgctggcggcgtgcctaatacatgcaagtcgagcggacgtttttgaagcttgcttcaaaaacgttagcggcggacgggtgagtaacacgtgggcaacctgccttatcgactgggataactccgggaaaccggggctaataccggataatatctagcacctcctggtgcaagattaaaagagggccttcgggctctcacggtgagatgggcccgcggcgcattagctagttggagaggtaatggctccccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaagggtttcggctcgtaaagctctgttatgagggaagaacacgtaccgttcgaatagggcggtaccttgacggtacctcatcagaaagccacggctaactacgtgccagcagccgcggtaatacgtaggtggcaagcgttgtccggaattattgggcgtaaagcgcgcgcaggcggccttttaagtctgatgtgaaatcttgcggctcaaccgcaagcggtcattggaaactgggaggcttgagtacagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacgggggcccgcacaagcagtggagcatgtggtttaattcgaagcaacgcgaagaaccttaccaggtcttgacatcctttgaccactctggagacagagcttccccttcgggggcaaagtgacaggtggtgcatggttgtcgtcagctcgtgtcgtgagatgttgggttaagtcccgcaacgagcgcaacccttgaccttagttgccagcatttagttgggcactctaaggtgactgccggtgacaaaccggaggaaggtggggatgacgtcaaatcatcatgccccttatgacctgggctacacacgtgctacaatggatggtacaaagggttgcgaagccgcgaggtgaagccaatcccataaagccattctcagttcggattgtaggctgcaactcgcctgcatgaagctggaattgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacctccttt

SEQ ID NO: 50 - DNA, 1536 bp, Enterobacter sp.
  source 1..1536: mol_type = genomic DNA
ttgaagagtttgatcatggctcagattgaacgctggcggcaggcctaacacatgcaagtcgaacggtagcacagagagcttgctcttgggtgacgagtggcggacgggtgagtaatgtctgggaaactgcccgatggagggggataactactggaaacggtagctaataccgcataacgtcgcaagaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattgtggttaataaccgcagtgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttagcagagatgctttagtgccttcgggaaccgtgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 51 - DNA, 1537 bp, Klebsiella aerogenes
  source 1..1537: mol_type = genomic DNA
ttgaagagtttgatcatggctcagattgaacgctggcggcaggcctaacacatgcaagtcgagcggtagcacagggagcttgctcctgggtgacgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttggtgattgacgttactcgcagaagaagcaccggctaactccgtgccagcagccgcggtaatacggggggtgcaagcgttaatcggaattactgggcgtaaagcgcacgcaggcggtttgtcaagtcggatgtgaaatccccgggctcaacctgggaactgcattcgaaacgggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccagtgacaaactggaggaaggtggggatgacgtcaagtcatcatggcccttacgagtagggctacacacgtgctacaatggcatatacaaagagaagcgacctcgcgagagcaagcggacctcacaaagtatgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 52 - DNA, 1517 bp, Arthrobacter sp.
  source 1..1517: mol_type = genomic DNA
acggagagtttgatcctggctcaggatgaacgctggcggcgtgcttaacacatgcaagtcgaacgatgatcccagcttgctgggggattagtggcgaacgggtgagtaacacgtgagtaacctgcccttgactctgggataagcctgggaaactgggtctaataccggatatgactgtctgacgcatgtcaggtggtggaaagcttttgtggttttggatggactcgcggcctatcagcttgttggtggggtaatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcgccggctaactacgtgccagcagccgcggtaatacgtagggcgcaagcgttatccggaattattgggcgtaaagagctcgtaggcggtttgtcgcgtctgctgtgaaagaccggggctcaactccggttctgcagtgggtacgggcagactagagtgcagtaggggagactggaattcctggtgtagcggtgaaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaatacctggaaacaggtgccccgcttgcggtcggtttacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccggcgattgggactaagtcgtaacaaggtagccgtaccggaaggtgcggctggatcacctccttt

SEQ ID NO: 53 - DNA, 1406 bp, Pseudomonas fragi
  source 1..1406: mol_type = genomic DNA
tgaagagtttgatcatggctcagattgaacgctggcggcaggcctaacacatgcaagtcgagcggtagagagaagcttgcttctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatg

SEQ ID NO: 54 - DNA, 1136 bp, Methylobacterium adhaesivum
  source 1..1136: mol_type = genomic DNA
cttgagagtttgatcctggctcagagcgaacgctggcggcaggcttaacacatgcaagtcgagcgggcaccttcgggtgtcagcggcagacgggtgagtaacacgtgggaacgtacccttcggttcggaataacgctgggaaactagcgctaataccggatacgcccttttggggaaaggtttactgccgaaggatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccattcaagtcgggggtgaaagcctgtggctcaaccacagaattgccttcgatactgtttggcttgagtttggtagaggttggtggaactgcgagtgtagaggtgaaattcgtagatattcgcaagaacaccagtggcgaaggcggccaactggaccaatactgacgctgaggcgcgaaagcgtggggagcaaacaggattagataccctggtagtccacgccgtaaacgatgaatgctagctgttggggtgcttgcacctcagtagcgcagctaacgctttaagcattccgcctggggagtacggtcgcaagattaaaactcaaaggaattgacgggggcccgcacaagcggtggagcatgtggtttaattcgaagcaacgcgcagaaccttaccatcccttgacatgtcgtgccatccggagagatccggggttcccttcggggacgcgaacacaggtgctgcatggctgtcgtcagctcgtgtcgtgagatgttgggttaagtcccgcaacgagcgcaacccacgtccttagttgccatcatttagttgggcactctagggagactgccggtgataagccgcgaggaaggtgtggatgacgtc

SEQ ID NO: 55 - DNA, 1374 bp, Bacillus megaterium
  source 1..1374: mol_type = genomic DNA
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgct

SEQ ID NO: 56 - DNA, 1554 bp, Paenibacillus lautus
  source 1..1554: mol_type = genomic DNA
attggagagtttgatcctggctcaggacgaacgctggcggcgtgcctaatacatgcaagtcgagcggacctgatggagtgcttgcactcctgatggttagcggcggacgggtgagtaacacgtaggcaacctgccctcaagactgggataactaccggaaacggtagctaataccggataatttatttcacagcattgtggaataatgaaagacggagcaatctgtcacttggggatgggcctgcggcgcattagctagttggtggggtaacggctcaccaaggcgacgatgcgtagccgacctgagagggtgaacggccacactgggactgagacacggcccagactcctacgggaggcagcagtagggaatcttccgcaatgggcgaaagcctgacggagcaacgccgcgtgagtgatgaaggttttcggatcgtaaagctctgttgccaaggaagaacgtcttctagagtaactgctaggagagtgacggtacttgagaagaaagccccggctaactacgtgccagcagccgcggtaatacgtagggggcaagcgttgtccggaattattgggcgtaaagcgcgcgcaggcggttctttaagtctggtgtttaaacccgaggctcaacttcgggtcgcactggaaactggggaacttgagtgcagaagaggagagtggaattccacgtgtagcggtgaaatgcgtagatatgtggaggaacaccagtggcgaaggcgactctctgggctgtaactgacgctgaggcgcgaaagcgtggggagcaaacaggattagataccctggtagtccacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaagtcttgacatccctctgaatcctctagagatagaggcggccttcgggacagaggtgacaggtggtgcatggttgtcgtcagctcgtgtcgtgagatgttgggttaagtcccgcaacgagcgcaacccttgattttagttgccagcacatcatggtgggcactctagaatgactgccggtgacaaaccggaggaaggcggggatgacgtcaaatcatcatgccccttatgacttgggctacacacgtactacaatggctggtacaacgggaagcgaagccgcgaggtggagccaatcctataaaagccagtctcagttcggattgcaggctgcaactcgcctgcatgaagtcggaattgctagtaatcgcggatcagcatgccgcggtgaatacgttcccgggtcttgtacacaccgcccgtcacaccacgagagtttacaacacccgaagtcggtggggtaacccgcaagggagccagccgccgaaggtggggtagatgattggggtgaagtcgtaacaaggtagccgtatcggaaggtgcggctggatcacctccttt

SEQ ID NO: 57 - DNA, 1550 bp, Bacillus mycoides
  source 1..1550: mol_type = genomic DNA
attggagagtttgatcctggctcaggatgaacgctggcggcgtgcctaatacatgcaagtcgagcgaatggattaagagcttgctcttatgaagttagcggcggacgggtgagtaacacgtgggtaacctgcccataagactgggataactccgggaaaccggggctaataccggataacattttgcaccgcatggtgcgaaattcaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaacctttttggagccagccgcctaaggtgggacagatgattggggtgaagtcgtaacaaggtagccgtatcggaaggtgcggctggatcacctccttt

SEQ ID NO: 58 - DNA, 1060 bp, Janthinobacterium svalbardensis
  source 1..1060: mol_type = genomic DNA
aatgacggtacctgaagaataagcaccggctaactacgtgccagcagccgcggtaatacgtagggtgcaagcgttaatcggaattactgggcgtaaagcgtgcgcaggcggttttgtaagtctgatgtgaaatccccgggctcaacctgggaattgcattggagactgcaaggctagaatctggcagaggggggtagaattccacgtgtagcagtgaaatgcgtagatatgtggaggaacaccgatggcgaaggcagccccctgggtcaagattgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatggctggaatcctcgagagattggggagtgctcgaaagagaaccagtacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagcgccgccaacccgcgagggggagctaatcgcagaaagtgtatcgtagtccggattgtagtctgcaactcgactgcatgaagttggaatcgctagtaatcgcggatcagcatgtcgcggtgaatacgttcccgggtcttgtacacaccgcccgtcacaccatgggagcgggttttaccagaagtaggtagcttaaccgtaaggagggcgcttaccacggtaggattcgtgactggggtgaagtcgtaacaaggtagccgtatcggaaggtgcggctggatcacctccttt

SEQ ID NO: 59 - DNA, 1538 bp, Kosakonia cowanii
  source 1..1538: mol_type = genomic DNA
ttgaagagtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatgcggttaataaccgcgtcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaacttggcagagatgccttggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcgcatacaaagagaagcgatctcgcgagagccagcggacctcataaagtgcgtcgtagtccggattggagtctgcaactcgactccatgaagtcggaatcgctagtaatcgtgaatcagaatgtcacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 60 - DNA, 1547 bp, Bacillus simplex
  source 1..1547: mol_type = genomic DNA
tcggagagtttgatcctggctcaggacgaacgctggcggcgtgcctaatacatgcaagtcgagcgaatcgatgggagcttgctccctgagattagcggcggacgggtgagtaacacgtgggcaacctgcctataagactgggataacttcgggaaaccggagctaataccggatacgttcttttctcgcatgagagaagatggaaagacggttttgctgtcacttatagatgggcccgcggcgcattagctagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgaagaaggccttcgggtcgtaaagttctgttgttagggaagaacaagtaccagagtaactgctggtaccttgacggtacctaaccagaaagccacggctaactacgtgccagcagccgcggtaatacgtaggtggcaagcgttgtccggaattattgggcgtaaagcgcgcgcaggtggttccttaagtctgatgtgaaagcccacggctcaaccgtggagggtcattggaaactggggaacttgagtgcagaagaggaaagtggaattccaagtgtagcggtgaaatgcgtagagatttggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaaacctgcgaaggtaagcgaatcccataaagccattctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtgaggtaacctttatggagccagccgcctaaggtgggacagatgattggggtgaagtcgtaacaaggtagccgtatcggaaggtgcggctggatcacctccttt

SEQ ID NO: 61 - DNA, 962 bp, Lelliottia sp.
  source 1..962: mol_type = genomic DNA
ggaaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttagcagagatgctttagtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 62 - DNA, 876 bp, Erwinia sp.
  source 1..876: mol_type = genomic DNA
tggctcagattgaacgctggcggcaggcctaacacatgcaagtcgaacggtagcacagaggagcttgctccttgggtgacgagtggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagtggggaggaaggcgttaaggttaataaccttggcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacgg

SEQ ID NO: 63 - DNA, 1532 bp, Pseudomonas azotoformans
  source 1..1532: mol_type = genomic DNA
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgttcttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 64 - DNA, 1024 bp, Hanseniaspora uvarum
  source 1..1024: mol_type = genomic DNA
gctcgagttcttgtttagatcttttacaataatgtgtatctttactgaagatgtgcgcttaattgcgctgcttctttagagtgtcgcagtgaaagtagtcttgcttgaatctcagtcaacgctacacacattggagtttttttactttaatttaattctttctgctttgaatcgaaaggttcaaggcaaaaaacaaacacaaacaattttattttattataattttttaaactaaaccaaaattcctaacggaaattttaaaataatttaaaactttcaacaacggatctcttggttctcgcatcgatgaagaacgtagcgaattgcgataagtaatgtgaattgcagatactcgtgaatcattgaatttttgaacgcacattgcgcccttgagcattctcaggggcatgcctgtttgagcgtcatttccttctcaaaagataatttattattttttggttgtgggcgatactcagggttagcttgaaattggagactgtttcagtcttttttaattcaacacttagcttctttggagacgctgttctcgctgtgatgtatttatggatttattcgttttactttacaagggaaatggtaacgtaccttaggcaaagggttgcttttaatattcatcaagtttgacctcaaatcaggtaggattacccgctgaacttaagcatatcaataagcggaggaaaagaaaccaactgggattaccttagtaacggcgagtgaagcggtaaaagctcaaatttgaaatctggtactttcagtgcccgagttgtaatttgtagaatttgtctttgattaggtccttgtctatgttccttggnancaggacgtcatagagggtgagaatcccgtttggcgaggataccttttctctgtaagactttttcgaanantcgagttgtttgggaatgcagctcaaagtgggtggtaaanttccatctaaagctaaatnttggcgagagaccgatagcgaacnagtacagtgatggaaagatgaaaaagaantttn

SEQ ID NO: 65 - DNA, 1550 bp, Bacillus sp.
  source 1..1550: mol_type = genomic DNA
attggagagtttgatcctggctcaggatgaacgctggcggcgtgcctaatacatgcaagtcgagcgaatggattaagagcttgctcttatgaagttagcggcggacgggtgagtaacacgtgggtaacctgcccataagactgggataactccgggaaaccggggctaataccggataacattttgaactgcatggttcgaaattgaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaacctttttggagccagccgcctaaggtgggacagatgattggggtgaagtcgtaacaaggtagccgtatcggaaggtgcggctggatcacctccttt

SEQ ID NO: 66 - DNA, 1034 bp, Hanseniaspora occidentalis
  source 1..1034: mol_type = genomic DNA
gattttttggggttgcttcgaacttgcagacagagtgtcgagacttgtgagcctgcgcttaattgcgcggcctagagtcgagtgcttgttattggctgcgagggacgagtgccttttgaaaaaatccattacacactgtgaagattttttttcatacattttacttctttggggctttcgagctccaaaggctataaacacaaaccaaacttttttttttattatttgttaatcaagaaattttcttattgaaattaaatattttaaaactttcaacaacggatctcttggttctcgcatcgatgaagaacgtagcgaattgcgataagtaatgtgaattgcagattctcgtgaatcattgaatttttgaacgcacattgcgccctctggtattccagggggcatgcctgtttgagcgtcatttccttctcaaaatctcgattttggttgtgagtgatactctgttacagggttaacttgaaagtgctattgccctagctactcttttttttacttgctaagaaaaagatttttggataatttcaatgtatttaggtatttataccgactttcattggatgctgagagtcttgtctaagcgcttttgtgagattgagcagaagggattaacagtattcataaagtttgacctcaaatcaggtaggattacccgctgaacttaagcatatcaataagcggaggaaaagaaaccaaccgggattgcctcagtaacggcgagtgaagcggcaaaagctcaaatttgaaatctggcactttcagtgtccgagttgtaatttgtagaagtagttttgggactggtccttatctatgtttcttggaacaggacgtcatagagggtgagancccgtatgatgaggcccccagtcctttgtaaaacgctncgaagagtcgagttgtttgggaatgcagctctaagtgggtngnaattnntctaaagctaaatnnnnnnnanacnntngcganagtacngtgatgnngatgannactttgaaananantgaaaagtacgtgaa

SEQ ID NO: 67-136 - skipped (70 entries)

SEQ ID NO: 137 - DNA, 1547 bp, Bacillus sp.
  source 1..1547: mol_type = genomic DNA
ttcggagagtttgatcctggctcaggacgaacgctggcggcgtgcctaatacatgcaagtcgagcggacagaagggagcttgctcccggatgttagcggcggacgggtgagtaacacgtgggtaacctgcctgtaagactgggataactccgggaaaccggagctaataccggatagttccttgaaccgcatggttcaaggatgaaagacggtttcggctgtcacttacagatggacccgcggcgcattagctagttggtggggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgcgagagtaactgctcgcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggacagaacaaagggctgcgagaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcgtaacaaggtagccgtatcggaaggtgcggctggatcacctccttt

SEQ ID NO: 138 - DNA, 841 bp, Bacillus clausii
  source 1..841: mol_type = genomic DNA
aacggagagtttgatcctggctcaggacgaacgctggcggcgtgcctaatacatgcaagtcgagcggacagaagggagcttgctcccggacgttagcggcggacgggtgagtaacacgtgggcaacctgccccttagactgggataactccgggaaaccggagctaataccggataatccctttctccacctggagagagggtgaaagatggcttcggctatcactaagggatgggcccgcggcgcattagctagttggtaaggtaacggcttaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgaggaaggccttcgggtcgtaaagctctgttgtgagggaagaagcggtgccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttag

SEQ ID NO: 139 - DNA, 1105 bp, Bacillus coagulans
  source 1..1105: mol_type = genomic DNA
gcctaatacatgcaagtcgtgcggaccttttaaaagcttgcttttaaaaggttagcggcgaacgggtgagtaacacgtgggcaacctgcctgtaagatcgggataatgccgggaaaccggggctaataccggatagttttttcctccgcatggaggaaaaaggaaagacggcttcggctgtcacttacagatgggcccgcggcgcattagcttgttggtggggtaacggctcaccaaggcaacgatgcgtagccgacctgagagggtgatcggccacattgggactgagacacggcccaaactcctacgggaggcagcagtagggaatcttccgcaatggacgaaagtctgacggagcaacgccgcgtgagtgaagaaggccttcgggtcgtaaaactctgttgccggggaagaacaagtgccgttcgaacagggcggcgccttgacggtacccggccagaaagccacggctaactacgtgccagcagccgcggtaatacgtaggtggcaagcgttgtccggaattattgggcgtaaagcgcgcgcaggcggcttcttaagtctgatgtgaaatcttgcggctcaaccgcaagcggtcattggaaactgggaggcttgagtgcagaagaggagagtggaattccacgtgtagcggtgaaatgcgtagagatgtggaggaacaccagtggcgaaggcggctctctggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacctccctggagacagggccttccccttcgggggacagagtgacaggtggtgcatggttgtcgtcagctcgtgtcgtgagatgttgggttaagtcccgcaacgagcgcaacccttgaccttagttgccagcattcag

SEQ ID NO: 140 - DNA, 1515 bp, Pseudomonas fluorescens
  source 1..1515: mol_type = genomic DNA
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggttcgttaagttggatgtgaaagccccgggctcaacctgggaactgcattcaaaactgacgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggatgggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaacgggaggacggttaccacggtgtgattcatgactggggtgaagtcgtaacaaggtagccgtaggggaacctgcggctggatcacctcctt

SEQ ID NO: 141 - DNA, 1479 bp, Ochrobactrum sp.
  source 1..1479: mol_type = genomic DNA
cttgagagtttgatcctggctcagaacgaacgctggcggcaggcttaacacatgcaagtcgagcgccccgcaaggggagcggcagacgggtgagtaacgcgtgggaatctaccttttgctacggaacaacagttggaaacgactgctaataccgtatgtgcccttcgggggaaagatttatcggcaaaggatgagcccgcgttggattagctagttggtgaggtaaaggctcaccaaggcgacgatccatagctggtctgagaggatgatcagccacactgggactgagacacggcccagactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggatttactgggcgtaaagcgcacgtaggcggatttttaagtcaggggtgaaatcccggggctcaaccccggaactgcctttgatactggaagtcttgagtatggtagaggtgagtggaattccgagtgtagaggtgaaattcgtagatattcggaggaacaccagtggcgaaggcggctcactggaccattactgacgctgaggtgcgaaagcgtggggagcaaacaggattagataccctggtagtccacgccgtaaacgatgaatgttagccgtcggggggtttacctttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacataccggtcgcggacacagagatgtgtctttcagttcggctggaccggatacaggtgctgcatggctgtcgtcagctcgtgtcgtgagatgttgggttaagtcccgcaacgagcgcaaccctcgcctttagttgccagcatttagttgggcactctaaagggactgccagtgataagctggaggaaggtggggatgacgtcaagtcctcatggcccttacgggctgggctacacacgtgctacaatggtggtgacagtgggcagcaagcacgcgagtgtgagctaatctccaaaagccatctcagttcggattgcactctgcaactcgagtgcatgaagttggaatcgctagtaatcgcggatcagcatgccgcggtgaatacgttcccgggccttgtacacaccgcccgtcacaccatgggagttggttttacccgaaggcactgtgctaaccgcaaggaggcaggtgaccacggtagggtcagcgactggggtgaagtcgtaacaaggtagccgtaggggaacctgcggctggatcacctccttt

SEQ ID NO: 142 - DNA, 1549 bp, Bacillus megaterium
  source 1..1549: mol_type = genomic DNA
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctccttt

SEQ ID NO: 143 - DNA, 1537 bp, Erwinia rhapontici
  source 1..1537: mol_type = genomic DNA
ttgaagagtttgatcatggctcagattgaacgctggcggcaggcctaacacatgcaagtcgaacggtagcacagagagcttgctcttgggtgacgagtggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagtggggaggaaggcgatgaagttaatagcttcgtcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccacggaattcggcagagatgccttagtgccttcgggaaccgtgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 144 - DNA, 1532 bp, Pseudomonas fragi
  source 1..1532: mol_type = genomic DNA
tgaagagtttgatcatggctcagattgaacgctggcggcaggcctaacacatgcaagtcgagcggtagagagaagcttgcttctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgactgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 145 - DNA, 1136 bp, Methylobacterium adhaesivum; identical to SEQ ID NO: 54
  source 1..1136: mol_type = genomic DNA

SEQ ID NO: 146 - DNA, 1547 bp, Bacillus sp.
  source 1..1547: mol_type = genomic DNA
aacggagagtttgatcctggctcaggacgaacgctggcggcgtgcctaatacatgcaagtcgagcggacagaagggagcttgctcccggacgttagcggcggacgggtgagtaacacgtgggcaacctgccccttagactgggataactccgggaaaccggagctaataccggataatccctttctccacctggagagagggtgaaagatggcttcggctatcactaggggatgggcccgcggcgcattagctagttggtaaggtaacggcttaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgaggaaggctttcgggtcgtaaagctctgttgtgagggaagaagcggtaccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacggggacccgcacaagcagtggagcatgtggtttaattcgaagcaacgcgaagaaccttaccaggtcttgacatcctttgaccacccaagagattgggcttccccttcgggggcaaagtgacaggtggtgcatggttgtcgtcagctcgtgtcgtgagatgttgggttaagtcccgcaacgagcgcaacccttgatcttagttgccagcattgagttgggcactctaaggtgactgccggtgacaaaccggaggaaggtggggatgacgtcaaatcatcatgccccttatgacctgggctacacacgtgctacaatggatggtacaaagggcagcgaaaccgcgaggtgaagccaatcccataaagccattctcagttcggattgcaggctgcaactcgcctgcatgaagccggaattgctagtaatcgcggatcagcatgccgcggtgaatacgttcccgggtcttgtacacaccgcccgtcacaccacgagagtttgtaacacccgaagtcggtgaggcaaccttttggagccagccgcctaaggtgggacaaatgattggggtgaagtcgtaacaaggtagccgtatcggaaggtgcggctggatcacctccttt

SEQ ID NO: 147 - DNA, 1547 bp, Bacillus clausii
  source 1..1547: mol_type = genomic DNA
aacggagagtttgatcctggctcaggacgaacgctggcggcgtgcctaatacatgcaagtcgagcggacagaagggagcttgctcccggacgttagcggcggacgggtgagtaacacgtgggcaacctgccccttagactgggataactccgggaaaccggagctaataccggataatccctttctccacctggagagagggtgaaagatggcttcggctatcactaagggatgggcccgcggcgcattagctagttggtaaggtaacggcttaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgaggaaggccttcgggtcgtaaagctctgttgtgagggaagaagcggtaccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacggggacccgcacaagcagtggagcatgtggtttaattcgaagcaacgcgaagaaccttaccaggtcttgacatcctttgaccacccaagagattgggcttccccttcgggggcaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggcagcgaaaccgcgaggtgaagccaatcccataaagccattctcagttcggattgcaggctgcaactcgcctgcatgaagccggaattgctagtaatcgcggatcagcatgccgcggtgaatacgttcccgggtcttgtacacaccgcccgtcacaccacgagagtttgtaacacccgaagtcggtgaggcaaccttttggagccagccgcctaaggtgggacaaatgattggggtgaagtcgtaacaaggtagccgtatcggaaggtgcggctggatcacctccttt

SEQ ID NO: 148 - DNA, 1548 bp, Bacillus clausii
  source 1..1548: mol_type = genomic DNA
acggagagtttgatcctggctcaggacgaacgctggcggcgtgcctaatacatgcaagtcgagcggagtttcaagaagcttgctttttgaaacttagcggcggacgggtgagtaacacgtgggcaacctgccccttagactgggataactccgggaaaccggagctaataccggataatccctttctccacctggagagagggtgaaagatggcttcggctatcactaagggatgggcccgcggcgcattagctagttggtaaggtaacggcttaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgaggaaggccttcgggtcgtaaagctctgttgtgagggaagaagcggtaccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacggggacccgcacaagcagtggagcatgtggtttaattcgaagcaacgcgaagaaccttaccaggtcttgacatcctttgaccacccaagagattgggcttccccttcgggggcaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggcagcgaagccgcgaggtgaagccaatcccataaagccattctcagttcggattgcaggctgcaactcgcctgcatgaagccggaattgctagtaatcgcggatcagcatgccgcggtgaatacgttcccgggtcttgtacacaccgcccgtcacaccacgagagtttgtaacacccgaagtcggtgaggcaaccttttggagccagccgcctaaggtgggacaaatgattggggtgaagtcgtaacaaggtagccgtatcggaaggtgcggctggatcacctccttt

SEQ ID NO: 149 - DNA, 1480 bp, Microbacterium sp.
  source 1..1480: mol_type = genomic DNA
tacggagagtttgatcctggctcaggatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagctctcatcgcatggtgggggttggaaagattttttggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggaccattccacggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatacaccagaacgggccagaaatggtcaactctttggacactggtgaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaaggagccgtcgaaggtgggatcggtaattaggactaagtcgtaacaaggtagccgtaccggaaggtgcggctggatcacctccttt

SEQ ID NO: 150 - DNA, 992 bp, Enterococcus faecium
  source 1..992: mol_type = genomic DNA
tgcagtcgtacgcttctttttccnccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgnnctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaggaattgacgggggcccgcacagcggtggagcatgnngnttanngancacgcgananntacnnnctnacatcnttgacnctctanagatagagcttcccttcggggcaagtgacng

SEQ ID NO: 151 - DNA, 881 bp, Bacillus velezensis
  source 1..881: mol_type = genomic DNA
cgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggtgttctttgaaaact

SEQ ID NO: 152 - DNA, 1563 bp, Lactobacillus plantarum
  source 1..1563: mol_type = genomic DNA
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctt

SEQ ID NO: 153 - DNA, 1546 bp, Bacillus velezensis
  source 1..1546: mol_type = genomic DNA
tagtgggtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 154 - DNA, 1285 bp, Bacillus subtilis
  source 1..1285: mol_type = genomic DNA
gtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 155 - DNA, 1563 bp, Lactobacillus plantarum; identical to SEQ ID NO: 152
  source 1..1563: mol_type = genomic DNA

SEQ ID NO: 156 - DNA, 1265 bp, Bacillus subtilis
  source 1..1265: mol_type = genomic DNA
cgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 157 - DNA, 1548 bp, Leuconostoc mesenteroides
  source 1..1548: mol_type = genomic DNA
attgagagtttgatcctggctcaggatgaacgctggcggcgtgcctaatacatgcaagtcgaacgcacagcgaaaggtgcttgcacctttcaagtgagtggcgaacgggtgagtaacacgtggacaacctgcctcaaggctggggataacatttggaaacagatgctaataccgaataaaacttagtgtcgcatgacaaaaagttaaaaggcgcttcggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtatgggaagaacagctagaataggaaatgattttagtttgacggtaccataccagaaagggacggctaaatacgtgccagcagccgcggtaatacgtatgtcccgagcgttatccggatttattgggcgtaaagcgagcgcagacggtttattaagtctgatgtgaaagcccggagctcaactccggaatggcattggaaactggttaacttgagtgcagtagaggtaagtggaactccatgtgtagcggtggaatgcgtagatatatggaagaacaccagtggcgaaggcggcttactggactgcaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggattgtagtctgcaactcgactacatgaagtcggaatcgctagtaatcgcggatcagcacgccgcggtgaatacgttcccgggtcttgtacacaccgcccgtcacaccatgggagtttgtaatgcccaaagccggtggcctaaccttttaggaaggagccgtctaaggcaggacagatgactggggtgaagtcgtaacaaggtagccgtaggagaacctgcggctggatcacctccttt

SEQ ID NO: 158 - DNA, 1421 bp, Lactobacillus brevis
  source 1..1421: mol_type = genomic DNA
atctgcccagaagcaggggataacacttggaaacaggtgctaataccgtataacaacaaaatccgcatggattttgtttgaaaggtggcttcggctatcacttctggatgatcccgcggcgtattagttagttggtgaggtaaaggcccaccaagacgatgatacgtagccgacctgagagggtaatcggccacattgggactgagacacggcccaaactcctacgggaggcagcagtagggaatcttccacaatggacgaaagtctgatggagcaatgccgcgtgagtgaagaagggtttcggctcgtaaaactctgttgttaaagaagaacacctttgagagtaactgttcaagggttgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggcttaaccggagaagtgcatcggaaactgggagacttgagtgcagaagaggacagtggaactccatgtgtagcggtggaatgcgtagatatatggaagaacaccagtggcgaaggcggctgtctagtctgtaactgacgctgaggctcgaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttctgccaatcttagagataagacgttcccttcggggacagaatgacaggtggtgcatggttgtcgtcagctcgtgtcgtgagatgttgggttaagtcccgcaacgagcgcaacccttattatcagttgccagcattcagttgggcactctggtgagactgccggtgacaaaccggaggaaggtggggatgacgtcaaatcatcatgccccttatgacctgggctacacacgtgctacaatggacggtacaacgagttgcgaagtcgtgaggctaagctaatctcttaaagccgttctcagttcggattgtaggctgcaactcgcctacatgaagttggaatcgctagtaatcgcggatcagcatgccgcggtgaatacgttcccgggccttgtacacaccgcccgtcacaccatgagagtttgtaacacccaaagccggtgagataaccttcgggagtcagccgtctaaggtgggacagatgattagggtgaagtcgtaacaaggtagccgtaggagaacctgcggctggatcacctcctt

SEQ ID NO: 159 - DNA, 1388 bp, Lactobacillus paracasei
  source 1..1388: mol_type = genomic DNA
tgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctccttt

SEQ ID NO: 160 - DNA, 1219 bp, Lactobacillus paracasei
  source 1..1219: mol_type = genomic DNA
acacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctccttt

SEQ ID NO: 161 - DNA, 1546 bp, Lactococcus garvieae
  source 1..1546: mol_type = genomic DNA
aatgagagtttgatcctggctcaggacgaacgctggcggcgtgcctaatacatgcaagtcgagcgatgattaaagatagcttgctatttttatgaagagcggcgaacgggtgagtaacgcgtgggaaatctgccgagtagcgggggacaacgtttggaaacgaacgctaataccgcataacaatgagaatcgcatgattcttatttaaaagaagcaattgcttcactacttgatgatcccgcgttgtattagctagttggtagtgtaaaggactaccaaggcgatgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaaactctgttgttagagaagaacgttaagtagagtggaaaattacttaagtgacggtatctaaccagaaagggacggctaactacgtgccagcagccgcggtaatacgtaggtcccaagcgttgtccggatttattgggcgtaaagcgagcgcaggtggtttcttaagtctgatgtaaaaggcagtggctcaaccattgtgtgcattggaaactgggagacttgagtgcaggagaggagagtggaattccatgtgtagcggtgaaatgcgtagatatatggaggaacaccggaggcgaaagcggctctctggcctgtaactgacactgaggctcgaaagcgtggggagcaaacaggattagataccctggtagtccacgccgtaaacgatgagtgctagctgtagggagctataagttctctgtagcgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatactcgtgatatccttagagataaggagttccttcgggacacgggatacaggtggtgcatggttgtcgtcagctcgtgtcgtgagatgttgggttaagtcccgcaacgagcgcaacccttattactagttgccatcattaagttgggcactctagtgagactgccggtgataaaccggaggaaggtggggatgacgtcaaatcatcatgccccttatgacctgggctacacacgtgctacaatggatggtacaacgagtcgccaacccgcgagggtgcgctaatctcttaaaaccattctcagttcggattgcaggctgcaactcgcctgcatgaagtcggaatcgctagtaatcgcggatcagcacgccgcggtgaatacgttcccgggccttgtacacaccgcccgtcacaccacggaagttgggagtacccaaagtaggttgcctaaccgcaaggagggcgcttcctaaggtaagaccgatgactggggtgaagtcgtaacaaggtagccgtatcggaaggtgcggctggatcacctccttt

SEQ ID NO: 162 - DNA, 1546 bp, Lactococcus garvieae; identical to SEQ ID NO: 161
  source 1..1546: mol_type = genomic DNA

SEQ ID NO: 163 - DNA, 1263 bp, Lactobacillus plantarum
  source 1..1263: mol_type = genomic DNA
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatgg

SEQ ID NO: 164 - DNA, 1457 bp, Pediococcus pentosaceus
  source 1..1457: mol_type = genomic DNA
atgagagtttgatcttggctcaggatgaacgctggcggcgtgcctaatacatgcaagtcgaacgaacttccgttaattgattatgacgtacttgtactgattgagattttaacacgaagtgagtggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggcaaaggctcaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgagaccgcgaggttaagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaac

SEQ ID NO: 165 - DNA, 1415 bp, Pichia kudriavzevii
  source 1..1415: mol_type = genomic DNA
tccgtaggtgaacctgcggaaggatcattactgtgatttagtactacactgcgtgagcggaacgaaaacaacaacacctaaaatgtggaatatagcatatagtcgacaagagaaatctacgaaaaacaaacaaaactttcaacaacggatctcttggttctcgcatcgatgaagagcgcagcgaaatgcgatacctagtgtgaattgcagccatcgtgaatcatcgagttcttgaacgcacattgcgcccctcggcattccggggggcatgcctgtttgagcgtcgtttccatcttgcgcgtgcgcagagttgggggagcggagcggacgacgtgtaaagagcgtcggagctgcgactcgcctgaaagggagcgaagctggccgagcgaactagactttttttcagggacgcttggcggccgagagcgagtgttgcgagacaacaaaaagctcgacctcaaatcaggtaggaatacccgctgaacttaagcatatcaataagcggaggaaaagaaaccaacagggattgcctcagtagcggcgagtgaagcggcaagagctcagatttgaaatcgtgctttgcggcacgagttgtagattgcaggttggagtctgtgtggaaggcggtgtccaagtcccttggaacagggcgcccaggagggtgagagccccgtgggatgccggcggaagcagtgaggcccttctgacgagtcgagttgtttgggaatgcagctccaagcgggtggtaaattccatctaaggctaaatactggcgagagaccgatagcgaacaagtactgtgaaggaaagatgaaaagcactttgaaaagagagtgaaacagcacgtgaaattgttgaaagggaagggtattgcgcccgacatggggattgcgcaccgctgcctctcgtgggcggcgctctgggctttccctgggccagcatcggttcttgctgcaggagaaggggttctggaacgtggctcttcggagtgttatagccagggccagatgctgcgtgcggggaccgaggactgcggccgtgtaggtcacggatgctggcagaacggcgcaacaccgcccgtcttgaaacatggaccaaggagtctaacgtctatgcgagtgtttgggtgtgaaacccgtacgcgtaatgaaagtgaacgtaggtcggaccccctgccctcggggaggggagcacgatcgaccgatcccgatgtttatcggaaggatttgagtaggagcatagctgttgggacccgaaagatggtgaactatgcctgaatagggtgaagccagaggaaactctggtggaggctcgtagcggttctgacgtgcaaatcgatcgtcgaatttgggtataggggcgaaagactaatcgaaccatctagtagctggttcctgccgaagtttccctcagga

SEQ ID NO: 166 - DNA, 980 bp, Bacillus sp.
  source 1..980: mol_type = genomic DNA
tcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaccctagagatagggcttcccttcgggg

SEQ ID NO: 167 - DNA, 680 bp, Bacillus atrophaeus
  source 1..680: mol_type = genomic DNA
tgcagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaa

SEQ ID NO: 168 - DNA, 644 bp, Bacillus sp.
  source 1..644: mol_type = genomic DNA
tgcagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaatggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtg

SEQ ID NO: 169 - DNA, 787 bp, Bacillus tequilensis
  source 1..787: mol_type = genomic DNA
tgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgangagcgaaagcgtggggagcgaacaggattagataccctggtagtccacgccgtaaacgatgagtgctaagtgtta

SEQ ID NO: 170 - DNA, 914 bp, Rhodosporidium babjevae
  source 1..914: mol_type = genomic DNA
ttacttggagtccgaactctcactttttaaccctgtgcatctgttaattggaatagtagctcttcggagtgaaccaccattcacttataaaacacaaagtctatgaatgtatacaaatttataacaaaacaaaactttcaacaacggatctcttggctctcgcatcgatgaagaacgcagcgaaatgcgatacgtaatgtgaattgcagaattcagtgaatcatcgaatctttgaacgcaccttgcgctccttggtattccgaggagcatgcctgtttgagtgtcatgaaatcttcaacccacctctttcttagtgaatctggtggtgcttggtttctgagcgctgctctgcttcggcttagctcgttcgtaatgcattagcatccgcaaccgaacttcggattgacttggcgtaatagactattcgctgaggattctagtttactagagccgagttgggttaaaggaagctcctaatcctaaagtctattttttgattagatctcaaatcaggtaggactacccgctgaacttaagcatatcaataagcggaggaaaagaaactaacaaggattcccctagtagcggcgagcgaagcgggaagagctcaaatttataatctggcaccttcggtgtccgagttgtaatctctagaagtgttttccgcgttggaccgcacacaagtctgttggaatacagcggcatagtggtgaaacccccgtatatggtgcggacgcccagcgctttgtgatacactttcaatgagtcgagttgtttgggaatgcagctcaaattgggtggtaaattccatctaaagctaaatattggcgagagaccgatagcgaacaagtaccgtgagggaaagatgaaaagcactttggaaagagagttaacagtacgtgaaattgttggaa

SEQ ID NO: 171 - DNA, 927 bp, Candida santamariae
  source 1..927: mol_type = genomic DNA
gggggcatcagtattcagttgtcagaggtgaaattcttggatttactgaagactaactactgcgaaagcatttgccaaggacgttttcattaatcaagaacgaaagttaggggatcgaagatgatcagataccgtcgtagtcttaaccataaactatgccgactagggatcgggtgttgttctttttttgacgcactcggcaccttacgagaaatcaaagtctttgggttctggggggagtatggtcgcaaggctgaaacttaaaggaattgacggaagggcaccaccaggagtggagcctgcggcttaatttgactcaacacggggaaactcaccaggtccagacacaataaggattgacagattgagagctctttcttgattttgtgggtggtggtgcatggccgttcttagttggtggagtgatttgtctgcttaattgcgataacgaacgagaccttaacctactaaatagtgctgctagcttttgctggtatagtcacttcttagagggactatcgatttcaagtcgatggaagtttgaggcaataacaggtctgtgatgcccttagacgttctgggccgcacgcgcgctacactgacggagccagcgagttctaaccttggccgagaggtctgggtaatcttgtgaaactccgtcgtgctggggatagagcattgtaattattgctcttcaacgaggaattcctagtaagcgcaagtcatcagcttgcgttgattacgtccctgccctttgtacacaccgcccgtcgctactaccgattgaatggcttagtgaggcttccggattggtttaaagaagggggcaactccatcttggaaccgaaaagctagtcaaacttggtcatttagaggaagtaaaagtcgtaacaaggtttccgtaggtgaacctgcggaaggatcatt

SEQ ID NO: 172 - DNA, 619 bp, Weissella cibaria
  source 1..619: mol_type = genomic DNA
gatttgaagagcttgctcagatatgacgatggacattgcaaagagtggcgaacgggtgagtaacacgtgggaaacctacctcttagcaggggataacatttggaaacagatgctaataccgtataacaatagcaaccgcatggttgctacttaaaagatggttctgctatcactaagagatggtcccgcggtgcattagttagttggtgaggtaatggctcaccaagacgatgatgcatagccgagttgagagactgatcggccacaatgggactgagacacggcccatactcctacgggaggcagcagtagggaatcttccacaatgggcgaaagcctgatggagcaacgccgcgtgtgtgatgaagggtttcggctcgtaaaacactgttgtaagagaagaatgacattgagagtaactgttcaatgtgtgacggtatcttaccagaaaggaacggctaaatacgtgccagcagccgcggtaatacgtatgttccaagcgttatccggatttattgggcgtaaagcgagcgcagacggttatttaagtctgaagtgaaagccctcagctcaactgaggaattgctttggaaactggatgacttgagtgcagtagagg

SEQ ID NO: 173 - DNA, 663 bp, Leuconostoc mesenteroides
  source 1..663: mol_type = genomic DNA
aacgcacagcgaaaggtgcttgcacctttcaagtgagtggcgaacgggtgagtaacacgtggacaacctgcctcaaggctggggataacatttggaaacagatgctaataccgaataaaacttagtgtcgcatgacaaaaagttaaaaggcgcttcggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtatgggaagaacagctagaataggaaatgattttagtttgacggtaccataccagaaagggacggctaaatacgtgccagcagccgcggtaatacgtatgtcccgagcgttatccggatttattgggcgtaaagcgagcgcagacggtttattaagtctgatgtgaaagcccggagctcaactccggaatggcattggaaactggttaacttgagtgcagtagaggtaagtggaactccatgtgtagcggtggaatgcgtagatatatggaagaacacc

SEQ ID NO: 174 - DNA, 1548 bp, Leuconostoc mesenteroides; identical to SEQ ID NO: 3
  source 1..1548: mol_type = genomic DNA

SEQ ID NO: 175 - DNA, 650 bp, Leuconostoc mesenteroides
  source 1..650: mol_type = genomic DNA
gcagtcgaacgcacagcgaaaggtgcttgcacctttcaagtgagtggcgaacgggtgagtaacacgtggacaacctgcctcaaggctggggataacatttggaaacagatgctaataccgaataaaactcagtgtcgcatgacacaaagttaaaaggcgctttggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtacgggaagaacagctagaatagggaatgattttagtttgacggtaccataccagaaagggacggctaaatacgtgccagcagccgcggtaatacgtatgtcccgagcgttatccggatttattgggcgtaaagcgagcgcagacggttgattaagtctgatgtgaaagcccggagctcaactccggaatggcattggaaactggttaacttgagtgcagtagaggtaagtggaactccatgtgtagcggtggaatgcg

SEQ ID NO: 176 - DNA, 697 bp, Leuconostoc mesenteroides
  source 1..697: mol_type = genomic DNA
gtcgaacgcacagcgaaaggtgcttgcacctttcaagtgagtggcgaacgggtgagtaacacgtggacaacctgcctcaaggctggggataacatttggaaacagatgctaataccgaataaaactcagtgtcgcatgacacaaagttaaaaggcgctttggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtacgggaagaacagctagaatagggaatgattttagtttgacggtaccataccagaaagggacggctaaatacgtgccagcagccgcggtaatacgtatgtcccgagcgttatccggatttattgggcgtaaagcgagcgcagacggttgattaagtctgatgtgaaagcccggagctcaactccggaatggcattggaaactggttaacttgagtgcagtagaggtaagtggaactccatgtgtagcggtggaatgcgtagatatatggaagaacaccagtggcgaaggcggcttactggactgtaac

SEQ ID NO: 177 - DNA, 1457 bp, Pediococcus pentosaceus; identical to SEQ ID NO: 164
  source 1..1457: mol_type = genomic DNA

SEQ ID NO: 178 - DNA, 741 bp, Pediococcus pentosaceus
  source 1..741: mol_type = genomic DNA
tgcagtcgaacgaacttccgttaattgattatgacgtacttgtactgattgagattttaacacgaagtgagtggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggcaaaggctcaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggct

SEQ ID NO: 179 - DNA, 705 bp, Pediococcus pentosaceus
  source 1..705: mol_type = genomic DNA
agtcgaacgaacttccgttaattgattatgacgtacttgtactgattgagattttaacacgaagtgagtggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggcaaaggctcaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaag

SEQ ID NO: 180 - DNA, 673 bp, Pediococcus pentosaceus
  source 1..673: mol_type = genomic DNA
tcgaacgaacttccgttaattgattatgacgtacttgtactgattgagattttaacacgaagtgagtggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggcaaaggctcaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcg

SEQ ID NO: 181 - DNA, 679 bp, Pediococcus pentosaceus
  source 1..679: mol_type = genomic DNA
tgcagtcgaacgcatttccgttaaaagaatcagaagtgcttgcacggaagatgattttaacaatgaaatgagtggcgaacgggtgagtaacacgtgggtaacctgcccagaagagggggataacacttggaaacaggtgctaataccgcataataaagaaaaccgcatggttttcctttaaaagatggtttcggctatcacttctggatggacccgcggcgtattagctagttggtaaggtaaaggcttaccaaggcagtgatacgtagccgacctgagagggtaatcggccacattgggactgagacacggcccagactcctacgggaggcagcagtagggaatcttccacaatggacgaaagtctgatggagcaacgccgcgtgagtgaagaagggtttcggctcgtaaaactctgttgttaaagaagaacgtgggtgagagtaactgttcacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgc

SEQ ID NO: 182 - DNA, 658 bp, Pediococcus pentosaceus
  source 1..658: mol_type = genomic DNA
agtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtg

SEQ ID NO: 183 - DNA, 1532 bp, Pseudomonas fragi
  source 1..1532: mol_type = genomic DNA
tgaagagtttgatcatggctcagattgaacgctggcggcaggcctaacacatgcaagtcgagcggtagagagaagcttgcttctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 184 - DNA, 1532 bp, Pseudomonas fragi
  source 1..1532: mol_type = genomic DNA
tgaagagtttgatcatggctcagattgaacgctggcggcaggcctaacacatgcaagtcgagcggtagagagaagcttgcttctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggtattaacctaatacgttagtactttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacataatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 185 - DNA, 1406 bp, Pseudomonas fragi; identical to SEQ ID NO: 53
  source 1..1406: mol_type = genomic DNA

SEQ ID NO: 186 - DNA, 1532 bp, Pseudomonas fragi
  source 1..1532: mol_type = genomic DNA
tgaagagtttgatcatggctcagattgaacgctggcggcaggcctaacacatgcaagtcgagcggtagagagaagcttgcttctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttggtgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccctcgggaggacggttaccacggtgtgattcatgactggggtgaagtcgtaacaaggtagccgtaggggaacctgcggctggatcacctcctt

SEQ ID NO: 187 - DNA, 1486 bp, Pseudomonas fragi
  source 1..1486: mol_type = genomic DNA
tgaagagtttgatcatggctcagattgaacgctggcggcaggcctaacacatgcaagtcgagcggtagagagaagcttgcttctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgactgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccttcgggaggacggttaccacggtgtgattcatgactggggtgaagt

SEQ ID NO: 188 - DNA, 1516 bp, Pseudomonas fragi
  source 1..1516: mol_type = genomic DNA
tgaatcaagcaattcgtgtgggtgcttgtggagtcagactgatagtcaacaagattatcagcatcacaagttactccgccggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 189 - DNA, 1532 bp, Pseudomonas fluorescens; identical to SEQ ID NO: 63
  source 1..1532: mol_type = genomic DNA

SEQ ID NO: 190 - DNA, 1532 bp, Pseudomonas fluorescens; identical to SEQ ID NO: 63
  source 1..1532: mol_type = genomic DNA

SEQ ID NO: 191 - DNA, 1532 bp, Pseudomonas fluorescens
  source 1..1532: mol_type = genomic DNA
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 192 - DNA, 1412 bp, Pseudomonas fluorescens
  source 1..1412: mol_type = genomic DNA
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtg

SEQ ID NO: 193 - DNA, 1532 bp, Pseudomonas fluorescens; identical to SEQ ID NO: 63
  source 1..1532: mol_type = genomic DNA

SEQ ID NO: 194 - DNA, 1532 bp, Pseudomonas fluorescens; identical to SEQ ID NO: 63
  source 1..1532: mol_type = genomic DNA

SEQ ID NO: 195 - DNA, 1539 bp, Rahnella sp.
  source 1..1539: mol_type = genomic DNA
ttgaagagtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgcaagagcaaagtgggggaccttcgggcctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaagggttcagtgttaatagcactgtgcattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 196 - DNA, 1447 bp, Rahnella sp.
  source 1..1447: mol_type = genomic DNA
tgacgagcggcggacgggtgagtaatgtctgggaaactgcctgatggagggggataactactggaaacggtagctaataccgcatgacgtcgcaagaccaaagtgggggaccttcgggcctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcgttgcagttaatagctgcaacgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 197 - DNA, 1392 bp, Rahnella sp.
  source 1..1392: mol_type = genomic DNA
ttgaagagtttgatcatggctcagattgaacgctggcggcaggcctaacacatgcaagtcgagcggtagcacaggagagcttgctctccgggtgacgagcggcggacgggtgagtaatgtctgggaaactgcctgatggagggggataactactggaaacggtagctaataccgcatgatgtcgcaagaccaaagtgggggaccttcgggcctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcgttgcagttaatagctgcaacgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgta

SEQ ID NO: 198 - DNA, 1444 bp, Rahnella sp.
  source 1..1444: mol_type = genomic DNA
cgagcggcggacgggtgagtaatgtctgggaaactgcctgatggagggggataactactggaaacggtagctaataccgcatgacgtcgcaagaccaaagtgggggaccttcgggcctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcgttgcagttaatagctgcagcgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 199 - DNA, 1426 bp, Rahnella sp.
  source 1..1426: mol_type = genomic DNA
gtaatgtctgggaaactgcctgatggagggggataactactggaaacggtagctaataccgcatgatgtcgcaagaccaaagtgggggaccttcgggcctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcgttgcagttaatagctgcaacgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 200 - DNA, 1539 bp, Rahnella sp.; identical to SEQ ID NO: 22
  source 1..1539: mol_type = genomic DNA

SEQ ID NO: 201 - protein, 5 aa, synthetic construct
LPXTG

SEQ ID NO: 202 - DNA, 20 bp, synthetic construct
  source 1..20: mol_type = other DNA
ttcgtcgggaagtgatggtg

SEQ ID NO: 203 - DNA, 20 bp, synthetic construct
  source 1..20: mol_type = other DNA
cttggtcgtggcatcagtct

SEQ ID NO: 204 - DNA, 20 bp, synthetic construct
  source 1..20: mol_type = other DNA
tatgcccattgaccgcaagg

SEQ ID NO: 205 - DNA, 20 bp, synthetic construct
  source 1..20: mol_type = other DNA
agcaagcttcctggtttggg

SEQ ID NO: 206 - DNA, 22 bp, synthetic construct
  source 1..22: mol_type = other DNA
atggcaaagtatttcacatcgg

SEQ ID NO: 207 - DNA, 30 bp, synthetic construct
  source 1..30: mol_type = other DNA
ttaaagtaagtttttgatttctttcacctt

SEQ ID NO: 208 - DNA, 18 bp, synthetic construct
  source 1..18: mol_type = other DNA
ggcgttgtccatccaatg

SEQ ID NO: 209 - DNA, 19 bp, synthetic construct
  source 1..19: mol_type = other DNA
caggagaattgctgttccc

(19) United States
(12) Patent Application Publication
(10) Pub. No.: US 2025/0008995 A1
(43) Pub. Date: Jan. 9, 2025

(54) COMPOSITIONS AND METHODS FOR PRODUCING ENHANCED CROPS WITH PROBIOTICS

(71) Applicant: Solarea Bio, Inc., Cambridge, MA (US)

(72) Inventors: Alicia Eve Ballok, Natick, MA (US); Josephine James Kennedy, Somerville, MA (US); Gerardo V. Toledo, Hopkinton, MA (US); Eric Michael Schott, West Roxbury, MA (US); Tracy Mincer, Jupiter, FL (US); Rachel Ariel Raymond, McLean, VA (US); Luis Arturo Montes de Oca Valeriano, Cambridge, MA (US)

(21) Appl. No.: 18/747,158

(22) Filed: Jun. 18, 2024

Related U.S. Application Data
(63) Continuation of application No. PCT/US22/53401, filed on Dec. 19, 2022.
(60) Provisional application No. 63/291,913, filed on Dec. 20, 2021.

(51) Int. Cl. A23L 19/00; A01N 63/20; A01N 63/27; A01N 63/30; A01P 21/00

(52) U.S. Cl. CPC A23L 19/05; A01N 63/20; A01N 63/27; A01N 63/30; A01P 21/00

(57) ABSTRACT

The present invention relates to the identification of a group of microorganisms, which are relatively abundant in the microbial communities associated with fruits and vegetables typically consumed raw and therefore transient or permanent members of the human microbiota. The consumption of mixtures of these microbes at relevant doses produces a beneficial health effect in the host. The present invention also relates to methods of using these microbes to increase the presence of beneficial microbes in crops.

### CROSS REFERENCE TO RELATED APPLICATIONS

This application claims the benefit of U.S. Provisional Application Nos: 63/291,913 filed Dec. 20, 2021, which is hereby incorporated in its entirety by reference for all purposes.

### SEQUENCE LISTING

The instant application contains a Sequence Listing which has been submitted via Patent Center and is hereby incorporated by reference in its entirety. Said XML, created on XXX, is named XXX, and is XXX in size.

### BACKGROUND OF THE INVENTION

### Field of the Invention

The invention relates to methods and compositions useful for producing crops with enhanced microbial content, which are beneficial to the consumer of the crop and to the crop itself.

### Description of the Related Art

Daily consumption of fresh fruits, vegetables, seeds and other plant-derived ingredients of salads and juices is recognized as part of a healthy diet and associated with weight loss, weight management and overall healthy lifestyles. This is demonstrated clinically and epidemiologically in the “China Study” (Campbell, T. C. and Campbell T. M. 2006. The China Study: startling implications for diet, weight loss and long-term health. Benbella books. pp 419) where a lower incidence of cardiovascular diseases, cancer and other inflammatory-related indications were observed in rural areas where diets are whole food plant-based. The benefit from these is thought to be derived from the vitamins, fiber, antioxidants and other molecules that are thought to benefit the microbial flora through the production of prebiotics. These can be in the form of fermentation products from the breakdown of complex carbohydrates and other plant-based polymers. There has been no clear mechanistic association between microbes in whole food plant-based diets and the benefits conferred by such a diet. The role of these microbes as probiotics, capable of contributing to gut colonization and thereby influencing a subject's microbiota composition in response to a plant-based diet, has been underappreciated.

While endophytic bacteria and fungi are ubiquitous in plants, the quantity, diversity, and species found are not always equivalent. Farming practices, growing region, and plant species characteristics, among other factors, influence the microbial content of any given plant. What is needed to optimize the probiotic effect of raw fruits and vegetables are methods and compositions for enhancing the microbial content of edible plants including vine crops, leafy vegetables, cruciferous vegetables, cucurbits, root vegetables, and berries from perennial and annual bushes.

Strawberries, for example, represent one of the crops most consumed worldwide and with the need of very intense use of agrochemicals to control fungal pathogens. In recent years the use of methyl bromide commonly used to treat soils was banned and therefore alternative agents for pathogen control are needed. In addition, it is desirable to increase the nutritional value of the harvested fruit and extend shelf life. One of the components that can increase the nutritional value of strawberries is represented by bacteria and fungi colonizing internal and external tissues known as the Edible Plant Microbiome.

Novel methods and compositions are needed for increasing nutritional value of edible plants, such as vine crops, leafy vegetables, cruciferous vegetables, cucurbits, root vegetables, and berries from perennial and annual bushes while also improving the health of the plant and the half-life of the harvested plant tissues for consumption.

### SUMMARY

Disclosed herein are nutritive food products comprising at least a portion of an edible vine crop plant, wherein at least a portion of the edible vine crop plant comprises a nutriobiotic comprising at least one heterologous microbe. In certain embodiments, the edible vine crop plant is selected from cranberries and grapes. In certain aspects, disclosed herein are nutritive food products comprising at least a portion of an edible leafy vegetable plant, wherein the at least a portion of the edible leafy vegetable plant comprises a nutriobiotic comprising at least one heterologous microbe. In certain embodiments, the edible leafy vegetable plant is selected from the group consisting of romaine lettuce, spinach, iceberg lettuce, and arugula. In certain aspects, disclosed herein are nutritive food products comprising at least a portion of an edible cruciferous vegetable, wherein the at least a portion of the cruciferous vegetable comprises a nutriobiotic comprising at least one heterologous microbe. In certain embodiments, the edible cruciferous vegetable is selected from the group consisting of broccoli, cauliflower, and brussel sprouts. In certain aspects, disclosed herein are nutritive food products comprising at least a portion of an edible cucurbit plant, wherein the at least a portion of the cucurbit plant comprises a nutriobiotic comprising at least one heterologous microbe. In certain embodiments, the edible cucurbit plant is selected from the group consisting of watermelon, melon, cucumber and squash.

In certain aspects, disclosed herein are nutritive food products comprising at least a portion of an edible root vegetable plant, wherein the at least a portion of the edible root vegetable plant comprises a nutriobiotic comprising at least one heterologous microbe. In certain embodiments, the edible root vegetable plant is selected from the group consisting of carrot, beet and radish.

In certain aspects, disclosed herein are nutritive food products comprising at least a portion of an edible perennial or annual bush, wherein the at least a portion of the edible perennial or annual bush comprises a nutriobiotic comprising at least one heterologous microbe. In certain embodiments, the edible perennial or annual bush is a berry bush. In certain embodiments, the berry bush is selected from: strawberry, blackberry, raspberry, and blueberry.

In certain embodiments, the edible plant comprises a diversified microbial ecology comprising at least one heterologous microbe that benefits growth of the edible plant. In certain embodiments, the edible plant comprises a diversified microbial ecology comprising at least two heterologous microbes that synergistically benefit growth of the edible plant. In certain embodiments, the edible plant comprises a diversified microbial ecology comprising at least one heterologous microbe that improve resistance to the edible plant to abiotic stress selected from temperature and moisture level. In certain embodiments, the edible plant comprises a diversified microbial ecology comprising at least two heterologous microbes that synergistically improve resistance to the edible plant to abiotic stress selected from temperature and moisture level. In certain embodiments, the edible plant comprises a diversified microbial ecology comprising at least two heterologous microbes that synergistically benefit growth of the edible plant. In certain embodiments, the at least a portion of the edible plant is obtained from the edible plant under conditions such that the diversified microbial ecology is substantially retained in the at least a portion of the edible plant. In certain embodiments, the diversified microbial ecology produces a heterologous metabolite or enhance the production of endogenous metabolites in a tissue of the edible plant. In certain embodiments, the edible plant comprises detectable amounts of the heterologous microbe. In certain embodiments, the at least a portion of the edible plant comprises detectable amounts of heterologous microbes that colonize the edible plant.

In certain aspects, disclosed herein are nutritive food products comprising a macerated preparation derived from at least a portion of an edible plant selected from the group consisting of: a vine crop, a leafy vegetable, a cucurbit, a root vegetable, and a perennial and annual bush, wherein the at least a portion of the edible plant comprises a diversified microbial ecology comprising at least one heterologous microbe.

In certain embodiments of the nutritive food products disclosed herein, the heterologous microbe comprises a microbial species selected from any one of the species shown in Table B. In certain embodiments, the heterologous microbe comprises a microbial species selected from any one of the species shown in Table E. In certain embodiments, the heterologous microbe comprises a nucleic acid sequence that has at least 97% identity to any one of the sequences shown in Table F. In certain embodiments, the heterologous microbe comprises a nucleic acid sequence selected from any one of the sequences shown in Table F. In certain embodiments, the at least a portion of the edible plant comprises a part of the plant selected from: a berry, a root, and a leaf.

In certain aspects, disclosed herein are seeds or seedlings of an edible plant having deposited on an exterior surface of the seed or seedling a formulation comprising an heterologous microbe, wherein the heterologous microbe is deposited on an exterior surface of the seed or seedling in an amount effective to colonize the plant, the formulation further comprising at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient; wherein the edible plant is selected from the group consisting of: a vine crop, a leafy vegetable, a cucurbit, a root vegetable, and a perennial and annual bush.

In certain aspects, disclosed herein are seeds or seedlings of an edible plant having deposited on an exterior surface of the seed or seedling a formulation comprising an heterologous microbe, wherein the heterologous microbe is deposited on an exterior surface of the seed or seedling in an amount effective to colonize the plant, the formulation further comprising a polymeric and/or adhesive substance; wherein the edible plant is selected from the group consisting of: a vine crop, a leafy vegetable, a cucurbit, a root vegetable, and a perennial and annual bush.

In certain aspects, disclosed herein are formulations comprising a heterologous microbe and a polymeric and/or adhesive substance.

In certain embodiments of the seed or formulation disclosed herein, the polymeric substance comprises a vinyl pyrrolidone/vinyl acetate copolymer. In certain embodiments, the vinyl pyrrolidone/vinyl acetate copolymer comprises a Agrimer VA 6 polymer. In certain embodiments, the formulation is formulated as a spray. In certain embodiments, the heterologous microbe comprises a microbial species selected from any one of the species shown in Table B. In certain embodiments, the heterologous microbe comprises a microbial species selected from any one of the species shown in Table E. In certain embodiments, the heterologous microbe comprises a nucleic acid sequence that has at least 97% identity to any one of the sequences shown in Table F. In certain embodiments, the heterologous microbe comprises a nucleic acid sequence selected from any one of the sequences shown in Table F.

In certain aspects, disclosed herein are methods of modulating the microbial composition of at least a portion of an edible plant comprising heterologously depositing an heterologous microbe to the edible plant, seed, seedling, or seed-associated soil environment in an amount effective to alter the microbial composition of the at least a portion of the edible plant produced by the edible plant relative to a reference edible plant, seed, seedling, or seed-associated soil environment not comprising the heterologous microbe.

In certain aspects, disclosed herein are edible plants having deposited on an exterior surface of a flower or fruit of the edible plant a formulation comprising an heterologous microbe, wherein the heterologous microbe is deposited on the exterior surface of the flower or fruit in an amount effective to colonize the edible plant. In certain embodiments, the edible plant is selected from the group consisting of: a vine crop, and a perennial and annual bush. In certain embodiments, the edible plant further comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, a humectant, plant penetration aid, and a nutrient. In certain embodiments, the heterologous microbe is deposited on the edible plant by applying a formulation comprising the heterologous microbe as a liquid bolus. In certain embodiments, the heterologous microbe is deposited on the edible plant by applying a formulation comprising the heterologous microbe as a spray. In certain embodiments, the formulation comprising the heterologous microbe comprises at least one member selected from the group consisting of an agriculturally compatible carrier, a tackifier, a microbial stabilizer, a fungicide, an antibacterial agent, an herbicide, a nematicide, an insecticide, a plant growth regulator, a humectant, plant penetration aid, a rodenticide, and a nutrient. In certain embodiments, the formulation comprises a synthetic polymeric and/or adhesive substance. In certain embodiments, polymeric substance comprises a vinyl pyrrolidone/vinyl acetate copolymer, an alkoxylated polyol ester, or a modified Tween 20 (polyoxyethylene/polyoxypropylene/sorbitan monolaurate) polymer. In certain embodiments, the vinyl pyrrolidonc/vinyl acetate copolymer comprises a Agrimer VA 6 polymer, a Croda Tween L-1010 adjuvant, or an ATPlus UEP-100 adjuvant. In certain embodiments, the formulation comprises a natural polymeric and/or adhesive substance. In certain embodiments, the polymeric substance comprises xanthan gum.

In certain embodiments, the heterologous microbe is deposited on the edible plant by spraying the formulation comprising the heterologous microbe. In certain embodiments, the heterologous microbe comprises a microbial species selected from any one of the species shown in Table B. In certain embodiments, the heterologous microbe comprises a microbial species selected from any one of the species shown in Table E. In certain embodiments, the heterologous microbe comprises a nucleic acid sequence that has at least 97% identity to any one of the sequences shown in Table F. In certain embodiments, the heterologous microbe comprises a nucleic acid sequence selected from any one of the sequences shown in Table F. In certain embodiments, the heterologous microbe comprises a microbial species of a defined microbial assemblage (DMA) of Table H. In certain embodiments, the heterologous microbe comprises a DMA of Table H. In certain embodiments, the amount of heterologous microbe effective to colonize the edible plant comprises at least 1×10⁴ CFU/gram of flower or fruit. In certain embodiments, the edible plant is a berry. In certain embodiments, the berry is of a berry bush selected from: strawberry, blackberry, raspberry, and blueberry.

In certain aspects, disclosed herein are nutritive food products comprising at least a portion of the edible plant described herein; and wherein the at least a portion of the edible plant comprises a nutriobiotic comprising at least one of the heterologous microbe.

In certain aspects, disclosed herein are methods of modulating the microbial composition of at least a portion of an edible plant comprising heterologously depositing a heterologous microbe to the flower or fruit of the edible plant in an amount effective to alter the microbial composition of the at least a portion of the edible plant produced by the edible plant relative to a reference edible plant not comprising the heterologous microbe.

In certain embodiments, the amount of heterologous microbe effective to colonize the edible plant comprises at least 1×10⁴ CFU/gram of flower or fruit. In certain embodiments, the heterologous microbe is deposited on the edible plant by spraying a formulation comprising lyophilized heterologous microbes. In certain embodiments, the formulation comprises a vinyl pyrrolidone/vinyl acetate copolymer, an alkoxylated polyol ester, or a modified Tween 20 (polyoxyethylene/polyoxypropylene/sorbitan monolaurate) polymer. In certain embodiments, the vinyl pyrrolidone/vinyl acetate copolymer, alkoxylated polyol ester, or modified Tween 20 (polyoxyethylene/polyoxypropylene/sorbitan monolaurate) polymer comprises a Agrimer VA 6 polymer, a Croda Tween L-1010 adjuvant, an ATPlus UEP-100 adjuvant, or combinations thereof. In certain embodiments, the formulation comprises xanthan gum. In certain embodiments, the at least a portion of the edible plant comprises mature fruit. In certain embodiments, the mature fruit comprises at least 1×10⁴ CFU/gram of mature fruit. In certain embodiments, the mature fruit comprises at least 1×10⁴ CFU/gram of mature fruit at least 7 days after depositing the heterologous microbe on the edible plant. In certain embodiments, the mature fruit comprises at least 1×10⁴ CFU/gram of mature fruit at least 7 days after depositing the heterologous microbe on the fruit. In certain embodiments, the mature fruit comprises at least 1×10⁴ CFU/gram of mature fruit at least 17 days after depositing the heterologous microbe on the flower.

In certain aspects, disclosed herein are methods of altering the microbial flora of a subject, the method comprising administering to the subject and effective amount of at least a portion of the edible plant, or nutritive food product disclosed herein.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 A-L show plots depicting the diversity of microbial species detected in samples taken from 12 plants usually consumed raw by humans.

FIG. 1A shows bacterial diversity observed in a green chard.

FIG. 1B shows bacterial diversity in red cabbage.

FIG. 1C shows bacterial diversity in romaine lettuce.

FIG. 1D shows bacterial diversity in celery sticks.

FIG. 1E shows bacterial diversity observed in butterhead lettuce grown hydroponically.

FIG. 1F shows bacterial diversity in organic baby spinach.

FIG. 1G shows bacterial diversity in green crisp gem lettuce

FIG. 1H shows bacterial diversity in red oak leaf lettuce.

FIG. 1I shows bacterial diversity in green oak leaf lettuce.

FIG. 1J shows bacterial diversity in cherry tomatoes.

FIG. 1K shows bacterial diversity in crisp red gem lettuce.

FIG. 1L shows bacterial diversity in broccoli juice.

FIG. 2 A-C show graphs depicting the taxonomic composition of microbial samples taken from broccoli heads (FIG. 2A), blueberries (FIG. 2B), and pickled olives (FIG. 2C).

FIG. 3 shows a schematic describing a gut simulator experiment. The experiment comprised an in vitro system that represents various sections of the gastrointestinal tract. Isolates of interest are incubated in the presence of conditions that mimic particular stresses in the gastro-intestinal tract (such as low pH or bile salts), or heat shock. After incubation, surviving populations were recovered. Utilizing this system, the impact of various stressors alone or in combination with probiotic cocktails of interest on the microbial ecosystem is tested.

FIG. 4. Shows a fragment recruitment plot sample for the shotgun sequencing on fermented cabbage comparing to the reference genome of strain DP3 Leuconostoc mesenteroides-like and the 18× coverage indicating the isolated strain was represented in the environmental sample and it was largely genetically homogeneous.

FIG. 5. Genome-wide metabolic model for a DMA formulated in silico with 3 DP strains and one genome from a reference in NCBI. The predicted fluxes for acetate, propionate and butyrate under a nutrient-replete and plant fiber media are indicated.

FIG. 6. DMA experimental validation for a combination of strains DP3 and DP9 under nutrient replete and plant fiber media showing that the strains showed synergy for increased SCFA production only under plant fiber media but not under rich media.

FIG. 7A shows the relative microbial profiles in banana pulp. Relative abundances of microbial profiles at the genus level in SBP samples. Bacterial DNA was isolated from each SBP and sequenced using HiSeq X. Sequencing reads were trimmed and filtered based on quality. Filtered reads were mapped to plant genome database to discard the reads derived from plant. The remaining sequencing reads were classified by Kraken2 with Kraken database to assign taxonomy of each read. Relative abundance of each taxonomy was computed by Bracken using taxonomic labels assigned by Kraken2. Shannon diversity was calculated by using Vegan package in R. The number of reads at the genus level assigned by Kraken2 was used as an input.

FIG. 7B shows the relative microbial profiles in green olives. Relative abundances of microbial profiles at the genus level in SBP samples. Bacterial DNA was isolated from each SBP and sequenced using HiSeq X. Sequencing reads were trimmed and filtered based on quality. Filtered reads were mapped to plant genome database to discard the reads derived from plant. The remaining sequencing reads were classified by Kraken2 with Kraken database to assign taxonomy of each read. Relative abundance of each taxonomy was computed by Bracken using taxonomic labels assigned by Kraken2. Shannon diversity was calculated by using Vegan package in R. The number of reads at the genus level assigned by Kraken2 was used as an input.

FIG. 7C shows the relative microbial profiles in blueberries. Relative abundances of microbial profiles at the genus level in SBP samples. Bacterial DNA was isolated from each SBP and sequenced using HiSeq X. Sequencing reads were trimmed and filtered based on quality. Filtered reads were mapped to plant genome database to discard the reads derived from plant. The remaining sequencing reads were classified by Kraken2 with Kraken database to assign taxonomy of each read. Relative abundance of each taxonomy was computed by Bracken using taxonomic labels assigned by Kraken2. Shannon diversity was calculated by using Vegan package in R. The number of reads at the genus level assigned by Kraken2 was used as an input.

FIG. 8 shows a diagram demonstrating the genera common between a typical human gut microbiome and genera typically found in edible plants.

FIG. 9A-B. Microbial abundance is greater in organically grown strawberries than in conventionally grown strawberries. Organic and conventional strawberries were blended with PBS, and the resulting material was filtered through meshes with pore sizes ranging from ˜1 mm to 40 μm. The filtrate was centrifuged to concentrate microbial cells, and the concentrated material was serially diluted and plated onto four different agar media types (MRS, TSA, PDA, YPD) under both acrobic and anaerobic conditions. (FIG. 9A) Colony forming units (CFUs) were counted, and average CFU/g was calculated. Error bars represent the standard deviation of two technical replicates. The dotted line indicates the limit of detection (5×10¹ CFU/g). (FIG. 9B) A visual comparison of organic vs conventional strawberry preparations plated onto agar media showed greater abundance of microbes in the organic preparation.

FIG. 9C-D. Microbial diversity differs in organically vs conventionally grown blackberries. Organic and conventional blackberries were blended with PBS, and the resulting material was filtered through meshes with pore sizes ranging from ˜1 mm to 40 um. The filtrate was centrifuged to concentrate microbial cells, and the concentrated material was serially diluted and plated onto four different agar media types under both aerobic and anaerobic conditions. (FIG. 9C) Colony forming units (CFUs) were counted, and average CFU/g was calculated. Error bars represent the standard deviation of two technical replicates. The dotted line indicates the limit of detection (5×10¹ CFU/g). (FIG. 9D) A visual comparison of organic vs conventional blackberry preparations plated onto agar media showed that colony morphologies are distinct, indicating that the microbes present are different.

FIG. 10 shows a gene pathway analysis in 57 bacterial strains displaying the groups of enzymes relevant for plant fiber degradation and the potential role these can have to build defined microbial assemblages by incorporating the plant fiber and the microorganisms producing fermentable substrates from the plant fibers. An important group of enzymes, glycosyl hydrolases, are shown in green bars.

FIG. 11 demonstrates the results of dilution plating technique for colonization. DP102 inoculated plants (bottom) and mock treatment control (top) were diluted and plated on PDA containing chlorotetracycline. An aliquot of 5 μl for each 10-fold dilution was applied to a plate an held vertically to distribute the liquid along its length.

FIG. 12 demonstrates PCR detection of microbes on plants using species-specific primers. FIG. 12A shows PCR assay Controls. Primers were tested against microbial genomic DNA (positive control) and each mock-treated plant type to verify primer specificity. FIG. 12B shows the results of PCR assays for exemplary strains. Primers were tested against genomic DNA from the microbe of interest and other microbes to verify specificity. On the left gel, bands are visible in the DP102 control well and the DMA #1 lettuce well. DMA #1 contains DP102. For the center gel, bands are seen with DP5 positive control and the arugula samples with DMA #3 and DMA #4 treatment, both of which contain DP5. The gel on the right DP100 is detected from arugula treated with DP100 as well as the positive controls. The use of PCR probes for specific strains allows to detect colonization in the plant tissues and to confirm counts based on colony forming units.

FIG. 13A demonstrates the effects of seed polymer coating in combination with microbe inoculation and shows the effects of microbial inoculation and polymer coating on the colonization and biomass of arugula seedlings. The left graph demonstrates the level of colonization of these plants with each treatment. FIG. 13B demonstrates the effects of seed polymer coating in combination with microbe inoculation and shows the effects of microbial inoculation and polymer coating on the colonization and biomass of Outredgeous lettuce seedlings. FIG. 13C demonstrates the effects of seed polymer coating in combination with microbe inoculation and shows the effects of microbial inoculation and polymer coating on the colonization and biomass of Little Gem lettuce seedlings. FIG. 13D demonstrates the effects of seed polymer coating in combination with microbe inoculation and shows the effects of microbial inoculation and polymer coating on the colonization and biomass of Black Seeded Simpson lettuce seedlings.

FIG. 14 demonstrates the effect of increasing inoculum on plant colonization level. Arugula seeds were inoculated with DP100 at levels from 1×10³ up to 1×10⁷ CFU/seed (dark gray bars) and compared to the CFU/g microbial output on the resultant seedlings.

FIG. 15A shows the levels of colonization of seedlings with single microbes or DMAs on a variety of plant types after seed inoculation and demonstrates colonization of seedlings with Debaryomyces hansenii DP5 expressed as average CFU per gram plant material. FIG. 15B shows the levels of colonization of seedlings with single microbes or DMAs on a variety of plant types after seed inoculation and demonstrates colonization of seedlings with Lactobacillus plantarum DP100 expressed as average CFU per gram plant material. FIG. 15C shows the levels of colonization of seedlings with single microbes or DMAs on a variety of plant types after seed inoculation and demonstrates colonization of seedlings with Leuconostoc mesenteroides DP93 expressed as average CFU per gram plant material. FIG. 15D shows the levels of colonization of seedlings with single microbes or DMAs on a variety of plant types after seed inoculation and demonstrates colonization of seedlings with DMA #2 expressed as average CFU per gram plant material.

FIG. 16A demonstrates colonization of seedlings with DMAs. Eight seed-types were inoculated with DMAs and colonization was examined and demonstrates colonization of seedlings with DMA #3. FIG. 16B demonstrates colonization of seedlings with DMAs. Eight seed-types were inoculated with DMAs and colonization was examined and demonstrates colonization of seedlings with DMA #4. FIG. 16C demonstrates colonization of seedlings with DMAs. Eight seed-types were inoculated with DMAs and colonization was examined and demonstrates colonization of seedlings with DMA #5. FIG. 16D demonstrates colonization of seedlings with DMAs. Eight seed-types were inoculated with DMAs and colonization was examined and demonstrates colonization of seedlings with DMA #6.

FIG. 17A demonstrates colonization and weights of hydroponically grown lettuces and shows the average colonization of per plant (dark grey bars) relative to the original seed inoculum (light gray bars). FIG. 17B demonstrates colonization and weights of hydroponically grown lettuces and shows box and whisker plots of lettuce plant masses. In general plant mass was unchanged by treatment type regardless of whether colonization was successful. FIG. 17C demonstrates colonization and weights of hydroponically grown lettuces and is a histogram depicting aggregate plant masses. The total mass of 12 plants per treatment was measured. Differences in total yield can be seen between lettuce types but not within each group.

FIG. 18 provides a microbial preparation of seeds can enhance tomato plant growth.

FIG. 19A shows germination rates under heat stress. Germination rates for each lettuce variety are displayed as percent germination (of 18 seeds) over time. FIG. 19B demonstrates total plant survival under heat stress. FIG. 19C demonstrates pro-Hex aggregate weights under heat stress. The total weight of all Outredgeous and Black Seeded Simpson lettuce plants harvested at 35 days post planting.

FIG. 20A shows that Little Gem seeds treated with microbes result in larger and more healthy plants when subjected to abiotic (heat) stress. Photographs of mature plants from mock-treated (left) and single microbe or DMA-treated seeds (right). FIG. 20B shows that Little Gem potted plant masses grown with heat stress. Box and whisker plot of masses from five lettuce plants harvested (left) and a histogram of aggregate plant masses (right).

FIG. 21A-D are graphs depicting the concentration of heterologous after application of DMA 5 to fruits with natural and synthetic polymers. DMA #5, consisting of L. plantarum and P. kudriavzevii, was mixed with a variety of polymers and applied to growing strawberries in a liquid spray. FIG. 21A) L. plantarum CFUs per fruit at start (0 hours), two days, and 7 days post administration. FIG. 21B) L. plantarum CFUs per gram at start (0 hours), two days, and 7 days post administration. FIG. 21C) P. kudriavzevii CFUs per fruit at start (0 hours), two days, and 7 days post administration. FIG. 21D) P. kudriavzevii CFUs per gram at start (0 hours), two days, and 7 days post administration. Error bars indicate standard deviation (n=2 per timepoint). ND=not determined. Lyo DMA=lyophilized DMA #5.

FIG. 22A-C depicts DMA enrichment of fruits by direct application of a bolus to flowers of strawberry plants. DMA #5, consisting of a high concentration of L. plantarum and P. kudriavzevii, was applied to flowers in a liquid with ATPlus. FIG. 22A) Material was pipetted directly onto to the center (pistil-containing portion) of the flower, as indicated by the black circle. FIG. 22B) Abundance of L. plantarum after DMA application to the flower (0 hours) and yield on the resultant strawberry fruit (24 days after DMA administration).

FIG. 22C) Abundance of P. kudriavzevii after DMA application to the flower (0 hours) and yield on the resultant fruit (24 days after DMA administration). Error bars represent standard deviation (n=7-8).

FIG. 23 depicts DMA Enrichment of Strawberries by Spray Application to Flowers. DMA #5, consisting of L. plantarum and P. kudriavzevii, was applied to the flowers of strawberry plants in a liquid spray with ATPlus. The bar graph illustrates the abundance of each DMA component per fruit 25 days after application to flowers. Error bars represent standard deviation (n=2).

FIG. 24A depicts DMA enrichment of strawberry fruits with three microbe preparation methods. DMA #5 (L. plantarum and P. kudriavzevii)/ATPlus polymer solutions were prepared from broth culture (dark gray), from washed microbes (light gray), or from lyophilized microbes (black). Microbes were applied to strawberries and microbial abundance was measured on the fruit up to 7 days after application. The bar graphs illustrate the abundance of L. plantarum and P. kudriavzevii 0, 2, and 7 days after DMA application, expressed as CFU/fruit. component Error bars represent standard deviation (n=2).

FIG. 24B depicts DMA abundance in strawberry fruit after inoculation of flower using three different microbe preparation methods. DMA #5 (L. plantarum and P. kudriavzevii)/ATPlus polymer solutions were prepared from broth culture (medium gray), from washed microbes (light gray) or lyophilized microbes (dark gray). Microbes were applied by spraying the strawberry plant flowers, and microbial abundance was measured on the flowers (0 days) and the resulting fruits (17/22 days after inoculation). The bar graphs illustrate the abundance of L. plantarum and P. kudriavzevii 0, 2, and 7 days after DMA application, expressed as CFU/fruit (or flower) or CFU/g plant tissue. Error bars represent standard deviation (n=2).

FIG. 25 depicts high titers of DMA microbes can be achieved with high titer application to fruits. DMA #5, consisting of lyophilized L. plantarum and P. kudriavzevii, was applied to flowers in a liquid spray with ATPlus. The application was with a high concentration of microbes. FIG. 25A) Microbial abundance expressed as CFU/fruit of L. plantarum (circles) vs. P. kudriavzevii (squares) at the time of administration (0 days) and one week later (7 days). Error bars represent standard deviation (n=8). FIG. 25B) Microbial abundance expressed as CFU/gr of L. plantarum (circles) vs. P. kudriavzevii (squares) at the start (0 days) and one week after administration (7 days). Error bars represent standard deviation (n=8).

### DETAILED DESCRIPTION

### Advantages and Utility

Edible crops contain a microbiota which is consumed and become transient, or permanent, members of the gut microbiome of the consumer. These comprise plant-associated bacteria and fungi that can serve as beneficial or pathogen roles. Most of what is known about the plant microbiota and the gut microbiota is for pathogens. The plant microbiota changes with the agricultural practices, for example organic farming promotes a greater diversity and abundance of microbes compared to conventional farming practices.

The plant microbiome can be enhanced to contain relevant members of the human gut by enriching the fresh fruits or vegetables during farming with beneficial microorganism. One example of this is the use of lactic acid bacteria that can be enhanced in strawberries or spinach to create a nutritive food product where the consumer of the produce will receive a beneficial dose of probiotics that can improve wellness.

In addition to the beneficial microbiota enrichment there are other upgrading aspects for the crop by the inoculation and incorporation onto the edible tissues a target microbiota. For example, crop color in strawberries can be enhanced using Methylobacteria producing pigments. This can give the fruits a red color that can be more desirable for the consumer. In addition, there are other sensory features such as volatile compounds produced by yeast that can contribute to the fruit's aroma.

Methods and compositions for improving the microbial content of edible plants allow enhanced health benefits of consuming said edible plants. Consuming beneficial microbes at effective amounts as part of food eliminates the extraneous step of taking separately formulated probiotics, which is inconvenient and can be difficult to remember. Additionally, by enhancing the microbial content of the plants themselves, differences between similar plants, due to growth conditions, etc, can be reduced.

### Definitions

Terms used in the claims and specification are defined as set forth below unless otherwise specified.

The term “ameliorating” refers to any therapeutically beneficial result in the treatment of a disease state, e.g., a metabolic disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and includes but is not limited to humans, non-human primates, canines, felines, murines, bovines, equines, and porcines.

The term “plant” or “plant component” as used herein includes entire plants, portions of plants which are generally known or known to those of skill in the art, which include, but are not limited to, roots, leaves, stems, fruit, tubers.

As used herein, the term “derived from” includes microbes immediately taken from an environmental sample and also microbes isolated from an environmental source and subsequently grown in pure culture.

The term “percent identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. In some aspects, percent identity is defined with respect to a region useful for characterizing phylogenetic similarity of two or more organisms, including two or more microorganisms. Percent identity, in these circumstances can be determined by identifying such sequences within the context of a larger sequence, that can include sequences introduced by cloning or sequencing manipulations such as, e.g., primers, adapters, etc., and analyzing the percent identity in the regions of interest, without including in those analyses introduced sequences that do not inform phylogenetic similarity.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al.).

One example of an algorithm that is suitable for determining percent sequence identity and sequence similarity is the BLAST algorithm, which is described in Altschul et al. J. Mol. Biol. 215:403-410 (1990). Software for performing BLAST analyses is publicly available through the National Center for Biotechnology Information.

The term “sufficient amount” means an amount sufficient to produce a desired effect, e.g., an amount sufficient to alter the microbial content of a subject's microbiota.

The term “therapeutically effective amount” is an amount that is effective to ameliorate a symptom of a disease. A therapeutically effective amount can be a “prophylactically effective amount” as prophylaxis can be considered therapy.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, inhibiting substantially, slowing, or reversing the progression of a condition, substantially ameliorating clinical or aesthetical symptoms of a condition.

As used herein, the term “preventing” includes completely or substantially reducing the likelihood or occurrence or the severity of initial clinical or aesthetical symptoms of a condition.

As used herein, the term “about” includes variation of up to approximately +/−10% and that allows for functional equivalence in the product.

As used herein, the term “colony-forming unit” or “CFU” is an individual cell that is able to clone itself into an entire colony of identical cells.

As used herein all percentages are weight percent unless otherwise indicated.

As used herein, “viable organisms” are organisms that are capable of growth and multiplication. In some embodiments, viability can be assessed by numbers of colony-forming units that can be cultured. In some embodiments, viability can be assessed by other means, such as quantitative polymerase chain reaction.

The term “derived from” includes material isolated from the recited source, and materials obtained using the isolated materials (e.g., cultures of microorganisms made from microorganisms isolated from the recited source).

“Microbiota” refers to the community of microorganisms that occur (sustainably or transiently) in and on an animal or plant subject, typically a mammal such as a human, including eukaryotes, archaea, bacteria, and viruses (including bacterial viruses i.e., phage).

“Microbiome” refers to the genetic content of the communities of microbes that live in and on the human body, both sustainably and transiently, including eukaryotes, archaea, bacteria, and viruses (including bacterial viruses (i.e., phage), wherein “genetic content” includes genomic DNA, RNA such as ribosomal RNA, the epigenome, plasmids, and all other types of genetic information.

“Pure culture” as used herein indicates a microbe grown under conditions such that the resulting microbial culture is largely homogeneous, and largely free of contaminants.

As used herein, a Defined Microbial Assemblage (DMA) is a rationally designed synthetic consortium of heterogeneous microbes, and an optional plant fiber.

The term “subject” refers to any animal subject including humans, laboratory animals (e.g., primates, rats, mice), livestock (e.g., cows, sheep, goats, pigs, turkeys, and chickens), and household pets (e.g., dogs, cats, and rodents). The subject may be suffering from a dysbiosis, including, but not limited to, an infection due to a gastrointestinal pathogen or may be at risk of developing or transmitting to others an infection due to a gastrointestinal pathogen.

The “colonization” of a host organism includes the non-transitory residence of a bacterium or other microscopic organism. As used herein, “reducing colonization” of a host subject's gastrointestinal tract (or any other microbial niche) by a pathogenic bacterium includes a reduction in the residence time of the pathogen in the gastrointestinal tract as well as a reduction in the number (or concentration) of the pathogen in the gastrointestinal tract or adhered to the luminal surface of the gastrointestinal tract. Measuring reductions of adherent pathogens may be demonstrated, e.g., by a biopsy sample, or reductions may be measured indirectly, e.g., by measuring the pathogenic burden in the stool of a mammalian host.

A “combination” of two or more bacteria includes the physical co-existence of the two bacteria, either in the same material or product or in physically connected products, as well as the temporal co-administration or co-localization of the two bacteria.

The term “nutritive food product”, as used herein, refers to a food product comprising at least a portion of one or more edible plants comprising a nutriobiotic comprising at least one heterologous microbe.

As used herein “heterologous microbe” designates organisms to be administered that are not naturally present in the same proportions as in the therapeutic composition as in subjects to be treated with the therapeutic composition. These can be organisms that are not normally present in individuals in need of the composition described herein, or organisms that are not present in sufficient proportion in said individuals. These organisms can comprise a synthetic composition of organisms derived from separate plant sources or can comprise a composition of organisms derived from the same plant source, or a combination thereof.

As used herein “heterologous metabolite” refers to a metabolite present in a plant or seed colonized with a heterologous microbe, where the metabolite is not normally present and/or not naturally present in the same proportion as a reference plant not colonized with the heterologous microbe.

Controlled-release refers to delayed release of an agent, from a composition or dosage form in which the agent is released according to a desired profile in which the release occurs after a period of time.

The term “nutriobiotic” is a composition of a single microbe or a combination of two or more that are both beneficial to a plant when applied prior or during farming and/or provides a probiotic benefit to a mammal that consumes the final product.

The term “diversified microbial ecology” includes nutriobiotic compositions and optionally endogenous microbes that confer benefits, including agricultural benefits to the plant and/or probiotic benefits to a mammal that consumes the product.

Throughout this application, various embodiments of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range.

It is appreciated that certain features of the invention, which are, for clarity, described in the context of separate embodiments, can also be provided in combination in a single embodiment. Conversely, various features of the invention, which are, for brevity, described in the context of a single embodiment, can also be provided separately or in any suitable subcombination or as suitable in any other described embodiment of the invention. Certain features described in the context of various embodiments are not to be considered essential features of those embodiments, unless the embodiment is inoperative without those elements.

It must be noted that, as used in the specification and the appended claims, the singular forms “a,” “an” and “the” include plural referents unless the context clearly dictates otherwise.

As used herein GOS indicates one or more galacto-oligosaccharides and FOS indicates one or more fructo-oligosaccharide.

The following abbreviations are used in this specification and/or Figures: ac=acetic acid; but=butyric acid; ppa=propionic acid.

### Compositions of the Invention

In certain embodiments, compositions of the invention comprise probiotic compositions formulated for administration or consumption, with a prebiotic and any necessary or useful excipient. In other embodiments, compositions of the invention comprise probiotic compositions formulated for consumption without a prebiotic. Probiotic compositions of the invention are, in some embodiments, isolated from foods normally consumed raw and isolated for cultivation. In some embodiments, microbes are isolated from different foods normally consumed raw, but multiple microbes from the same food source may be used.

It is known to those of skill in the art how to identify microbial strains. Bacterial strains are commonly identified by 16S rRNA gene sequence. Fungal species can be identified by sequence of the internal transcribed space (ITS) regions of rDNA or the 18S rRNA gene sequence.

One of skill in the art will recognize that the 16S rRNA gene and the ITS region comprise a small portion of the overall genome, and so sequence of the entire genome (whole genome sequence) may also be obtained and compared to known species.

Additionally, multi-locus sequence typing (MLST) is known to those of skill in the art. This method uses the sequences of 7 known bacterial genes, typically 7 housekeeping genes, to identify bacterial species based upon sequence identity of known species as recorded in the publicly available PubMLST database. Housekeeping genes are genes involved in basic cellular functions.

In certain embodiments, bacterial entities of the invention are identified by comparison of the 16S rRNA sequence to those of known bacterial species, as is well understood by those of skill in the art. In certain embodiments, fungal species of the invention are identified based upon comparison of the ITS sequence to those of known species (Schoch et al PNAS 2012). In certain embodiments, microbial strains of the invention are identified by whole genome sequencing and subsequent comparison of the whole genome sequence to a database of known microbial genome sequences. While microbes identified by whole genome sequence comparison, in some embodiments, are described and discussed in terms of their closest defined genetic match, as indicated by 16S rRNA sequence, it should be understood that these microbes are not identical to their closest genetic match and are novel microbial entities. This can be shown by examining the Average Nucleotide Identity (ANI) of microbial entities of interest as compared to the reference strain that most closely matches the genome of the microbial entity of interest. ANI is further discussed in Example 6.

In other embodiments, microbial entities described herein are functionally equivalent to previously described strains with homology at the 16S rRNA or ITS region. In certain embodiments, functionally equivalent bacterial strains have 95% identity at the 16S rRNA region and functionally equivalent fungal strains have 95% identity at the ITS region. In certain embodiments, functionally equivalent bacterial strains have 96% identity at the 16S rRNA region and functionally equivalent fungal strains have 96% identity at the ITS region. In certain embodiments, functionally equivalent bacterial strains have 97% identity at the 16S rRNA region and functionally equivalent fungal strains have 97% identity at the ITS region. In certain embodiments, functionally equivalent bacterial strains have 98% identity at the 16S rRNA region and functionally equivalent fungal strains have 98% identity at the ITS region. In certain embodiments, functionally equivalent bacterial strains have 99% identity at the 16S rRNA region and functionally equivalent fungal strains have 99% identity at the ITS region. In certain embodiments, functionally equivalent bacterial strains have 99.5% identity at the 16S rRNA region and functionally equivalent fungal strains have 99.5% identity at the ITS region. In certain embodiments, functionally equivalent bacterial strains have 100% identity at the 16S rRNA region and functionally equivalent fungal strains have 100% identity at the ITS region.

16S rRNA sequences for strains tolerant of metformin or with probiotic potential (described in Table E) are found in Table F. 16S rRNA is one way to classify bacteria into operational taxonomic units (OTUs). Bacterial strains with 97% sequence identity at the 16S rRNA locus are considered to belong to the same OTU. A similar calculation can be done with fungi using the ITS locus in place of the bacterial 16S rRNA sequence. It is well within the level of ordinary skill of one in the art to isolate these species following the teachings of this specification. The successful isolation of these species can be determined by 16S sequence comparison to the reference sequences of these species provided herein (e.g., in Table F). In other embodiments, a person of ordinary skill can determine that substitutions for these novel species may be made using either or both of the most closely matching species by 16S (such as to the reference sequences of these species provided herein, e.g., in Table F) or ANI sequence comparison. Further it is within the level of ordinary skill to distinguish operable from inoperable substitutions by assembling a substituted DMA and assaying for any one of the activities set forth, e.g., in any one of the working examples provided in this specification.

In some embodiments, the invention provides an enhanced or cultured probiotic composition for the enhancement of microbial content of edible plants comprising a mixture of Pediococcus pentosaceus and/or Leuconostoc mesenteroides, or a Lactobacillus species combined with non-lactic acid bacteria isolated or identified from samples described in Table A or described in Table B. In some embodiments, the invention provides an enhanced or cultured probiotic composition for the enhancement of microbial content of edible plants. In some embodiments, the invention provides an enhanced or cultured probiotic composition for the enhancement of microbial content of edible plants comprising a mixture of Pediococcus pentosaceus and/or Leuconostoc mesenteroides, or a Lactobacillus species. In some embodiments, the invention provides a fermented probiotic composition for the enhancement of microbial content of edible plants comprising a mixture of Pediococcus pentosaceus and/or Leuconostoc mesenteroides or a Lactobacillus species and at least one non-lactic acid bacterium, preferably a bacterium classified as a gamma proteobacterium or a filamentous fungus or yeast. Some embodiments comprise the fermented probiotic being in a capsule or microcapsule adapted for enteric delivery. In some embodiments, the probiotic regimen complements an anti-diabetic regimen.

The compositions disclosed herein are derived from edible plants and can comprise a mixture of microorganisms, comprising bacteria, fungi, archaea, and/or other endogenous or heterologous microorganisms, all of which work together to form a microbial ecosystem with a role for each of its members.

In some embodiments, species of interest are isolated from plant-based food sources normally consumed raw. These isolated compositions of microorganisms from individual plant sources can be combined to create a new mixture of organisms. Particular species from individual plant sources can be selected and mixed with other species cultured from other plant sources, which have been similarly isolated and grown. In some embodiments, species of interest are grown in pure cultures before being prepared for consumption or administration. In some embodiments, the organisms grown in pure culture are combined to form a synthetic combination of organisms.

In some embodiments, the microbial composition comprises proteobacteria or gamma proteobacteria. In some embodiments, the microbial composition comprises several species of Pseudomonas. In some embodiments, species from another genus are also present. In some embodiments, a species from the genus Duganella is also present. In some embodiments of said microbial composition, the population comprises at least three unique isolates selected from the group consisting of Pseudomonas, Acinetobacter, Aeromonas, Curtobacterium, Escherichia, Lactobacillus, Leuconostoc, Pediococcus, Serratia, Streptococcus, and Stenotrophomonas. In some embodiments of said microbial composition, the population comprises at least two unique isolates selected from the group consisting of Pseudomonas, Acinetobacter, Aeromonas, Curtobacterium, Escherichia, Lactobacillus, Leuconostoc, Pediococcus, Serratia, Streptococcus, and Stenotrophomonas. In some embodiments, the bacteria are selected based upon their ability to modulate production of one or more branch chain fatty acids, short chain fatty acids, and/or flavones in a mammalian gut.

In some embodiments the microbial compositions comprises several species of the yeast genera belonging to Debaromyces, Pichia, and Hanseniaspora.

In some embodiments, microbial compositions comprise isolates that are capable of modulating production or activity of the enzymes involved in fatty acid metabolism, such as acetolactate synthase I, N-acetylglutamate synthase, acetate kinase, Acetyl-CoA synthetase, acetyl-CoA hydrolase, Glucan 1,4-alpha-glucosidase, or Bile acid symporter Acr3.

In some embodiments, the administered microbial compositions colonize the treated mammal's digestive tract. In some embodiments, these colonizing microbes comprise bacterial assemblages present in whole food plant-based diets. In some embodiments, these colonizing microbes comprise Pseudomonas with a diverse species denomination that is present and abundant in whole food plant-based diets. In some embodiments, these colonizing microbes reduce free fatty acids absorbed into the body of a host by absorbing the free fatty acids in the gastrointestinal tract of mammals. In some embodiments, these colonizing microbes comprise genes encoding metabolic functions related to desirable health outcomes such as increased efficacy of anti-diabetic treatments, lowered BMI, lowered inflammatory metabolic indicators, etc.

### Prebiotics

Prebiotics, in accordance with the teachings of this invention, comprise compositions that promote the growth of beneficial bacteria in the intestines. Prebiotic substances can be consumed by a relevant probiotic, or otherwise assist in keeping the relevant probiotic alive or stimulate its growth. When consumed in an effective amount, prebiotics also beneficially affect a subject's naturally-occurring gastrointestinal microflora and thereby impart health benefits apart from just nutrition. Prebiotic foods enter the colon and serve as substrate for the endogenous bacteria, thereby indirectly providing the host with energy, metabolic substrates, and essential micronutrients. The body's digestion and absorption of prebiotic foods is dependent upon bacterial metabolic activity, which salvages energy for the host from nutrients that escaped digestion and absorption in the small intestine.

Prebiotics help probiotics flourish in the gastrointestinal tract, and accordingly, their health benefits are largely indirect. Metabolites generated by colonic fermentation by intestinal microflora, such as short-chain fatty acids, can play important functional roles in the health of the host. Prebiotics can be useful agents for enhancing the ability of intestinal microflora to provide benefits to their host.

Prebiotics, in accordance with the embodiments of this invention, include, without limitation, mucopolysaccharides, oligosaccharides, polysaccharides, amino acids, vitamins, nutrient precursors, proteins, and combinations thereof.

According to particular embodiments, compositions comprise a prebiotic comprising a dietary fiber, including, without limitation, polysaccharides and oligosaccharides. These compounds have the ability to increase the number of probiotics, and augment their associated benefits. For example, an increase of beneficial Bifidobacteria likely changes the intestinal pH to support the increase of Bifidobacteria, thereby decreasing pathogenic organisms.

Non-limiting examples of oligosaccharides that are categorized as prebiotics in accordance with particular embodiments include galactooligosaccharides, fructooligosaccharides, inulins, isomalto-oligosaccharides, lactilol, lactosucrose, lactulose, pyrodextrins, soy oligosaccharides, transgalacto-oligosaccharides, and xylo-oligosaccharides.

According to other particular embodiments, compositions comprise a prebiotic comprising an amino acid.

Prebiotics are found naturally in a variety of foods including, without limitation, cabbage, bananas, berries, asparagus, garlic, wheat, oats, barley (and other whole grains), flaxseed, tomatoes, Jerusalem artichoke, onions and chicory, greens (e.g., dandelion greens, spinach, collard greens, chard, kale, mustard greens, turnip greens), and legumes (e.g., lentils, kidney beans, chickpeas, navy beans, white beans, black beans). Generally, according to particular embodiments, compositions comprise a prebiotic present in a sweetener composition or functional sweetened composition in an amount sufficient to promote health and wellness.

In particular embodiments, prebiotics also can be added to high-potency sweeteners or sweetened compositions. Non-limiting examples of prebiotics that can be used in this manner include fructooligosaccharides, xylooligosaccharides, galactooligosaccharides, and combinations thereof.

Many prebiotics have been discovered from dietary intake including, but not limited to: antimicrobial peptides, polyphenols, Okara (soybean pulp by product from the manufacturing of tofu), polydextrose, lactosucrose, malto-oligosaccharides, gluco-oligosaccharides (GOS), fructo-oligosaccharides (FOS), xantho-oligosaccharides, soluble dietary fiber in general. Types of soluble dietary fiber include, but are not limited to, psyllium, pectin, or inulin. Phytoestrogens (plant-derived isoflavone compounds that have estrogenic effects) have been found to have beneficial growth effects of intestinal microbiota through increasing microbial activity and microbial metabolism by increasing the blood testosterone levels, in humans and farm animals. Phytoestrogen compounds include but are not limited to: Oestradiol, Daidzein, Formononetin, Biochainin A, Genistein, and Equol.

Dosage for the compositions described herein are deemed to be “effective doses,” indicating that the probiotic or prebiotic composition is administered in a sufficient quantity to alter the physiology of a subject in a desired manner. In some embodiments, the desired alterations include reducing obesity, and or metabolic syndrome, and sequelae associated with these conditions. In some embodiments, the desired alterations are promoting rapid weight gain in livestock. In some embodiments, the prebiotic and probiotic compositions are given in addition to an anti-diabetic regimen.

### Nutritive Food Products

In certain aspects, described herein are nutritive food products comprising at least a portion of one or more edible plants comprising a nutriobiotic comprising at least one of heterologous microbe. The heterologous microbe of the nutritive food products described herein can comprise a microbial species selected from any one of the species shown in Table B or Table E. The heterologous microbe can comprise a nucleic acid sequence that has at least 80%, at least 85%, at least 90%, at least 91%, at least 92%, at least 93%, at least 94%, at least 95%, at least 96%, at least 97% at least 98%, at least 99%, at least 99.5%, or 100% identity to any one of the sequences shown in Table F.

In certain embodiments, the nutritive food product comprises a macerated preparation derived from at least a portion of one or more edible plants selected from the group consisting of: a vine crop, a leafy vegetable, a cucurbit, a root vegetable, and a perennial and annual bush, wherein the at least a portion of the edible plant comprises a diversified microbial ecology comprising at least one heterologous microbe. The nutritive food products can comprise at least a portion of one or more vine crop plants, wherein the edible vine crop plant is cranberries and/or grapes. The nutritive food product can comprise at least a portion of one or more edible leafy vegetable plants, wherein the edible leafy vegetable plant is romaine lettuce, spinach, iceberg lettuce, and/or arugula. The nutritive food product can comprise at least a portion of one or more edible cruciferous vegetables, wherein the edible cruciferous vegetable is broccoli, cauliflower, and/or brussels sprouts. The nutritive food product can comprise at least a portion of one or more edible cucurbit plants, wherein the edible cucurbit plant is watermelon, melon, cucumber and/or squash. The nutritive food product can comprise at least a portion of one or more edible root vegetable plants, wherein the edible root vegetable plant is carrot, beet and/or radish. The nutritive food product can comprise at least a portion of one or more edible perennial or annual bushes, wherein the edible perennial or annual bush is a berry bush, and optionally, wherein the berry bush is strawberry, blackberry, raspberry, and/or blueberry.

### Seeds and Seedlings

In certain aspects, described herein are seeds or seedlings of an edible plant having deposited on an exterior surface of the seed or seedling a formulation comprising an heterologous microbe, wherein the heterologous microbe is deposited on an exterior surface of the seed or seedling in an amount effective to colonize the plant, the formulation further comprising at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, and a nutrient; wherein the edible plant is selected from the group consisting of: a vine crop, a leafy vegetable, a cucurbit, a root vegetable, and a perennial and annual bush.

In certain aspects, described herein are seeds or seedlings of an edible plant having deposited on an exterior surface of the seed or seedling a formulation comprising an heterologous microbe, wherein the heterologous microbe is deposited on an exterior surface of the seed or seedling in an amount effective to colonize the plant, the formulation further comprising a polymeric and/or adhesive substance; wherein the edible plant is selected from the group consisting of: a vine crop, a leafy vegetable, a cucurbit, a root vegetable, and a perennial and annual bush.

### Edible Plants

In certain aspects, described herein are edible plant having deposited on an exterior surface of a flower or berry of the edible plant a formulation comprising a heterologous microbe, wherein the heterologous microbe is deposited on the exterior surface of the flower or berry in an amount effective to colonize the edible plant. In certain embodiments, the edible plant is selected from the group consisting of: a vine crop, a leafy vegetable, a cucurbit, a root vegetable, and a perennial and annual bush. In certain embodiments, the edible plant further comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, a humectant, a plant penetration aid and a nutrient. In certain embodiments, the heterologous microbe is deposited on the edible plant by applying a formulation described herein comprising the heterologous microbe as a liquid bolus. In certain aspects, the heterologous microbe comprises a microbial species selected from any one of the species shown in Table B and/or Table E. In certain embodiments, the heterologous microbe comprises a nucleic acid sequence that has at least 80%, at least 85%, at least 90%, at least 91%, at least 92%, at least 93%, at least 94%, at least 95%, at least 96%, at least 97% at least 98%, at least 99%, at least 99.5%, or 100% identity to any one of the sequences shown in Table F. In certain embodiments, the heterologous microbe comprises a microbial species of a defined microbial assemblage (DMA) of Table H. In certain embodiments, the heterologous microbe comprises a DMA of Table H.

In certain embodiments, the amount of heterologous microbe effective to colonize the edible plant comprises at least 1×10⁴ CFU/gram, 1×10⁵ CFU/gram, 1×10⁶ CFU/gram, 1×10⁷ CFU/gram, 1×10⁸ CFU/gram, 1×10⁹ CFU/gram, or 1×10¹⁰ CFU/gram, of flower or fruit.

### Formulations and Additional Ingredients

In certain aspects, described herein are formulations for treating plants for microbial augmentation, wherein the formulation comprises a heterologous microbe described herein and an additional ingredient. Additional ingredients include ingredients to improve handling, preservatives, antioxidants, and the like. In an embodiment, the compositions include microcrystalline cellulose or silicone dioxide. Preservatives can include, for example, benzoic acid, alcohols, for example, ethyl alcohol, and hydroxybenzoates. Antioxidants can include, for example, butylated hydroxyanisole (BHA), butylated hydroxytoluene (BHT), tocopherols (e.g., Vitamin E), and ascorbic acid (Vitamin C). In some embodiments, an additional agreement is an agriculturally acceptable carrier or excipient.

The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in a composition of the invention. Water-in-oil emulsions can also be used to formulate a composition that includes the purified population (see, for example, U.S. Pat. No. 7,485,451, which is incorporated herein by reference in its entirety). Suitable formulations that may be prepared include wettable powders, granules, gels, agar strips or pellets, thickeners, biopolymers, and the like, microencapsulated particles, and the like, liquids such as aqueous flowables, aqueous suspensions, water-in-oil emulsions, etc. In certain embodiments, the formulation is formulated as a spray. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, plant elements, sugar cane bagasse, hulls or stalks from grain processing, ground plant material or wood from building site refuse, sawdust or small fibers from recycling of paper, fabric, or wood. Other suitable formulations will be known to those skilled in the art.

In an embodiment, the formulation can include a tackifier or adherent. Such agents are useful for combining the complex population of the invention with carriers that can contain other compounds (e.g., control agents that are not biologic), to yield a coating composition. Such compositions help create coatings around the plant or plant element to maintain contact between the endophyte and other agents with the plant or plant element. In one embodiment, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, carragennan, PGA, other biopolymers, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers. Other examples of adherent compositions that can be used in the synthetic preparation include those described in EP 0818135, CA 1229497, WO 2013090628, EP 0192342, WO 2008103422 and CA 1041788, each of which is incorporated herein by reference in its entirety.

It is also contemplated that the formulation may further comprise an anti-caking agent.

The formulation can also contain a surfactant, wetting agent, emulsifier, stabilizer, or anti-foaming agent. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision), polysorbate 20, polysorbate 80, Tween 20, Tween 80, Scattics, Alktest TW20, Canarcel, Peogabsorb 80, Triton X-100, Conco NI, Dowfax 9N, Igebapl CO, Makon, Neutronyx 600, Nonipol NO, Plytergent B, Renex 600, Solar NO, Sterox, Serfonic N, T-DET-N, Tergitol NP, Triton N, IGEPAL CA-630, Nonident P-40, Pluronic. In one embodiment, the surfactant is present at a concentration of between 0.01% v/v to 10% v/v. In another embodiment, the surfactant is present at a concentration of between 0.1% v/v to 1% v/v. An example of an anti-foaming agent would be Antifoam-C.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the population used and should promote the ability of the endophyte population to survive application on the seeds and to survive desiccation. Examples of suitable desiccants include one or more of trehalose, sucrose, glycerol, and methylene glycol. Other suitable desiccants include, but are not limited to, non-reducing sugars and sugar alcohols (e.g., mannitol or sorbitol). The amount of desiccant introduced into the formulation can range from 5% to 50% by weight/volume, for example, between 10% to 40%, between 15% and 35%, or between 20% and 30%.

In some cases, it is advantageous for the formulation to contain agents such as a fungicide, an anticomplex agent, an herbicide, a nematicide, an insecticide, a plant growth regulator, a rodenticide, a bactericide, a virucide, or a nutrient. Such agents are ideally compatible with the agricultural plant element or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

In certain embodiments, the formulation comprising the heterologous microbe comprises at least one member selected from the group consisting of an agriculturally compatible carrier, a tackifier, a microbial stabilizer, a fungicide, an antibacterial agent, an herbicide, a nematicide, an insecticide, a plant growth regulator, a humectant, plant penetration aid, a rodenticide, and a nutrient. In certain embodiments, the formulation comprises a synthetic polymeric and/or adhesive substance. In certain embodiments, the polymeric substance comprises a vinyl pyrrolidone/vinyl acetate copolymer, an alkoxylated polyol ester, or a modified Tween 20 (polyoxyethylene/polyoxypropylene/sorbitan monolaurate) polymer. In certain embodiments, the vinyl pyrrolidone/vinyl acetate copolymer comprises a Agrimer VA 6 polymer, a Croda Tween L-1010 adjuvant, or an ATPlus UEP-100 adjuvant. In certain embodiments, the formulation comprises a natural polymeric and/or adhesive substance. In certain embodiments, the polymeric substance comprises xanthan gum.

In the liquid form, for example, solutions or suspensions, endophyte populations of the present invention can be mixed or suspended in water or in aqueous solutions. Suitable liquid diluents or carriers include water, aqueous solutions, petroleum distillates, or other liquid carriers.

Solid compositions can be prepared by dispersing the endophyte populations of the invention in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils (such as soybean oil, maize (corn) oil, and cottonseed oil), glycerol, ethylene glycol, polyethylene glycol, propylene glycol, polypropylene glycol, etc.

In an embodiment, the formulation is ideally suited for coating of a population of endophytes onto plant elements. The endophytes populations described in the present invention are capable of conferring many fitness benefits to the host plants. The ability to confer such benefits by coating the populations on the surface of plant elements has many potential advantages, particularly when used in a commercial (agricultural) scale.

The endophyte populations herein can be combined with one or more of the agents described above to yield a formulation suitable for combining with an agricultural plant element, seedling, or other plant element. Endophyte populations can be obtained from growth in culture, for example, using a synthetic growth medium. In addition, endophytes can be cultured on solid media, for example on petri dishes, scraped off and suspended into the preparation. Endophytes at different growth phases can be used. For example, endophytes at lag phase, early-log phase, mid-log phase, late-log phase, stationary phase, early death phase, or death phase can be used. Endophytic spores may be used for the present invention, for example but not limited to: arthospores, sporangispores, conidia, chlamydospores, pycnidiospores, endospores, zoospores.

### Vine Crops

Edible vine crops include cranberries and grapes. A nutritive food product comprising at least a portion of an edible vine crop plant, wherein at least a portion of the edible vine crop plant comprises a nutriobiotic comprising at least one heterologous microbe. In certain embodiments, the edible vine crop plant is selected from cranberries and grapes.

### Leafy Vegetables

In certain aspects, described herein is a nutritive food product comprising at least a portion of an edible leafy vegetable plant, wherein the at least a portion of the edible leafy vegetable plant comprises a nutriobiotic comprising at least one heterologous microbe. In certain embodiments, the edible leafy vegetable plant is selected from the group consisting of romaine lettuce, spinach, iceberg lettuce, and arugula.

### Cucurbits

In certain aspects, described herein is nutritive food product comprising at least a portion of an edible part of a cucurbit, wherein the at least a portion of the cucurbit comprises a nutriobiotic comprising at least one heterologous microbe. In certain embodiments, the edible cucurbit is selected from the group consisting of water melon, melon, squash and cucumber.

### Cruciferous Vegetables

In certain aspects, described herein is nutritive food product comprising at least a portion of an edible cruciferous vegetable, wherein the at least a portion of the cruciferous vegetable comprises a nutriobiotic comprising at least one heterologous microbe. In certain embodiments, the edible cruciferous vegetable is selected from the group consisting of broccoli, cauliflower, and brussel sprouts.

### Root Vegetables

A nutritive food product comprising at least a portion of an edible root vegetable plant, wherein the at least a portion of the edible root vegetable plant comprises a nutriobiotic comprising at least one heterologous microbe. In certain embodiments, the edible root vegetable plant is selected from the group consisting of carrot, beet and radish.

### Perennial and Annual Bush

A nutritive food product comprising at least a portion of an edible perennial or annual bush, wherein the at least a portion of the edible perennial or annual bush comprises a nutriobiotic comprising at least one heterologous microbe. In certain embodiments, the nutritive food product of claim 9, wherein the edible perennial or annual bush is a berry bush. In certain embodiments, the berry bush is selected from: strawberry, blackberry, raspberry and blueberry.

### Hydroponics

In an embodiment nutriobiotics can be applied to coated seeds to be germinated and grown hydroponically. This is relevant for indoor farming of fruits such as strawberries and vegetables such as lettuce. Seeds are planted in rockwool and exposed to a suitable growth media with nutrients required for plants including macro and micronutrients.

The nutrient solutions can be designed to optimize the use of the nutriobiotics where they can provide nitrogen nutrition in the case of using nitrogen fixing bacteria. Other nutritional requirements as in the case of vitamins can be provided by the nutriobiotics.

In another embodiment indoor and vertical farming can be enhanced by the application of nutriobiotics to the indoor crop where the crop was germinated and grown using an artificial illumination system over water tanks filled with nutrient solution. The crops can be grown to maturity and harvested.

In some embodiments the nutriobiotics are applied as seed coat in combination of a suitable seed coat polymer.

In other embodiments the nutriobiotics are applied in the nutrient solution and contacted with the crop through the root system.

In another embodiment the nutriobiotics are applied in the rockwool or substrate where the seed is being germinated.

For the indoor farming of strawberries or other fruits, the nutriobiotics can be applied during the flowering stage of the crop directly onto the flowers and with the use of a suitable delivery system such as agricultural polymers, or binding agents to improve the adhesion to the flower tissues.

In another embodiment the nutriobiotic is applied as a foliar product that can be used in combination with any of the other application modalities including seed coats, flower, germination substrate or nutrient solution. The foliar applications can be done at weekly intervals until the crop is harvested.

In another embodiment nutriobiotics can be applied in combination of a conventional agricultural product that can include agrochemicals, plant growth promoting agents or pesticides.

In another embodiment nutriobiotics can be applied to improved seeds that have been specifically selected or bred for growth in hydroponic systems.

The nutriobiotics dose ranges from 1×10³ to 1×10⁹ CFU/seed, 1×10⁴ to 1×10⁹ CFU/ml in nutrient solution, 1×10³ to 1×10⁹ CFU/cm² of foliar biomass or 1×10³ to 1×10⁹ CFU/flower.

### Tomatoes

Tomatoes are a very important crop with a wide range of varieties farmed around the world. Tomatoes are grown using different systems and it is critical to offer the most robust growth during the early stages. To enhance plant vigor and promote growth nutriobiotics can applied as seed coats to provide improved plant health that will result in higher yields. In one embodiment tomato seeds are coated with a suitable agricultural polymer and germinated on peat moss, potting soil, and combinations of these with perlite, vermiculite, turface or other suitable germination substrate. The seedlings are then transplanted to soil or into 1-to-5-gallon pots for growth. In one embodiment the seedlings are further treated with foliar applications of nutriobiotics. The fruits are colonized by the nutriobiotics but it is possible to detect the product in other plant tissues such as leaves, stems and roots.

### Abiotic Stress Tolerance Through Application of Nutriobiotics (Heat)

Due to climate change, there is a relative increase in, drought, excessive rainfall, heat waves, and exposure to this stress for crops can cause significant losses. To protect against this abiotic stress, it is desirable to have crops resilient to these stressors and that can protect seedlings during germination and plants during growth and production. In one embodiment to protect lettuce seeds can be coated with DP3, DP5 or DP95 to improve germination under heat stress where the percent of germinated plants increases compared to non-treated plants.

In another embodiment a combination of strains from Table E can create a nutriobiotic that can be applied with a suitable seed coat polymer.

In another embodiment the nutriobiotics increase the overall plant yield in a leafy green measured as wet weight at harvest.

In another embodiment the plant appearance and size is improved by the use of a nutriobiotic compared to a non-treated plant after growth and prior to harvest.

In another embodiment seeds can be coated with a combination of strains from Table E with or without polymer to create a nutriobiotic that enhances germination in drought.

In another embodiment seeds can be coated with a combination of strains from Table E with or without polymer to create a nutriobiotic that enhances germination in excessive rain.

In another embodiment seeds can be coated with a combination of strains from Table E with or without polymer to create a nutriobiotic that enhances plant growth in drought.

In another embodiment seeds can be coated with a combination of strains from Table E with or without polymer to create a nutriobiotic that enhances plant growth in excessive rain.

In some embodiments the nutriobiotics are applied as seed coat in combination of a suitable seed coat polymer. In other embodiments no polymer is used. The nutriobiotics dose ranges from 1×10³ to 1×10⁹ CFU/seed. In some embodiments DMA #3, #4, #5, and #6, or any single microbe or DMA made of microbes from Table E are used. As an example, application of 1×10⁷ microbes to seeds results in 1×10⁶ to 1×10⁸ CFUs per gram of microgreen.

In other embodiments the nutriobiotics are applied in the nutrient solution and contacted with the crop through the root system. The nutriobiotics dose ranges from 1×10⁴ to 1×10⁹ CFU/ml in nutrient solution.

In further embodiments the nutribiotics are applied to the growth substrate (eg. soil, peat, gel). The nutriobiotics dose ranges from 1×10⁴ to 1×10⁹ CFU/g in growth substrate.

In another embodiment nutriobiotics can be applied to improved seeds that have been specifically selected or bred for growth in microgreen systems.

In another embodiment the nutriobiotic is applied as a foliar product that can be used in combination with any of the other application modalities including seed coats, flower, germination substrate or nutrient solution. The foliar applications can be done at weekly intervals until the crop is harvested. The nutriobiotics dose ranges from 1×10³ to 1×10⁹ CFU/cm² of foliar biomass.

### Polymer Coating

In some embodiments the invention provides a cultured nutriobiotic for the enhancement of microbial content of edible plants including a single or multiple bacteria applied to a seed with a polymeric or adhesive substance as a seed coating to enhance growth of the resultant plant. The nutriobiotics dose ranges from 1×10³ to 1×10⁹ CFU/seed. The seed coating is added as 10-50% weight of the total material added to the seeds.

Polymers can include vinyl pyrrolidone/vinyl acetate copolymers. As an example, suitable polymers include but are not limited to polymers produce by Ashland® (e.g., Agrimer VA 6W). Polymers can be applied to Arugula, Little Gem lettuce, and Black Seeded Simpson lettuce seeds prior to planting and can improve seedling biomass by 20-500%.

In other embodiments the invention provides a cultured nutriobiotic for the enhancement of microbial content of edible plants for probiotic benefit through use of a polymeric or adhesive substance added as part of a formulated spray to enhance microbial survival on fruits, flowers and leaves. The nutriobiotics dose ranges from 1×10³ to 1×10⁹ CFU/cm² of foliar biomass or 1×10³ to 1×10⁹ CFU/flower. The substance is added as 1-50% weight of the total material added to the spray.

In other embodiments the invention provides a cultured nutriobiotic for the enhancement of microbial content of edible plants through use of a polymeric or adhesive substance added as part of a formulated spray to reduce growth of plant pathogens on fruits, flowers and leaves. The nutriobiotics dose ranges from 1×10³ to 1×10⁹ CFU/cm² of foliar biomass or 1×10³ to 1×10⁹ CFU/flower. The substance is added as 1-50% weight of the total material added to the spray.

In other embodiments the invention provides a cultured nutriobiotic for the enhancement of microbial content of edible plants through use of a polymeric or adhesive substance added as part of a formulated spray to enhance growth of fruits, flowers and leaves. The nutriobiotics dose ranges from 1×10³ to 1×10⁹ CFU/cm² of foliar biomass or 1×10³ to 1×10⁹ CFU/flower. The substance is added as 1-50% weight of the total material added to the spray.

In some embodiments the invention provides a cultured nutriobiotic for the enhancement of microbial content of edible plants including a single or multiple bacteria applied to a seed with a polymeric or adhesive substance as a seed coating to enhance growth of the resultant plant.

In other embodiments seed coating is used to enhance growth of the nutriobiotic on the plant and improve the probiotic benefit.

As an example, DMA #2, including L. plantarum, L. brevis, L. mesenteroides and P. kudriavzevii, applied to Little Gem Lettuce seeds with a polymer coating improved colonization of the seedling 4-fold and seedling biomass by 30% over the polymer coating alone.

As a further example, DMA #2, including L. plantarum, L. brevis, L. mesenteroides and P. kudriavzevii, applied to arugula seeds with a polymer coating improved colonization of the seedling 3-fold.

As a further example, DMA #2, including L. plantarum, L. brevis, L. mesenteroides and P. kudriavzevii, applied to Outredgeous seeds with a polymer coating improved colonization of the seedling by 60% and improved biomass over the polymer control by 97%.

As a further example, DMA #2, including L. plantarum, L. brevis, L. mesenteroides and P. kudriavzevii, applied to Outredgcous seeds with a polymer coating improved colonization of the seedling by 30%, improved biomass over the polymer control by 26%, and improved biomass over the non-polymer coated, DMA #2 treated control by 10%.

As a further example, DP100, made of L. plantarum applied to Outredgeous seeds with a polymer coating improved colonization of the seedling by 96%, improved biomass over the polymer control by 88%.

As a further example, DP100, made of L. plantarum applied to Black Seeded Simpson seeds with a polymer coating improved colonization of the seedling by 3.5-fold, improved biomass over the polymer control by 265%, and improved biomass over the non-polymer coated, DP-100 treated control by over 245%.

As a further example, DP100, made of L. plantarum applied to Little Gem seeds with a polymer coating improved colonization of the seedling by 96%, and improved biomass over the non-polymer coated, DP100-treated control by 88%.

As a further example, DP97, made of L. garvieae applied to Black Seeded Simpson seeds with a polymer coating improved colonization of the seedling by 12-fold, improved biomass over the polymer control by 30%, and improved biomass over the non-polymer coated, DP-100 treated control by over 40%.

### Specificity of Microbes on Edible Plants

In some embodiments probiotic microbes applied to fibrous plant material such as salad greens provides consumer benefit over conventional probiotic treatment through introduction of a substrate for protective transport and replication within the digestive tract. Application of probiotic microbes to seeds and replication of microbes on the resultant plants is demonstrated herein (Examples 13-16). Numerous probiotic microbes have been described, each with specific benefits that can be tailored to a given disorder or deficiency. In other embodiments, microbe or DMA Nutriobiotics used to enhance plants can be tailored for these disorders and deficiencies. In example 15, specificity between beneficial microbe and plant substrate for consumption was observed. For microbes applied to greens for use in salad such as arugula and varieties of lettuce, probiotic species selection is a critical component of the art.

As an example, Lactobacillus plantarum (DP100) is a bacterium that is commercially sold as a probiotic. Application of this microbe to seeds results in robust replication on Arugula crops where application of 1×10⁷ bacteria per seed results in 1×10⁸ CFUs per gram of green. Consumption of a salad containing 10-100 g of treated greens would provide microbial CFUs equivalent to current L. plantarum probiotics. This was not true of Outredgeous lettuce where replication of the microbe was 100-fold lower.

As a further example, Leuconostoc mesenteroides (DP93), a bacterium that is generally recognized as safe (GRAS), used in dairy fermentations, and is under investigation as a probiotic with potential use in hypercholesterolemia. Application of this microbe to seeds results in robust replication on Arugula and Little Gem lettuce crops, where application of 1×10⁷ bacteria per seed results in 1×10⁷ CFUs per gram of green. Consumption of a salad containing 100 g of treated greens would provide microbial CFUs equivalent to current L. plantarum probiotics.

As a further example, Debaryomyces hansenii (DP5) is a yeast that has been described as providing human benefit through described immunomodulation and reduction of pathogenic fungi on foods. Application of this microbe to seeds results in robust replication on crops including Arugula, Tomato plants and multiple types of lettuce, where application of 1×10⁶ yeast per seeds results in 1×10⁷ CFUs per gram of green. Consumption of a salad containing 100 g of treated greens would provide microbial CFUs equivalent to commercial yeast probiotics. This would not be true of Black Seeded Simpson lettuce where replication of the microbe was 10-fold lower.

As an example, DMA #2 is comprised of three lactic acid bacteria and a yeast that is under investigation as a therapeutic for bone health. Application of this DMA to seeds results in robust replication on Arugula and Little Gem lettuce crops where application of 1×10⁷ bacteria per seed results in 1×10⁷ CFUs per gram of green. Consumption of a salad containing 100 g of treated greens would provide microbial CFUs equivalent to current L. plantarum probiotics. This was not true of Outredgeous lettuce, where replication of the yeast portion of the DMA was poor or Black Seeded Simpson lettuce, where replication of the lactic acid bacteria was poor.

In other embodiments specific nutriobiotics of single microbes or DMAs from Table E are combined with specifically selected microgreens for maximum microbial replication and consumer benefit. For microbes applied to microgreens such as broccoli, arugula, radishes, cabbage, kale and beet or any conventional vegetable or herbaceous microgreens, probiotic species selection is a critical component of the art.

As an example, Broccoli microgreens are maximally colonized by DMA #5 and DMA #6 while colonization by DMA #3 and DMA #4 was 10-fold lower.

As a further example, Daikon radish microgreens were maximally colonized by DMA #4 whereas DMA #3 colonized to a level that was 10-fold lower and DMA #6 did not colonize at all.

As a further example, Arugula microgreens were maximally colonized by DMA #5 whereas DMA #4 colonized to a level that was 500-fold lower.

### Methods of the Invention

Described herein are methods of modulating the microbial composition of at least a portion of an edible plant comprising heterologously depositing a heterologous microbe to at least a portion of the edible plant (e.g., the seed, seedling, flower, and/or fruit, e.g., berry, of the edible plant) or seed-associated soil environment, in an amount effective to alter the composition of the at least a portion of the edible plant produced by the edible plant relative to a reference edible plant, seed, seedling, or seed-associated soil environment not comprising the heterologous microbe. In certain aspects, the edible plant is selected from a vine crop, a leafy vegetable, a cruciferous vegetable, cucurbits, a root vegetable, and a perennial and annual bush. The amount of heterologous microbe effective to alter the microbial composition the edible plant comprises at least 1×10⁴ CFU/gram, at least 1×10⁵ CFU/gram, at least 1×10⁶ CFU/gram, at least 1×10⁷ CFU/gram, at least 1×10⁸ CFU/gram, at least 1×10⁹ CFU/gram of edible plant tissue.

In certain embodiments, the at least a portion of the edible plant comprises mature fruit. In certain embodiments, the mature fruit comprises at least 1×10⁴ CFU/gram of mature fruit. In certain embodiments, the mature fruit comprises at least 1×10⁴ CFU/gram of mature fruit at least 7 days after depositing the heterologous microbe on the edible plant. In certain embodiments, the mature fruit comprises at least 1×10⁴ CFU/gram of mature fruit at least 7 days after depositing the heterologous microbe on the fruit. In certain embodiments, the mature fruit comprises at least 1×10⁴ CFU/gram of mature fruit at least 17 days after depositing the heterologous microbe on the flower.

Probiotics can be applied to plants of interest by several methods. These methods include, but are not limited to seed treatment, osmopriming, hydropriming, foliar application, soil inoculation, hydroponic inoculation, acroponic inoculation, vector-mediated inoculation root wash, seedling soak, wound inoculation, and injection. These methods are further described in the examples section. Inoculation methods vary by plant type. For vine crops, inoculation can be performed by osmopriming/hydropriming of seeds, foliar application, soil inoculation, vector-mediated inoculation, wound inoculation, and injection. For leafy vegetables, inoculation can be performed by osmopriming/hydropriming of seeds, foliar application, soil inoculation, hydroponic/acroponic inoculation, root wash inoculation and seedling soak inoculation. For cucurbits, inoculation can be performed by osmopriming/hydropriming of seeds, foliar application, soil inoculation, hydroponic/acroponic inoculation, root wash inoculation, seedling soak inoculation, wound inoculation, and injection. For root vegetables, inoculation can be performed by osmopriming/hydropriming of seeds, foliar application, soil inoculation, hydroponic/acroponic inoculation, root wash inoculation, seedling soak inoculation, wound inoculation, and injection. For perennial and annual bushes, inoculation can be performed by osmopriming/hydropriming of seeds, foliar application, soil inoculation, hydroponic/acroponic inoculation, vector-mediated inoculation, root wash inoculation, seedling soak inoculation, wound inoculation, and injection.

In certain aspects, described herein are methods of altering the microbial flora of a subject, the method comprising administering to the subject and effective amount of at least a portion of the edible plant, or nutritive food product described herein. In certain embodiments, the alteration of the microbial flora of the subject improves the health of the subject, reduces the severity of one or more symptoms of a condition or disease in the subject, and/or prevent the onset of one or more conditions or diseases in the subject. In certain embodiments, the methods result in desirable health outcomes such as, but not limited to increased efficacy of anti-diabetic treatments, lowered BMI, lowered inflammatory metabolic indicators, etc.

Included within the scope of this disclosure are methods for use of enhanced plants to enhance wellness in a subject in need thereof. These methods utilize the enhanced plants as a nutritive food product.

These methods optionally are used in combination with other treatments to reduce one or more symptoms of diabetes, obesity, digestive distress, chronic inflammation, bone density loss, and/or metabolic syndrome. Any suitable treatment for the reduction of symptoms of diabetes, obesity, digestive distress, chronic inflammation, bone density loss, and/or metabolic syndrome can be used. In some embodiments, the additional treatment is administered before, during, or after consumption of the microbially enhanced edible plant composition, or any combination thereof. In an embodiment, when diabetes, obesity, digestive distress, chronic inflammation, bone density loss, and/or metabolic syndrome are not completely or substantially completely eliminated by consumption of the microbially enhanced edible plant composition, the additional treatment is administered after prebiotic treatment is terminated. The additional treatment is used on an as-needed basis.

In an embodiment, a subject to be treated for one or more symptoms of obesity, digestive distress, chronic inflammation, bone density loss, and/or metabolic syndrome is a human. In an embodiment the human subject is a preterm newborn, a full-term newborn, an infant up to one year of age, a young child (e.g., 1 yr to 12 yrs), a teenager, (e.g., 13-19 yrs), an adult (e.g., 20-64 yrs), a pregnant woman, or an elderly adult (65 yrs and older).

### ADDITIONAL EMBODIMENTS

Provided below are enumerated embodiments describing specific embodiments of the invention:
Embodiment 1: A probiotic composition comprising a plurality of viable microbes, comprising
   a. At least one microbe classified as a gamma proteobacterium, fungus, or lactic acid bacterium, optionally selected from Table B or Table E, and
   b. At least one prebiotic, optionally wherein the prebiotic is a fiber; and
   c. An agriculturally acceptable carrier
Embodiment 2: The probiotic composition of embodiment 1, wherein the probiotic composition comprises a filamentous fungus or yeast.
Embodiment 3: The probiotic composition of embodiment 1, wherein the probiotic composition comprises a lactic acid bacterium.
Embodiment 4: The probiotic composition of embodiment 1, wherein the probiotic composition is substantially similar to that of an edible plant component that is beneficial for human health.
Embodiment 5: The probiotic of embodiment 1, wherein the plurality of purified microbes is present at an amount effective to improve the microbial content of an edible plant.
Embodiment 6: The probiotic composition of embodiment 1, wherein the plurality of purified viable microbes produces more short chain fatty acids than the individual microbial entities grown in isolation.
Embodiment 7: The probiotic composition of embodiment 1, applied to an edible portion of a plant, wherein the probiotic composition increases the amount of beneficial microbes in the edible portion of the plant treated with the probiotic composition.
Embodiment 8: The probiotic composition of embodiment 1, wherein the microbial entities comprising the probiotic composition are amplified within a tissue of an edible plant.
Embodiment 9: A method of improving the nutritional value of a first plant component, comprising i) applying to a second plant component an effective amount of a plurality of viable microbes, ii) allowing the first plant component to mature, and iii) harvesting the first plant component, wherein the plurality of microbes is present in the first plant component at harvest at higher amounts than in the first plant component allowed to mature without the addition of the effective amount of the plurality of microbes.
Embodiment 10: The method of embodiment 9, wherein the plurality of microbes comprises two or more microbes listed in Table B or Table E.
Embodiment 11: The method of embodiment 9, wherein the plurality of microbes comprises three or more microbes listed in Table B or Table E.
Embodiment 12: The method of embodiment 9, wherein the first plant component is a fruit.
Embodiment 13: The method of embodiment 9, wherein the first plant component is a stem, leaf, root or tuber.
Embodiment 14: The method of embodiment 9, wherein the second plant component is a flower.
Embodiment 15: The method of embodiment 9, wherein the second plant component is a seed.
Embodiment 16: The method of embodiment 9, wherein the second plant component is a root.
Embodiment 17: The method of embodiment 9, wherein the second plant component is a leaf.
Embodiment 18: The method of embodiment 9, wherein the second plant component is a stem.
Embodiment 19: The method of embodiment 9, wherein the second plant component is a seedling.
Embodiment 20: The method of embodiment 9, further comprising improving a facet of the first plant component for human consumption.
Embodiment 21: The method of embodiment 20, wherein the improved facet is selected from the group consisting of: plant growth, germination efficiency, abiotic stress tolerance, nutritional value, taste, smell, texture, digestibility, and shelf-life.
Embodiment 22: An agricultural seed preparation prepared by the method of embodiment 9.
Embodiment 23: A plant component wherein the microbial content of the plant component comprises higher microbial diversity or higher amounts by viable count or direct microscopy, as compared to a reference sample.
Embodiment 24: The method of embodiment 9, wherein the plurality of viable microbes is obtained from a plant species or plant component other than the seeds to which the plurality of microbes is applied.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

### Example 1: Microbial Preparations and Metagenomic Analyses

A sample set of 15 vegetables typically eaten raw was selected to analyze the microbial communities by whole genome shotgun sequencing and comparison to microbial databases. The 15 fruits and vegetable samples are shown in Table A and represent ingredients in typical salads or eaten fresh. The materials were sourced at the point of distribution in supermarkets selling both conventional and organic farmed vegetables, either washed and ready to cat or without washing.

The samples were divided into 50 g portions, thoroughly rinsed with tap water and blended for 30 seconds on phosphate buffer pH 7.4 (PBS) in a household blender. The resulting slurry was strained by serial use of a coarse household sieve and then a fine household sieve followed by filtration through a 40 μm sieve. The cell suspension containing the plant microbiota, chloroplasts and plant cell debris was centrifuged at slow speed (100×g) 5 minutes for removing plant material and the resulting supernatant centrifuged at high speed (4000× g) 10 minutes to pellet microbial cells. The pellet was resuspended in a plant cell lysis buffer containing a chelator such as EDTA 10 mM to reduce divalent cation concentration to less than, and a non-ionic detergent to lyse the plant cells without destroying the bacterial cells. The lysed material was washed by spinning down the microbial cells at 4000× g for 10 minutes, and then resuspended in PBS and repelleted as above. For sample #12 (broccoli) the cell pellet was washed and a fraction of the biomass separated and only the top part of the pellet collected. This was deemed “broccoli juice” for analyses. The resulting microbiota prep was inspected under fluorescence microscopy with DNA stains to visualize plant and microbial cells based on cell size and DNA structure (nuclei for plants) and selected for DNA isolation based on a minimum ratio of 9:1 microbe to plant cells. The DNA isolation was based on the method reported by Marmur (Journal of Molecular Biology 3, 208-218; 1961), or using commercial DNA extraction kits based on magnetic beads such as Thermo Charge Switch resulting in a quality suitable for DNA library prep and free of PCR inhibitors.

The DNA was used to construct a single read 150 base pair libraries and a total of 26 million reads sequenced per sample according to the standard methods done by CosmosID (www.cosmosid.com) for samples #1 to #12 or 300 base pair-end libraries and sequenced in an Illumina NextSeq instrument covering 4 Gigabases per sample for samples #13 to #15. The unassembled reads were then mapped to the CosmosID for first 12 samples or OneCodex for the last 3 samples databases containing 36,000 reference bacterial genomes covering representative members from diverse taxa. The mapped reads were tabulated and represented using a “sunburst” plot to display the relative abundance for each genome identified corresponding to that bacterial strain and normalized to the total of identified reads for each sample. In addition, phylogenetic trees were constructed based on the classification for each genome in the database with a curated review. There are genomes that have not been updated in the taxonomic classifier and therefore reported as unclassified here but it does not reflect a true lack of clear taxonomic position, it reflects only the need for manual curation and updating of those genomes in the taxonomic classifier tool.

In addition to the shotgun metagenomics survey relevant microbes were isolated from fruits and vegetables listed in Table A using potato dextrose agar or nutrient agar and their genomes sequenced to cover 50× and analyzed their metabolic potential by using genome-wide models. For example, a yeast isolated from blueberries was sequenced and its genome showed identity to Aureobasidium subglaciale assembled in contigs with an N50 of 71 Kb and annotated to code for 10, 908 genes. Similarly, bacterial genomes from the same sample were sequenced and annotated for strains with high identity to Pseudomonas and Rahnella.

TABLE A
Samples analyzed.

| Sample number | sample description |
|---|---|
| 1 | chard |
| 2 | red cabbage |
| 3 | organic romaine |
| 4 | organic celery |
| 5 | butterhead organic lettuce |
| 6 | organic baby spinach |
| 7 | crisp green gem lettuce |
| 8 | red oak leaf lettuce |
| 9 | green oak leaf lettuce |
| 10 | cherry tomato |
| 11 | crisp red gem lettuce |
| 12 | broccoli juice |
| 13 | broccoli head |
| 14 | blueberries |
| 15 | pickled olives |

### Results

For most samples, bacterial abundances of fresh material contain 10⁴ to 10⁸ microbes per gram of vegetable as estimated by direct microscopy counts or viable counts. Diverse cell morphologies were observed including rods, elongated rods, cocci and fungal hyphae. Microorganisms were purified from host cells, DNA was isolated and sequenced using a shotgun approach mapping reads to 35,000 bacterial genomes using a k-mer method. All samples were dominated by gamma proteobacteria, primarily Pseudomonadacea, presumably largely endophytes as some samples were triple washed before packaging. Pseudomonas cluster was the dominant genera for several samples with 10-90% of the bacterial relative abundance detected per sample and mapped to a total of 27 different genomes indicating it is a diverse group. A second relevant bacterial strain identified was Duganella zoogloeoides ATCC 25935 as it was present in almost all the samples ranging from 1-6% of the bacterial relative abundance detected per sample or can reach 29% of the bacterial relative abundance detected per sample in organic romaine. Red cabbage was identified to contain a relatively large proportion of lactic acid bacteria as it showed 22% Lactobacillus crispatus, a species commercialized as probiotic and recognized relevant in vaginal healthy microbial community. Another vegetable containing lactic acid bacteria was red oak leaf lettuce containing 1.5% of the bacterial relative abundance detected per sample Lactobacillus reuteri. Other bacterial species recognized as probiotics included Bacillus, Bacteroidetes, Propionibacterium and Streptococcus. A large proportion of the abundant taxa in most samples was associated with plant microbiota and members recognized to act as biocontrol agents against fungal diseases or growth promoting agents such as Pseudomonas fluorescens. The aggregated list of unique bacteria detected by the k-mer method is 318 (Table B).

Blueberries contain a mixture of bacteria and fungi dominated by Pseudomonas and Propionibacterium but the yeast Aureobasidium was identified as a relevant member of the community. A lesser abundant bacterial species was Rahnella. Pickled olives are highly enriched in lactic acid bacteria after being pickled in brine allowing the endogenous probiotic populations to flourish by acidifying the environment and eliminating most of the acid-sensitive microbes including bacteria and fungi. This resulted in a large amount of Lactobacillus species and Pediococcus recognized as probiotics and related to obesity treatment.

The shotgun sequencing method allows for the analysis of the metagenome including genes coding for metabolic reactions involved in the assimilation of nutrient, fermentative processes to produce short chain fatty acids, flavonoids and other relevant molecules in human nutrition.

TABLE B
Bacteria identified in a 15 sample survey identified by whole genome
matching to reference genomes. The fruits and vegetables were selected
based on their recognition as part of the whole food plant-based diet
and some antidiabetic and anti -obesogenic properties. There is general
recognition of microbes in these vegetables relevant for plant health
but not previously recognized for their use in human health.

| | Strain | |
|---|---|---|
| Strain identified by k-mer based on entire genome | number | Collection |
| Acinetobacter baumannii | — | |
| Acinetobacter soli | — | |
| Acinetobacter 41764 Branch | — | |
| Acinetobacter 41930 Branch | — | |
| Acinetobacter 41981 Branch | — | |
| Acinetobacter 41982 Branch | — | |
| Acinetobacter baumannii 348935 | — | |
| Acinetobacter baumannii 40298 Branch | — | |
| Acinetobacter beijerinckii 41969 Branch | — | |
| Acinetobacter beijerinckii CIP 110307 | CIP 110307 | WFCC |
| Acinetobacter bohemicus ANC 3994 | — | |
| Acinetobacter guillouiae 41985 Branch | — | |
| Acinetobacter guillouiae 41986 Branch | — | |
| Acinetobacter gyllenbergii 41690 Branch | — | |
| Acinetobacter haemolyticus TG19602 | — | |
| Acinetobacter harbinensis strain HITLi 7 | — | |
| Acinetobacter johnsonii 41886 Branch | — | |
| Acinetobacter johnsonii ANC 3681 | — | |
| Acinetobacter junii 41994 Branch | — | |
| Acinetobacter lwoffii WJ10621 | — | |
| Acinetobacter sp 41945 Branch | — | |
| Acinetobacter sp 41674 Branch | — | |
| Acinetobacter sp 41698 Branch | — | |
| Acinetobacter sp ETR1 | — | |
| Acinetobacter sp NIPH 298 | — | |
| Acinetobacter tandoii 41859 Branch | — | |
| Acinetobacter tjernbergiae 41962 Branch | — | |
| Acinetobacter towneri 41848 Branch | — | |
| Acinetobacter venetianus VE C3 | — | |
| Actinobacterium LLX17 | — | |
| Aeromonas bestiarum strain CECT 4227 | CECT 4227 | CECT |
| Aeromonas caviae strain CECT 4221 | CECT 4221 | CECT |
| Aeromonas hydrophila 4AK4 | — | |
| Aeromonas media 37528 Branch | — | |
| Aeromonas media strain ARB 37524 Branch | — | |
| Aeromonas salmonicida subsp 37538 Branch | — | |
| Aeromonas sp ZOR0002 | — | |
| Agrobacterium 22298 Branch | — | |
| Agrobacterium 22301 Branch | — | |
| Agrobacterium 22313 Branch | — | |
| Agrobacterium 22314 Branch | — | |
| Agrobacterium sp ATCC 31749 | ATCC 31749 | ATCC |
| Agrobacterium tumefaciens 22306 Branch | | |
| Agrobacterium tumefaciens strain MEJ076 | — | |
| Agrobacterium tumefaciens strain S2 | — | |
| Alkanindiges illinoisensis DSM 15370 | DSM 15370 | WFCC |
| alpha proteobacterium L41A | — | |
| Arthrobacter 20515 Branch | — | |
| Arthrobacter arilaitensis Re117 | — | |
| Arthrobacter chlorophenolicus A6 | — | |
| Arthrobacter nicotinovorans 20547 Branch | — | |
| Arthrobacter phenanthrenivorans Sphe3 | — | |
| Arthrobacter sp 20511 Branch | — | |
| Arthrobacter sp PAO19 | — | |
| Arthrobacter sp W1 | — | |
| Aureimonas sp. Leaf427 | — | |
| Aureobasidium pullulans | — | |
| Bacillaceae Family 24 4101 12691 Branch | — | |
| Bacillus sp. LL01 | — | |
| Bacillus 12637 Branch | — | |
| Bacillus aerophilus strain C772 | — | |
| Bacillus thuringiensis serovar 12940 Branch | — | |
| Brevundimonas nasdae strain TPW30 | — | |
| Brevundimonas sp 23867 Branch | — | |
| Brevundimonas sp EAKA | — | |
| Buchnera aphidicola str 28655 Branch | — | |
| Burkholderiales Order 15 6136 Node 25777 | — | |
| Buttiauxella agrestis 35837 Branch | — | |
| Candidatus Burkholderia verschuerenii | — | |
| Carnobacterium 5833 Branch | — | |
| Carnobacterium maltaromaticum ATCC 35586 | ATCC 35586 | ATCC |
| Chryseobacterium 285 Branch | — | |
| Chryseobacterium daeguense DSM 19388 | DSM 19388 | WFCC |
| Chryseobacterium formosense | — | |
| Chryseobacterium sp YR005 | — | |
| Clavibacter 20772 Branch | — | |
| Clostridium diolis DSM 15410 | DSM 15410 | WFCC |
| Comamonas sp B 9 | — | |
| Curtobacterium flaccumfaciens 20762 Branch | — | |
| Curtobacterium flaccumfaciens UCD AKU | — | |
| Curtobacterium sp UNCCL17 | — | |
| Deinococcus aquatilis DSM 23025 | DSM 23025 | WFCC |
| Debaromyces hansenii ATCC 36239 | ATCC 25935 | ATCC |
| Duganella zoogloeoides ATCC 25935 | | |
| Dyadobacter 575 Branch | — | |
| Elizabethkingia anophelis | — | |
| Empedobacter falsenii strain 282 | — | |
| Enterobacter sp 638 | — | |
| Enterobacteriaceae Family 9 3608 Node 35891 | — | |
| Enterobacteriaceae Family 9 593 Node 36513 | — | |
| Epilithonimonas lactis | — | |
| Epilithonimonas tenax DSM 16811 | DSM 16811 | WFCC |
| Erwinia 35491 Branch | — | |
| Erwinia amylovora 35816 Branch | — | |
| Erwinia pyrifoliae 35813 Branch | — | |
| Erwinia tasmaniensis Et1 99 | DSM 17950 | WFCC |
| Escherichia coli ISC11 | — | |
| Exiguobacterium 13246 Branch | — | |
| Exiguobacterium 13260 Branch | — | |
| Exiguobacterium sibiricum 255 15 | DSM 17290 | WFCC |
| Exiguobacterium sp 13263 Branch | — | |
| Exiguobacterium undae 13250 Branch | — | |
| Exiguobacterium undae DSM 14481 | DSM 14481 | WFCC |
| Flavobacterium 237 Branch | — | |
| Flavobacterium aquatile LMG 4008 | LMG 4008 | WFCC |
| Flavobacterium chungangense LMG 26729 | LMG 26729 | WFCC |
| Flavobacterium daejeonense DSM 17708 | DSM 17708 | WFCC |
| Flavobacterium hibernum strain DSM 12611 | DSM 12611 | WFCC |
| Flavobacterium hydatis | — | |
| Flavobacterium johnsoniae UW101 | ATCC 17061D-5 | ATCC |
| Flavobacterium reichenbachii | — | |
| Flavobacterium soli DSM 19725 | DSM 19725 | WFCC |
| Flavobacterium sp 238 Branch | — | |
| Flavobacterium sp EM1321 | — | |
| Flavobacterium sp MEB061 | — | |
| Hanseniaspora uvarum ATCC 18859 | — | |
| Hanseniaspora occidentalis ATCC 32053 | | |
| Herminiimonas arsenicoxydans | | |
| Hymenobacter swuensis DY53 | — | |
| Janthinobacterium 25694 Branch | — | |
| Janthinobacterium agaricidamnosum | DSM 9628 | WFCC |
| Janthinobacterium lividum strain RIT308 | — | |
| Janthinobacterium sp RA13 | — | |
| Kocuria 20614 Branch | — | |
| Kocuria rhizophila 20623 Branch | — | |
| Lactobacillus acetotolerans | — | |
| Lactobacillus brevis | — | |
| Lactobacillus buchneri | — | |
| Lactobacillus futsaii | — | |
| Lactobacillus kefiranofaciens | — | |
| Lactobacillus panis | — | |
| Lactobacillus parafarraginis | — | |
| Lactobacillus plantarum | — | |
| Lactobacillus rapi | — | |
| Lactobacillus crispatus 5565 Branch | — | |
| Lactobacillus plantarum WJL | — | |
| Lactobacillus reuteri 5515 Branch | — | |
| Leuconostoc mesenteroides ATCC 8293 | — | |
| Luteibacter sp 9135 | | |
| Massilia timonae CCUG 45783 | — | |
| Methylobacterium extorquens 23001 Branch | — | |
| Methylobacterium sp 22185 Branch | — | |
| Methylobacterium sp 285MFTsu5 1 | — | |
| Methylobacterium sp 88A | — | |
| Methylotenera versatilis 7 | — | |
| Microbacterium laevaniformans OR221 | — | |
| Microbacterium oleivorans | — | |
| Microbacterium sp MEJ108Y | — | |
| Microbacterium sp UCD TDU | — | |
| Microbacterium testaceum StLB037 | — | |
| Micrococcus luteus strain RIT304 | NCTC 2665 | NCTC |
| Mycobacterium abscessus 19573 Branch | — | |
| Neosartorya fischeri | — | |
| Oxalobacteraceae bacterium AB 14 | — | |
| Paenibacillus sp FSL 28088 Branch | — | |
| Paenibacillus sp FSL H7 689 | — | |
| Pantoea sp. SL1 M5 | — | |
| Pantoea 36041 Branch | — | |
| Pantoea agglomerans strain 4 | — | |
| Pantoea agglomerans strain 4 | — | |
| Pantoea agglomerans strain LMAE 2 | — | |
| Pantoea agglomerans Tx10 | — | |
| Pantoea sp 36061 Branch | — | |
| Pantoea sp MBLJ3 | — | |
| Pantoea sp SL1 M5 | — | |
| Paracoccus sp PAMC 22219 | — | |
| Patulibacter minatonensis DSM 18081 | DSM 18081 | WFCC |
| Pectobacterium carotovorum subsp carotovorum | — | |
| strain 28625 Branch | | |
| Pediococcus ethanolidurans | — | |
| Pediococcus pentosaceus ATCC 33314 | — | |
| Pedobacter 611 Branch | | |
| Pedobacter agri PB92 | — | |
| Pedobacter borealis DSM 19626 | DSM 19626 | WFCC |
| Pedobacter kyungheensis strain KACC 16221 | — | |
| Pedobacter sp R20 19 | — | |
| Periglandula ipomoeae | — | |
| Pichia kudriavzevii | — | |
| Planomicrobium glaciei CHR43 | | |
| Propionibacterium acnes | — | |
| Propionibacterium 20955 Branch | — | |
| Propionibacterium acnes 21065 Branch | — | |
| Pseudomonas fluorescens | — | |
| Pseudomonas sp. DSM 29167 | — | |
| Pseudomonas sp. Leaf15 | — | |
| Pseudomonas syringae | — | |
| Pseudomonas 39524 Branch | — | |
| Pseudomonas 39642 Branch | — | |
| Pseudomonas 39733 Branch | — | |
| Pseudomonas 39744 Branch | — | |
| Pseudomonas 39791 Branch | — | |
| Pseudomonas 39821 Branch | — | |
| Pseudomonas 39834 Branch | — | |
| Pseudomonas 39875 Branch | — | |
| Pseudomonas 39880 Branch | — | |
| Pseudomonas 39889 Branch | — | |
| Pseudomonas 39894 Branch | — | |
| Pseudomonas 39913 Branch | — | |
| Pseudomonas 39931 Branch | — | |
| Pseudomonas 39942 Branch | — | |
| Pseudomonas 39979 Branch | — | |
| Pseudomonas 39996 Branch | — | |
| Pseudomonas 40058 Branch | — | |
| Pseudomonas 40185 Branch | — | |
| Pseudomonas abietaniphila strain KF717 | — | |
| Pseudomonas chlororaphis strain EA105 | — | |
| Pseudomonas cremoricolorata DSM 17059 | DSM 17059 | WFCC |
| Pseudomonas entomophila L48 | — | |
| Pseudomonas extremaustralis 14 3 substr 14 3b | — | |
| Pseudomonas fluorescens BBc6R8 | — | |
| Pseudomonas fluorescens BS2 | ATCC 12633 | ATCC |
| Pseudomonas fluorescens EGD AQ6 | — | |
| Pseudomonas fluorescens strain AU 39831 Branch | — | |
| Pseudomonas fluorescens strain AU10973 | — | |
| Pseudomonas fluorescens strain AU14440 | — | |
| Pseudomonas fragi B25 | NCTC 10689 | NCTC |
| Pseudomonas frederiksbergensis strain SI8 | — | |
| Pseudomonas fulva strain MEJ086 | — | |
| Pseudomonas fuscovaginae 39768 Branch | — | |
| Pseudomonas gingeri NCPPB 3146 | NCPPB 3146 | NCPPB |
| Pseudomonas lutea | — | |
| Pseudomonas luteola XLDN4 9 | — | |
| Pseudomonas mandelii JR 1 | — | |
| Pseudomonas moraviensis R28 S | — | |
| Pseudomonas mosselii SJ10 | — | |
| Pseudomonas plecoglossicida NB 39639 Branch | — | |
| Pseudomonas poae RE*1 1 14 | — | |
| Pseudomonas pseudoalcaligenes AD6 | — | |
| Pseudomonas psychrophila HA 4 | — | |
| Pseudomonas putida DOT T1E | — | |
| Pseudomonas putida strain KF703 | — | |
| Pseudomonas putida strain MC4 5222 | — | |
| Pseudomonas rhizosphaerae | — | |
| Pseudomonas rhodesiae strain FF9 | — | |
| Pseudomonas sp 39813 Branch | — | |
| Pseudomonas simiae strain 2 36 | — | |
| Pseudomonas simiae strain MEB105 | — | |
| Pseudomonas sp 11 12A | — | |
| Pseudomonas sp 2 922010 | — | |
| Pseudomonas sp CF149 | — | |
| Pseudomonas sp Eur1 9 41 | — | |
| Pseudomonas sp LAMO17WK12 I2 | — | |
| Pseudomonas sp PAMC 25886 | — | |
| Pseudomonas sp PTA1 | — | |
| Pseudomonas sp R62 | — | |
| Pseudomonas sp WCS374 | — | |
| Pseudomonas synxantha BG33R | — | |
| Pseudomonas synxantha BG33R | — | |
| Pseudomonas syringae 39550 Branch | — | |
| Pseudomonas syringae 39596 Branch | — | |
| Pseudomonas syringae 40123 Branch | — | |
| Pseudomonas syringae CC 39499 Branch | — | |
| Pseudomonas syringae pv panici str LMG 2367 | — | |
| Pseudomonas syringae strain mixed | — | |
| Pseudomonas tolaasii 39796 Branch | — | |
| Pseudomonas tolaasii PMS117 | — | |
| Pseudomonas veronii 1YdBTEX2 | — | |
| Pseudomonas viridiflava CC1582 | — | |
| Pseudomonas viridiflava strain LMCA8 | — | |
| Pseudomonas viridiflava TA043 | — | |
| Pseudomonas viridiflava UASWS0038 | — | |
| Rahnella 35969 Branch | — | |
| Rahnella 35970 Branch | — | |
| Rahnella 35971 Branch | — | |
| Rahnella aquatilis HX2 | — | |
| Rahnella sp WP5 | — | |
| Raoultella ornithinolytica | — | |
| Rhizobiales Order 22324 Branch | — | |
| Rhizobium sp YR528 | — | |
| Rhodococcus fascians A76 | — | |
| Rhodococcus sp BS 15 | — | |
| Saccharomyces cerevisiae | DSM 10542 | WFCC |
| Sanguibacter keddieii DSM 10542 | | |
| Serratia fonticola AU 35657 Branch | — | |
| Serratia fonticola AU AP2C | — | |
| Serratia liquefaciens ATCC 27592 | ATCC 27592 | ATCC |
| Serratia sp H 35589 Branch | — | |
| Shewanella 37294 Branch | — | |
| Shewanella baltica 37301 Branch | — | |
| Shewanella baltica 37315 Branch | — | |
| Shewanella baltica OS 37308 Branch | — | |
| Shewanella baltica OS 37312 Branch | — | |
| Shewanella baltica OS185 | — | |
| Shewanella baltica OS223 | — | |
| Shewanella baltica OS678 | — | |
| Shewanella oneidensis MR 1 | — | |
| Shewanella putrefaciens HRCR 6 | — | |
| Shewanella sp W3 18 1 | — | |
| Sphingobacterium sp ML3W | — | |
| Sphingobium japonicum BiD32 | — | |
| Sphingobium xenophagum 24443 Branch | — | |
| Sphingomonas echinoides ATCC 14820 | ATCC 14820 | ATCC |
| Sphingomonas parapaucimobilis NBRC 15100 | ATCC 51231 | ATCC |
| Sphingomonas paucimobilis NBRC 13935 | ATCC 29837 | ATCC |
| Sphingomonas phyllosphaerae 5 2 | — | |
| Sphingomonas sp 23777 Branch | — | |
| Sphingomonas sp STIS6 2 | — | |
| Staphylococcus 6317 Branch | — | |
| Staphylococcus equorum UMC CNS 924 | — | |
| Staphylococcus sp 6275 Branch | — | |
| Staphylococcus sp 6240 Branch | — | |
| Staphylococcus sp OJ82 | — | |
| Staphylococcus xylosus strain LSR 02N | — | |
| Stenotrophomonas 14028 Branch | — | |
| Stenotrophomonas 42816 Branch | — | |
| Stenotrophomonas maltophilia 42817 Branch | — | |
| Stenotrophomonas maltophilia PML168 | — | |
| Stenotrophomonas maltophilia strain ZBG7B | — | |
| Stenotrophomonas rhizophila | — | |
| Stenotrophomonas sp RIT309 | — | |
| Streptococcus gallolyticus subsp | — | |
| gallolyticus TX20005 | | |
| Streptococcus infantarius subsp | — | |
| infantarius 2242 Branch | | |
| Streptococcus infantarius subsp | ATCC BAA 102 | ATCC |
| infantarius ATCC BAA 102 | | |
| Streptococcus macedonicus ACA DC 198 | ATCC BAA-249 | ATCC |
| Streptomyces olindensis | — | |
| Variovorax paradoxus 110B | — | |
| Variovorax paradoxus ZNC0006 | — | |
| Variovorax sp CF313 | — | |
| Vibrio fluvialis 44473 Branch | — | |
| Xanthomonas campestris 37936 Branch | — | |
| Xanthomonas campestris pv raphani 756C | — | |

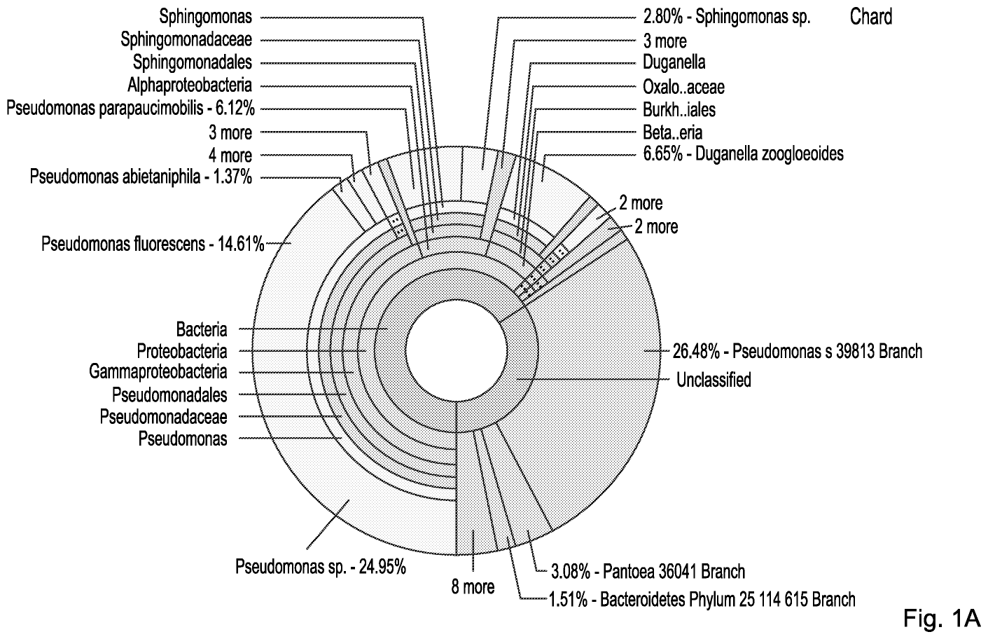

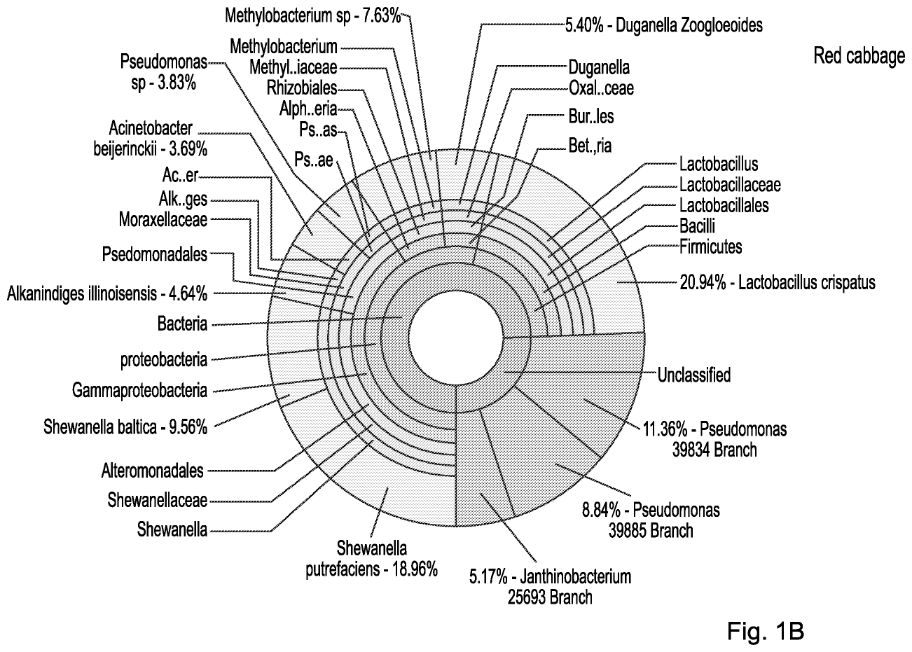

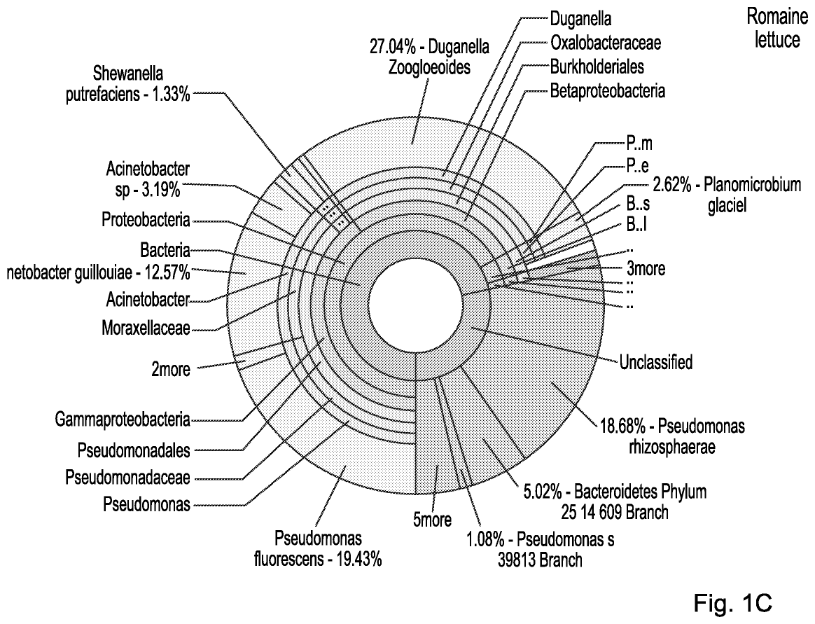

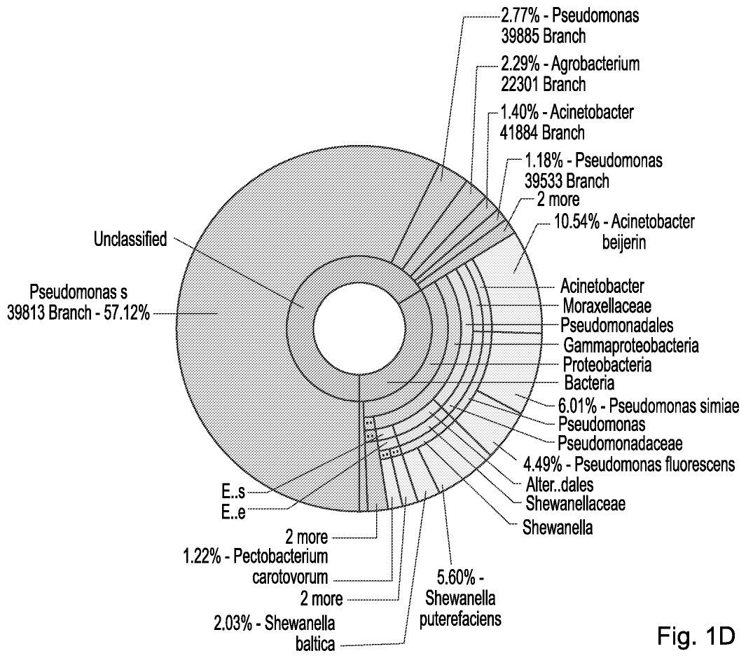

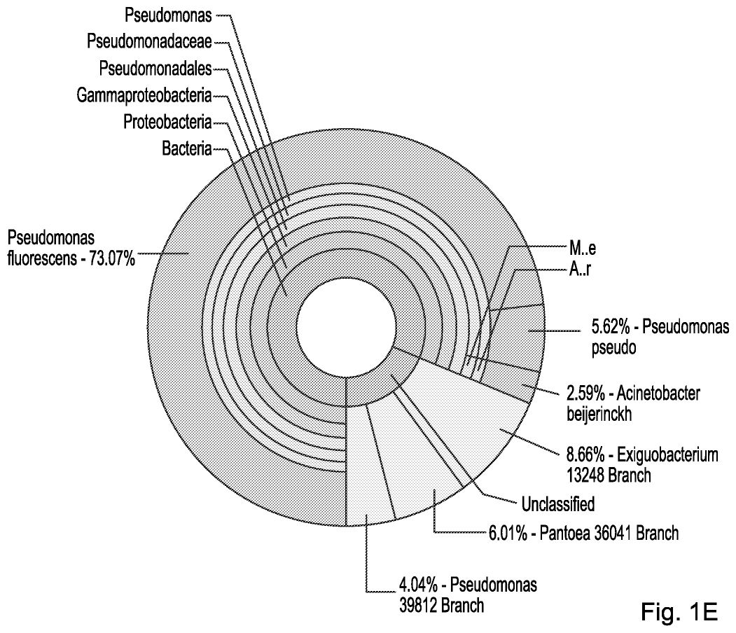

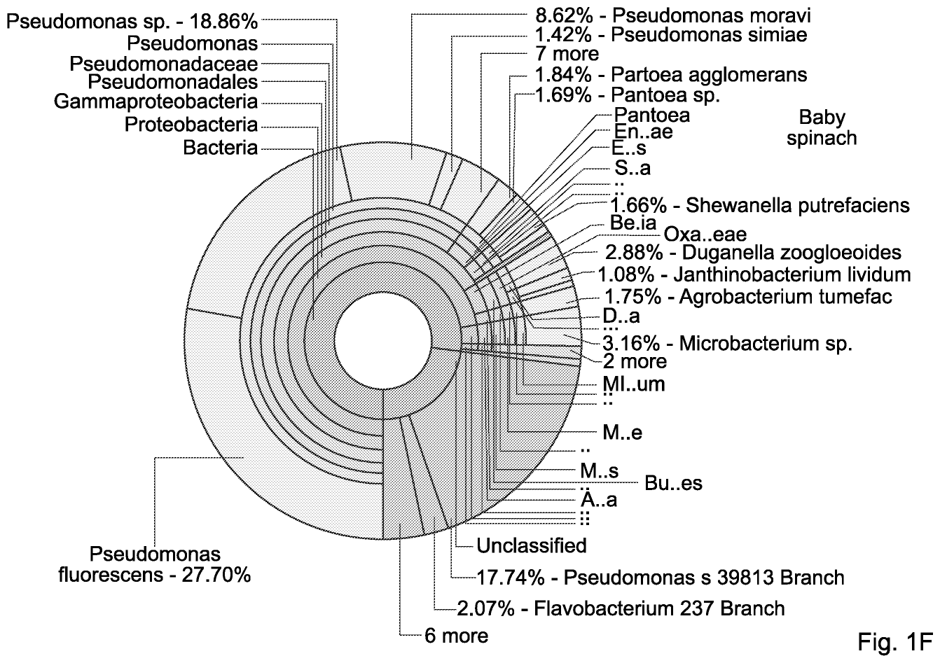

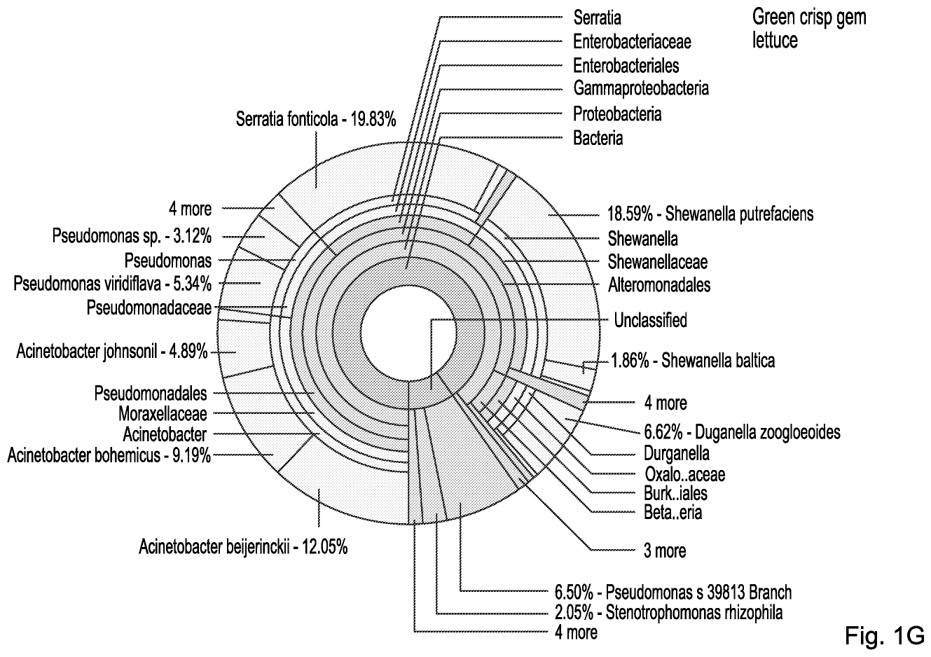

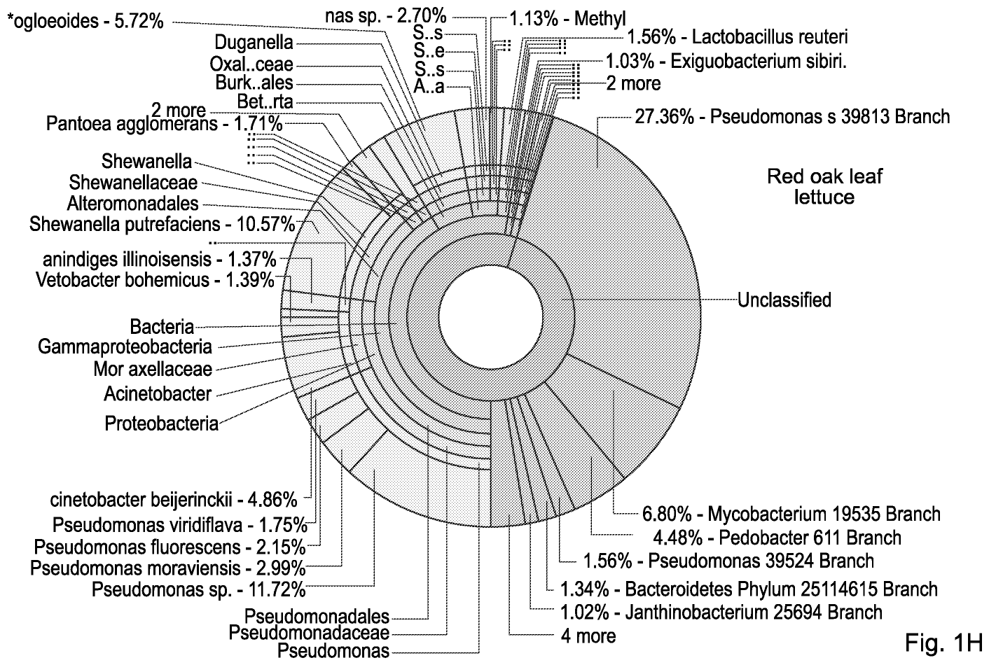

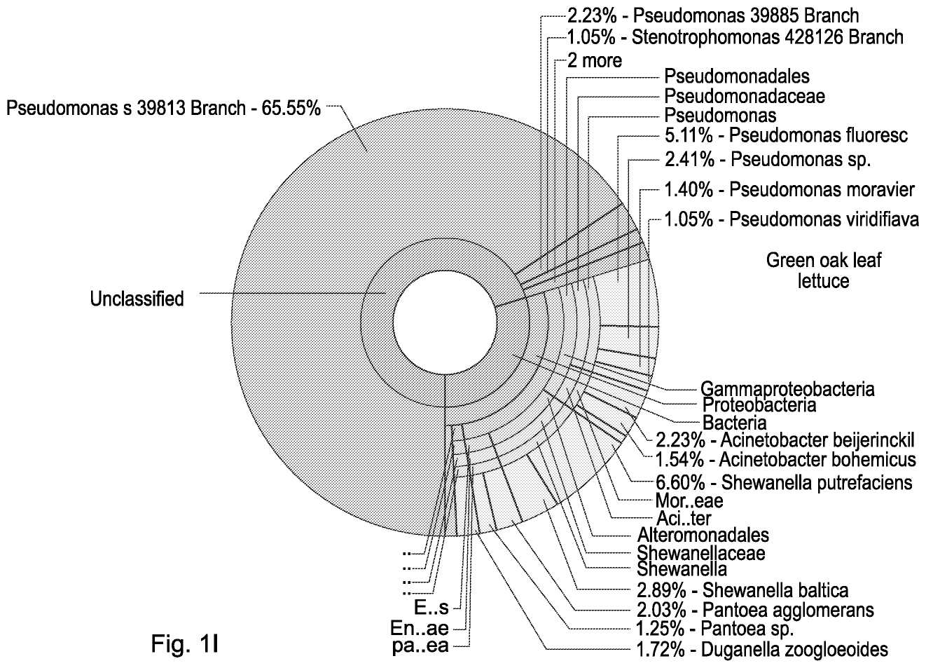

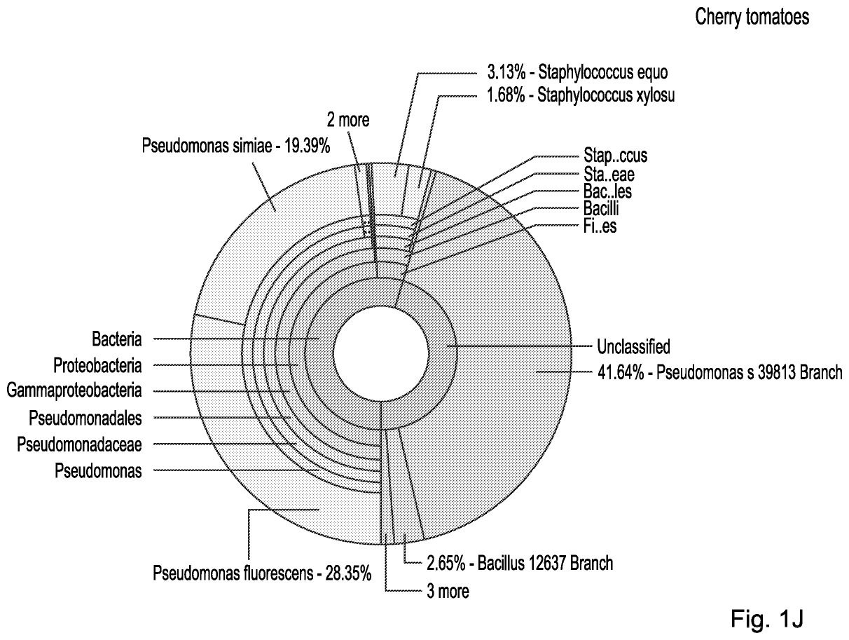

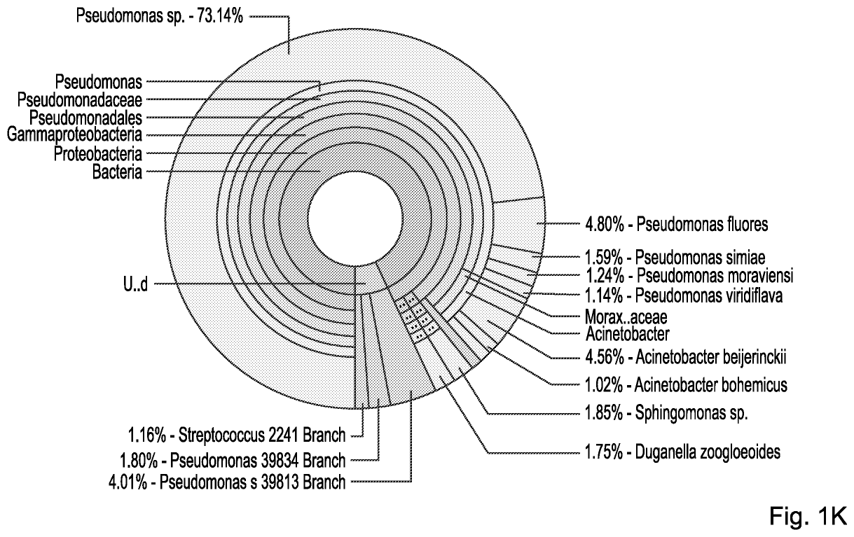

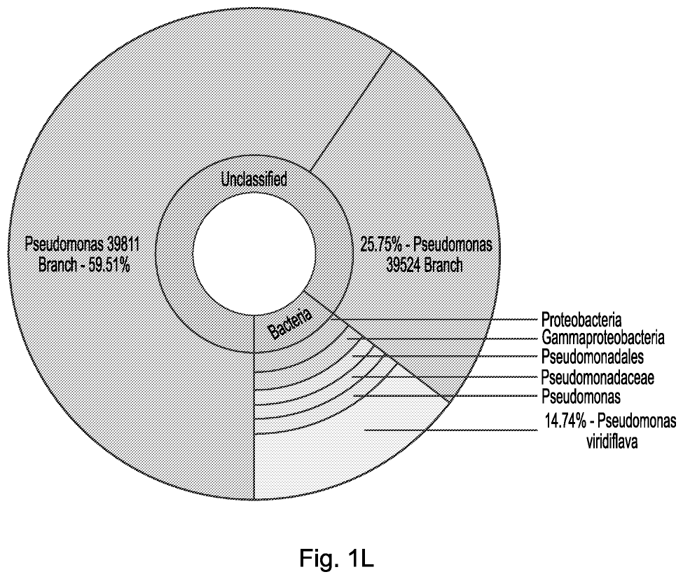

FIG. 1 shows bacterial diversity observed in a set of 12 plant-derived samples as seen by a community reconstruction based on mapping the reads from a shotgun sequencing library into the full genomes of a database containing 36,000 genomes by the k-mer method (CosmosID). The display corresponds to a sunburst plot constructed with the relative abundance for each corresponding genome identified and their taxonomic classification. The genomes identified as unclassified have not been curated in the database with taxonomic identifiers and therefore not assigned to a group. This does not represent novel taxa and it is an artifact of the database updating process.

More specifically, FIG. 1A shows bacterial diversity observed in a green chard. The dominant group is gamma proteobacteria with different Pseudomonas species. The members of the group “unclassified” are largely gamma proteobacteria not included in the hierarchical classification as an artifact of the database annotation.

FIG. 1B shows bacterial diversity in red cabbage. There is a large abundance of Lactobacillus in the sample followed by a variety of Pseudomonas and Shewanella.

FIG. 1C shows bacterial diversity in romaine lettuce. Pseudomonas and Duganella are the dominant groups. A member of the Bacteroidetes was also identified.

FIG. 1D shows bacterial diversity in celery sticks. This sample was dominated by a Pseudomonas species that was not annotated yet into the database and therefore appeared as “unclassified” same for Agrobacterium and Acinetobacter.

FIG. 1E shows bacterial diversity observed in butterhead lettuce grown hydroponically. The sample contains relatively low bacterial complexity dominated by Pseudomonas fluorescens and other groups. Also, there was a 9% abundance of Exiguobacterium.

FIG. 1F shows bacterial diversity in organic baby spinach. The samples were triple-washed before distribution at the point of sale and therefore it is expected that must of the bacteria detected here are endophytes. Multiple Pseudomonas species observed in this sample including P. fluorescens and other shown as “unclassified.”

FIG. 1G shows bacterial diversity in green crisp gem lettuce. This variety of lettuce showed clear dominance of gamma protcobacteria and with Pseudomonas, Shewanella, Serratia as well as other groups such as Duganella.

FIG. 1H shows bacterial diversity in red oak leaf lettuce. There is a relative high diversity represented in this sample with members of Lactobacillus, Microbacterium, Bacteroidetes, Exiguobacterium and a variety of Pseudomonas.

FIG. 1I shows bacterial diversity in green oak leaf lettuce. It is dominated by a single Pseudomonas species including fluorescens and mostly gamma proteobacteria.

FIG. 1J shows bacterial diversity in cherry tomatoes. It is dominated by 3 species of Pseudomonas comprising more than 85% of the total diversity of which P. fluorescens comprised 28% of bacterial diversity.

FIG. 1K shows bacterial diversity in crisp red gem lettuce. Dominance by a single Pseudomonas species covering 73% of the bacterial diversity, of which P. fluorescens comprised 5% of bacterial diversity.

FIG. 1L shows bacterial diversity in broccoli juice. The sample is absolutely dominated by 3 varieties of Pseudomonas.

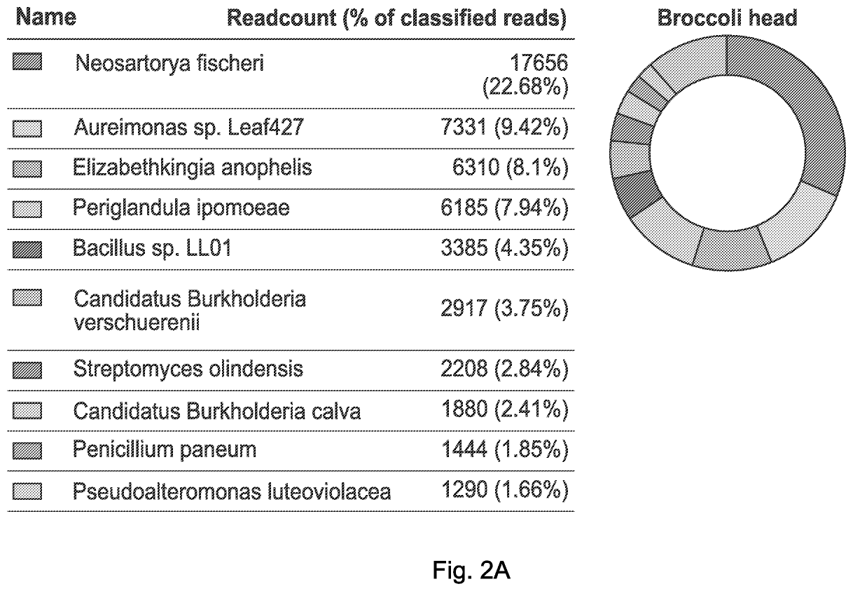

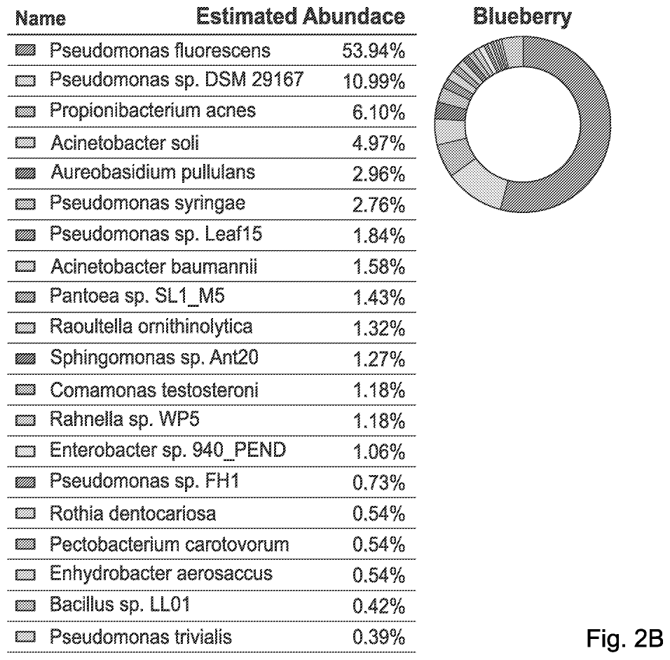

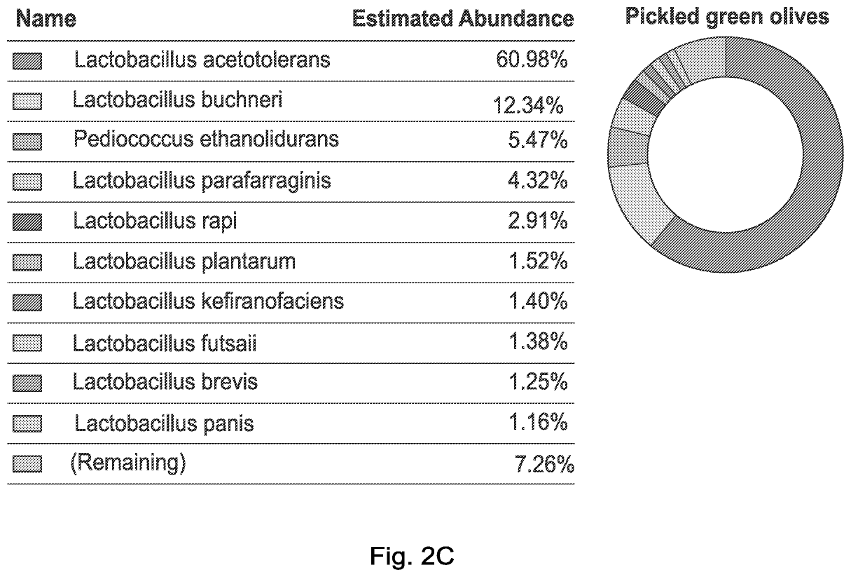

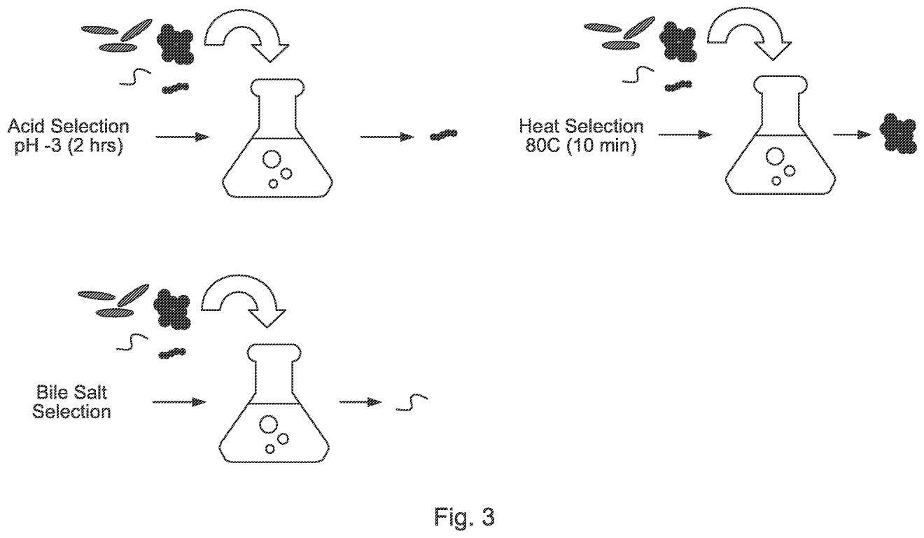

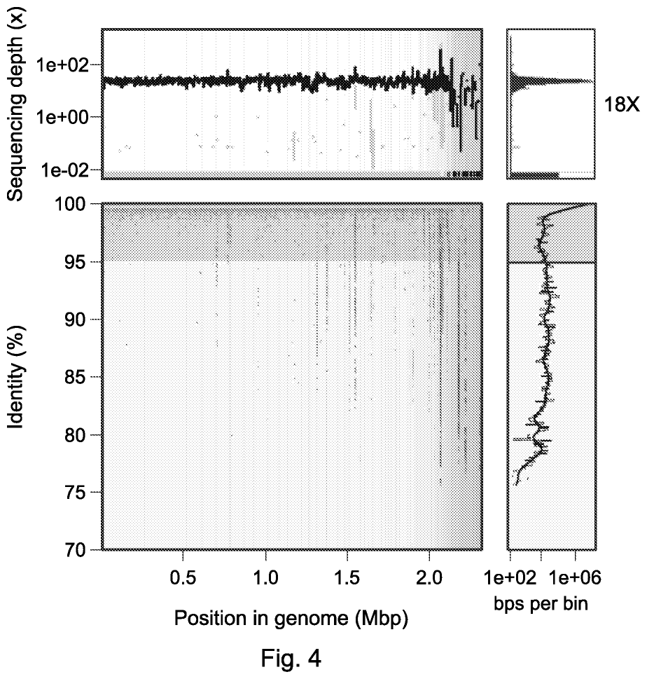

FIG. 2 shows taxonomic composition of blueberries, pickled olives and broccoli head. More specifically, FIG. 2A shows taxonomic composition of broccoli head showing a diversity of fungi and bacteria distinct from the broccoli juice dominated by few Pseudomonas species.

FIG. 2B shows taxonomic composition of blueberries including seeds and pericarp (peel) as seen by shotgun sequencing showing dominance of Pseudomonas and strains isolated and sequenced.

FIG. 2C shows taxonomic composition of pickled olives showing a variety of lactic acid bacteria present and dominant. Some of the species are recognized as probiotics.

### Example 2: In Silico Modeling Outputs for Different Assemblages and DMA Formulation

To generate in silico predictions for the effect of different microbial assemblages with a human host a genome-wide metabolic analysis was performed with formulated microbial communities selected from the Agora collection (Magbustoddir et al. 2016) Generation of genome-scale metabolic reconstructions for 773 members of the human gut microbiota. Nat. Biotech. 35, 81-89) and augmented with the genomes of bacterial members detected in the present survey. These simulations predict the “fermentative power” of each assemblage when simulated under different nutritional regimes including relatively high carbon availability (carbon replete) or carbon limited conditions when using plant fibers such as inulin, oligofructose and others as carbon source. Example 2.1. Metabolites in samples.

The method used for DNA sequencing the sample-associated microbiomes enabled to search for genes detected in the different vegetables related to propionate, butyrate, acetate and bile salt metabolism. This was done by mapping the reads obtained in the samples to reference genes selected for their intermediate role in the synthesis or

TABLE C
Predicted Metabolites Present in Sample Organisms

| NAME OF | ASSOCIATED | GENE | | E.C. | |
|---|---|---|---|---|---|
| ENZYME | METABOLITE | SYMBOL | PATHWAY | NUMBER | COMMENTS |
| ACETOLACTATE | (S)-2- | | BUTANOATE | 2.2.1.6 | BUTYRATE |
| SYNTHASE I | ACETOLACTATE | | METABOLISM | | PRODUCTION |
| ACETATE | PROPIONATE | ACKA | PROPANOATE | 2.7.2.1 | PROPIONATE |
| KINASE | | | METABOLISM | | |
| ACETYL-COA | PROPIONATE | AACS | PROPANOATE | 6.2.1.1 | PROPIONATE |
| SYNTHETASE | | | METABOLISM | | |
| ACETYL-COA | ACETATE | | PYRUVATE | 3.1.2.1 | ACETATE |
| HYDROLASE | | | METABOLISM | | |
| BILE SALT | BILE SALTS | ACR3 | BILE SALT | | BILE SALT |
| TRANSPORTER | | | TRANSPORT | | TOLERANCE |

degradation of these metabolites. There were organisms present in some of the 515 analyzed samples that matched the target pathways indicating their metabolic potential to produce desirable metabolites. Table C shows Metabolites in samples.

### DMA Formulation

Microbes in nature generally interact with multiple other groups and form consortia that work in synergy exchanging metabolic products and substrates resulting in thermodynamically favorable reactions as compared to the individual metabolism. For example, in the human colon, the process for plant fiber depolymerization, digestion and fermentation into butyrate is achieved by multiple metabolic groups working in concert. This metabolic synergy is reproduced in the DMA concept where strains are selected to be combined based on their ability to synergize to produce an increased amount of SCFA when grown together and when exposed to substrates such as plant fibers.

To illustrate this process, a set of 99 bacterial and fungal strains were isolated from food sources and their genomes were sequenced. The assembled and annotated genomes were then used to formulate in silico assemblages considering the human host as one of the metabolic members. Assuming a diet composed of lipids, different carbohydrates and proteins the metabolic fluxes were predicted using an unconstrained model comparing the individual strain production of acetate, propionate and butyrate and compared to the metabolic fluxes with the assemblage.

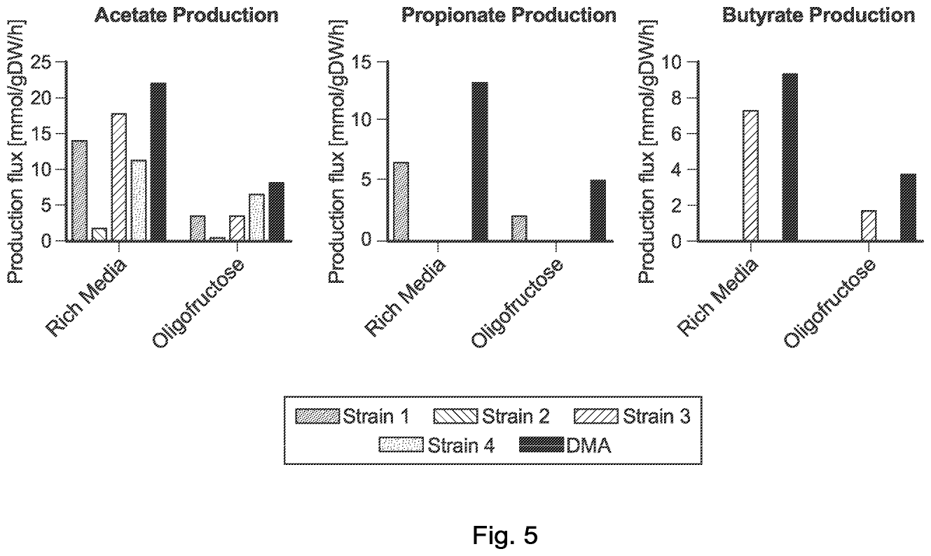

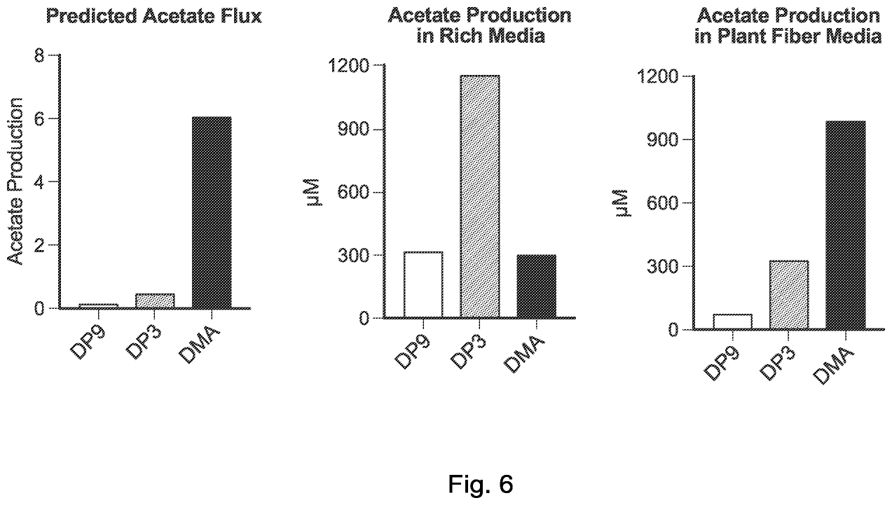

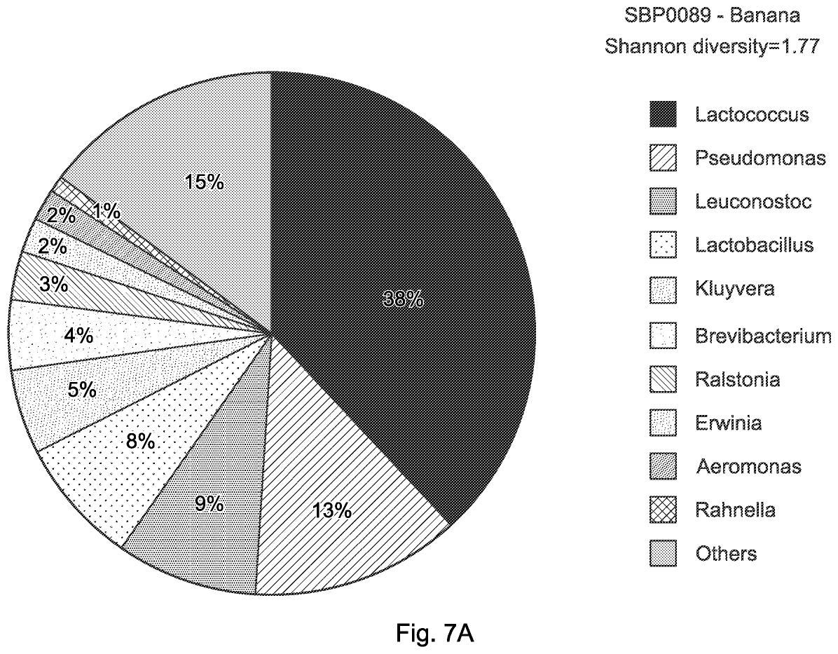

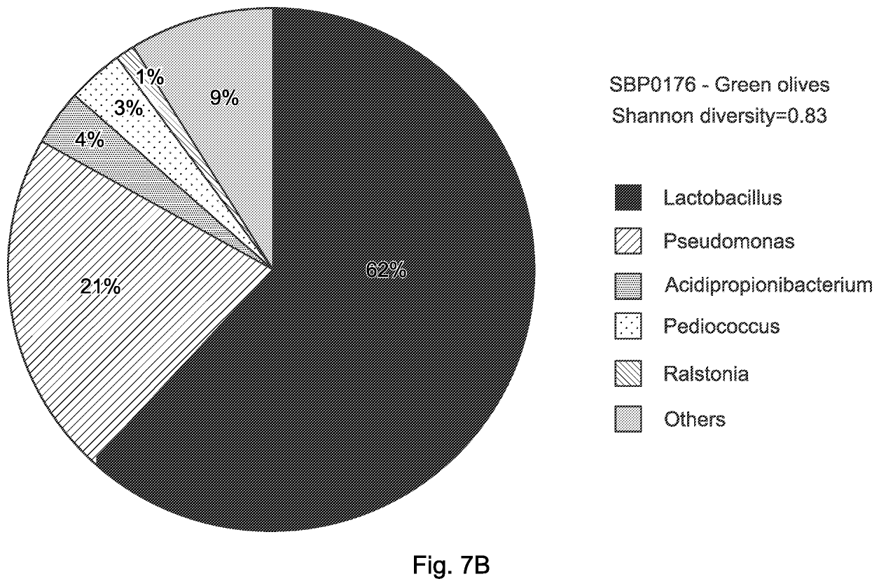

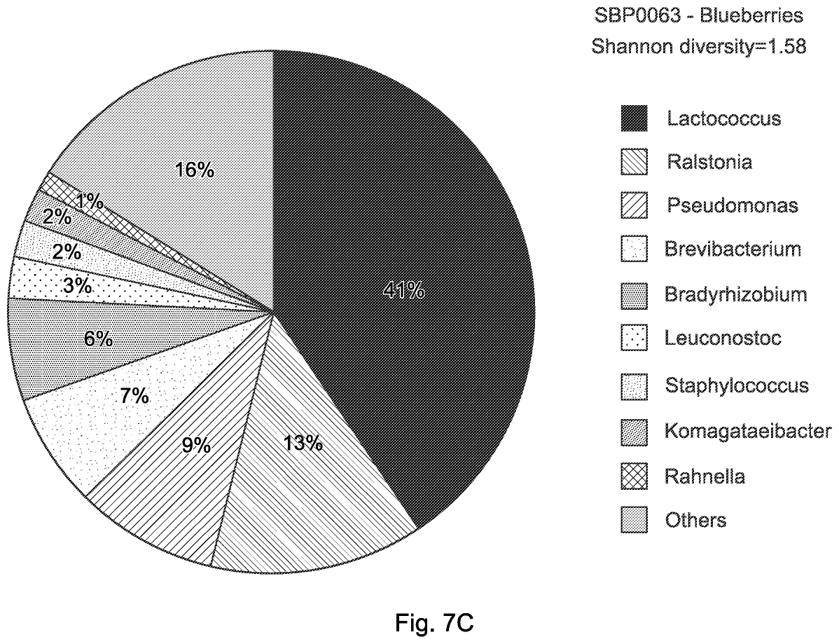

In the first model, 4 strains were combined into a DMA. Strains 1˜4 are predicted to produce acetate as single cultures but the combination into a DMA predicts the flux will increase when modeled on replete media and the flux decreases when modeled on plant fibers. Strain 4 is predicted to utilize the fibers better than the other 3 to produce acetate. Strain 1 is the only member of the assemblage predicted to produce propionate and when modeled with the other 3 strains the predicted flux doubles in replete media and quadruples in the fiber media illustrating the potential metabolic synergy from the assemblage. Strain 3 is the only member of the assemblage predicted to produce butyrate and when modeled with the other 3 strains the predicted flux increase slightly in replete media and doubled in the fiber media illustrating the potential metabolic synergy from the assemblage. Results are shown in FIG. 5.

TABLE D
Strains from first DMA model.

Strain 1 - DP6 Bacillus cereus-like
Strain 2 - DP9 Pediococcus pentosaceus-like
Strain 3 - Clostridium butyricum DSM 10702
Strain 4 - DP1 Pseudomonas fluorescens-like

Substrate availability plays an important role in the establishment of synergistic interactions. Carbon limitation in presence of plant fibers favors fiber depolymerization and fermentation to produce SCFA. Conversely carbon replete conditions will prevent the establishment of synergistic metabolism to degrade fibers as it is not favored thermodynamically when the energy available from simple sugars is available. To illustrate this, we formulated a DMA containing two strains of lactic acid bacteria and run a metabolic prediction assuming a limited media with plant fibers. According to the model, Leuconostoc predicted flux is higher than Pediococcus and the DMA flux increases five times on the combined strains. When tested in the lab and measured by gas chromatography, the acetate production increases 3 times compared to the single strains. However, when grown on carbon replete media with available simple sugars, acetate production is correspondingly higher compared to the plant fiber media but there is no benefit of synergistic acetate production when the two strains are grown together into a DMA.

In addition to acetate, propionate, and butyrate some strains produce other isomers. For example, strains DP1 related to Pseudomonas fluorescens and DP5 related to Debaromyces hansenii (yeast) produce isobutyrate when grown in carbon-replete media as single strains, however there is metabolic synergy when tested together as DMA measured as an increase in the isobutyric acid production.

To describe experimentally the process of DMA validation the following method is applied to find other candidates applicable to other products:
1. Define a suitable habitat where microbes are with desirable attributes are abundant based on ecological hypotheses. For example, fresh vegetables are known to have anti-inflammatory effects when consumed in a whole-food plant based diet, and therefore, it is likely they harbor microbes that can colonize the human gut.
2. Apply a selection filter to isolate and characterize only those microbes capable of a relevant gut function. For example, tolerate acid shock, bile salts and low oxygen. In addition, strains need to be compatible with target therapeutic drugs. In type 2 diabetes metformin is a common first line therapy.
3. Selected strains are then cultivated in vitro and their genomes sequenced at 100×coverage to assemble, annotate and use in predictive genome-wide metabolic models.
4. Metabolic fluxes are generated with unconstrained models that consider multiple strains and the human host to determine the synergistic effects from multiple strains when it is assumed they are co-cultured under a simulated substrate conditions.
5. Predicted synergistic combinations are then tested in the laboratory for validation. Single strains are grown to produce a biomass and the spent growth media removed after reaching late log phase. The washed cells are then combined in Defined Microbial Assemblages with 2-10 different strains per DMA and incubated using a culture media with plant fibers as substrates to produce short chain fatty acids to promote gut health.
6. The DMAs are then analyzed by gas chromatography to quantify the short chain fatty acid production where the synergistic effect produces an increased production in the combined assemblage as compared to the individual contributions.

### Example 3: Gut Simulation Experiments

The experiment comprises an in vitro, system that mimics various sections of the gastrointestinal tract. Isolates of interest are incubated in the presence of conditions that mimic particular stresses in the gastro-intestinal tract (such as low pH or bile salts), heat shock, or metformin. After incubation, surviving populations are recovered. Utilizing this system, the impact of various oral anti-diabetic therapies alone or in combination with probiotic cocktails of interest on the microbial ecosystem can be tested. Representative isolates are shown in Table E. Sequences associated with the isolates of Table E are shown in Table F.

TABLE E
Strains isolated from edible plants, listed with heat shock
tolerance, acid shock tolerance, and isolation temperature.

| Strain | Heat | Isolation | Acid Shock | | |
|---|---|---|---|---|---|
| Number | Shock | Temperature | (pH 3) 2 hr | Genus | Species |
| DP39 | No | 25 | No | Agrobacterium | tumefaciens |
| DP14 | No | 25 | Yes | Arthrobacter | luteolus |
| DP52 | No | 25 | No | Arthrobacter | sp. |
| DP28 | No | 25 | Yes | Aureobasidium | pullulans |
| DP4 | No | 25 | No | Aureobasidium | pullulans |
| DP10 | Yes | 25 | No | Bacillus | velezensis |
| DP13 | No | 25 | Yes | Bacillus | mycoides |
| DP48 | Yes | 25 | No | Bacillus | paralicheniformis |
| DP49 | Yes | 25 | No | Bacillus | gibsonii |
| DP55 | Yes | 25 | No | Bacillus | megaterium |
| DP57 | Yes | 25 | No | Bacillus | mycoides |
| DP6 | Yes | 25 | No | Bacillus | cereus |
| DP60 | Yes | 25 | No | Bacillus | simplex |
| DP65 | No | 25 | No | Bacillus | sp. |
| DP67 | Yes | 25 | No | Bacillus | sp. |
| DP68 | Yes | 25 | No | Bacillus | atrophaeus |
| DP69 | Yes | 25 | No | Bacillus | sp. |
| DP70 | No | 25 | No | Bacillus | tequilensis |
| DP72 | Yes | 25 | No | Bacillus | sp. |
| DP73 | Yes | 37 | No | Bacillus | clausii |
| DP74 | Yes | 25 | No | Bacillus | coagulans |
| DP81 | Yes | 37 | No | Bacillus | sp. |
| DP82 | Yes | 37 | No | Bacillus | clausii |
| DP83 | Yes | 37 | No | Bacillus | clausii |
| DP86 | No | 30 | No | Bacillus | velezensis |
| DP88 | No | 30 | No | Bacillus | velezensis |
| DP89 | No | 30 | No | Bacillus | subtilis |
| DP92 | No | 30 | No | Bacillus | subtilis |
| DP77 | Yes | 25 | No | Bacillus | megaterium |
| DP21 | No | 25 | No | Candida | santamariae |
| DP41 | Yes | 37 | No | Corynebacterium | mucifaciens |
| DP47 | No | 25 | Yes | Cronobacter | dublinensis |
| DP15 | No | 25 | No | Curtobacterium | sp. |
| DP19 | No | 25 | No | Curtobacterium | pusillum |
| DP5 | No | 37 | No | Debaromyces | hansenii |
| DP50 | No | 25 | No | Enterobacter | sp. |
| DP85 | No | 30 | No | Enterococcus | faecium |
| DP23 | No | 25 | No | Erwinia | billingiae |
| DP33 | No | 25 | No | Erwinia | persicinus |
| DP62 | No | 25 | No | Erwinia | sp. |
| DP78 | No | 25 | No | Erwinia | rhapontici |
| DP24 | No | 25 | No | Filobasidium | globisporum |
| DP32 | No | 25 | No | Hafnia | paralvei |
| DP2 | No | 37 | No | Hanseniaspora | opuntiae |
| DP64 | No | 25 | No | Hanseniaspora | uvarum |
| DP66 | No | 25 | No | Hanseniaspora | occidentalis |
| DP8 | No | 25 | No | Hanseniaspora | opuntiae |
| DP44 | No | 25 | No | Herbaspirillum | sp. |
| DP43 | No | 25 | No | Janthinobacterium | sp. |
| DP58 | No | 25 | No | Janthinobacterium | svalbardensis |
| DP51 | No | 25 | No | Klebsiella | aerogenes |
| DP59 | No | 25 | No | Kosakonia | cowanii |
| DP100 | No | 30 | No | Lactobacillus | plantarum |
| DP87 | No | 30 | No | Lactobacillus | plantarum |
| DP90 | No | 30 | No | Lactobacillus | plantarum |
| DP94 | No | 30 | No | Lactobacillus | brevis |
| DP95 | No | 30 | No | Lactobacillus | paracasei |
| DP96 | No | 30 | No | Lactobacillus | paracasei |
| DP97 | No | 30 | No | Lactococcus | garvieae |
| DP98 | No | 30 | No | Lactococcus | garvieae |
| DP61 | No | 25 | No | Lelliottia | sp. |
| DP3 | No | 25 | No | Leuconostoc | mesenteroides |
| DP93 | No | 30 | No | Leuconostoc | mesenteroides |
| DP26 | No | 25 | No | Methylobacterium | sp. |
| DP54 | No | 25 | No | Methylobacterium | adhaesivum |
| DP80 | No | 25 | No | Methylobacterium | adhaesivum |
| DP12 | No | 25 | Yes | Microbacterium | sp. |
| DP30 | No | 25 | Yes | Microbacterium | testaceum |
| DP84 | No | 25 | No | Microbacterium | sp. |
| DP76 | No | 25 | No | Ochrobactrum | sp. |
| DP56 | Yes | 25 | No | Paenibacillus | lautus |
| DP35 | No | 25 | Yes | Pantoea | ananatis |
| DP36 | No | 25 | Yes | Pantoea | vagans |
| DP40 | No | 37 | No | Pantoea | sp. |
| DP46 | No | 25 | Yes | Pantoea | agglomerans |
| DP101 | No | 30 | No | Pediococcus | pentosaceus |
| DP9 | No | 25 | No | Pediococcus | pentosaceus |
| DP102 | No | 30 | No | Pichia | krudriavzevii |
| DP7 | No | 25 | No | Pichia | fermentans |
| DP34 | No | 25 | Yes | Plantibacter | flavus |
| DP29 | No | 25 | Yes | Pseudoclavibacter | helvolus |
| DP1 | No | 25 | No | Pseudomonas | fluorescens |
| DP11 | No | 25 | No | Pseudomonas | putida |
| DP18 | No | 25 | No | Pseudomonas | sp. |
| DP37 | No | 25 | No | Pseudomonas | rhodesiae |
| DP42 | No | 37 | No | Pseudomonas | lundensis |
| DP53 | No | 25 | No | Pseudomonas | fragi |
| DP63 | No | 25 | Yes | Pseudomonas | azotoformans |
| DP75 | No | 37 | No | Pseudomonas | fluorescens |
| DP79 | No | 25 | No | Pseudomonas | fragi |
| DP17 | No | 25 | No | Rahnella | aquatilis |
| DP22 | No | 25 | No | Rahnella | sp. |
| DP38 | No | 25 | No | Rhodococcus | sp. |
| DP71 | No | 25 | No | Rhodosporidium | babjevae |
| DP45 | No | 25 | No | Sanguibacter | keddieii |
| DP27 | No | 25 | No | Sphingomonas | sp. |
| DP31 | No | 25 | Yes | Sporisorium | reilianum |
| DP20 | No | 25 | No | Stenotrophomonas | rhizophila |
| DP99 | No | 30 | No | Weissella | cibaria |

TABLE F

| Seq ID | | |
|---|---|---|
| No. | Description | Sequence |
| 1 | DP1 16S | AGTCAGACATGCAAGTCGAGCGGTAGAGAGAAGCTTGCTTCTCTTGAGAG |
| | rRNA | CGGCGGACGGGTGAGTAAAGCCTAGGAATCTGCCTGGTAGTGGGGGATAA |
| | | CGTTCGGAAACGGACGCTAATACCGCATACGTCCTACGGGAGAAAGCAGG |
| | | GGACCTTCGGGCCTTGCGCTATCAGATGAGCCTAGGTCGGATTAGCTAGTT |
| | | GGTGAGGTAATGGCTCACCAAGGCGACGATCCGTAACTGGTCTGAGAGGA |
| | | TGATCAGTCACACTGGAACTGAGACACGGTCCAGACTCCTACGGGAGGCA |
| | | GCAGTGGGGAATATTGGACAATGGGCGAAAGCCTGATCCAGCCATGCCGC |
| | | GTGTGTGAAGAAGGTCTTCGGATTGTAAAGCACTTTAAGTTGGGAGGAAG |
| | | GGCATTAACCTAATACGTTAGTGTTTTGACGTTACCGACAGAATAAGCACC |
| | | GGCTAACTCTGTGCCAGCAGCCGCGGTAATACAGAGGGTGCAAGCGTTAA |
| | | TCGGAATTACTGGGCGTAAAGCGCGCGTAGGTGGTTTGTTAAGTTGGATG |
| | | TGAAATCCCCGGGCTCAACCTGGGAACTGCATTCAAAACTGACTGACTAG |
| | | AGTATGGTAGAGGGTGGTGGAATTTCCTGTGTAGCGGTGAAATGCGTAGA |
| | | TATAGGAAGGAACACCAGTGGCGAAGGCGACCACCTGGACTAATACTGAC |
| | | ACTGAGGTGCGAAAGCGTGGGGAGCAAACAGGATTAGATACCCTGGTAGT |
| | | CCACGCCGTAAACGATGTCAACTAGCCGTTGGGAGCCTTGAGCTCTTAGT |
| | | GGCGCAGCTAACGCATTAAGTTGACCGCCTGGGGAGTACGGCCGCAAGGT |
| | | TAAAACTCAAATGAATTGACGGGGGCCCGCACAAGCGGTGGAGCATGTGG |
| | | TTTAATTCGAAGCAACGCGAAGAACCTTACCAGGCCTTGACATCCAATGA |
| | | ACTTTCTAGAGATAGATTGGTGCCTTCGGGAACATTGAGACAGGTGCTGC |
| | | ATGGCTGTCGTCAGCTCGTGTCGTGAGATGTTGGGTTAAGTCCCGTAACGA |
| | | GCGCAACCCTTGTCCTTAGTTACCAGCACGTAATGGTGGGCACTCTAAGG |
| | | AGACTGCCGGTGACAAACCGGAGGAAGGTGGGGATGACGTCAAGTCATC |
| | | ATGGCCCTTACGGCCTGGGCTACACACGTGCTACAATGGTCGGTACAGAG |
| | | GGTTGCCAAGCCGCGAGGTGGAGCTAATCCCATAAAACCGATCGTAGTCC |
| | | GGATCGCAGTCTGCAACTCGACTGCGTGAAGTCGGAATCGCTAGTAATCG |
| | | CGAATCAGAATGTCGCGGTGAATACGTTCCCGGGCCTTGTACACACCGCC |
| | | CGTCACACCATGGGAGTGGGTTGCACCAGAAGTAGCTAGTCTAACCTTCG |
| | | GGAGGACGGTTACCACGGTGTGATTCATGACTGGGGTGAAGTCGTAACAA |
| | | GGTAGCCGTAGGGGAACCTGCGGCTGGATCACCTCCTT |
| 2 | DP2 ITS | TTGTTGCTCGAGTTCTTGTTTAGATCTTTTACAATAATGTGTATCTTTAATG |
| | sequence | AAGATGNGNGCTTAATTGCGCTGCTTTATTAGAGTGTCGCAGTAGAAGTA |
| | | GTCTTGCTTGAATCTCAGTCAACGTTTACACACATTGGAGTTTTTTTACTTT |
| | | AATTTAATTCTTTCTGCTTTGAATCGAAAGGTTCAAGGCAAAAAACAAAC |
| | | ACAAACAATTTTATTTTATTATAATTTTTTAAACTAAACCAAAATTCCTAA |
| | | CGGAAATTTTAAAATAATTTAAAACTTTCAACAACGGATCTCTTGGTTCTC |
| | | GCATCGATGAAAAACGTACCGAATTGCGATAAGTAATGTGAATTGCAAAT |
| | | ACTCGTGAATCATTGAATTTTTGAACGCACATTGCGCCCTTGAGCATTCTC |
| | | AAGGGCATGCCTGTTTGAGCGTCATTTCCTTCTCAAAAAATAATTTTTTAT |
| | | TTTTTGGTTGTGGGCGATACTCAGGGTTAGCTTGAAATTGGAGACTGTTTC |
| | | AGTCTTTTTTAATTCAACACTTANCTTCTTTGGAGACGCTGTTCTCGCTGTG |
| | | ATGTATTTATGGATTTATTCGTTTTACTTTACAAGGGAAATGGTAATGTAC |
| | | CTTAGGCAAAGGGTTGCTTTTAATATTCATCAAGTTTGACCTCAAATCAGG |
| | | TAGGATTACCCGCTGAACTTAAGCATATCAATAAGCGGAGGAAAAGAAAC |
| | | CAACTGGGATTACCTTAGTAACGGCGAGTGAAGCGGTAAAAGCTCAAATT |
| | | TGAAATCTGGTACTTTCAGTGCCCGAGTTGTAATTTGTAGAATTTGTCTTT |
| | | GATTAGGTCCTTGTCTATGTTCCTTGGAACAGGACGTCATAGAGGGTGAG |
| | | ANTCCCGTTTGNNGAGGATACCTTTTCTCTGTANNACTTTTTCNAAGAGTC |
| | | GAGTTGNTTGGGAATGCAGCTCAAANNGGGTNGNAAATTCCATCTAAAGC |
| | | TAAATATTNGNCNAGAGACCGANAGCGACANTACAGNGATGGAAAGANG |
| | | AAA |
| 3 | DP3 16S | ATTGAGAGTTTGATCCTGGCTCAGGATGAACGCTGGCGGCGTGCCTAATA |
| | rRNA | CATGCAAGTCGAACGCACAGCGAAAGGTGCTTGCACCTTTCAAGTGAGTG |
| | | GCGAACGGGTGAGTAACACGTGGACAACCTGCCTCAAGGCTGGGGATAAC |
| | | ATTTGGAAACAGATGCTAATACCGAATAAAACTCAGTGTCGCATGACACA |
| | | AAGTTAAAAGGCGCTTTGGCGTCACCTAGAGATGGATCCGCGGTGCATTA |
| | | GTTAGTTGGTGGGGTAAAGGCCTACCAAGACAATGATGCATAGCCGAGTT |
| | | GAGAGACTGATCGGCCACATTGGGACTGAGACACGGCCCAAACTCCTACG |
| | | GGAGGCTGCAGTAGGGAATCTTCCACAATGGGCGAAAGCCTGATGGAGCA |
| | | ACGCCGCGTGTGTGATGAAGGCTTTCGGGTCGTAAAGCACTGTTGTACGG |
| | | GAAGAACAGCTAGAATAGGGAATGATTTTAGTTTGACGGTACCATACCAG |
| | | AAAGGGACGGCTAAATACGTGCCAGCAGCCGCGGTAATACGTATGTCCCG |
| | | AGCGTTATCCGGATTTATTGGGCGTAAAGCGAGCGCAGACGGTTGATTAA |
| | | GTCTGATGTGAAAGCCCGGAGCTCAACTCCGGAATGGCATTGGAAACTGG |
| | | TTAACTTGAGTGCAGTAGAGGTAAGTGGAACTCCATGTGTAGCGGTGGAA |
| | | TGCGTAGATATATGGAAGAACACCAGTGGCGAAGGCGGCTTACTGGACTG |
| | | TAACTGACGTTGAGGCTCGAAAGTGTGGGTAGCAAACAGGATTAGATACC |
| | | CTGGTAGTCCACACCGTAAACGATGAACACTAGGTGTTAGGAGGTTTCCG |
| | | CCTCTTAGTGCCGAAGCTAACGCATTAAGTGTTCCGCCTGGGGAGTACGA |
| | | CCGCAAGGTTGAAACTCAAAGGAATTGACGGGGACCCGCACAAGCGGTG |
| | | GAGCATGTGGTTTAATTCGAAGCAACGCGAAGAACCTTACCAGGTCTTGA |
| | | CATCCTTTGAAGCTTTTAGAGATAGAAGTGTTCTCTTCGGAGACAAAGTGA |
| | | CAGGTGGTGCATGGTCGTCGTCAGCTCGTGTCGTGAGATGTTGGGTTAAGT |
| | | CCCGCAACGAGCGCAACCCTTATTGTTAGTTGCCAGCATTCAGATGGGCA |
| | | CTCTAGCGAGACTGCCGGTGACAAACCGGAGGAAGGCGGGGACGACGTC |
| | | AGATCATCATGCCCCTTATGACCTGGGCTACACACGTGCTACAATGGCGTA |
| | | TACAACGAGTTGCCAACCCGCGAGGGTGAGCTAATCTCTTAAAGTACGTC |
| | | TCAGTTCGGATTGTAGTCTGCAACTCGACTACATGAAGTCGGAATCGCTAG |
| | | TAATCGCGGATCAGCACGCCGCGGTGAATACGTTCCCGGGTCTTGTACAC |
| | | ACCGCCCGTCACACCATGGGAGTTTGTAATGCCCAAAGCCGGTGGCCTAA |
| | | CCTTTTAGGAAGGAGCCGTCTAAGGCAGGACAGATGACTGGGGTGAAGTC |
| | | GTAACAAGGTAGCCGTAGGAGAACCTGCGGCTGGATCACCTCCTTT |
| 4 | DP4 ITS | CTTTGTTGTTAAAACTACCTTGTTGCTTTGGCGGGACCGCTCGGTCTCGAG |
| | sequence | CCGCTGGGGATTCGTCCCAGGCGAGCGCCCGCCAGAGTTAAACCAAACTC |
| | | TTGTTATTTAACCGGTCGTCTGAGTTAAAATTTTGAATAAATCAAAACTTT |
| | | CAACAACGGATCTCTTGGTTCTCGCATCGATGAAGAACGCAGCGAAATGC |
| | | GATAAGTAATGTGAATTGCAGAATTCAGTGAATCATCGAATCTTTGAACG |
| | | CACATTGCGCCCCTTGGTATTCCGAGGGGCATGCCTGTTCGAGCGTCATTA |
| | | CACCACTCAAGCTATGCTTGGTATTGGGCGTCGTCCTTAGTTGGGCGCGCC |
| | | TTAAAGACCTCGGCGAGGCCACTCCGGCTTTAGGCGTAGTAGAATTTATTC |
| | | GAACGTCTGTCAAAGGAGAGGAACTCTGCCGACTGAAACCTTTATTTTTCT |
| | | AGGTTGACCTCGGATCAGGTAGGGATACCCGCTGAACTTAAGCATATCAA |
| | | TAAGCGGAGGAAAAGAAACCAACAGGGATTGCCCTAGTAACGGCGAGTG |
| | | AAGCGGCAACAGCTCAAATTTGAAAGCTAGCCTTCGGGTTCGCATTGTAA |
| | | TTTGTAGAGGATGATTTGGGGAAGCCGCCTGTCTAAGTTCCTTGGAACAG |
| | | GACGTCATAGAGGGTGAGAATCCCGTATGTGACAGGAAATGGCACCCTAT |
| | | GTAAATCTCCTTCGACGAGTCGAGTTGTTTGGGAATGCAGCTCTAAATGGG |
| | | AGGTAAATTTCTTCTAAAGCTAAATATTGGCGAGAGACCGATAGCGCACA |
| | | AGTAGAGTGATCGAAAGATGAAAAGCACTTTGGAAAGAGAGTTAAAAAG |
| | | CACGTGAAATTGTTGAAAGGGAAGCGCTTGCAATCAGACTTGTTTAAACT |
| | | GTTCGGCCGGT |
| 5 | DP5 ITS | GCGCTTATTGCGCGGCGAAAAAACCTTACACACAGTGTTTTTTGTTATTAC |
| | sequence | ANNAACTTTTGCTTTGGTCTGGACTAGAAATAGTTTGGGCCAGAGGTTACT |
| | | AAACTAAACTTCAATATTTATATTGAATTGTTATTTATTTAATTGTCAATTT |
| | | GTTGATTAAATTCAAAAAATCTTCAAAACTTTCAACAACGGATCTCTTGGT |
| | | TCTCGCATCGATGAAGAACGCAGCGAAATGCGATAAGTAATATGAATTGC |
| | | AGATTTTCGTGAATCATCGAATCTTTGAACGCACATTGCGCCCTCTGGTAT |
| | | TCCAGAGGGCATGCCTGTTTGAGCGTCATTTCTCTCTCAAACCTTCGGGTT |
| | | TGGTATTGAGTGATACTCTTAGTCGAACTAGGCGTTTGCTTGAAATGTATT |
| | | GGCATGAGTGGTACTGGATAGTGCTATATGACTTTCAATGTATTAGGTTTA |
| | | TCCAACTCGTTGAATAGTTTAATGGTATATTTCTCGGTATTCTAGGCTCGG |
| | | CCTTACAATATAACAAACAAGTTTGACCTCAAATCAGGTAGGATTACCCG |
| | | CTGAACTTAAGCATATCAATAAGCGGAGGAAAAGAAACCAACAGGGATT |
| | | GCCTTAGTAACGGCGAGTGAAGCGGCAAAAGCTCAAATTTGAAATCTGGC |
| | | ACCTTCGGTGTCCGAGTTGTAATTTGAAGAAGGTAACTTTGGAGTTGGCTC |
| | | TTGTCTATGTTCCTTGGAACAGGACGTCACAGAGGGTGAGAATCCCGTGC |
| | | GATGAGATGCCCAATTCTATGTAAAGTGCTTTCGAAGAGTCGAGTTGTTTG |
| | | GGAATGCAGCTCTAAGTGGGTGGTAAATTCCATCTAAAGCTAAATATTGG |
| | | CGAGAGACCGATAGCGAACAAGTACAGTGATGGAAAGATGAAAAGAACT |
| | | TTGAAAAGAGAGTGAAAAAGTACGTGAAATTGTTGAAAGGGAAAGGGCT |
| | | TGAGATCAGACTTGGTATTTTGCGATCCTTTCCTTCTTGGTTGGGTTCCTCG |
| | | CAGCTTACTGGGNCAGCATCGGTTTGGATGG |
| 6 | DP6 16S | GAAAGGCGGCTTCGGCTGTCACTTATGGATGGACCCGCGTCGCATTAGCT |
| | rRNA | AGTTGGTGAGGTAACGGCTCACCAAGGCAACGATGCGTAGCCGACCTGAG |
| | | AGGGTGATCGGCCACACTGGGACTGAGACACGGCCCAGACTCCTACGGGA |
| | | GGCAGCAGTAGGGAATCTTCCGCAATGGACGAAAGTCTGACGGAGCAAC |
| | | GCCGCGTGAGTGATGAAGGCTTTCGGGTCGTAAAACTCTGTTGTTAGGGA |
| | | AGAACAAGTGCTAGTTGAATAAGCTGCACCTTGACGGTACCTAACCAGAA |
| | | AGCCACGGCTAACTACGTGCCAGCAGCCGCGGTAATACGTAGGTGGCAAG |
| | | CGTTATCCGGAATTATTGGGCGTAAAGCGCGCGCAGGTGGTTTCTTAAGTC |
| | | TGATGTGAAAGCCCACGGCTCAACCGTGGAGGGTCATTGGAAACTGGGAG |
| | | ACTTGAGTGCAGAAGAGGAAAGTGGAATTCCATGTGTAGCGGTGAAATGC |
| | | GTAGAGATATGGAGGAACACCAGTGGCGAAGGCGACTTTCTGGTCTGTAA |
| | | CTGACACTGAGGCGCGAAAGCGTGGGGAGCAAACAGGATTAGATACCCT |
| | | GGTAGTCCACGCCGTAAACGATGAGTGCTAAGTGTTAGAGGGTTTCCGCC |
| | | CTTTAGTGCTGAAGTTAACGCATTAAGCACTCCGCCTGGGGAGTACGGCC |
| | | GCAAGGCTGAAACTCAAAGGAATTGACGGGGGCCCGCACAAGCGGTGGA |
| | | GCATGTGGTTTAATTCGAAGCAACGCGAAGAACCTTACCAGGTCTTGACA |
| | | TCCTCTGAAAACCCTAGAGATAGGGCTTCTCCTTCGGGAGCAGAGTGACA |
| | | GGTGGTGCATGGTTGTCGTCAGCTCGTGTCGTGAGATGTTGGGTTAAGTCC |
| | | CGCAACGAGCGCAACCCTTGATCTTAGTTGCCATCATTAAGTTGGGCACTC |
| | | TAAGGTGACTGCCGGTGACAAACCGGAGGAAGGTGGGGATGACGTCAAA |
| | | TCATCATGCCCCTTATGACCTGGGCTACACACGTGCTACAATGGACGGTAC |
| | | AAAGAGCTGCAAGACCGCGAGGTGGAGCTAATCTCATAAAACCGTTCTCA |
| | | GTTCGGATTGTAGGCTGCAACTCGCCTACATGAAGCTGGAATCGCTAGTA |
| | | ATCGCGGATCAGCAT |
| 7 | DP7 ITS | CCACNCTGCGTGGGCGACACGAAACACCGAAACCGAACGCACGCCGTCA |
| | | AGCAAGAAATCCACAAAACTTTCAACAACGGATCTCTTGGTTCTCGCATC |
| | | GATGAAGAGCGCAGCGAAATGCGATACCTAGTGTGAATTGCAGCCATCGT |
| | | GAATCATCGAGTTCTTGAACGCACATTGCGCCCGCTGGTATTCCGGCGGGC |
| | | ATGCCTGTCTGAGCGTCGTTTCCTTCTTGGAGCGGAGCTTCAGACCTGGCG |
| | | GGCTGTCTTTCGGGACGGCGCGCCCAAAGCGAGGGGCCTTCTGCGCGAAC |
| | | TAGACTGTGCGCGCGGGGCGGCCGGCGAACTTATACCAAGCTCGACCTCA |
| | | GATCAGGCAGGAGTACCCGCTGAACTTAAGCATATCAATAAGCGGAGGAA |
| | | AAGAAACCAACAGGGATTGCCCCAGTAGCGGCGAGTGAAGCGGCAAAAG |
| | | CTCAGATTTGGAATCGCTTCGGCGAGTTGTGAATTGCAGGTTGGCGCCTCT |
| | | GCGGCGGCGGCGGTCCAAGTCCCTTGGAACAGGGCGCCATTGAGGGTGAG |
| | | AGCCCCGTGGGACCGTTTGCCTATGCTCTGAGGCCCTTCTGACGAGTCGAG |
| | | TTGTTTGGGAATGCAGCTCTAAGCGGGTGGTAAATTCCATCTAAGGCTAA |
| | | ATACTGGCGAGAGACCGATAGCGAACAAGTACTGTGAAGGAAAGATGAA |
| | | AAGCACTTTGAAAAGAGAGTGAAACAGCACGTGAAATTGTTGAAAGGGA |
| | | AGGGTATTGCGCCCGACATGGAGCGTGCGCACCGCTGCCCCTCGTGGGCG |
| | | GCGCTCTGGGCGTGCTCTGGGCCAGCATCGGTTTTTGCCGCGGGAGAAGG |
| | | GCGGCGGGCATGTAGCTCTTC |
| 8 | DP8 ITS | GTTGCTCGAGTTCTTGTTTAGATCTTTTACNATAATGTGTATCTTTAATGAA |
| | | GATGTGCGCTTAATTGCGCTGCTTTATTAGAGTGTCGCAGTAGAAGTAGTC |
| | | TTGCTTGAATCTCAGTCAACGTTTACACACATTGGAGTTTTTTTACTTTAAT |
| | | TTAATTCTTTCTGCTTTGAATCGAAAGGTTCAAGGCAAAAAACAAACACA |
| | | AACAATTTTATTTTATTATAATTTTTTAAACTAAACCAAAATTCCTAACGG |
| | | AAATTTTAAAATAATTTAAAACTTTCAACAACGGATCTCTTGGTTCTCGCA |
| | | TCGATGAAAAACGTAGCGAATTGCGATAAGTAATGTGAATTGCAAATACT |
| | | CGTGAATCATTGAATTTTTGAACGCACATTGCGCCCTTGAGCATTCTCAAG |
| | | GGCATGCCTGTTTGAGCGTCATTTCCTTCTCAAAAGATAATTTTTTATTTTT |
| | | TGGTTGTGGGCGATACTCAGGGTTAGCTTGAAATTGGAGACTGTTTCAGTC |
| | | TTTTTTAATTCAACACTTANCTTCTTTGGAGACGCTGTTCTCGCTGTGATGT |
| | | ATTTATGGATTTATTCGTTTTACTTTACAAGGGAAATGGTAATGTACCTTA |
| | | GGCAAAGGGTTGCTTTTAATATTCATCAAGTTTGACCTCAAATCAGGTAGG |
| | | ATTACCCGCTGAACTTAAGCATATCAATAAGCGGAGGAAAAGAAACCAAC |
| | | TGGGATTACCTTAGTAACGGCGAGTGAAGCGGTAAAAGCTCAAATTTGAA |
| | | ATCTGGTACTTTCANNGCCCGAGTTGTAATTTGTAGAATTTGTCTTTGATT |
| | | AGGTCCTTGTCTATGTTCCTTGGANCAGGACGTCATANAGGGTGANTCCCN |
| | | TTTGGCGANGANACCTTTTCTCTGTANACTTTTTCNANAGTCGAGTTGTTT |
| | | NGGATGCAGCTCNAAGTGGGGNGG |
| 9 | DP9 16S | ATGAGAGTTTGATCTTGGCTCAGGATGAACGCTGGCGGCGTGCCTAATAC |
| | rRNA | ATGCAAGTCGAACGAACTTCCGTTAATTGATTATGACGTACTTGTACTGAT |
| | | TGAGATTTTAACACGAAGTGAGTGGCGAACGGGTGAGTAACACGTGGGTA |
| | | ACCTGCCCAGAAGTAGGGGATAACACCTGGAAACAGATGCTAATACCGTA |
| | | TAACAGAGAAAACCGCATGGTTTTCTTTTAAAAGATGGCTCTGCTATCACT |
| | | TCTGGATGGACCCGCGGCGTATTAGCTAGTTGGTGAGGCAAAGGCTCACC |
| | | AAGGCAGTGATACGTAGCCGACCTGAGAGGGTAATCGGCCACATTGGGAC |
| | | TGAGACACGGCCCAGACTCCTACGGGAGGCAGCAGTAGGGAATCTTCCAC |
| | | AATGGACGCAAGTCTGATGGAGCAACGCCGCGTGAGTGAAGAAGGGTTTC |
| | | GGCTCGTAAAGCTCTGTTGTTAAAGAAGAACGTGGGTAAGAGTAACTGTT |
| | | TACCCAGTGACGGTATTTAACCAGAAAGCCACGGCTAACTACGTGCCAGC |
| | | AGCCGCGGTAATACGTAGGTGGCAAGCGTTATCCGGATTTATTGGGCGTA |
| | | AAGCGAGCGCAGGCGGTCTTTTAAGTCTAATGTGAAAGCCTTCGGCTCAA |
| | | CCGAAGAAGTGCATTGGAAACTGGGAGACTTGAGTGCAGAAGAGGACAG |
| | | TGGAACTCCATGTGTAGCGGTGAAATGCGTAGATATATGGAAGAACACCA |
| | | GTGGCGAAGGCGGCTGTCTGGTCTGCAACTGACGCTGAGGCTCGAAAGCA |
| | | TGGGTAGCGAACAGGATTAGATACCCTGGTAGTCCATGCCGTAAACGATG |
| | | ATTACTAAGTGTTGGAGGGTTTCCGCCCTTCAGTGCTGCAGCTAACGCATT |
| | | AAGTAATCCGCCTGGGGAGTACGACCGCAAGGTTGAAACTCAAAAGAATT |
| | | GACGGGGGCCCGCACAAGCGGTGGAGCATGTGGTTTAATTCGAAGCTACG |
| | | CGAAGAACCTTACCAGGTCTTGACATCTTCTGACAGTCTAAGAGATTAGA |
| | | GGTTCCCTTCGGGGACAGAATGACAGGTGGTGCATGGTTGTCGTCAGCTC |
| | | GTGTCGTGAGATGTTGGGTTAAGTCCCGCAACGAGCGCAACCCTTATTACT |
| | | AGTTGCCAGCATTAAGTTGGGCACTCTAGTGAGACTGCCGGTGACAAACC |
| | | GGAGGAAGGTGGGGACGACGTCAAATCATCATGCCCCTTATGACCTGGGC |
| | | TACACACGTGCTACAATGGATGGTACAACGAGTCGCGAGACCGCGAGGTT |
| | | AAGCTAATCTCTTAAAACCATTCTCAGTTCGGACTGTAGGCTGCAACTCGC |
| | | CTACACGAAGTCGGAATCGCTAGTAATCGCGGATCAGCATGCCGCGGTGA |
| | | ATACGTTCCCGGGCCTTGTACACACCGCCCGTCACACCATGAGAGTTTGTA |
| | | ACACCCAAAGCCGGTGGGGTAACCTTTTAGGAGCTAGCCGTCTAAGGTGG |
| | | GACAGATGATTAGGGTGAAGTCGTAACAAGGTAGCCGTAGGAGAACCTGC |
| | | GGCTGGATCACCTCCTT |
| 10 | DP10 16S | CAGATAGTTGGTGAGGTAACGGCTCACCAAGGCAACGATGCGTAGCCGAC |
| | rRNA | CTGAGAGGGTGATCGGCCACACTGGGACTGAGACACGGCCCAGACTCCTA |
| | | CGGGAGGCAGCAGTAGGGAATCTTCCGCAATGGACGAAAGTCTGACGGA |
| | | GCAACGCCGCGTGAGTGATGAAGGTTTTCGGATCGTAAAGCTCTGTTGTTA |
| | | GGGAAGAACAAGTGCCGTTCAAATAGGGCGGCACCTTGACGGTACCTAAC |
| | | CAGAAAGCCACGGCTAACTACGTGCCAGCAGCCGCGGTAATACGTAGGTG |
| | | GCAAGCGTTGTCCGGAATTATTGGGCGTAAAGGGCTCGCAGGCGGTTTCT |
| | | TAAGTCTGATGTGAAAGCCCCCGGCTCAACCGGGGAGGGTCATTGGAAAC |
| | | TGGGGAACTTGAGTGCAGAAGAGGAGAGTGGAATTCCACGTGTAGCGGTG |
| | | AAATGCGTAGAGATGTGGAGGAACACCAGTGGCGAAGGCGACTCTCTGGT |
| | | CTGTAACTGACGCTGAGGAGCGAAAGCGTGGGGAGCGAACAGGATTAGA |
| | | TACCCTGGTAGTCCACGCCGTAAACGATGAGTGCTAAGTGTTAGGGGGTT |
| | | TCCGCCCCTTAGTGCTGCAGCTAACGCATTAAGCACTCCGCCTGGGGAGTA |
| | | CGGTCGCAAGACTGAAACTCAAAGGAATTGACGGGGGCCCGCACAAGCG |
| | | GTGGAGCATGTGGTTTAATTCGAAGCAACGCGAAGAACCTTACCAGGTCT |
| | | TGACATCCTCTGACAATCCTAGAGATAGGACGTCCCCTTCGGGGGCAGAG |
| | | TGACAGGTGGTGCATGGTTGTCGTCAGCTCGTGTCGTGAGATGTTGGGTTA |
| | | AGTCCCGCAACGAGCGCAACCCTTGATCTTAGTTGCCAGCATTCAGTTGGG |
| | | CACTCTAAGGTGACTGCCGGTGACAAACCGGAGGAAGGTGGGGATGACGT |
| | | CAAATCATCATGCCCCTTATGACCTGGGCTACACACGTGCTACAATGGAC |
| | | AGAACAAAGGGCAGCGAAACCGCGAGGTTAAGCCAATCCCACAAATCTG |
| | | TTCTCAGTTCGGATCGCAGTCTGCAACTCGACTGCGTGAAGCTGGAATCGC |
| | | TAGTAATCGCGGATCAGCATGCCGCGGTGAATACGTTCCCGGGCCTTGTA |
| | | CACACCGCCCGTCACACCACGAGAGTTTGTAACACCCGAAGTCGGTGAGG |
| | | TAACCTTTTAGGAGCCAGCCGCCGAAGGTGGGACAGATGATTGGGGTGAA |
| | | GTCGTAACAAGGTAGCCGTATCGGAAGGTGCGGCTGGATCACCTCCTTT |
| 11 | DP11 16S | TGAAGAGTTTGATCATGGCTCAGATTGAACGCTGGCGGCAGGCCTAACAC |
| | rRNA | ATGCAAGTCGAGCGGTAGAGAGAAGCTTGCTTCTCTTGAGAGCGGCGGAC |
| | | GGGTGAGTAATGCCTAGGAATCTGCCTGGTAGTGGGGGATAACGTTCGGA |
| | | AACGGACGCTAATACCGCATACGTCCTACGGGAGAAAGCAGGGGACCTTC |
| | | GGGCCTTGCGCTATCAGATGAGCCTAGGTCGGATTAGCTAGTTGGTGAGG |
| | | TAATGGCTCACCAAGGCGACGATCCGTAACTGGTCTGAGAGGATGATCAG |
| | | TCACACTGGAACTGAGACACGGTCCAGACTCCTACGGGAGGCAGCAGTGG |
| | | GGAATATTGGACAATGGGCGAAAGCCTGATCCAGCCATGCCGCGTGTGTG |
| | | AAGAAGGTCTTCGGATTGTAAAGCACTTTAAGTTGGGAGGAAGGGTTGTA |
| | | GATTAATACTCTGCAATTTTGACGTTACCGACAGAATAAGCACCGGCTAA |
| | | CTCTGTGCCAGCAGCCGCGGTAATACAGAGGGTGCAAGCGTTAATCGGAA |
| | | TTACTGGGCGTAAAGCGCGCGTAGGTGGTTCGTTAAGTTGGATGTGAAAG |
| | | CCCCGGGCTCAACCTGGGAACTGCATTCAAAACTGACGAGCTAGAGTATG |
| | | GTAGAGGGTGGTGGAATTTCCTGTGTAGCGGTGAAATGCGTAGATATAGG |
| | | AAGGAACACCAGTGGCGAAGGCGACCACCTGGACTGATACTGACACTGA |
| | | GGTGCGAAAGCGTGGGGAGCAAACAGGATTAGATACCCTGGTAGTCCACG |
| | | CCGTAAACGATGTCAACTAGCCGTTGGAATCCTTGAGATTTTAGTGGCGCA |
| | | GCTAACGCATTAAGTTGACCGCCTGGGGAGTACGGCCGCAAGGTTAAAAC |
| | | TCAAATGAATTGACGGGGGCCCGCACAAGCGGTGGAGCATGTGGTTTAAT |
| | | TCGAAGCAACGCGAAGAACCTTACCAGGCCTTGACATCCAATGAACTTTC |
| | | CAGAGATGGATGGGTGCCTTCGGGAACATTGAGACAGGTGCTGCATGGCT |
| | | GTCGTCAGCTCGTGTCGTGAGATGTTGGGTTAAGTCCCGTAACGAGCGCA |
| | | ACCCTTGTCCTTAGTTACCAGCACGTTATGGTGGGCACTCTAAGGAGACTG |
| | | CCGGTGACAAACCGGAGGAAGGTGGGGATGACGTCAAGTCATCATGGCCC |
| | | TTACGGCCTGGGCTACACACGTGCTACAATGGTCGGTACAAAGGGTTGCC |
| | | AAGCCGCGAGGTGGAGCTAATCCCATAAAACCGATCGTAGTCCGGATCGC |
| | | AGTCTGCAACTCGACTGCGTGAAGTCGGAATCGCTAGTAATCGCGAATCA |
| | | GAATGTCGCGGTGAATACGTTCCCGGGCCTTGTACACACCGCCCGTCACAT |
| | | CCCACACGAATTGCTTG |
| 12 | DP12 16S | TACGGAGAGTTTGATCCTGGCTCAGGATGAACGCTGGCGGCGTGCTTAAC |
| | rRNA | ACATGCAAGTCGAACGGTGAAGCCAAGCTTGCTTGGTGGATCAGTGGCGA |
| | | ACGGGTGAGTAACACGTGAGCAACCTGCCCTGGACTCTGGGATAAGCGCT |
| | | GGAAACGGCGTCTAATACTGGATATGAGCCTTCATCGCATGGTGGGGGTT |
| | | GGAAAGATTTTTTGGTCTGGGATGGGCTCGCGGCCTATCAGCTTGTTGGTG |
| | | AGGTAATGGCTCACCAAGGCGTCGACGGGTAGCCGGCCTGAGAGGGTGAC |
| | | CGGCCACACTGGGACTGAGACACGGCCCAGACTCCTACGGGAGGCAGCA |
| | | GTGGGGAATATTGCACAATGGGCGAAAGCCTGATGCAGCAACGCCGCGTG |
| | | AGGGATGACGGCCTTCGGGTTGTAAACCTCTTTTAGCAGGGAAGAAGCGA |
| | | AAGTGACGGTACCTGCAGAAAAAGCGCCGGCTAACTACGTGCCAGCAGCC |
| | | GCGGTAATACGTAGGGCGCAAGCGTTATCCGGAATTATTGGGCGTAAAGA |
| | | GCTCGTAGGCGGTTTGTCGCGTCTGCTGTGAAATCCCGAGGCTCAACCTCG |
| | | GGCCTGCAGTGGGTACGGGCAGACTAGAGTGCGGTAGGGGAGATTGGAA |
| | | TTCCTGGTGTAGCGGTGGAATGCGCAGATATCAGGAGGAACACCGATGGC |
| | | GAAGGCAGATCTCTGGGCCGTAACTGACGCTGAGGAGCGAAAGGGTGGG |
| | | GAGCAAACAGGCTTAGATACCCTGGTAGTCCACCCCGTAAACGTTGGGAA |
| | | CTAGTTGTGGGGACCATTCCACGGTTTCCGTGACGCAGCTAACGCATTAAG |
| | | TTCCCCGCCTGGGGAGTACGGCCGCAAGGCTAAAACTCAAAGGAATTGAC |
| | | GGGGACCCGCACAAGCGGCGGAGCATGCGGATTAATTCGATGCAACGCG |
| | | AAGAACCTTACCAAGGCTTGACATACACCAGAACGGGCCAGAAATGGTCA |
| | | ACTCTTTGGACACTGGTGAACAGGTGGTGCATGGTTGTCGTCAGCTCGTGT |
| | | CGTGAGATGTTGGGTTAAGTCCCGCAACGAGCGCAACCCTCGTTCTATGTT |
| | | GCCAGCACGTAATGGTGGGAACTCATGGGATACTGCCGGGGTCAACTCGG |
| | | AGGAAGGTGGGGATGACGTCAAATCATCATGCCCCTTATGTCTTGGGCTTC |
| | | ACGCATGCTACAATGGCCGGTACAAAGGGCTGCAATACCGTGAGGTGGAG |
| | | CGAATCCCAAAAAGCCGGTCCCAGTTCGGATTGAGGTCTGCAACTCGACC |
| | | TCATGAAGTCGGAGTCGCTAGTAATCGCAGATCAGCAACGCTGCGGTGAA |
| | | TACGTTCCCGGGTCTTGTACACACCGCCCGTCAAGTCATGAAAGTCGGTAA |
| | | CACCTGAAGCCGGTGGCCCAACCCTTGTGGAGGGAGCCGTCGAAGGTGGG |
| | | ATCGGTAATTAGGACTAAGTCGTAACAAGGTAGCCGTACCGGAAGGTGCG |
| | | GCTGGATCACCTCCTTT |
| 13 | DP13 16S | AGTTAGCGGCGGACGGGTGAGTAACACGTGGGTAACCTGCCTATAAGACT |
| | rRNA | GGGATAACTCCGGGAAACCGGGGCTAATACCGGATAACATTTTGCACCGC |
| | | ATGGTGCGAAATTGAAAGGCGGCTTCGGCTGTCACTTATAGATGGACCTG |
| | | CGGCGCATTAGCTAGTTGGTGAGGTAACGGCTCACCAAGGCGACGATGCG |
| | | TAGCCGACCTGAGAGGGTGATCGGCCACACTGGGACTGAGACACGGCCCA |
| | | GACTCCTACGGGAGGCAGCAGTAGGGAATCTTCCGCAATGGACGAAAGTC |
| | | TGACGGAGCAACGCCGCGTGAACGATGAAGGCTTTCGGGTCGTAAAGTTC |
| | | TGTTGTTAGGGAAGAACAAGTGCTAGTTGAATAAGCTGGCACCTTGACGG |
| | | TACCTAACCAGAAAGCCACGGCTAACTACGTGCCAGCAGCCGCGGTAATA |
| | | CGTAGGTGGCAAGCGTTATCCGGAATTATTGGGCGTAAAGCGCGCGCAGG |
| | | TGGTTTCTTAAGTCTGATGTGAAAGCCCACGGCTCAACCGTGGAGGGTCAT |
| | | TGGAAACTGGGAGACTTGAGTGCAGAAGAGGAAAGTGGAATTCCATGTGT |
| | | AGCGGTGAAATGCGTAGAGATATGGAGGAACACCAGTGGCGAAGGCGAC |
| | | TTTCTGGTCTGCAACTGACACTGAGGCGCGAAAGCGTGGGGAGCAAACAG |
| | | GATTAGATACCCTGGTAGTCCACGCCGTAAACGATGAGTGCTAAGTGTTA |
| | | GAGGGTTTCCGCCCTTTAGTGCTGAAGTTAACGCATTAAGCACTCCGCCTG |
| | | GGGAGTACGGCCGCAAGGCTGAAACTCAAAGGAATTGACGGGGGCCCGC |
| | | ACAAGCGGTGGAGCATGTGGTTTAATTCGAAGCAACGCGAAGAACCTTAC |
| | | CAGGTCTTGACATCCTCTGAAAACCCTAGAGATAGGGCTTCCCCTTCGGGG |
| | | GCAGAGTGACAGGTGGTGCATGGTTGTCGTCAGCTCGTGTCGTGAGATGT |
| | | TGGGTTAAGTCCCGCAACGAGCGCAACCCTTGATCTTAGTTGCCATCATTA |
| | | AGTTGGGCACTCTAAGGTGACTGCCGGTGACAAACCGGAGGAAGGTGGG |
| | | GATGACGTCAAATCATCATGCCCCTTATGACCTGGGCTACACACGTGCTAC |
| | | AATGGACGGTACAAAGAGTCGCAAGACCGCGAGGTGGAGCTAATCTCATA |
| | | AAACCGTTCTCAGTTCGGATTGTAGGCTGCAACTCGCCTACATGAAGCTGG |
| | | AATCGCTAGTAATCGCGGATCAGCATGCCGCGGTGAATACGTTCCCGGGC |
| | | CTTGTACACACCGCCCGTCACACCACGAGAGTTTGTAACACCCGAAGTCG |
| | | GTGGGGTAACCTTTTGGAGCCAGCCGCCTAAGGTGGGACAGATGATTGGG |
| | | GTGAAGTCGTAACAAGGTAGCCGTATCGGAAGGTGCGGCTGGATCACCTC |
| | | CTTT |
| 14 | DP14 16S | TACGGAGAGTTTGATCCTGGCTCAGGATGAACGCTGGCGGCGTGCTTAAC |
| | rRNA | ACATGCAAGTCGAACGATGACTTCTGTGCTTGCACAGAATGATTAGTGGC |
| | | GAACGGGTGAGTAACACGTGAGTAACCTGCCCTTAACTTCGGGATAAGCC |
| | | TGGGAAACCGGGTCTAATACCGGATACGACCTCCTGGCGCATGCCATGGT |
| | | GGTGGAAAGCTTTAGCGGTTTTGGATGGACTCGCGGCCTATCAGCTTGTTG |
| | | GTTGGGGTAATGGCCCACCAAGGCGACGACGGGTAGCCGGCCTGAGAGG |
| | | GTGACCGGCCACACTGGGACTGAGACACGGCCCAGACTCCTACGGGAGGC |
| | | AGCAGTGGGGAATATTGCACAATGGGCGAAAGCCTGATGCAGCGACGCC |
| | | GCGTGAGGGATGACGGCCTTCGGGTTGTAAACCTCTTTCAGCAGGGAAGA |
| | | AGCGAAAGTGACGGTACCTGCAGAAGAAGCGCCGGCTAACTACGTGCCA |
| | | GCAGCCGCGGTAATACGTAGGGCGCAAGCGTTATCCGGAATTATTGGGCG |
| | | TAAAGAGCTCGTAGGCGGTTTGTCGCGTCTGCTGTGAAAGCCCGGGGCTC |
| | | AACCCCGGGTCTGCAGTGGGTACGGGCAGACTAGAGTGCAGTAGGGGAG |
| | | ACTGGAATTCCTGGTGTAGCGGTGAAATGCGCAGATATCAGGAGGAACAC |
| | | CGATGGCGAAGGCAGGTCTCTGGGCTGTAACTGACGCTGAGGAGCGAAAG |
| | | CATGGGGAGCGAACAGGATTAGATACCCTGGTAGTCCATGCCGTAAACGT |
| | | TGGGCACTAGGTGTGGGGGACATTCCACGTTTTCCGCGCCGTAGCTAACG |
| | | CATTAAGTGCCCCGCCTGGGGAGTACGGCCGCAAGGCTAAAACTCAAAGG |
| | | AATTGACGGGGGCCCGCACAAGCGGCGGAGCATGCGGATTAATTCGATGC |
| | | AACGCGAAGAACCTTACCAAGGCTTGACATGAACCGGTAAGACCTGGAAA |
| | | CAGGTCCCCCACTTGTGGCCGGTTTACAGGTGGTGCATGGTTGTCGTCAGC |
| | | TCGTGTCGTGAGATGTTGGGTTAAGTCCCGCAACGAGCGCAACCCTCGTTC |
| | | TATGTTGCCAGCGGGTTATGCCGGGGACTCATAGGAGACTGCCGGGGTCA |
| | | ACTCGGAGGAAGGTGGGGACGACGTCAAATCATCATGCCCCTTATGTCTT |
| | | GGGCTTCACGCATGCTACAATGGCCGGTACAAAGGGTTGCGATACTGTGA |
| | | GGTGGAGCTAATCCCAAAAAGCCGGTCTCAGTTCGGATTGAGGTCTGCAA |
| | | CTCGACCTCATGAAGTTGGAGTCGCTAGTAATCGCAGATCAGCAACGCTG |
| | | CGGTGAATACGTTCCCGGGCCTTGTACACACCGCCCGTCAAGTCACGAAA |
| | | GTTGGTAACACCCGAAGCCGGTGGCCTAACCCCTTGTGGGAGGGAGCCGT |
| | | CGAAGGTGGGACCGGCGATTGGGACTAAGTCGTAACAAGGTAGCCGTACC |
| | | GGAAGGTGCGGCTGGATCACCTCCTTT |
| 15 | DP15 16S | TACGGAGAGTTTGATCCTGGCTCAGGACGAACGCTGGCGGCGTGCTTAAC |
| | rRNA | ACATGCAAGTCGAACGATGATCAGGAGCTTGCTCCTGTGATTAGTGGCGA |
| | | ACGGGTGAGTAACACGTGAGTAACCTGCCCCTGACTCTGGGATAAGCGTT |
| | | GGAAACGACGTCTAATACTGGATATGATCACTGGCCGCATGGTCTGGTGG |
| | | TGGAAAGATTTTTTGGTTGGGGATGGACTCGCGGCCTATCAGCTTGTTGGT |
| | | GAGGTAATGGCTCACCAAGGCGACGACGGGTAGCCGGCCTGAGAGGGTG |
| | | ACCGGCCACACTGGGACTGAGACACGGCCCAGACTCCTACGGGAGGCAGC |
| | | AGTGGGGAATATTGCACAATGGGCGAAAGCCTGATGCAGCAACGCCGCGT |
| | | GAGGGATGACGGCCTTCGGGTTGTAAACCTCTTTTAGTAGGGAAGAAGCG |
| | | AAAGTGACGGTACCTGCAGAAAAAGCACCGGCTAACTACGTGCCAGCAGC |
| | | CGCGGTAATACGTAGGGTGCAAGCGTTGTCCGGAATTATTGGGCGTAAAG |
| | | AGCTCGTAGGCGGTTTGTCGCGTCTGCTGTGAAATCCCGAGGCTCAACCTC |
| | | GGGCTTGCAGTGGGTACGGGCAGACTAGAGTGCGGTAGGGGAGATTGGA |
| | | ATTCCTGGTGTAGCGGTGGAATGCGCAGATATCAGGAGGAACACCGATGG |
| | | CGAAGGCAGATCTCTGGGCCGTAACTGACGCTGAGGAGCGAAAGCGTGG |
| | | GGAGCGAACAGGATTAGATACCCTGGTAGTCCACGCCGTAAACGTTGGGC |
| | | GCTAGATGTAGGGACCTTTCCACGGTTTCTGTGTCGTAGCTAACGCATTAA |
| | | GCGCCCCGCCTGGGGAGTACGGCCGCAAGGCTAAAACTCAAAGGAATTGA |
| | | CGGGGGCCCGCACAAGCGGCGGAGCATGCGGATTAATTCGATGCAACGCG |
| | | AAGAACCTTACCAAGGCTTGACATACACCGGAAACGGCCAGAGATGGTCG |
| | | CCCCCTTGTGGTCGGTGTACAGGTGGTGCATGGTTGTCGTCAGCTCGTGTC |
| | | GTGAGATGTTGGGTTAAGTCCCGCAACGAGCGCAACCCTCGTTCTATGTTG |
| | | CCAGCGCGTTATGGCGGGGACTCATAGGAGACTGCCGGGGTCAACTCGGA |
| | | GGAAGGTGGGGATGACGTCAAATCATCATGCCCCTTATGTCTTGGGCTTCA |
| | | CGCATGCTACAATGGCCGGTACAAAGGGCTGCGATACCGTAAGGTGGAGC |
| | | GAATCCCAAAAAGCCGGTCTCAGTTCGGATTGAGGTCTGCAACTCGACCT |
| | | CATGAAGTCGGAGTCGCTAGTAATCGCAGATCAGCAACGCTGCGGTGAAT |
| | | ACGTTCCCGGGCCTTGTACACACCGCCCGTCAAGTCATGAAAGTCGGTAA |
| | | CACCCGAAGCCGGTGGCCTAACCCTTGTGGAAGGAGCCGTCGAAGGTGGG |
| | | ATCGGTGATTAGGACTAAGTCGTAACAAGGTAGCCGTACCGGAAGGTGCG |
| | | GCTGGATCACCTCCTTT |
| 17 | DP17 16S | GTGATTGACGTTACTCGCAGAAGAAGCACCGGCTAACTCCGTGCCAGCAG |
| | rRNA | CCGCGGTAATACGGAGGGTGCAAGCGTTAATCGGAATTACTGGGCGTAAA |
| | | GCGCACGCAGGCGGTTTGTTAAGTCAGATGTGAAATCCCCGCGCTTAACG |
| | | TGGGAACTGCATTTGAAACTGGCAAGCTAGAGTCTTGTAGAGGGGGGTAG |
| | | AATTCCAGGTGTAGCGGTGAAATGCGTAGAGATCTGGAGGAATACCGGTG |
| | | GCGAAGGCGGCCCCCTGGACAAAGACTGACGCTCAGGTGCGAAAGCGTG |
| | | GGGAGCAAACAGGATTAGATACCCTGGTAGTCCACGCTGTAAACGATGTC |
| | | GACTTGGAGGTTGTGCCCTTGAGGCGTGGCTTCCGGAGCTAACGCGTTAA |
| | | GTCGACCGCCTGGGGAGTACGGCCGCAAGGTTAAAACTCAAATGAATTGA |
| | | CGGGGGCCCGCACAAGCGGTGGAGCATGTGGTTTAATTCGATGCAACGCG |
| | | AAGAACCTTACCTACTCTTGACATCCACGGAATTCGCCAGAGATGGCTTA |
| | | GTGCCTTCGGGAACCGTGAGACAGGTGCTGCATGGCTGTCGTCAGCTCGT |
| | | GTTGTGAAATGTTGGGTTAAGTCCCGCAACGAGCGCAACCCTTATCCTTTG |
| | | TTGCCAGCACGTAATGGTGGGAACTCAAAGGAGACTGCCGGTGATAAACC |
| | | GGAGGAAGGTGGGGATGACGTCAAGTCATCATGGCCCTTACGAGTAGGGC |
| | | TACACACGTGCTACAATGGCATATACAAAGAGAAGCGAACTCGCGAGAGC |
| | | AAGCGGACCTCATAAAGTATGTCGTAGTCCGGATTGGAGTCTGCAACTCG |
| | | ACTCCATGAAGTCGGAATCGCTAGTAATCGTAGATCAGAATGCTACGG |
| 18 | DP18 16S | TGAAGAGTTTGATCATGGCTCAGATTGAACGCTGGCGGCAGGCCTAACAC |
| | rRNA | ATGCAAGTCGAGCGGATGAAAGGAGCTTGCTCCTGGATTCAGCGGCGGAC |
| | | GGGTGAGTAATGCCTAGGAATCTGCCTGGTAGTGGGGGACAACGTTTCGA |
| | | AAGGAACGCTAATACCGCATACGTCCTACGGGAGAAAGCAGGGGACCTTC |
| | | GGGCCTTGCGCTATCAGATGAGCCTAGGTCGGATTAGCTAGTTGGTGAGG |
| | | TAATGGCTCACCAAGGCGACGATCCGTAACTGGTCTGAGAGGATGATCAG |
| | | TCACACTGGAACTGAGACACGGTCCAGACTCCTACGGGAGGCAGCAGTGG |
| | | GGAATATTGGACAATGGGCGAAAGCCTGATCCAGCCATGCCGCGTGTGTG |
| | | AAGAAGGTCTTCGGATTGTAAAGCACTTTAAGTTGGGAGGAAGGGCAGTA |
| | | AATTAATACTTTGCTGTTTTGACGTTACCGACAGAATAAGCACCGGCTAAC |
| | | TCTGTGCCAGCAGCCGCGGTAATACAGAGGGTGCAAGCGTTAATCGGAAT |
| | | TACTGGGCGTAAAGCGCGCGTAGGTGGTTTGTTAAGTTGAATGTGAAATC |
| | | CCCGGGCTCAACCTGGGAACTGCATCCAAAACTGGCAAGCTAGAGTATGG |
| | | TAGAGGGTGGTGGAATTTCCTGTGTAGCGGTGAAATGCGTAGATATAGGA |
| | | AGGAACACCAGTGGCGAAGGCGACCACCTGGACTGATACTGACACTGAG |
| | | GTGCGAAAGCGTGGGGAGCAAACAGGATTAGATACCCTGGTAGTCCACGC |
| | | CGTAAACGATGTCAACTAGCCGTTGGGAGCCTTGAGCTCTTAGTGGCGCA |
| | | GCTAACGCATTAAGTTGACCGCCTGGGGAGTACGGCCGCAAGGTTAAAAC |
| | | TCAAATGAATTGACGGGGGCCCGCACAAGCGGTGGAGCATGTGGTTTAAT |
| | | TCGAAGCAACGCGAAGAACCTTACCAGGCCTTGACATCCAATGAACTTTC |
| | | CAGAGATGGATTGGTGCCTTCGGGAACATTGAGACAGGTGCTGCATGGCT |
| | | GTCGTCAGCTCGTGTCGTGAGATGTTGGGTTAAGTCCCGTAACGAGCGCA |
| | | ACCCTTGTCCTTAGTTACCAGCACGTTATGGTGGGCACTCTAAGGAGACTG |
| | | CCGGTGACAAACCGGAGGAAGGTGGGGATGACGTCAAGTCATCATGGCCC |
| | | TTACGGCCTGGGCTACACACGTGCTACAATGGTCGGTACAAAGGGTTGCC |
| | | AAGCCGCGAGGTGGAGCTAATCCCATAAAACCGATCGTAGTCCGGATCGC |
| | | AGTCTGCAACTCGACTGCGTGAAGTCGGAATCGCTAGTAATCGCGAATCA |
| | | GAATGTCGCGGTGAATACGTTCCCGGGCCTTGTACACACCGCCCGTCACA |
| | | CCATGGGAGTGGGTTGCACCAGAAGTAGCTAGTCTAACCTTCGGGAGGAC |
| | | GGTTACCACGGTGTGATTCATGACTGGGGTGAAGTCGTAACAAGGTAGCC |
| | | GTAGGGGAACCTGCGGCTGGATCACCTCCTT |
| 19 | DP19 16S | TACGGAGAGTTTGATCCTGGCTCAGGACGAACGCTGGCGGCGTGCTTAAC |
| | rRNA | ACATGCAAGTCGAACGATGATGCCCAGCTTGCTGGGTGGATTAGTGGCGA |
| | | ACGGGTGAGTAACACGTGAGTAACCTGCCCCTGACTCTGGGATAAGCGTT |
| | | GGAAACGACGTCTAATACTGGATATGACTGCCGGCCGCATGGTCTGGTGG |
| | | TGGAAAGATTTTTTGGTTGGGGATGGACTCGCGGCCTATCAGCTTGTTGGT |
| | | GAGGTAATGGCTCACCAAGGCGACGACGGGTAGCCGGCCTGAGAGGGTG |
| | | ACCGGCCACACTGGGACTGAGACACGGCCCAGACTCCTACGGGAGGCAGC |
| | | AGTGGGGAATATTGCACAATGGGCGAAAGCCTGATGCAGCAACGCCGCGT |
| | | GAGGGATGACGGCCTTCGGGTTGTAAACCTCTTTTAGTAGGGAAGAAGGG |
| | | AGCTTGCTCTTGACGGTACCTGCAGAAAAAGCACCGGCTAACTACGTGCC |
| | | AGCAGCCGCGGTAATACGTAGGGTGCAAGCGTTGTCCGGAATTATTGGGC |
| | | GTAAAGAGCTCGTAGGCGGTTTGTCGCGTCTGCTGTGAAATCCCGAGGCT |
| | | CAACCTCGGGCTTGCAGTGGGTACGGGCAGACTAGAGTGCGGTAGGGGAG |
| | | ATTGGAATTCCTGGTGTAGCGGTGGAATGCGCAGATATCAGGAGGAACAC |
| | | CGATGGCGAAGGCAGATCTCTGGGCCGTAACTGACGCTGAGGAGCGAAA |
| | | GCATGGGGAGCGAACAGGATTAGATACCCTGGTAGTCCATGCCGTAAACG |
| | | TTGGGCGCTAGATGTAGGGACCTTTCCACGGTTTCTGTGTCGTAGCTAACG |
| | | CATTAAGCGCCCCGCCTGGGGAGTACGGCCGCAAGGCTAAAACTCAAAGG |
| | | AATTGACGGGGGCCCGCACAAGCGGCGGAGCATGCGGATTAATTCGATGC |
| | | AACGCGAAGAACCTTACCAAGGCTTGACATACACCGGAAACGGCCAGAG |
| | | ATGGTCGCCCCCTTGTGGTCGGTGTACAGGTGGTGCATGGTTGTCGTCAGC |
| | | TCGTGTCGTGAGATGTTGGGTTAAGTCCCGCAACGAGCGCAACCCTCGTTC |
| | | TATGTTGCCAGCGCGTTATGGCGGGGACTCATAGGAGACTGCCGGGGTCA |
| | | ACTCGGAGGAAGGTGGGGATGACGTCAAATCATCATGCCCCTTATGTCTT |
| | | GGGCTTCACGCATGCTACAATGGCCGGTACAAAGGGCTGCGATACCGTAA |
| | | GGTGGAGCGAATCCCAAAAAGCCGGTCTCAGTTCGGATTGAGGTCTGCAA |
| | | CTCGACCTCATGAAGTCGGAGTCGCTAGTAATCGCAGATCAGCAACGCTG |
| | | CGGTGAATACGTTCCCGGGCCTTGTACACACCGCCCGTCAAGTCATGAAA |
| | | GTCGGTAACACCCGAAGCCGGTGGCCTAACCCTTGTGGAAGGAGCCGTCG |
| | | AAGGTGGGATCGGTGATTAGGACTAAGTCGTAACAAGGTAGCCGTACCGG |
| | | AAGGTGCGGCTGGATCACCTCCTTT |
| 20 | DP20 16S | TGAAGAGTTTGATCCTGGCTCAGAGTGAACGCTGGCGGTAGGCCTAACAC |
| | rRNA | ATGCAAGTCGAACGGCAGCACAGTAAGAGCTTGCTCTTATGGGTGGCGAG |
| | | TGGCGGACGGGTGAGGAATACATCGGAATCTACCTTTTCGTGGGGGATAA |
| | | CGTAGGGAAACTTACGCTAATACCGCATACGACCTTCGGGTGAAAGCAGG |
| | | GGACCTTCGGGCCTTGCGCGGATAGATGAGCCGATGTCGGATTAGCTAGT |
| | | TGGCGGGGTAAAGGCCCACCAAGGCGACGATCCGTAGCTGGTCTGAGAGG |
| | | ATGATCAGCCACACTGGAACTGAGACACGGTCCAGACTCCTACGGGAGGC |
| | | AGCAGTGGGGAATATTGGACAATGGGCGCAAGCCTGATCCAGCCATACCG |
| | | CGTGGGTGAAGAAGGCCTTCGGGTTGTAAAGCCCTTTTGTTGGGAAAGAA |
| | | AAGCAGTCGGCTAATACCCGGTTGTTCTGACGGTACCCAAAGAATAAGCA |
| | | CCGGCTAACTTCGTGCCAGCAGCCGCGGTAATACGAAGGGTGCAAGCGTT |
| | | ACTCGGAATTACTGGGCGTAAAGCGTGCGTAGGTGGTTGTTTAAGTCTGTT |
| | | GTGAAAGCCCTGGGCTCAACCTGGGAATTGCAGTGGATACTGGGCGACTA |
| | | GAGTGTGGTAGAGGGTAGTGGAATTCCCGGTGTAGCAGTGAAATGCGTAG |
| | | AGATCGGGAGGAACATCCATGGCGAAGGCAGCTACCTGGACCAACACTG |
| | | ACACTGAGGCACGAAAGCGTGGGGAGCAAACAGGATTAGATACCCTGGT |
| | | AGTCCACGCCCTAAACGATGCGAACTGGATGTTGGGTGCAATTTGGCACG |
| | | CAGTATCGAAGCTAACGCGTTAAGTTCGCCGCCTGGGGAGTACGGTCGCA |
| | | AGACTGAAACTCAAAGGAATTGACGGGGGCCCGCACAAGCGGTGGAGTA |
| | | TGTGGTTTAATTCGATGCAACGCGAAGAACCTTACCTGGTCTTGACATGTC |
| | | GAGAACTTTCCAGAGATGGATTGGTGCCTTCGGGAACTCGAACACAGGTG |
| | | CTGCATGGCTGTCGTCAGCTCGTGTCGTGAGATGTTGGGTTAAGTCCCGCA |
| | | ACGAGCGCAACCCTTGTCCTTAGTTGCCAGCACGTAATGGTGGGAACTCT |
| | | AAGGAGACCGCCGGTGACAAACCGGAGGAAGGTGGGGATGACGTCAAGT |
| | | CATCATGGCCCTTACGACCAGGGCTACACACGTACTACAATGGTAGGGAC |
| | | AGAGGGCTGCAAACCCGCGAGGGCAAGCCAATCCCAGAAACCCTATCTCA |
| | | GTCCGGATTGGAGTCTGCAACTCGACTCCATGAAGTCGGAATCGCTAGTA |
| | | ATCGCAGATCAGCATTGCTGCGGTGAATACGTTCCCGGGCCTTGTACACAC |
| | | CGCCCGTCACACCATGGGAGTTTGTTGCACCAGAAGCAGGTAGCTTAACC |
| | | TTCGGGAGGGCGCTTGCCACGGTGTGGCCGATGACTGGGGTGAAGTCGTA |
| | | ACAAGGTAGCCGTATCGGAAGGTGCGGCTGGATCACCTCCTTT |
| 22 | DP22 16S | TTGAAGAGTTTGATCATGGCTCAGATTGAACGCTGGCGGCAGGCCTAACA |
| | rRNA | CATGCAAGTCGAGCGGTAGCACAGGAGAGCTTGCTCTCCGGGTGACGAGC |
| | | GGCGGACGGGTGAGTAATGTCTGGGAAACTGCCTGATGGAGGGGGATAA |
| | | CTACTGGAAACGGTAGCTAATACCGCATGACGTCGCAAGACCAAAGTGGG |
| | | GGACCTTCGGGCCTCACGCCATCGGATGTGCCCAGATGGGATTAGCTAGT |
| | | AGGTGAGGTAATGGCTCACCTAGGCGACGATCCCTAGCTGGTCTGAGAGG |
| | | ATGACCAGCCACACTGGAACTGAGACACGGTCCAGACTCCTACGGGAGGC |
| | | AGCAGTGGGGAATATTGCACAATGGGCGCAAGCCTGATGCAGCCATGCCG |
| | | CGTGTGTGAAGAAGGCCTTAGGGTTGTAAAGCACTTTCAGCGAGGAGGAA |
| | | GGCGTTGCAGTTAATAGCTGCAGCGATTGACGTTACTCGCAGAAGAAGCA |
| | | CCGGCTAACTCCGTGCCAGCAGCCGCGGTAATACGGAGGGTGCAAGCGTT |
| | | AATCGGAATTACTGGGCGTAAAGCGCACGCAGGCGGTTTGTTAAGTCAGA |
| | | TGTGAAATCCCCGAGCTTAACTTGGGAACTGCATTTGAAACTGGCAAGCT |
| | | AGAGTCTTGTAGAGGGGGGTAGAATTCCAGGTGTAGCGGTGAAATGCGTA |
| | | GAGATCTGGAGGAATACCGGTGGCGAAGGCGGCCCCCTGGACAAAGACT |
| | | GACGCTCAGGTGCGAAAGCGTGGGGAGCAAACAGGATTAGATACCCTGGT |
| | | AGTCCACGCTGTAAACGATGTCGACTTGGAGGTTGTGCCCTTGAGGCGTG |
| | | GCTTCCGGAGCTAACGCGTTAAGTCGACCGCCTGGGGAGTACGGCCGCAA |
| | | GGTTAAAACTCAAATGAATTGACGGGGGCCCGCACAAGCGGTGGAGCATG |
| | | TGGTTTAATTCGATGCAACGCGAAGAACCTTACCTACTCTTGACATCCAGA |
| | | GAATTCGCTAGAGATAGCTTAGTGCCTTCGGGAACTCTGAGACAGGTGCT |
| | | GCATGGCTGTCGTCAGCTCGTGTTGTGAAATGTTGGGTTAAGTCCCGCAAC |
| | | GAGCGCAACCCTTATCCTTTGTTGCCAGCACGTAATGGTGGGAACTCAAA |
| | | GGAGACTGCCGGTGATAAACCGGAGGAAGGTGGGGATGACGTCAAGTCA |
| | | TCATGGCCCTTACGAGTAGGGCTACACACGTGCTACAATGGCATATACAA |
| | | AGAGAAGCGAACTCGCGAGAGCAAGCGGACCTCATAAAGTATGTCGTAGT |
| | | CCGGATTGGAGTCTGCAACTCGACTCCATGAAGTCGGAATCGCTAGTAAT |
| | | CGTAGATCAGAATGCTACGGTGAATACGTTCCCGGGCCTTGTACACACCG |
| | | CCCGTCACACCATGGGAGTGGGTTGCAAAAGAAGTAGGTAGCTTAACCTT |
| | | CGGGAGGGCGCTTACCACTTTGTGATTCATGACTGGGGTGAAGTCGTAAC |
| | | AAGGTAACCGTAGGGGAACCTGCGGTTGGATCACCTCCTT |
| 23 | DP23 16S | TTGAAGAGTTTGATCATGGCTCAGATTGAACGCTGGCGGCAGGCCTAACA |
| | rRNA | CATGCAAGTCGAACGGTAGCACAGAGAGCTTGCTCTTGGGTGACGAGTGG |
| | | CGGACGGGTGAGTAATGTCTGGGAAACTGCCCGATGGAGGGGGATAACTA |
| | | CTGGAAACGGTAGCTAATACCGCATAACGTCTTCGGACCAAAGTGGGGGA |
| | | CCTTCGGGCCTCACACCATCGGATGTGCCCAGATGGGATTAGCTAGTAGG |
| | | TGGGGTAATGGCTCACCTAGGCGACGATCCCTAGCTGGTCTGAGAGGATG |
| | | ACCAGCCACACTGGAACTGAGACACGGTCCAGACTCCTACGGGAGGCAGC |
| | | AGTGGGGAATATTGCACAATGGGCGCAAGCCTGATGCAGCCATGCCGCGT |
| | | GTATGAAGAAGGCCTTCGGGTTGTAAAGTACTTTCAGCGGGGAGGAAGGC |
| | | GATACGGTTAATAACCGTGTCGATTGACGTTACCCGCAGAAGAAGCACCG |
| | | GCTAACTCCGTGCCAGCAGCCGCGGTAATACGGAGGGTGCAAGCGTTAAT |
| | | CGGAATTACTGGGCGTAAAGCGCACGCAGGCGGTCTGTCAAGTCAGATGT |
| | | GAAATCCCCGGGCTTAACCTGGGAACTGCATTTGAAACTGGCAGGCTTGA |
| | | GTCTCGTAGAGGGGGGTAGAATTCCAGGTGTAGCGGTGAAATGCGTAGAG |
| | | ATCTGGAGGAATACCGGTGGCGAAGGCGGCCCCCTGGACGAAGACTGAC |
| | | GCTCAGGTGCGAAAGCGTGGGGAGCAAACAGGATTAGATACCCTGGTAGT |
| | | CCACGCTGTAAACGATGTCGACTTGGAGGTTGTGCCCTTGAGGCGTGGCTT |
| | | CCGGAGCTAACGCGTTAAGTCGACCGCCTGGGGAGTACGGCCGCAAGGTT |
| | | AAAACTCAAATGAATTGACGGGGGCCCGCACAAGCGGTGGAGCATGTGGT |
| | | TTAATTCGATGCAACGCGAAGAACCTTACCTGGCCTTGACATCCACAGAA |
| | | TTCGGCAGAGATGCCTTAGTGCCTTCGGGAACTGTGAGACAGGTGCTGCA |
| | | TGGCTGTCGTCAGCTCGTGTTGTGAAATGTTGGGTTAAGTCCCGCAACGAG |
| | | CGCAACCCTTATCCTTTGTTGCCAGCGATTCGGTCGGGAACTCAAAGGAG |
| | | ACTGCCGGTGATAAACCGGAGGAAGGTGGGGATGACGTCAAGTCATCATG |
| | | GCCCTTACGGCCAGGGCTACACACGTGCTACAATGGCGCATACAAAGAGA |
| | | AGCGACCTCGCGAGAGCAAGCGGACCTCATAAAGTGCGTCGTAGTCCGGA |
| | | TCGGAGTCTGCAACTCGACTCCGTGAAGTCGGAATCGCTAGTAATCGTAG |
| | | ATCAGAATGCTACGGTGAATACGTTCCCGGGCCTTGTACACACCGCCCGTC |
| | | ACACCATGGGAGTGGGTTGCAAAAGAAGTAGGTAGCTTAACCTTCGGGAG |
| | | GGCGCTTACCACTTTGTGATTCATGACTGGGGTGAAGTCGTAACAAGGTA |
| | | ACCGTAGGGGAACCTGCGGTTGGATCACCTCCTT |
| 24 | DP24 18S | CGGGGAATTAGGGTTCGATTCCGGAGAGGGAGCCTGAGAAACGGCTACCA |
| | rRNA | CATCCAAGGAAGGCAGCAGGCGCGCAAATTACCCAATCCCGACACGGGG |
| | | AGGTAGTGACAATAAATAACAATACAGGGCCCTTTGGGTCTTGTAATTGG |
| | | AATGAGTACAATTTAAATCCCTTAACGAGGAACAATTGGAGGGCAAGTCT |
| | | GGTGCCAGCAGCCGCGGTAATTCCAGCTCCAATAGCGTATATTAAAGTTG |
| | | TTGCAGTTAAAAAGCTCGTAGTTGAACTTCAGGCTTGGCGGGGTGGTCTGC |
| | | CTCACGGTATGTACTATCCGGCTGAGCCTTACCTCCTGGTGAGCCTGCATG |
| | | TCGTTTATTCGGTGTGTAGGGGAACCAGGAATTTTACTTTGAAAAAATTAG |
| | | AGTGTTCAAAGCAGGCATATGCCCGAATACATTAGCATGGAATAATAGAA |
| | | TAGGACGTGCGGTTCTATTTTGTTGGTTTCTAGGATCGCCGTAATGATTAA |
| | | TAGGGACGGTTGGGGGCATTAGTATTCAGTTGCTAGAGGTGAAATTCTTA |
| | | GATTTACTGAAGACTAACTACTGCGAAAGCATTTGCCAAGGACGTTTTCAT |
| | | TAATCAAGAACGAAGGTTAGGGGATCAAAAACGATTAGATACCGTTGTAG |
| | | TCTTAACAGTAAACTATGCCGACTAGGGATCGGGCCACGTTCATCTTTTGA |
| | | CTGGCTCGGCACCTTACGAGAAATCAAAGTCTTTGGGTTCTGGGGGGAGT |
| | | ATGGTCGCAAGGCTGAAACTTAAAGGAATTGACGGAAGGGCACCACCAG |
| | | GCGTGGAGCCTGCGGCTTAATTTGACTCAACACGGGGAAACTCACCAGGT |
| | | CCAGACATAGTAAGGATTGACAGATTGATAGCTCTTTCTTGATTCTATGGG |
| | | TGGTGGTGCATGGCCGTTCTTAGTTGGTGGAGTGATTTGTCTGGTTAATTC |
| | | CGATAACGAACGAGACCTTAACCTGCTAAATAGTCCGGCCGGCTTCGGCT |
| | | GGTCGCTGACTTCTTAGAGGGACTAACAGCGTTTAGCTGTTGGAAGTTTGA |
| | | GGCAATAACAGGTCTGTGATGCCCTTAGATGTTCTGGGCCGCACGCGCGC |
| | | TACACTGACTGAGCCAGCGAGTTTATAACCTTGGCCGAAAGGTCTGGGTA |
| | | ATCTTGTGAAACTCAGTCGTGCTGGGGATAGAGCATTGCAATTATTGCTCT |
| | | TCAACGAGGAATGCCTAGTAAGCGTGAGTCATCAGCTCACGTTGATTACG |
| | | TCCCTGCCCTTTGTACACACCGCCCGTCGCTACTACCGATTGAATGGCTTA |
| | | GTGAGATCTCCGGATTGGCTTTGGGAAGCTGGCAACGGCTACCTATTGCTG |
| | | AAAAGCTGATCAAACTTGGTCATTTAGAGGAAGTAAAAGTCGTAACAAGG |
| | | TTTCCGTAGGTGAACCTGCGGAAGGATCATT |
| 26 | DP26 16S | CTTGAGAGTTTGATCCTGGCTCAGAGCGAACGCTGGCGGCAGGCTTAACA |
| | rRNA | CATGCAAGTCGAGCGGGCATCTTCGGATGTCAGCGGCAGACGGGTGAGTA |
| | | ACACGTGGGAACGTACCCTTCGGTTCGGAATAACGCTGGGAAACTAGCGC |
| | | TAATACCGGATACGCCCTTTTGGGGAAAGGTTTACTGCCGAAGGATCGGC |
| | | CCGCGTCTGATTAGCTAGTTGGTGGGGTAACGGCCTACCAAGGCGACGAT |
| | | CAGTAGCTGGTCTGAGAGGATGATCAGCCACACTGGGACTGAGACACGGC |
| | | CCAGACTCCTACGGGAGGCAGCAGTGGGGAATATTGGACAATGGGCGCA |
| | | AGCCTGATCCAGCCATGCCGCGTGAGTGATGAAGGCCTTAGGGTTGTAAA |
| | | GCTCTTTTGTCCGGGACGATAATGACGGTACCGGAAGAATAAGCCCCGGC |
| | | TAACTTCGTGCCAGCAGCCGCGGTAATACGAAGGGGGCTAGCGTTGCTCG |
| | | GAATCACTGGGCGTAAAGGGCGCGTAGGCGGCCATTCAAGTCGGGGGTGA |
| | | AAGCCTGTGGCTCAACCACAGAATTGCCTTCGATACTGTTTGGCTTGAGTA |
| | | TGGTAGAGGTTGGTGGAACTGCGAGTGTAGAGGTGAAATTCGTAGATATT |
| | | CGCAAGAACACCGGTGGCGAAGGCGGCCAACTGGACCATTACTGACGCTG |
| | | AGGCGCGAAAGCGTGGGGAGCAAACAGGATTAGATACCCTGGTAGTCCA |
| | | CGCCGTAAACGATGAATGCCAGCTGTTGGGGTGCTTGCACCTCAGTAGCG |
| | | CAGCTAACGCTTTAAGCATTCCGCCTGGGGAGTACGGTCGCAAGATTAAA |
| | | ACTCAAAGGAATTGACGGGGGCCCGCACAAGCGGTGGAGCATGTGGTTTA |
| | | ATTCGAAGCAACGCGCAGAACCTTACCATCCCTTGACATGGCATGTTACCC |
| | | GGAGAGATTCGGGGTCCACTTCGGTGGCGTGCACACAGGTGCTGCATGGC |
| | | TGTCGTCAGCTCGTGTCGTGAGATGTTGGGTTAAGTCCCGCAACGAGCGC |
| | | AACCCACGTCCTTAGTTGCCATCATTCAGTTGGGCACTCTAGGGAGACTGC |
| | | CGGTGATAAGCCGCGAGGAAGGTGTGGATGACGTCAAGTCCTCATGGCCC |
| | | TTACGGGATGGGCTACACACGTGCTACAATGGCGGTGACAGTGGGACGCG |
| | | AAGGAGCGATCTGGAGCAAATCCCCAAAAACCGTCTCAGTTCAGATTGCA |
| | | CTCTGCAACTCGAGTGCATGAAGGCGGAATCGCTAGTAATCGTGGATCAG |
| | | CATGCCACGGTGAATACGTTCCCGGGCCTTGTACACACCGCCCGTCACACC |
| | | ATGGGAGTTGGTCTTACCCGACGGCGCTGCGCCAACCGCAAGGAGGCAGG |
| | | CGACCACGGTAGGGTCAGCGACTGGGGTGAAGTCGTAACAAGGTAGCCGT |
| | | AGGGGAACCTGCGGCTGGATCACCTCCTTT |
| 27 | DP27 16S | CTTGAGAGTTTGATCCTGGCTCAGAACGAACGCTGGCGGCATGCCTAACA |
| | rRNA | CATGCAAGTCGAACGATGCTTTCGGGCATAGTGGCGCACGGGTGCGTAAC |
| | | GCGTGGGAATCTGCCCTCAGGTTCGGAATAACAGCTGGAAACGGCTGCTA |
| | | ATACCGGATGATATCGCAAGATCAAAGATTTATCGCCTGAGGATGAGCCC |
| | | GCGTTGGATTAGGTAGTTGGTGGGGTAAAGGCCTACCAAGCCGACGATCC |
| | | ATAGCTGGTCTGAGAGGATGATCAGCCACACTGGGACTGAGACACGGCCC |
| | | AGACTCCTACGGGAGGCAGCAGTGGGGAATATTGGACAATGGGCGCAAG |
| | | CCTGATCCAGCAATGCCGCGTGAGTGATGAAGGCCCTAGGGTTGTAAAGC |
| | | TCTTTTACCCGGGAAGATAATGACTGTACCGGGAGAATAAGCCCCGGCTA |
| | | ACTCCGTGCCAGCAGCCGCGGTAATACGGAGGGGGCTAGCGTTGTTCGGA |
| | | ATTACTGGGCGTAAAGCGCACGTAGGCGGCTTTGTAAGTCAGAGGTGAAA |
| | | GCCTGGAGCTCAACTCCAGAACTGCCTTTGAGACTGCATCGCTTGAATCCA |
| | | GGAGAGGTCAGTGGAATTCCGAGTGTAGAGGTGAAATTCGTAGATATTCG |
| | | GAAGAACACCAGTGGCGAAGGCGGCTGACTGGACTGGTATTGACGCTGAG |
| | | GTGCGAAAGCGTGGGGAGCAAACAGGATTAGATACCCTGGTAGTCCACGC |
| | | CGTAAACGATGATAACTAGCTGTCCGGGCACTTGGTGCTTGGGTGGCGCA |
| | | GCTAACGCATTAAGTTATCCGCCTGGGGAGTACGGCCGCAAGGTTAAAAC |
| | | TCAAAGGAATTGACGGGGGCCTGCACAAGCGGTGGAGCATGTGGTTTAAT |
| | | TCGAAGCAACGCGCAGAACCTTACCAGCGTTTGAC |
| 28 | DP28 18S | ATAGTCGGGGGCATCAGTATTCAATTGTCAGAGGTGAAATTCTTGGATTTA |
| | rRNA | TTGAAGACTAACTACTGCGAAAGCATTTGCCAAGGATGTTTTCATTAATCA |
| | | GTGAACGAAAGTTAGGGGATCGAAGACGATCAGATACCGTCGTAGTCTTA |
| | | ACCATAAACTATGCCGACTAGGGATCGGGCGATGTTATCATTTTGACTCGC |
| | | TCGGCACCTTACGAGAAATCAAAGTCTTTGGGTTCTGGGGGGAGTATGGT |
| | | CGCAAGGCTGAAACTTAAAGAAATTGACGGAAGGGCACCACCAGGCGTG |
| | | GAGCCTGCGGCTTAATTTGACTCAACACGGGGAAACTCACCAGGTCCAGA |
| | | CACAATAAGGATTGACAGATTGAGAGCTCTTTCTTGATTTTGTGGGTGGTG |
| | | GTGCATGGCCGTTCTTAGTTGGTGGAGTGATTTGTCTGCTTAATTGCGATA |
| | | ACGAACGAGACCTTAACCTGCTAAATAGCCCGGCCCGCTTTGGCGGGTCG |
| | | CCGGCTTCTTAGAGGGACTATCGGCTCAAGCCGATGGAAGTTTGAGGCAA |
| | | TAACAGGTCTGTGATGCCCTTAGATGTTCTGGGCCGCACGCGCGCTACACT |
| | | GACAGAGCCAACGAGTTCATTTCCTTGCCCGGAAGGGTTGGGTAATCTTGT |
| | | TAAACTCTGTCGTGCTGGGGATAGAGCATTGCAATTATTGCTCTTCAACGA |
| | | GGAATGCCTAGTAAGCGTACGTCATCAGCGTGCGTTGATTACGTCCCTGCC |
| | | CTTTGTACACACCGCCCGTCGCTACTACCGATTGAATGGCTGAGTGAGGCC |
| | | TTCGGACTGGCCCAGGGAGGTCGGCAACGACCACCCAGGGCCGGAAAGTT |
| | | GGTCAAACTCCGTCATTTAGAGGAAGTAAAAGTCGTAACAAGGTTTCCGT |
| | | AGGTGAACCTGCGGAAGGATCA |
| 29 | DP29 16S | TACGGAGAGTTTGATCCTGGCTCAGGACGAACGCTGGCGGCGTGCTTAAC |
| | rRNA | ACATGCAAGTCGAACGATGAAGCCCAGCTTGCTGGGTTGATTAGTGGCGA |
| | | ACGGGTGAGTAACACGTGAGCAACGTGCCCATAACTCTGGGATAACCTCC |
| | | GGAAACGGTGGCTAATACTGGATATCTAACACGATCGCATGGTCTGTGTTT |
| | | GGAAAGATTTTTTGGTTATGGATCGGCTCACGGCCTATCAGCTTGTTGGTG |
| | | AGGTAATGGCTCACCAAGGCGACGACGGGTAGCCGGCCTGAGAGGGTGA |
| | | CCGGCCACACTGGGACTGAGACACGGCCCAGACTCCTACGGGAGGCAGCA |
| | | GTGGGGAATATTGCACAATGGGCGAAAGCCTGATGCAGCAACGCCGCGTG |
| | | AGGGATGACGGCATTCGGGTTGTAAACCTCTTTTAGTAGGGAAGAAGCGA |
| | | AAGTGACGGTACCTGCAGAAAAAGCACCGGCTAACTACGTGCCAGCAGCC |
| | | GCTGTAATACGTAGGGTGCAAGCGTTGTCCGGAATTATTGGGCGTAAAGA |
| | | GCTCGTAGGCGGTTTGTCGCGTCTGCTGTGAAATCCCGAGGCTCAACCTCG |
| | | GGTCTGCAGTGGGTACGGGCAGACTAGAGTGTGGTAGGGGAGATTGGAAT |
| | | TCCTGGTGTAGCGGTGGAATGCGCAGATATCAGGAGGAACACCGATGGCG |
| | | AAGGCAGATCTCTGGGCCATTACTGACGCTGAGGAGCGAAAGCATGGGGA |
| | | GCGAACAGGATTAGATACCCTGGTAGTCCATGCCGTAAACGTTGGGCGCT |
| | | AGATGTGGGGACCATTCCACGGTTTCCGTGTCGTAGCTAACGCATTAAGC |
| | | GCCCCGCCTGGGGAGTACGGCCGCAAGGCTAAAACTCAAAGGAATTGACG |
| | | GGGGCCCGCACAAGCGGCGGAGCATGCGGATTAATTCGATGCAACGCGA |
| | | AGAACCTTACCAAGGCTTGACATATACCGGAAACGTTCAGAAATGTTCGC |
| | | C |
| 30 | DP30 16S | TACGGAGAGTTTGATCCTGGCTCAGGATGAACGCTGGCGGCGTGCTTAAC |
| | rRNA | ACATGCAAGTCGAACGGTGAAGCCAAGCTTGCTTGGTGGATCAGTGGCGA |
| | | ACGGGTGAGTAACACGTGAGCAACCTGCCCTGGACTCTGGGATAAGCGCT |
| | | GGAAACGGCGTCTAATACTGGATATGAGACGTGATCGCATGGTCGTGTTT |
| | | GGAAAGATTTTTCGGTCTGGGATGGGCTCGCGGCCTATCAGCTTGTTGGTG |
| | | AGGTAATGGCTCACCAAGGCGTCGACGGGTAGCCGGCCTGAGAGGGTGAC |
| | | CGGCCACACTGGGACTGAGACACGGCCCAGACTCCTACGGGAGGCAGCA |
| | | GTGGGGAATATTGCACAATGGGCGAAAGCCTGATGCAGCAACGCCGCGTG |
| | | AGGGATGACGGCCTTCGGGTTGTAAACCTCTTTTAGCAGGGAAGAAGCGA |
| | | AAGTGACGGTACCTGCAGAAAAAGCGCCGGCTAACTACGTGCCAGCAGCC |
| | | GCGGTAATACGTAGGGCGCAAGCGTTATCCGGAATTATTGGGCGTAAAGA |
| | | GCTCGTAGGCGGTTTGTCGCGTCTGCTGTGAAATCCCGAGGCTCAACCTCG |
| | | GGCCTGCAGTGGGTACGGGCAGACTAGAGTGCGGTAGGGGAGATTGGAA |
| | | TTCCTGGTGTAGCGGTGGAATGCGCAGATATCAGGAGGAACACCGATGGC |
| | | GAAGGCAGATCTCTGGGCCGTAACTGACGCTGAGGAGCGAAAGGGTGGG |
| | | GAGCAAACAGGCTTAGATACCCTGGTAGTCCACCCCGTAAACGTTGGGAA |
| | | CTAGTTGTGGGGACCATTCCACGGTTTCCGTGACGCAGCTAACGCATTAAG |
| | | TTCCCCGCCTGGGGAGTACGGCCGCAAGGCTAAAACTCAAAGGAATTGAC |
| | | GGGGACCCGCACAAGCGGCGGAGCATGCGGATTAATTCGATGCAACGCG |
| | | AAGAACCTTACCAAGGCTTGACATATACGAGAACGGGCCAGAAATGGTCA |
| | | ACTCTTTGGACACTCGTAAACAGGTGGTGCATGGTTGTCGTCAGCTCGTGT |
| | | CGTGAGATGTTGGGTTAAGTCCCGCAACGAGCGCAACCCTCGTTCTATGTT |
| | | GCCAGCACGTAATGGTGGGAACTCATGGGATACTGCCGGGGTCAACTCGG |
| | | AGGAAGGTGGGGATGACGTCAAATCATCATGCCCCTTATGTCTTGGGCTTC |
| | | ACGCATGCTACAATGGCCGGTACAAAGGGCTGCAATACCGTGAGGTGGAG |
| | | CGAATCCCAAAAAGCCGGTCCCAGTTCGGATTGAGGTCTGCAACTCGACC |
| | | TCATGAAGTCGGAGTCGCTAGTAATCGCAGATCAGCAACGCTGCGGTGAA |
| | | TACGTTCCCGGGTCTTGTACACACCGCCCGTCAAGTCATGAAAGTCGGTAA |
| | | CACCTGAAGCCGGTGGCCCAACCCTTGTGGAGGGAGCCGTCGAAGGTGGG |
| | | ATCGGTAATTAGGACTAAGTCGTAACAAGGTAGCCGTACCGGAAGGTGCG |
| | | GCTGGATCACCTCCTTT |
| 31 | DP31 16S | CAGCCGGGGGCATTAGTATTTGCACGCTAGAGGTGAAATTCTTGGATTGT |
| | rRNA | GCAAAGACTTCCTACTGCGAAAGCATTTGCCAAGAATGTTTTCATTAATCA |
| | | AGAACGAAGGTTAGGGTATCGAAAACGATTAGATACCGTTGTAGTCTTAA |
| | | CAGTAAACTATGCCGACTCCGAATCGGTCGATGCTCATTTCACTGGCTCGA |
| | | TCGGCGCGGTACGAGAAATCAAAGTTTTTGGGTTCTGGGGGGAGTATGGT |
| | | CGCAAGGCTGAAACTTAAAGAAATTGACGGAAGGGCACCACCAGGAGTG |
| | | GAGCCTGCGGCTTAATTTGACTCAACACGGGAAAACTCACCGGGTCCGGA |
| | | CATAGTAAGGATTGACAGATTGATGGCGCTTTCATGATTCTATGGGTGGTG |
| | | GTGCATGGCCGTTCTTAGTTGGTGGAGTGATTTGTCTGGTTAATTCCGATA |
| | | ACGAACGAGACCTTGACCTGCTAAATAGACGGGTTGACATTTTGTTGGCC |
| | | CCTTATGTCTTCTTAGAGGGACAATCGACCGTCTAGGTGATGGAGGCAAA |
| | | AGGCAATAACAGGTCTGTGATGCCCTTAGATGTTCCGGGCTGCACGCGCG |
| | | CTACACTGACAGAGACAACGAGTGGGGCCCCTTGTCCGAAATGACTGGGT |
| | | AAACTTGTGAAACTTTGTCGTGCTGGGGATGGAGCTTTGTAATTTTTGCTC |
| | | TTCAACGAGGAATTCCTAGTAAGCGCAAGTCATCAGCTTGCGTTGACTAC |
| | | GTCCCTGCCCTTTGTACACACCGCCCGTCGCTACTACCGATTGAATGGCTT |
| | | AGTGAGGACTTGGGAGAGTACATCGGGGAGCCAGCAATGGCACCCTGAC |
| | | GGCTCAAACTCTTACAAACTTGGTCATTTAGAGGAAGTAAAAGTCGTAAC |
| | | AAGGTATCTGTAGGTGAACCTGCAGATGGATCATTTC |
| 32 | DP32 16S | ACTGAGCATTGACGTTACTCGCAGAAGAAGCACCGGCTAACTCCGTGCCA |
| | rRNA | GCAGCCGCGGTAATACGGAGGGTGCAAGCGTTAATCGGAATTACTGGGCG |
| | | TAAAGCGCACGCAGGCGGTTTGTTAAGTCAGATGTGAAATCCCCGAGCTT |
| | | AACTTGGGAACTGCATTTGAAACTGGCAAGCTAGAGTCTTGTAGAGGGGG |
| | | GTAGAATTCCAGGTGTAGCGGTGAAATGCGTAGAGATCTGGAGGAATACC |
| | | GGTGGCGAAGGCGGCCCCCTGGACAAAGACTGACGCTCAGGTGCGAAAG |
| | | CGTGGGGAGCAAACAGGATTAGATACCCTGGTAGTCCACGCTGTAAACGA |
| | | TGTCGACTTGGAGGTTGTGCCCTTGAGGCGTGGCTTCCGGAGCTAACGCGT |
| | | TAAGTCGACCGCCTGGGGAGTACGGCCGCAAGGTTAAAACTCAAATGAAT |
| | | TGACGGGGGCCCGCACAAGCGGTGGAGCATGTGGTTTAATTCGATGCAAC |
| | | GCGAAGAACCTTACCTACTCTTGACATCCAGAGAATTCGCTAGAGATAGC |
| | | TTAGTGCCTTCGGGAACTCTGAGACAGGTGCTGCATGGCTGTCGTCAGCTC |
| | | GTGTTGTGAAATGTTGGGTTAAGTCCCGCAACGAGCGCAACCCTTATCCTT |
| | | TGTTGCCAGCGAGTAATGTCGGGAACTCAAAGGAGACTGCCGGTGATAAA |
| | | CCGGAGGAAGGTGGGGATGACGTCAAGTCATCATGGCCCTTACGAGTAGG |
| | | GCTACACACGTGCTACAATGGCATATACAAAGAGAAGCGAACTCGCGAGA |
| | | GCAAGCGGACCTCATAAAGTATGTCGTAGTCCGGATTGGAGTCTGCAACT |
| | | CGACTCCATGAAGTCGGAATCGCTAGTAATCGTAGATCAGAATGCTACGG |
| | | TGAATACGTTCCCGGGCCTTGTACACACCGCCCGTCACACCATGGGAGTG |
| | | GGTTGCAAAAGAAGTAGGTAGCTTAACCTTCGGGAGGGCGCTTACCACTT |
| | | TGTGATTCATGACTGGGGTGAAGTCGTAACAAGGTAACCGTAGGGGAACC |
| | | TGCGGTTGGATCACCTCCTT |
| 33 | DP33 16S | GGAGGAAGGCGTAGAGATCTGGAGGAATACCGGTGGCGAAGGCGGCCCC |
| | rRNA | CTGGACAAAGACTGACGCTCAGGTGCGAAAGCGTGGGGAGCAAACAGGA |
| | | TTAGATACCCTGGTAGTCCACGCCGTAAACGATGTCGACTTGGAGGTTGTG |
| | | CCCTTGAGGCGTGGCTTCCGGAGCTAACGCGTTAAGTCGACCGCCTGGGG |
| | | AGTACGGCCGCAAGGTTAAAACTCAAATGAATTGACGGGGGCCCGCACAA |
| | | GCGGTGGAGCATGTGGTTTAATTCGATGCAACGCGAAGAACCTTACCTGG |
| | | CCTTGACATCCACGGAATTCGGCAGAGATGCCTTAGTGCCTTCGGGAACC |
| | | GTGAGACAGGTGCTGCATGGCTGTCGTCAGCTCGTGTTGTGAAATGTTGG |
| | | GTTAAGTCCCGCAACGAGCGCAACCCTTATCCTTTGTTGCCAGCACGTAAT |
| | | GGTGGGAACTCAAAGGAGACTGCCGGTGATAAACCGGAGGAAGGTGGGG |
| | | ATGACGTCAAGTCATCATGGCCCTTACGGCCAGGGCTACACACGTGCTAC |
| | | AATGGCGCATACAAAGAGAAGCGACCTCGCGAGAGCAAGCGGACCTCAT |
| | | AAAGTGCGTCGTAGTCCGGATCGGAGTCTGCAACTCGACTCCGTGAAGTC |
| | | GGAATCGCTAGTAATCGTAGATCAGAATGCTACGGTGAATACGTTCCCGG |
| | | GCCTTGTACACACCGCCCGTCACACCATGGGAGTGGGTTGCAAAAGAAGT |
| | | AGGTAGCTTAACCTTCGGGAGGGCGCTTACCACTTTGTGATTCATGACTGG |
| | | GGTGAAGTCGTAACAAGGTAACCGTAGGGGAACCTGCGGTTGGATCACCT |
| | | CCTT |
| 34 | DP34 16S | TACGGAGAGTTTGATCCTGGCTCAGGACGAACGCTGGCGGCGTGCTTAAC |
| | rRNA | ACATGCAAGTCGAACGATGAAGCCCAGCTTGCTGGGTGGATTAGTGGCGA |
| | | ACGGGTGAGTAACACGTGAGTAACCTGCCCTTGACTCTGGGATAAGCGTT |
| | | GGAAACGACGTCTAATACCGGATACGAGCTTCCACCGCATGGTGAGTTGC |
| | | TGGAAAGAATTTTGGTCAAGGATGGACTCGCGGCCTATCAGCTTGTTGGT |
| | | GAGGTAATGGCTCACCAAGGCGACGACGGGTAGCCGGCCTGAGAGGGTG |
| | | ACCGGCCACACTGGGACTGAGACACGGCCCAGACTCCTACGGGAGGCAGC |
| | | AGTGGGGAATATTGCACAATGGGCGAAAGCCTGATGCAGCAACGCCGCGT |
| | | GAGGGACGACGGCCTTCGGGTTGTAAACCTCTTTTAGCAGGGAAGAAGCG |
| | | AAAGTGACGGTACCTGCAGAAAAAGCACCGGCTAACTACGTGCCAGCAGC |
| | | CGCGGTAATACGTAGGGTGCAAGCGTTGTCCGGAATTATTGGGCGTAAAG |
| | | AGCTCGTAGGCGGTTTGTCGCGTCTGCTGTGAAATCCCGAGGCTCAACCTC |
| | | GGGTCTGCAGTGGGTACGGGCAGACTAGAGTGCGGTAGGGGAGATTGGA |
| | | ATTCCTGGTGTAGCGGTGGAATGCGCAGATATCAGGAGGAACACCGATGG |
| | | CGAAGGCAGATCTCTGGGCCGCTACTGACGCTGAGGAGCGAAAGGGTGG |
| | | GGAGCAAACAGGCTTAGATACCCTGGTAGTCCACCCCGTAAACGTTGGGC |
| | | GCTAGATGTGGGGACCATTCCACGGTTTCCGTGTCGTAGCTAACGCATTAA |
| | | GCGCCCCGCCTGGGGAGTACGGCCGCAAGGCTAAAACTCAAAGGAATTGA |
| | | CGGGGGCCCGCACAAGCGGCGGAGCATGCGGATTAATTCGATGCAACGCG |
| | | AAGAACCTTACCAAGGCTTGACATATACGAGAACGGGCCAGAAATGGTCA |
| | | ACTCTTTGGACACTCGTAAACAGGTGGTGCATGGTTGTCGTCAGCTCGTGT |
| | | CGTGAGATGTTGGGTTAAGTCCCGCAACGAGCGCAACCCTCGTTCTATGTT |
| | | GCCAGCACGTAATGGTGGGAACTCATGGGATACTGCCGGGGTCAACTCGG |
| | | AGGAAGGTGGGGACGACGTCAAATCATCATGCCCCTTATGTCTTGGGCTT |
| | | CACGCATGCTACAATGGCCAGTACAAAGGGCTGCAATACCGTAAGGTGGA |
| | | GCGAATCCCAAAAAGCTGGTCCCAGTTCGGATTGAGGTCTGCAACTCGAC |
| | | CTCATGAAGTCGGAGTCGCTAGTAATCGCAGATCAGCAACGCTGCGGTGA |
| | | ATACGTTCCCGGGCCTTGTACACACCGCCCGTCAAGTCATGAAAGTCGGT |
| | | AACACCCGAAGCCAGTGGCCTAACCGCAAGGATGGAGCTGTCTAAGGTGG |
| | | GATCGGTAATTAGGACTAAGTCGTAACAAGGTAGCCGTACCGGAAGGTGC |
| | | GGCTGGATCACCTCCTTT |
| 35 | DP35 16S | TTGAAGAGTTTGATCATGGCTCAGATTGAACGCTGGCGGCAGGCCTAACA |
| | rRNA | CATGCAAGTCGGACGGTAGCACAGAGAGCTTGCTCTTGGGTGACGAGTGG |
| | | CGGACGGGTGAGTAATGTCTGGGGATCTGCCCGATAGAGGGGGATAACCA |
| | | CTGGAAACGGTGGCTAATACCGCATAACGTCGCAAGACCAAAGAGGGGG |
| | | ACCTTCGGGCCTCTCACTATCGGATGAACCCAGATGGGATTAGCTAGTAG |
| | | GCGGGGTAATGGCCCACCTAGGCGACGATCCCTAGCTGGTCTGAGAGGAT |
| | | GACCAGCCACACTGGAACTGAGACACGGTCCAGACTCCTACGGGAGGCAG |
| | | CAGTGGGGAATATTGCACAATGGGCGCAAGCCTGATGCAGCCATGCCGCG |
| | | TGTATGAAGAAGGCCTTCGGGTTGTAAAGTACTTTCAGCGGGGAGGAAGG |
| | | CGATGAGGTTAATAACCGCGTCGATTGACGTTACCCGCAGAAGAAGCACC |
| | | GGCTAACTCCGTGCCAGCAGCCGCGGTAATACGGAGGGTGCAAGCGTTAA |
| | | TCGGAATTACTGGGCGTAAAGCGCACGCAGGCGGTCTGTTAAGTCAGATG |
| | | TGAAATCCCCGGGCTTAACCTGGGAACTGCATTTGAAACTGGCAGGCTTG |
| | | AGTCTTGTAGAGGGGGGTAGAATTCCAGGTGTAGCGGTGAAATGCGTAGA |
| | | GATCTGGAGGAATACCGGTGGCGAAGGCGGCCCCCTGGACAAAGACTGA |
| | | CGCTCAGGTGCGAAAGCGTGGGGAGCAAACAGGATTAGATACCCTGGTAG |
| | | TCCACGCCGTAAACGATGTCGACTTGGAGGTTGTTCCCTTGAGGAGTGGCT |
| | | TCCGGAGCTAACGCGTTAAGTCGACCGCCTGGGGAGTACGGCCGCAAGGT |
| | | TAAAACTCAAATGAATTGACGGGGGCCCGCACAAGCGGTGGAGCATGTGG |
| | | TTTAATTCGATGCAACGCGAAGAACCTTACCTACTCTTGACATCCAGCGAA |
| | | CTTAGCAGAGATGCTTTGGTGCCTTCGGGAACGCTGAGACAGGTGCTGCA |
| | | TGGCTGTCGTCAGCTCGTGTTGTGAAATGTTGGGTTAAGTCCCGCAACGAG |
| | | CGCAACCCTTATCCTTTGTTGCCAGCGATTCGGTCGGGAACTCAAAGGAG |
| | | ACTGCCGGTGATAAACCGGAGGAAGGTGGGGATGACGTCAAGTCATCATG |
| | | GCCCTTACGAGTAGGGCTACACACGTGCTACAATGGCGCATACAAAGAGA |
| | | AGCGACCTCGCGAGAGCAAGCGGACCTCACAAAGTGCGTCGTAGTCCGGA |
| | | TCGGAGTCTGCAACTCGACTCCGTGAAGTCGGAATCGCTAGTAATCGTGG |
| | | ATCAGAATGCCACGGTGAATACGTTCCCGGGCCTTGTACACACCGCCCGT |
| | | CACACCATGGGAGTGGGTTGCAAAAGAAGTAGGTAGCTTAACCTTCGGGA |
| | | GGGCGCTTACCACTTTGTGATTCATTACTGGGGTGAAGTCGTAACAAGGTA |
| | | ACCGTAGGGGAACCTGCGGTTGGATCACCTCCTT |
| 36 | DP36 16S | TTGAAGAGTTTGATCATGGCTCAGATTGAACGCTGGCGGCAGGCCTAACA |
| | rRNA | CATGCAAGTCGGACGGTAGCACAGAGAGCTTGCTCTTGGGTGACGAGTGG |
| | | CGGACGGGTGAGTAATGTCTGGGGATCTGCCCGATAGAGGGGGATAACCA |
| | | CTGGAAACGGTGGCTAATACCGCATAACGTCGCAAGACCAAAGAGGGGG |
| | | ACCTTCGGGCCTCTCACTATCGGATGAACCCAGATGGGATTAGCTAGTAG |
| | | GCGGGGTAATGGCCCACCTAGGCGACGATCCCTAGCTGGTCTGAGAGGAT |
| | | GACCAGCCACACTGGAACTGAGACACGGTCCAGACTCCTACGGGAGGCAG |
| | | CAGTGGGGAATATTGCACAATGGGCGCAAGCCTGATGCAGCCATGCCGCG |
| | | TGTATGAAGAAGGCCTTCGGGTTGTAAAGTACTTTCAGCGGGGAGGAAGG |
| | | CGATGCGGTTAATAACCGCGTCGATTGACGTTACCCGCAGAAGAAGCACC |
| | | GGCTAACTCCGTGCCAGCAGCCGCGGTAATACGGAGGGTGCAAGCGTTAA |
| | | TCGGAATTACTGGGCGTAAAGCGCACGCAGGCGGTCTGTTAAGTCAGATG |
| | | TGAAATCCCCGGGCTTAACCTGGGAACTGCATTTGAAACTGGCAGGCTTG |
| | | AGTCTTGTAGAGGGGGGTAGAATTCCAGGTGTAGCGGTGAAATGCGTAGA |
| | | GATCTGGAGGAATACCGGTGGCGAAGGCGGCCCCCTGGACAAAGACTGA |
| | | CGCTCAGGTGCGAAAGCGTGGGGAGCAAACAGGATTAGATACCCTGGTAG |
| | | TCCACGCCGTAAACGATGTCGACTTGGAGGTTGTTCCCTTGAGGAGTGGCT |
| | | TCCGGAGCTAACGCGTTAAGTCGACCGCCTGGGGAGTACGGCCGCAAGGT |
| | | TAAAACTCAAATGAATTGACGGGGGCCCGCACAAGCGGTGGAGCATGTGG |
| | | TTTAATTCGATGCAACGCGAAGAACCTTACCTACTCTTGACATC |
| 37 | DP37 16S | TGAAGAGTTTGATCATGGCTCAGATTGAACGCTGGCGGCAGGCCTAACAC |
| | rRNA | ATGCAAGTCGAGCGGTAGAGAGAAGCTTGCTTCTCTTGAGAGCGGCGGAC |
| | | GGGTGAGTAATGCCTAGGAATCTGCCTGGTAGTGGGGGATAACGTTCGGA |
| | | AACGAACGCTAATACCGCATACGTCCTACGGGAGAAAGCAGGGGACCTTC |
| | | GGGCCTTGCGCTATCAGATGAGCCTAGGTCGGATTAGCTAGTTGGTGGGG |
| | | TAATGGCTCACCAAGGCGACGATCCGTAACTGGTCTGAGAGGATGATCAG |
| | | TCACACTGGAACTGAGACACGGTCCAGACTCCTACGGGAGGCAGCAGTGG |
| | | GGAATATTGGACAATGGGCGAAAGCCTGATCCAGCCATGCCGCGTGTGTG |
| | | AAGAAGGTCTTCGGATTGTAAAGCACTTTAAGTTGGGAGGAAGGGCCATT |
| | | ACCTAATACGTGATGGTTTTGACGTTACCGACAGAATAAGCACCGGCTAA |
| | | CTCTGTGCCAGCAGCCGCGGTAATACAGAGGGTGCAAGCGTTAATCGGAA |
| | | TTACTGGGCGTAAAGCGCGCGTAGGTGGTTTGTTAAGTTGGATGTGAAAT |
| | | CCCCGGGCTCAACCTGGGAACTGCATTCAAAACTGACTGACTAGAGTATG |
| | | GTAGAGGGTGGTGGAATTTCCTGTGTAGCGGTGAAATGCGTAGATATAGG |
| | | AAGGAACACCAGTGGCGAAGGCGACCACCTGGACTGATACTGACACTGA |
| | | GGTGCGAAAGCGTGGGGAGCAAACAGGATTAGATACCCTGGTAGTCCACG |
| | | CCGTAAACGATGTCAACTAGCCGTTGGGAGCCTTGAGCTCTTAGTGGCGC |
| | | AGCTAACGCATTAAGTTGACCGCCTGGGGAGTACGGCCGCAAGGTTAAAA |
| | | CTCAAATGAATTGACGGGGGCCCGCACAAGCGGTGGAGCATGTGGTTTAA |
| | | TTCGAAGCAACGCGAAGAACCTTACCAGGCCTTGACATCCAATGAACTTT |
| | | CTAGAGATAGATTGGTGCCTTCGGGAACATTGAGACAGGTGCTGCATGGC |
| | | TGTCGTCAGCTCGTGTCGTGAGATGTTGGGTTAAGTCCCGTAACGAGCGCA |
| | | ACCCTTGTCCTTAGTTACCAGCACGTAATGGTGGGCACTCTAAGGAGACTG |
| | | CCGGTGACAAACCGGAGGAAGGTGGGGATGACGTCAAGTCATCATGGCCC |
| | | TTACGGCCTGGGCTACACACGTGCTACAATGGTCGGTACAGAGGGTTGCC |
| | | AAGCCGCGAGGTGGAGCTAATCCCATAAAACCGATCGTAGTCCGGATCGC |
| | | AGTCTGCAACTCGACTGCGTGAAGTCGGAATCGCTAGTAATCGCGAATCA |
| | | GAATGTCGCGGTGAATACGTTCCCGGGCCTTGTACACACCGCCCGTCACA |
| | | CCATGGGAGTGGGTTGCACCAGAAGTAGCTAGTCTAACCTTCGGGGGGAC |
| | | GGTTACCACGGTGTGATTCATGACTGGGGTGAAGTCGTAACAAGGTAGCC |
| | | GTAGGGGAACCTGCGGCTGGATCACCTCCTT |
| 38 | DP38 16S | TACGGAGAGTTTGATCCTGGCTCAGGACGAACGCTGGCGGCGTGCTTAAC |
| | rRNA | ACATGCAAGTCGAGCGGTAAGGCCTTTCGGGGTACACGAGCGGCGAACGG |
| | | GTGAGTAACACGTGGGTGATCTGCCCTGCACTCTGGGATAAGCTTGGGAA |
| | | ACTGGGTCTAATACCGGATATGACCACAGCATGCATGTGTTGTGGTGGAA |
| | | AGATTTATCGGTGCAGGATGGGCCCGCGGCCTATCAGCTTGTTGGTGGGG |
| | | TAATGGCCTACCAAGGCGACGACGGGTAGCCGACCTGAGAGGGTGACCG |
| | | GCCACACTGGGACTGAGACACGGCCCAGACTCCTACGGGAGGCAGCAGTG |
| | | GGGAATATTGCACAATGGGCGGAAGCCTGATGCAGCGACGCCGCGTGAG |
| | | GGATGAAGGCCTTCGGGTTGTAAACCTCTTTCAGCAGGGACGAAGCGTGA |
| | | GTGACGGTACCTGCAGAAGAAGCACCGGCTAACTACGTGCCAGCAGCCGC |
| | | GGTAATACGTAGGGTGCGAGCGTTGTCCGGAATTACTGGGCGTAAAGAGT |
| | | TCGTAGGCGGTTTGTCGCGTCGTTTGTGAAAACCCGGGGCTCAACTTCGGG |
| | | CTTGCAGGCGATACGGGCAGACTTGAGTGTTTCAGGGGAGACTGGAATTC |
| | | CTGGTGTAGCGGTGAAATGCGCAGATATCAGGAGGAACACCGGTGGCGA |
| | | AGGCGGGTCTCTGGGAAACAACTGACGCTGAGGAACGAAAGCGTGGGTA |
| | | GCAAACAGGATTAGATACCCTGGTAGTCCACGCCGTAAACGGTGGGCGCT |
| | | AGGTGTGGGTTCCTTCCACGGGATCTGTGCCGTAGCTAACGCATTAAGCGC |
| | | CCCGCCTGGGGAGTACGGCCGCAAGGCTAAAACTCAAAGGAATTGACGG |
| | | GGGCCCGCACAAGCGGCGGAGCATGTGGATTAATTCGATGCAACGCGAAG |
| | | AACCTTACCTGGGTTTGACATACACCGGAAAACCGTAGAGATACGGTCCC |
| | | CCTTGTGGTCGGTGTACAGGTGGTGCATGGCTGTCGTCAGCTCGTGTCGTG |
| | | AGATGTTGGGTTAAGTCCCGCAACGAGCGCAACCCTTGTCTTATGTTGCCA |
| | | GCACGTAATGGTGGGGACTCGTAAGAGACTGCCGGGGTCAACTCGGAGGA |
| | | AGGTGGGGACGACGTCAAGTCATCATGCCCCTTATGTCCAGGGCTTCACA |
| | | CATGCTACAATGGCCAGTACAGAGGGCTGCGAGACCGTGAGGTGGAGCG |
| | | AATCCCTTAAAGCTGGTCTCAGTTCGGATCGGGGTCTGCAACTCGACCCCG |
| | | TGAAGTCGGAGTCGCTAGTAATCGCAGATCAGCAACGCTGCGGTGAATAC |
| | | GTTCCCGGGCCTTGTACACACCGCCCGTCACGTCATGAAAGTCGGTAACA |
| | | CCCGAAGCCGGTGGCCTAACCCCTTACGGGGAGGGAGCCGTCGAAGGTGG |
| | | GATCGGCGATTGGGACGAAGTCGTAACAAGGTAGCCGTACCGGAAGGTGC |
| | | GGCTGGATCACCTCCTTT |
| 39 | DP39 16S | CTTGAGAGTTTGATCCTGGCTCAGAACGAACGCTGGCGGCAGGCTTAACA |
| | rRNA | CATGCAAGTCGAACGCCCCGCAAGGGGAGTGGCAGACGGGTGAGTAACG |
| | | CGTGGGAATCTACCGTGCCCTGCGGAATAGCTCCGGGAAACTGGAATTAA |
| | | TACCGCATACGCCCTACGGGGGAAAGATTTATCGGGGTATGATGAGCCCG |
| | | CGTTGGATTAGCTAGTTGGTGGGGTAAAGGCCTACCAAGGCGACGATCCA |
| | | TAGCTGGTCTGAGAGGATGATCAGCCACATTGGGACTGAGACACGGCCCA |
| | | AACTCCTACGGGAGGCAGCAGTGGGGAATATTGGACAATGGGCGCAAGC |
| | | CTGATCCAGCCATGCCGCGTGAGTGATGAAGGCCTTAGGGTTGTAAAGCT |
| | | CTTTCACCGGAGAAGATAATGACGGTATCCGGAGAAGAAGCCCCGGCTAA |
| | | CTTCGTGCCAGCAGCCGCGGTAATACGAAGGGGGCTAGCGTTGTTCGGAA |
| | | TTACTGGGCGTAAAGCGCACGTAGGCGGATATTTAAGTCAGGGGTGAAAT |
| | | CCCAGAGCTCAACTCTGGAACTGCCTTTGATACTGGGTATCTTGAGTATGG |
| | | AAGAGGTAAGTGGAATTCCGAGTGTAGAGGTGAAATTCGTAGATATTCGG |
| | | AGGAACACCAGTGGCGAAGGCGGCTTACTGGTCCATTACTGACGCTGAGG |
| | | TGCGAAAGCGTGGGGAGCAAACAGGATTAGATACCCTGGTAGTCCACGCC |
| | | GTAAACGATGAATGTTAGCCGTCGGGCAGTATACTGTTCGGTGGCGCAGC |
| | | TAACGCATTAAACATTCCGCCTGGGGAGTACGGTCGCAAGATTAAAACTC |
| | | AAAGGAATTGACGGGGGCCCGCACAAGCGGTGGAGCATGTGGTTTAATTC |
| | | GAAGCAACGCGCAGAACCTTACCAGCTCTTGACATTCGGGGTTTGGGCAG |
| | | TGGAGACATTGTCCTTCAGTTAGGCTGGCCCCAGAACAGGTGCTGCATGG |
| | | CTGTCGTCAGCTCGTGTCGTGAGATGTTGGGTTAAGTCCCGCAACGAGCGC |
| | | AACCCTCGCCCTTAGTTGCCAGCATTTAGTTGGGCACTCTAAGGGGACTGC |
| | | CGGTGATAAGCCGAGAGGAAGGTGGGGATGACGTCAAGTCCTCATGGCCC |
| | | TTACGGGCTGGGCTACACACGTGCTACAATGGTGGTGACAGTGGGCAGCG |
| | | AGACAGCGATGTCGAGCTAATCTCCAAAAGCCATCTCAGTTCGGATTGCA |
| | | CTCTGCAACTCGAGTGCATGAAGTTGGAATCGCTAGTAATCGCAGATCAG |
| | | CATGCTGCGGTGAATACGTTCCCGGGCCTTGTACACACCGCCCGTCACACC |
| | | ATGGGAGTTGGTTTTACCCGAAGGTAGTGCGCTAACCGCAAGGAGGCAGC |
| | | TAACCACGGTAGGGTCAGCGACTGGGGTGAAGTCGTAACAAGGTAGCCGT |
| | | AGGGGAACCTGCGGCTGGATCACCTCCTTT |
| 40 | DP40 16S | TTGACGTTACCCGCAGAAGAAGCACCGGCTAACTCCGTGCCAGCAGCCGC |
| | rRNA | GGTAATACGGAGGGTGCAAGCGTTAATCGGAATTACTGGGCGTAAAGCGC |
| | | ACGCAGGCGGTCTGTTAAGTCAGATGTGAAATCCCCGGGCTTAACCTGGG |
| | | AACTGCATTTGAAACTGGCAGGCTTGAGTCTTGTAGAGGGGGGTAGAATT |
| | | CCAGGTGTAGCGGTGAAATGCGTAGAGATCTGGAGGAATACCGGTGGCGA |
| | | AGGCGGCCCCCTGGACAAAGACTGACGCTCAGGTGCGAAAGCGTGGGGA |
| | | GCAAACAGGATTAGATACCCTGGTAGTCCACGCCGTAAACGATGTCGACT |
| | | TGGAGGTTGTTCCCTTGAGGAGTGGCTTCCGGAGCTAACGCGTTAAGTCG |
| | | ACCGCCTGGGGAGTACGGCCGCAAGGTTAAAACTCAAATGAATTGACGGG |
| | | GGCCCGCACAAGCGGTGGAGCATGTGGTTTAATTCGATGCAACGCGAAGA |
| | | ACCTTACCTACTCTTGACATCCAGAGAACTTTCCAGAGATGGATTGGTGCC |
| | | TTCGGGAACTCTGAGACAGGTGCTGCATGGCTGTCGTCAGCTCGTGTTGTG |
| | | AAATGTTGGGTTAAGTCCCGCAACGAGCGCAACCCTTATCCTTTGTTGCCA |
| | | GCGCGTGATGGCGGGAACTCAAAGGAGACTGCCGGTGATAAACCGGAGG |
| | | AAGGTGGGGATGACGTCAAGTCATCATGGCCCTTACGAGTAGGGCTACAC |
| | | ACGTGCTACAATGGCGCATACAAAGAGAAGCGACCTCGCGAGAGCAAGC |
| | | GGACCTCACAAAGTGCGTCGTAGTCCGGATCGGAGTCTGCAACTCGACTC |
| | | CGTGAAGTCGGAATCGCTAGTAATCGTGGATCAGAATGCCACGGTGAATA |
| | | CGT |
| 41 | DP41 16S | GTGGAGAGTTTGATCCTGGCTCAGGATGAACGCTGGCGGCGTGCTTAACA |
| | rRNA | CATGCAAGTCGAACGGAAAGGCCCAAGCTTGCTTGGGTACTCGAGTGGCG |
| | | AACGGGTGAGTAACACGTGGGTGATCTGCCCTGCACTTCGGGATAAGCCT |
| | | GGGAAACTGGGTCTAATACCGGATAGGACGATGGTTTGGATGCCATTGTG |
| | | GAAAGTTTTTTCGGTGTGGGATGAGCTCGCGGCCTATCAGCTTGTTGGTGG |
| | | GGTAATGGCCTACCAAGGCGTCGACGGGTAGCCGGCCTGAGAGGGTGTAC |
| | | GGCCACATTGGGACTGAGATACGGCCCAGACTCCTACGGGAGGCAGCAGT |
| | | GGGGAATATTGCACAATGGGCGCAAGCCTGATGCAGCGACGCCGCGTGGG |
| | | GGATGACGGCCTTCGGGTTGTAAACTCCTTTCGCTAGGGACGAAGCGTTTT |
| | | GTGACGGTACCTGGAGAAGAAGCACCGGCTAACTACGTGCCAGCAGCCGC |
| | | GGTAATACGTAGGGTGCGAGCGTTGTCCGGAATTACTGGGCGTAAAGAGC |
| | | TCGTAGGTGGTTTGTCGCGTCGTTTGTGTAAGCCCGCAGCTTAACTGCGGG |
| | | ACTGCAGGCGATACGGGCATAACTTGAGTGCTGTAGGGGAGACTGGAATT |
| | | CCTGGTGTAGCGGTGGAATGCGCAGATATCAGGAGGAACACCGATGGCGA |
| | | AGGCAGGTCTCTGGGCAGTAACTGACGCTGAGGAGCGAAAGCATGGGTA |
| | | GCGAACAGGATTAGATACCCTGGTAGTCCATGCCGTAAACGGTGGGCGCT |
| | | AGGTGTGAGTCCCTTCCACGGGGTTCGTGCCGTAGCTAACGCATTAAGCG |
| | | CCCCGCCTGGGGAGTACGGCCGCAAGGCTAAAACTCAAAGGAATTGACGG |
| | | GGGCCCGCACAAGCGGCGGAGCATGTGGATTAATTCGATGCAACGCGAAG |
| | | AACCTTACCTGGGCTTGACATACACCAGATCGCCGTAGAGATACGGTTTCC |
| | | CTTTGTGGTTGGTGTACAGGTGGTGCATGGTTGTCGTCAGCTCGTGTCGTG |
| | | AGATGTTGGGTTAAGTCCCGCAACGAGCGCAACCCTTGTCTTATGTTGCCA |
| | | GCACGTGATGGTGGGGACTCGTGAGAGACTGCCGGGGTTAACTCGGAGGA |
| | | AGGTGGGGATGACGTCAAATCATCATGCCCCTTATGTCCAGGGCTTCACA |
| | | CATGCTACAATGGTCGGTACAACGCGCATGCGAGCCTGTGAGGGTGAGCG |
| | | AATCGCTGTGAAAGCCGGTCGTAGTTCGGATTGGGGTCTGCAACTCGACC |
| | | CCATGAAGTCGGAGTCGCTAGTAATCGCAGATCAGCAACGCTGCGGTGAA |
| | | TACGTTCCCGGGCCTTGTACACACCGCCCGTCACACCATGGGAGTGGGTTG |
| | | CAAAAGAAGTAGGTAGCTTAACCTTCGGGAGGGCGCTTACCACTTTGTGA |
| | | T |
| 42 | DP42 16S | TGAAGAGTTTGATCATGGCTCAGATTGAACGCTGGCGGCAGGCCTAACAC |
| | rRNA | ATGCAAGTCGAGCGGTAGAGAGGTGCTTGCACCTCTTGAGAGCGGCGGAC |
| | | GGGTGAGTAATACCTAGGAATCTGCCTGATAGTGGGGGATAACGTTCGGA |
| | | AACGGACGCTAATACCGCATACGTCCTACGGGAGAAAGCAGGGGACCTTC |
| | | GGGCCTTGCGCTATCAGATGAGCCTAGGTCGGATTAGCTAGTTGGTGAGG |
| | | TAATGGCTCACCAAGGCTACGATCCGTAACTGGTCTGAGAGGATGATCAG |
| | | TCACACTGGAACTGAGACACGGTCCAGACTCCTACGGGAGGCAGCAGTGG |
| | | GGAATATTGGACAATGGGCGAAAGCCTGATCCAGCCATGCCGCGTGTGTG |
| | | AAGAAGGTCTTCGGATTGTAAAGCACTTTAAGTTGGGAGGAAGGGCATTA |
| | | ACCTAATACGTTAGTGTCTTGACGTTACCGACAGAATAAGCACCGGCTAA |
| | | CTCTGTGCCAGCAGCCGCGGTAATACAGAGGGTGCAAGCGTTAATCGGAA |
| | | TTACTGGGCGTAAAGCGCGCGTAGGTGGTTTGTTAAGTTGAATGTGAAAT |
| | | CCCCGGGCTCAACCTGGGAACTGCATCCAAAACTGGCAAGCTAGAGTATG |
| | | GTAGAGGGTAGTGGAATTTCCTGTGTAGCGGTGAAATGCGTAGATATAGG |
| | | AAGGAACACCAGTGGCGAAGGCGACTACCTGGACTGATACTGACACTGAG |
| | | GTGCGAAAGCGTGGGGAGCAAACAGGATTAGATACCCTGGTAGTCCACGC |
| | | CGTAAACGATGTCAACTAGCCGTTGGGAACCTTGAGTTCTTAGTGGCGCA |
| | | GCTAACGCATTAAGTTGACCGCCTGGGGAGTACGGCCGCAAGGTTAAAAC |
| | | TCAAATGAATTGACGGGGGCCCGCACAAGCGGTGGAGCATGTGGTTTAAT |
| | | TCGAAGCAACGCGAAGAACCTTACCAGGCCTTGACATCCAATGAACTTTC |
| | | CAGAGATGGATTGGTGCCTTCGGGAACATTGAGACAGGTGCTGCATGGCT |
| | | GTCGTCAGCTCGTGTCGTGAGATGTTGGGTTAAGTCCCGTAACGAGCGCA |
| | | ACCCTTGTCCTTAGTTACCAGCACGTAATGGTGGGCACTCTAAGGAGACTG |
| | | CCGGTGACAAACCGGAGGAAGGTGGGGATGACGTCAAGTCATCATGGCCC |
| | | TTACGGCCTGGGCTACACACGTGCTACAATGGTCGGTACAAAGGGTTGCC |
| | | AAGCCGCGAGGTGGAGCTAATCCCATAAAACCGATCGTAGTCCGGATCGC |
| | | AGTCTGCAACTCGACTGCGTGAAGTCGGAATCGCTAGTAATCGTGAATCA |
| | | GAATGTCACGGTGAATACGTTCCCGGGCCTTGTACACACCGCCCGTCACA |
| | | CCATGGGAGTGGGTTGCACCAGAAGTAGCTAGTCTAACCCTCGGGAGGAC |
| | | GGTTACCACGGTGTGATTCATGACTGGGGTGAAGTCGTAACAAGGTAGCC |
| | | GTAGGGGAACCTGCGGCTGGATCACCTCCTT |
| 43 | DP43 16S | CTGAGTTTGATCCTGGCTCAGATTGAACGCTGGCGGCATGCCTTACACATG |
| | rRNA | CAAGTCGAACGGCAGCACGGAGCTTGCTCTGGTGGCGAGTGGCGAACGGG |
| | | TGAGTAATATATCGGAACGTACCCTGGAGTGGGGGATAACGTAGCGAAAG |
| | | TTACGCTAATACCGCATACGATCTAAGGATGAAAGTGGGGGATCGCAAGA |
| | | CCTCATGCTCGTGGAGCGGCCGATATCTGATTAGCTAGTTGGTAGGGTAA |
| | | AAGCCTACCAAGGCATCGATCAGTAGCTGGTCTGAGAGGACGACCAGCCA |
| | | CACTGGAACTGAGACACGGTCCAGACTCCTACGGGAGGCAGCAGTGGGG |
| | | AATTTTGGACAATGGGCGAAAGCCTGATCCAGCAATGCCGCGTGAGTGAA |
| | | GAAGGCCTTCGGGTTGTAAAGCTCTTTTGTCAGGGAAGAAACGGTGAGAG |
| | | CTAATATCTCTTGCTAATGACGGTACCTGAAGAATAAGCACCGGCTAACT |
| | | ACGTGCCAGCAGCCGCGGTAATACGTAGGGTGCAAGCGTTAATCGGAATT |
| | | ACTGGGCGTAAAGCGTGCGCAGGCGGTTTTGTAAGTCTGATGTGAAATCC |
| | | CCGGGCTCAACCTGGGAATTGCATTGGAGACTGCAAGGCTAGAATCTGGC |
| | | AGAGGGGGGTAGAATTCCACGTGTAGCAGTGAAATGCGTAGATATGTGGA |
| | | GGAACACCGATGGCGAAGGCAGCCCCCTGGGTCAAGATTGACGCTCATGC |
| | | ACGAAAGCGTGGGGAGCAAACAGGATTAGATACCCTGGTAGTCCACGCCC |
| | | TAAACGATGTCTACTAGTTGTCGGGTCTTAATTGACTTGGTAACGCAGCTA |
| | | ACGCGTGAAGTAGACCGCCTGGGGAGTACGGTCGCAAGATTAAAACTCAA |
| | | AGGAATTGACGGGGACCCGCACAAGCGGTGGATGATGTGGATTAATTCGA |
| | | TGCAACGCGAAAAACCTTACCTACCCTTGACATGGCTGGAATCCTTGAGA |
| | | GATCAGGGAGTGCTCGAAAGAGAACCAGTACACAGGTGCTGCATGGCTGT |
| | | CGTCAGCTCGTGTCGTGAGATGTTGGGTTAAGTCCCGCAACGAGCGCAAC |
| | | CCTTGTCATTAGTTGCTACGAAAGGGCACTCTAATGAGACTGCCGGTGAC |
| | | AAACCGGAGGAAGGTGGGGATGACGTCAAGTCCTCATGGCCCTTATGGGT |
| | | AGGGCTTCACACGTCATACAATGGTACATACAGAGCGCCGCCAACCCGCG |
| | | AGGGGGAGCTAATCGCAGAAAGTGTATCGTAGTCCGGATTGTAGTCTGCA |
| | | ACTCGACTGCATGAAGTTGGAATCGCTAGTAATCGCGGATCAGCATGTCG |
| | | CGGTGAATACGTTCCCGGGTCTTGTACACACCGCCCGTCACACCATGGGA |
| | | GCGGGTTTTACCAGAAGTAGGTAGCTTAACCGTAAGGAGGGCGCTTACCA |
| | | CGGTAGGATTCGTGACTGGGGTGAAGTCGTAACAAGGTAGCCGTATCGGA |
| | | AGGTGCGGCTGGATCACCTCCTTT |
| 44 | DP44 16S | TGGCGGCATGCCTTACACATGCAAGTCGAACGGCAGCATAGGAGCTTGCT |
| | rRNA | CCTGATGGCGAGTGGCGAACGGGTGAGTAATATATCGGAACGTGCCCTAG |
| | | AGTGGGGGATAACTAGTCGAAAGACTAGCTAATACCGCATACGATCTACG |
| | | GATGAAAGTGGGGGATCGCAAGACCTCATGCTCCTGGAGCGGCCGATATC |
| | | TGATTAGCTAGTTGGTGGGGTAAAAGCTCACCAAGGCGACGATCAGTAGC |
| | | TGGTCTGAGAGGACGACCAGCCACACTGGGACTGAGACACGGCCCAGACT |
| | | CCTACGGGAGGCAGCAGTGGGGAATTTTGGACAATGGGGGCAACCCTGAT |
| | | CCAGCAATGCCGCGTGAGTGAAGAAGGCCTTCGGGTTGTAAAGCTCTTTT |
| | | GTCAGGGAAGAAACGGTTCTGGATAATACCTAGGACTAATGACGGTACCT |
| | | GAAGAATAAGCACCGGCTAACTACGTGCCAGCAGCCGCGGTAATACGTAG |
| | | GGTGCAAGCGTTAATCGGAATTACTGGGCGTAAAGCGTGCGCAGGCGGTT |
| | | GTGTAAGTCAGATGTGAAATCCCCGGGCTCAACCTGGGAATTGCATTTGA |
| | | GACTGCACGGCTAGAGTGTGTCAGAGGGGGGTAGAATTCCACGTGTAGCA |
| | | GTGAAATGCGTAGATATGTGGAGGAATACCGATGGCGAAGGCAGCCCCCT |
| | | GGGATAACACTGACGCTCATGCACGAAAGCGTGGGGAGCAAACAGGATT |
| | | AGATACCCTGGTAGTCCACGCCCTAAACGATGTCTACTAGTTGTCGGGTCT |
| | | TAATTGACTTGGTAACGCAGCTAACGCGTGAAGTAGACCGCCTGGGGAGT |
| | | ACGGTCGCAAGATTAAAACTCAAAGGAATTGACGGGGACCCGCACAAGC |
| | | GGTGGATGATGTGGATTAATTCGATGCAACGCGAAAAACCTTACCTACCC |
| | | TTGACATGGATGGAATCCCGAAGAGATTTGGGAGTGCTCGAAAGAGAACC |
| | | ATCACACAGGTGCTGCATGGCTGTCGTCAGCTCGTGTCGTGAGATGTTGGG |
| | | TTAAGTCCCGCAACGAGCGCAACCCTTGTCATTAGTTGCTACGAAAGGGC |
| | | ACTCTAATGAGACTGCCGGTGACAAACCGGAGGAAGGTGGGGATGACGTC |
| | | AAGTCCTCATGGCCCTTATGGGTAGGGCTTCACACGTCATACAATGGTACA |
| | | TACAGAGGGCCGCCAACCCGCGAGGGGGAGCTAATCCCAGAAAGTGTATC |
| | | GTAGTCCGGATTGGAGTCTGCAACTCGACTCCATGAAGTTGGAATCGCTA |
| | | GTAATCGCGGATCAGCATGTCGCGGTGAATACGTTCCCGGGTCTTGTACAC |
| | | ACCGCCCGTCACACCATGGGAGCGGGTTTTACCAGAAGTGGGTAGCCTAA |
| | | CCGCAAGGAGGGCGCTCACCACGGTAGGATTCGTGACTGGGGTGAAGTCG |
| | | TAACAAGGTAGCCGTATCGGAAGGTGCGGCTGGATCACCTCCTTT |
| 45 | DP45 16S | TACGGAGAGTTTGATCCTGGCTCAGGACGAACGCTGGCGGCGTGCTTAAC |
| | rRNA | ACATGCAAGTCGAACGGTGACGCTAGAGCTTGCTCTGGTTGATCAGTGGC |
| | | GAACGGGTGAGTAACACGTGAGTAACCTGCCCTTGACTCTGGGATAACTC |
| | | CGGGAAACCGGGGCTAATACCGGATACGAGACGCGACCGCATGGTCGGC |
| | | GTCTGGAAAGTTTTTCGGTCAAGGATGGACTCGCGGCCTATCAGCTTGTTG |
| | | GTGAGGTAATGGCTCACCAAGGCGTCGACGGGTAGCCGGCCTGAGAGGGC |
| | | GACCGGCCACACTGGGACTGAGACACGGCCCAGACTCCTACGGGAGGCA |
| | | GCAGTGGGGAATATTGCACAATGGGCGAAAGCCTGATGCAGCGACGCCGC |
| | | GTGAGGGATGAAGGCCTTCGGGTTGTAAACCTCTTTCAGTAGGGAAGAAG |
| | | CGAAAGTGACGGTACCTGCAGAAGAAGCGCCGGCTAACTACGTGCCAGCA |
| | | GCCGCGGTAATACGTAGGGCGCAAGCGTTGTCCGGAATTATTGGGCGTAA |
| | | AGAGCTCGTAGGCGGTTTGTCGCGTCTGGTGTGAAAACTCAAGGCTCAAC |
| | | CTTGAGCTTGCATCGGGTACGGGCAGACTAGAGTGTGGTAGGGGTGACTG |
| | | GAATTCCTGGTGTAGCGGTGGAATGCGCAGATATCAGGAGGAACACCGAT |
| | | GGCGAAGGCAGGTCACTGGGCCACTACTGACGCTGAGGAGCGAAAGCAT |
| | | GGGGAGCGAACAGGATTAGATACCCTGGTAGTCCATGCCGTAAACGTTGG |
| | | GCACTAGGTGTGGGGCTCATTCCACGAGTTCCGCGCCGCAGCTAACGCAT |
| | | TAAGTGCCCCGCCTGGGGAGTACGGCCGCAAGGCTAAAACTCAAAGGAAT |
| | | TGACGGGGGCCCGCACAAGCGGCGGAGCATGCGGATTAATTCGATGCAAC |
| | | GCGAAGAACCTTACCAAGGCTTGACATACACCGGAATCATGCAGAGATGT |
| | | GTGCGTCTTCGGACTGGTGTACAGGTGGTGCATGGTTGTCGTCAGCTCGTG |
| | | TCGTGAGATGTTGGGTTAAGTCCCGCAACGAGCGCAACCCTCGTCCTATGT |
| | | TGCCAGCACGTTATGGTGGGGACTCATAGGAGACTGCCGGGGTCAACTCG |
| | | GAGGAAGGTGGGGATGACGTCAAATCATCATGCCCCTTATGTCTTGGGCT |
| | | TCACGCATGCTACAATGGCCGGTACAAAGGGCTGCGATACCGCGAGGTGG |
| | | AGCGAATCCCAAAAAGCCGGTCTCAGTTCGGATTGGGGTCTGCAACTCGA |
| | | CCCCATGAAGTCGGAGTCGCTAGTAATCGCAGATCAGCAACGCTGCGGTG |
| | | AATACGTTCCCGGGCCTTGTACACACCGCCCGTCAAGTCACGAAAGTCGG |
| | | TAACACCCGAAGCCGGTGGCCTAACCCCTTGTGGGATGGAGCCGTCGAAG |
| | | GTGGGATTGGCGATTGGGACTAAGTCGTAACAAGGTAGCCGTACCGGAAG |
| | | GTGCGGCTGGATCACCTCCTTT |
| 46 | DP46 16S | TTGAAGAGTTTGATCATGGCTCAGATTGAACGCTGGCGGCAGGCCTAACA |
| | rRNA | CATGCAAGTCGGACGGTAGCACAGAGGAGCTTGCTCCTTGGGTGACGAGT |
| | | GGCGGACGGGTGAGTAATGTCTGGGGATCTGCCCGATAGAGGGGGATAAC |
| | | CACTGGAAACGGTGGCTAATACCGCATAACGTCGCAAGACCAAAGAGGG |
| | | GGACCTTCGGGCCTCTCACTATCGGATGAACCCAGATGGGATTAGCTAGT |
| | | AGGCGGGGTAATGGCCCACCTAGGCGACGATCCCTAGCTGGTCTGAGAGG |
| | | ATGACCAGCCACACTGGAACTGAGACACGGTCCAGACTCCTACGGGAGGC |
| | | AGCAGTGGGGAATATTGCACAATGGGCGCAAGCCTGATGCAGCCATGCCG |
| | | CGTGTATGAAGAAGGCCTTCGGGTTGTAAAGTACTTTCAGCGGGGAGGAA |
| | | GGCGACAGGGTTAATAACCCTGTCGATTGACGTTACCCGCAGAAGAAGCA |
| | | CCGGCTAACTCCGTGCCAGCAGCCGCGGTAATACGGAGGGTGCAAGCGTT |
| | | AATCGGAATTACTGGGCGTAAAGCGCACGCAGGCGGTCTGTTAAGTCAGA |
| | | TGTGAAATCCCCGGGCTTAACCTGGGAACTGCATTTGAAACTGGCAGGCT |
| | | TTAGTCTTGTAGAGTGGGGTAGAATTCCAGGTGTAGCGGTGAAATGCGTA |
| | | GAGATGTGGAGGAACACCAGTGGCGAAGGCGGCTTTTTGGTCTGTAACTG |
| | | ACGCTGAGGCGCGAAAGCGTGGGGAGCAAACAGGATTAGATACCCTGGT |
| | | AGTCCACGCCGTAAACGATGAGTGCTAAGTGTT |
| 47 | DP47 16S | AGGGTGCAAGCGTTAATCGGAATTACTGGGCGTAAAGCGCGCGTAGGTGG |
| | rRNA | TTTGTTAAGTTGAATGTGAAATCCCCGGGCTCAACCTGGGAACTGCATTTG |
| | | AAACTGGCAAGCTAGAGTCTCGTAGAGGGGGGTAGAATTCCAGGTGTAGC |
| | | GGTGAAATGCGTAGAGATCTGGAGGAATACCGGTGGCGAAGGCGGCCCC |
| | | CTGGACGAAGACTGACGCTCAGGTGCGAAAGCGTGGGGAGCAAACAGGA |
| | | TTAGATACCCTGGTAGTCCACGCCGTAAACGATGTCAACTAGCCGTTGGA |
| | | AGCCTTGAGCTTTTAGTGGCGCAGCTAACGCATTAAGTTGACCGCCTGGG |
| | | GAGTACGGCCGCAAGGTTAAAACTCAAATGAATTGACGGGGGCCCGCACA |
| | | AGCGGTGGAGCATGTGGTTTAATTCGAAGCAACGCGAAGAACCTTACCAG |
| | | GCCTTGACATCCAATGAACTTTCTAGAGATAGATTGGTGCCTTCGGGAACA |
| | | TTGAGACAGGTGCTGCATGGCTGTCGTCAGCTCGTGTCGTGAGATGTTGGG |
| | | TTAAGTCCCGCAACGAGCGCAACCCTTGTCCTGTGTTGCCAGCGCGTAATG |
| | | GCGGGGACTCGCAGGAGACTGCCGGGGTCAACTCGGAGGAAGGTGGGGA |
| | | TGACGTCAAATCATCATGCCCCTTATGTCTTGGGCTTCACGCATGCTACAA |
| | | TGGCCGGTACAAAGGGCTGCAATACCGTGAGGTGGAGCGAATCCCAAAA |
| | | AGCCGGTCCCAGTTCGGATTGAGGTCTGCAACTCGACCTCATGAAGTCGG |
| | | AGTCGCTAGTAATCGCAGATCAGCAACGCTGCGGTGAATACGTTCCCGGG |
| | | TCTTGTACACACCGCCCGTCAAGTCATGAAAGTCGGTAACACCTGAAGCC |
| | | GGTGGCCCAACCCTTGTGGAGGGAGCCGTCGAAGGTGGGATCGGTAATTA |
| | | GGACTAAGT |
| 48 | DP48 16S | CATGGAGAGTTTGATCCTGGCTCAGGACGAACGCTGGCGGCGTGCCTAAT |
| | rRNA | ACATGCAAGTCGAGCGGACAGATGGGAGCTTGCTCCCTGATGTTAGCGGC |
| | | GGACGGGTGAGTAACACGTGGGTAACCTGCCTGTAAGACTGGGATAACTC |
| | | CGGGAAACCGGGGCTAATACCGGATGCTTGATTGAACCGCATGGTTCAAT |
| | | TATAAAAGGTGGCTTTTAGCTACCACTTACAGATGGACCCGCGGCGCATT |
| | | AGCTAGTTGGTGAGGTAACGGCTCACCAAGGCAACGATGCGTAGCCGACC |
| | | TGAGAGGGTGATCGGCCACACTGGGACTGAGACACGGCCCAGACTCCTAC |
| | | GGGAGGCAGCAGTAGGGAATCTTCCGCAATGGACGAAAGTCTGACGGAG |
| | | CAACGCCGCGTGAGTGATGAAGGTTTTCGGATCGTAAAACTCTGTTGTTAG |
| | | GGAAGAACAAGTACCGTTCGAATAGGGCGGTACCTTGACGGTACCTAACC |
| | | AGAAAGCCACGGCTAACTACGTGCCAGCAGCCGCGGTAATACGTAGGTGG |
| | | CAAGCGTTGTCCGGAATTATTGGGCGTAAAGCGCGCGCAGGCGGTTTCTT |
| | | AAGTCTGATGTGAAAGCCCCCGGCTCAACCGGGGAGGGTCATTGGAAACT |
| | | GGGGAACTTGAGTGCAGAAGAGGAGAGTGGAATTCCACGTGTAGCGGTG |
| | | AAATGCGTAGAGATGTGGAGGAACACCAGTGGCGAAGGCGACTCTCTGGT |
| | | CTGTAACTGACGCTGAGGCGCGAAAGCGTGGGGAGCGAACAGGATTAGA |
| | | TACCCTGGTAGTCCACGCCGTAAACGATGAGTGCTAAGTGTTAGAGGGTT |
| | | TCCGCCCTTTAGTGCTGCAGCAAACGCATTAAGCACTCCGCCTGGGGAGT |
| | | ACGGTCGCAAGACTGAAACTCAAAGGAATTGACGGGGGCCCGCACAAGC |
| | | GGTGGAGCATGTGGTTTAATTCGAAGCAACGCGAAGAACCTTACCAGGTC |
| | | TTGACATCCTCTGACAACCCTAGAGATAGGGCTTCCCCTTCGGGGGCAGA |
| | | GTGACAGGTGGTGCATGGTTGTCGTCAGCTCGTGTCGTGAGATGTTGGGTT |
| | | AAGTCCCGCAACGAGCGCAACCCTTGATCTTAGTTGCCAGCATTCAGTTGG |
| | | GCACTCTAAGGTGACTGCCGGTGACAAACCGGAGGAAGGTGGGGATGAC |
| | | GTCAAATCATCATGCCCCTTATGACCTGGGCTACACACGTGCTACAATGGG |
| | | CAGAACAAAGGGCAGCGAAGCCGCGAGGCTAAGCCAATCCCACAAATCT |
| | | GTTCTCAGTTCGGATCGCAGTCTGCAACTCGACTGCGTGAAGCTGGAATCG |
| | | CTAGTAATCGCGGATCAGCATGCCGCGGTGAATACGTTCCCGGGCCTTGT |
| | | ACACACCGCCCGTCACACCACGAGAGTTTGTAACACCCGAAGTCGGTGAG |
| | | GTAACCTTTTGGAGCCAGCCGCCGAAGGTGGGACAGATGATTGGGGTGAA |
| | | GTCGTAACAAGGTAGCCGTATCGGAAGGTGCGGCTGGATCACCTCCTTT |
| 49 | DP49 16S | TATGGAGAGTTTGATCCTGGCTCAGGACGAACGCTGGCGGCGTGCCTAAT |
| | rRNA | ACATGCAAGTCGAGCGGACGTTTTTGAAGCTTGCTTCAAAAACGTTAGCG |
| | | GCGGACGGGTGAGTAACACGTGGGCAACCTGCCTTATCGACTGGGATAAC |
| | | TCCGGGAAACCGGGGCTAATACCGGATAATATCTAGCACCTCCTGGTGCA |
| | | AGATTAAAAGAGGGCCTTCGGGCTCTCACGGTGAGATGGGCCCGCGGCGC |
| | | ATTAGCTAGTTGGAGAGGTAATGGCTCCCCAAGGCGACGATGCGTAGCCG |
| | | ACCTGAGAGGGTGATCGGCCACACTGGGACTGAGACACGGCCCAGACTCC |
| | | TACGGGAGGCAGCAGTAGGGAATCTTCCGCAATGGACGAAAGTCTGACGG |
| | | AGCAACGCCGCGTGAGTGATGAAGGGTTTCGGCTCGTAAAGCTCTGTTAT |
| | | GAGGGAAGAACACGTACCGTTCGAATAGGGCGGTACCTTGACGGTACCTC |
| | | ATCAGAAAGCCACGGCTAACTACGTGCCAGCAGCCGCGGTAATACGTAGG |
| | | TGGCAAGCGTTGTCCGGAATTATTGGGCGTAAAGCGCGCGCAGGCGGCCT |
| | | TTTAAGTCTGATGTGAAATCTTGCGGCTCAACCGCAAGCGGTCATTGGAA |
| | | ACTGGGAGGCTTGAGTACAGAAGAGGAGAGTGGAATTCCACGTGTAGCG |
| | | GTGAAATGCGTAGATATGTGGAGGAACACCAGTGGCGAAGGCGACTCTCT |
| | | GGTCTGTAACTGACGCTGAGGCGCGAAAGCGTGGGGAGCAAACAGGATT |
| | | AGATACCCTGGTAGTCCACGCCGTAAACGATGAGTGCTAGGTGTTAGGGG |
| | | TTTCGATGCCCGTAGTGCCGAAGTTAACACATTAAGCACTCCGCCTGGGG |
| | | AGTACGGCCGCAAGGCTGAAACTCAAAGGAATTGACGGGGGCCCGCACA |
| | | AGCAGTGGAGCATGTGGTTTAATTCGAAGCAACGCGAAGAACCTTACCAG |
| | | GTCTTGACATCCTTTGACCACTCTGGAGACAGAGCTTCCCCTTCGGGGGCA |
| | | AAGTGACAGGTGGTGCATGGTTGTCGTCAGCTCGTGTCGTGAGATGTTGG |
| | | GTTAAGTCCCGCAACGAGCGCAACCCTTGACCTTAGTTGCCAGCATTTAGT |
| | | TGGGCACTCTAAGGTGACTGCCGGTGACAAACCGGAGGAAGGTGGGGAT |
| | | GACGTCAAATCATCATGCCCCTTATGACCTGGGCTACACACGTGCTACAAT |
| | | GGATGGTACAAAGGGTTGCGAAGCCGCGAGGTGAAGCCAATCCCATAAA |
| | | GCCATTCTCAGTTCGGATTGTAGGCTGCAACTCGCCTGCATGAAGCTGGAA |
| | | TTGCTAGTAATCGCGGATCAGCATGCCGCGGTGAATACGTTCCCGGGCCTT |
| | | GTACACACCGCCCGTCACACCACGAGAGTTTGTAACACCCGAAGTCGGTG |
| | | AGGTAACCTTTTGGAGCCAGCCGCCGAAGGTGGGACAGATGATTGGGGTG |
| | | AAGTCGTAACAAGGTAGCCGTATCGGAAGGTGCGGCTGGATCACCTCCTT |
| | | T |
| 50 | DP50 16S | TTGAAGAGTTTGATCATGGCTCAGATTGAACGCTGGCGGCAGGCCTAACA |
| | rRNA | CATGCAAGTCGAACGGTAGCACAGAGAGCTTGCTCTTGGGTGACGAGTGG |
| | | CGGACGGGTGAGTAATGTCTGGGAAACTGCCCGATGGAGGGGGATAACTA |
| | | CTGGAAACGGTAGCTAATACCGCATAACGTCGCAAGACCAAAGTGGGGG |
| | | ACCTTCGGGCCTCACACCATCGGATGTGCCCAGATGGGATTAGCTAGTAG |
| | | GTGGGGTAATGGCTCACCTAGGCGACGATCCCTAGCTGGTCTGAGAGGAT |
| | | GACCAGCCACACTGGAACTGAGACACGGTCCAGACTCCTACGGGAGGCAG |
| | | CAGTGGGGAATATTGCACAATGGGCGCAAGCCTGATGCAGCCATGCCGCG |
| | | TGTATGAAGAAGGCCTTCGGGTTGTAAAGTACTTTCAGCGAGGAGGAAGG |
| | | CATTGTGGTTAATAACCGCAGTGATTGACGTTACTCGCAGAAGAAGCACC |
| | | GGCTAACTCCGTGCCAGCAGCCGCGGTAATACGGAGGGTGCAAGCGTTAA |
| | | TCGGAATTACTGGGCGTAAAGCGCACGCAGGCGGTCTGTCAAGTCGGATG |
| | | TGAAATCCCCGGGCTCAACCTGGGAACTGCATTCGAAACTGGCAGGCTAG |
| | | AGTCTTGTAGAGGGGGGTAGAATTCCAGGTGTAGCGGTGAAATGCGTAGA |
| | | GATCTGGAGGAATACCGGTGGCGAAGGCGGCCCCCTGGACAAAGACTGA |
| | | CGCTCAGGTGCGAAAGCGTGGGGAGCAAACAGGATTAGATACCCTGGTAG |
| | | TCCACGCCGTAAACGATGTCGACTTGGAGGTTGTGCCCTTGAGGCGTGGCT |
| | | TCCGGAGCTAACGCGTTAAGTCGACCGCCTGGGGAGTACGGCCGCAAGGT |
| | | TAAAACTCAAATGAATTGACGGGGGCCCGCACAAGCGGTGGAGCATGTGG |
| | | TTTAATTCGATGCAACGCGAAGAACCTTACCTACTCTTGACATCCACGGAA |
| | | TTTAGCAGAGATGCTTTAGTGCCTTCGGGAACCGTGAGACAGGTGCTGCA |
| | | TGGCTGTCGTCAGCTCGTGTTGTGAAATGTTGGGTTAAGTCCCGCAACGAG |
| | | CGCAACCCTTATCCTTTGTTGCCAGCGGTTCGGCCGGGAACTCAAAGGAG |
| | | ACTGCCAGTGATAAACTGGAGGAAGGTGGGGATGACGTCAAGTCATCATG |
| | | GCCCTTACGAGTAGGGCTACACACGTGCTACAATGGCATATACAAAGAGA |
| | | AGCGACCTCGCGAGAGCAAGCGGACCTCATAAAGTATGTCGTAGTCCGGA |
| | | TCGGAGTCTGCAACTCGACTCCGTGAAGTCGGAATCGCTAGTAATCGTAG |
| | | ATCAGAATGCTACGGTGAATACGTTCCCGGGCCTTGTACACACCGCCCGTC |
| | | ACACCATGGGAGTGGGTTGCAAAAGAAGTAGGTAGCTTAACCTTCGGGAG |
| | | GGCGCTTACCACTTTGTGATTCATGACTGGGGTGAAGTCGTAACAAGGTA |
| | | ACCGTAGGGGAACCTGCGGTTGGATCACCTCCTT |
| 51 | DP51 16S | TTGAAGAGTTTGATCATGGCTCAGATTGAACGCTGGCGGCAGGCCTAACA |
| | rRNA | CATGCAAGTCGAGCGGTAGCACAGGGAGCTTGCTCCTGGGTGACGAGCGG |
| | | CGGACGGGTGAGTAATGTCTGGGAAACTGCCTGATGGAGGGGGATAACTA |
| | | CTGGAAACGGTAGCTAATACCGCATAACGTCGCAAGACCAAAGAGGGGG |
| | | ACCTTCGGGCCTCTTGCCATCAGATGTGCCCAGATGGGATTAGCTAGTAGG |
| | | TGAGGTAATGGCTCACCTAGGCGACGATCCCTAGCTGGTCTGAGAGGATG |
| | | ACCAGCCACACTGGAACTGAGACACGGTCCAGACTCCTACGGGAGGCAGC |
| | | AGTGGGGAATATTGCACAATGGGCGCAAGCCTGATGCAGCCATGCCGCGT |
| | | GTATGAAGAAGGCCTTCGGGTTGTAAAGTACTTTCAGCGAGGAGGAAGGC |
| | | ATTAAGGTTAATAACCTTGGTGATTGACGTTACTCGCAGAAGAAGCACCG |
| | | GCTAACTCCGTGCCAGCAGCCGCGGTAATACGGGGGGTGCAAGCGTTAAT |
| | | CGGAATTACTGGGCGTAAAGCGCACGCAGGCGGTTTGTCAAGTCGGATGT |
| | | GAAATCCCCGGGCTCAACCTGGGAACTGCATTCGAAACGGGCAAGCTAGA |
| | | GTCTTGTAGAGGGGGGTAGAATTCCAGGTGTAGCGGTGAAATGCGTAGAG |
| | | ATCTGGAGGAATACCGGTGGCGAAGGCGGCCCCCTGGACAAAGACTGAC |
| | | GCTCAGGTGCGAAAGCGTGGGGAGCAAACAGGATTAGATACCCTGGTAGT |
| | | CCACGCCGTAAACGATGTCGACTTGGAGGTTGTGCCCTTGAGGCGTGGCTT |
| | | CCGGAGCTAACGCGTTAAGTCGACCGCCTGGGGAGTACGGCCGCAAGGTT |
| | | AAAACTCAAATGAATTGACGGGGGCCCGCACAAGCGGTGGAGCATGTGGT |
| | | TTAATTCGATGCAACGCGAAGAACCTTACCTACTCTTGACATCCAGAGAA |
| | | CTTTCCAGAGATGGATTGGTGCCTTCGGGAACTCTGAGACAGGTGCTGCAT |
| | | GGCTGTCGTCAGCTCGTGTTGTGAAATGTTGGGTTAAGTCCCGCAACGAGC |
| | | GCAACCCTTATCCTTTGTTGCCAGCGAGTAATGTCGGGAACTCAAAGGAG |
| | | ACTGCCAGTGACAAACTGGAGGAAGGTGGGGATGACGTCAAGTCATCATG |
| | | GCCCTTACGAGTAGGGCTACACACGTGCTACAATGGCATATACAAAGAGA |
| | | AGCGACCTCGCGAGAGCAAGCGGACCTCACAAAGTATGTCGTAGTCCGGA |
| | | TCGGAGTCTGCAACTCGACTCCGTGAAGTCGGAATCGCTAGTAATCGTAG |
| | | ATCAGAATGCTACGGTGAATACGTTCCCGGGCCTTGTACACACCGCCCGTC |
| | | ACACCATGGGAGTGGGTTGCAAAAGAAGTAGGTAGCTTAACCTTCGGGAG |
| | | GGCGCTTACCACTTTGTGATTCATGACTGGGGTGAAGTCGTAACAAGGTA |
| | | ACCGTAGGGGAACCTGCGGTTGGATCACCTCCTT |
| 52 | DP52 16S | ACGGAGAGTTTGATCCTGGCTCAGGATGAACGCTGGCGGCGTGCTTAACA |
| | rRNA | CATGCAAGTCGAACGATGATCCCAGCTTGCTGGGGGATTAGTGGCGAACG |
| | | GGTGAGTAACACGTGAGTAACCTGCCCTTGACTCTGGGATAAGCCTGGGA |
| | | AACTGGGTCTAATACCGGATATGACTGTCTGACGCATGTCAGGTGGTGGA |
| | | AAGCTTTTGTGGTTTTGGATGGACTCGCGGCCTATCAGCTTGTTGGTGGGG |
| | | TAATGGCCTACCAAGGCGACGACGGGTAGCCGGCCTGAGAGGGTGACCG |
| | | GCCACACTGGGACTGAGACACGGCCCAGACTCCTACGGGAGGCAGCAGTG |
| | | GGGAATATTGCACAATGGGCGCAAGCCTGATGCAGCGACGCCGCGTGAGG |
| | | GATGACGGCCTTCGGGTTGTAAACCTCTTTCAGTAGGGAAGAAGCGAAAG |
| | | TGACGGTACCTGCAGAAGAAGCGCCGGCTAACTACGTGCCAGCAGCCGCG |
| | | GTAATACGTAGGGCGCAAGCGTTATCCGGAATTATTGGGCGTAAAGAGCT |
| | | CGTAGGCGGTTTGTCGCGTCTGCTGTGAAAGACCGGGGCTCAACTCCGGTT |
| | | CTGCAGTGGGTACGGGCAGACTAGAGTGCAGTAGGGGAGACTGGAATTCC |
| | | TGGTGTAGCGGTGAAATGCGCAGATATCAGGAGGAACACCGATGGCGAA |
| | | GGCAGGTCTCTGGGCTGTAACTGACGCTGAGGAGCGAAAGCATGGGGAGC |
| | | GAACAGGATTAGATACCCTGGTAGTCCATGCCGTAAACGTTGGGCACTAG |
| | | GTGTGGGGGACATTCCACGTTTTCCGCGCCGTAGCTAACGCATTAAGTGCC |
| | | CCGCCTGGGGAGTACGGCCGCAAGGCTAAAACTCAAAGGAATTGACGGG |
| | | GGCCCGCACAAGCGGCGGAGCATGCGGATTAATTCGATGCAACGCGAAG |
| | | AACCTTACCAAGGCTTGACATGAACCGGTAATACCTGGAAACAGGTGCCC |
| | | CGCTTGCGGTCGGTTTACAGGTGGTGCATGGTTGTCGTCAGCTCGTGTCGT |
| | | GAGATGTTGGGTTAAGTCCCGCAACGAGCGCAACCCTCGTTCTATGTTGCC |
| | | AGCGCGTTATGGCGGGGACTCATAGGAGACTGCCGGGGTCAACTCGGAGG |
| | | AAGGTGGGGACGACGTCAAATCATCATGCCCCTTATGTCTTGGGCTTCACG |
| | | CATGCTACAATGGCCGGTACAAAGGGTTGCGATACTGTGAGGTGGAGCTA |
| | | ATCCCAAAAAGCCGGTCTCAGTTCGGATTGGGGTCTGCAACTCGACCCCA |
| | | TGAAGTCGGAGTCGCTAGTAATCGCAGATCAGCAACGCTGCGGTGAATAC |
| | | GTTCCCGGGCCTTGTACACACCGCCCGTCAAGTCACGAAAGTTGGTAACA |
| | | CCCGAAGCCGGTGGCCTAACCCTTGTGGGGGGAGCCGTCGAAGGTGGGAC |
| | | CGGCGATTGGGACTAAGTCGTAACAAGGTAGCCGTACCGGAAGGTGCGGC |
| | | TGGATCACCTCCTTT |
| 53 | DP53 16S | TGAAGAGTTTGATCATGGCTCAGATTGAACGCTGGCGGCAGGCCTAACAC |
| | rRNA | ATGCAAGTCGAGCGGTAGAGAGAAGCTTGCTTCTCTTGAGAGCGGCGGAC |
| | | GGGTGAGTAATACCTAGGAATCTGCCTGATAGTGGGGGATAACGTTCGGA |
| | | AACGGACGCTAATACCGCATACGTCCTACGGGAGAAAGCAGGGGACCTTC |
| | | GGGCCTTGCGCTATCAGATGAGCCTAGGTCGGATTAGCTAGTTGGTGAGG |
| | | TAATGGCTCACCAAGGCTACGATCCGTAACTGGTCTGAGAGGATGATCAG |
| | | TCACACTGGAACTGAGACACGGTCCAGACTCCTACGGGAGGCAGCAGTGG |
| | | GGAATATTGGACAATGGGCGAAAGCCTGATCCAGCCATGCCGCGTGTGTG |
| | | AAGAAGGTCTTCGGATTGTAAAGCACTTTAAGTTGGGAGGAAGGGCAGTT |
| | | ACCTAATACGTGATTGTCTTGACGTTACCGACAGAATAAGCACCGGCTAA |
| | | CTCTGTGCCAGCAGCCGCGGTAATACAGAGGGTGCAAGCGTTAATCGGAA |
| | | TTACTGGGCGTAAAGCGCGCGTAGGTGGTTTGTTAAGTTGAATGTGAAAT |
| | | CCCCGGGCTCAACCTGGGAACTGCATCCAAAACTGGCAAGCTAGAGTATG |
| | | GTAGAGGGTAGTGGAATTTCCTGTGTAGCGGTGAAATGCGTAGATATAGG |
| | | AAGGAACACCAGTGGCGAAGGCGACTACCTGGACTGATACTGACACTGAG |
| | | GTGCGAAAGCGTGGGGAGCAAACAGGATTAGATACCCTGGTAGTCCACGC |
| | | CGTAAACGATGTCAACTAGCCGTTGGGAGTCTTGAACTCTTAGTGGCGCA |
| | | GCTAACGCATTAAGTTGACCGCCTGGGGAGTACGGCCGCAAGGTTAAAAC |
| | | TCAAATGAATTGACGGGGGCCCGCACAAGCGGTGGAGCATGTGGTTTAAT |
| | | TCGAAGCAACGCGAAGAACCTTACCAGGCCTTGACATCCAATGAACTTTC |
| | | TAGAGATAGATTGGTGCCTTCGGGAACATTGAGACAGGTGCTGCATGGCT |
| | | GTCGTCAGCTCGTGTCGTGAGATGTTGGGTTAAGTCCCGTAACGAGCGCA |
| | | ACCCTTGTCCTTAGTTACCAGCACGTAATGGTGGGCACTCTAAGGAGACTG |
| | | CCGGTGACAAACCGGAGGAAGGTGGGGATGACGTCAAGTCATCATGGCCC |
| | | TTACGGCCTGGGCTACACACGTGCTACAATGGTCGGTACAAAGGGTTGCC |
| | | AAGCCGCGAGGTGGAGCTAATCCCATAAAACCGATCGTAGTCCGGATCGC |
| | | AGTCTGCAACTCGACTGCGTGAAGTCGGAATCGCTAGTAATCGTGAATCA |
| | | GAATGTCACGGTGAATACGTTCCCGGGCCTTGTACACACCGCCCGTCACA |
| | | CCATG |
| 54 | DP54 16S | CTTGAGAGTTTGATCCTGGCTCAGAGCGAACGCTGGCGGCAGGCTTAACA |
| | rRNA | CATGCAAGTCGAGCGGGCACCTTCGGGTGTCAGCGGCAGACGGGTGAGTA |
| | | ACACGTGGGAACGTACCCTTCGGTTCGGAATAACGCTGGGAAACTAGCGC |
| | | TAATACCGGATACGCCCTTTTGGGGAAAGGTTTACTGCCGAAGGATCGGC |
| | | CCGCGTCTGATTAGCTAGTTGGTGGGGTAACGGCCTACCAAGGCGACGAT |
| | | CAGTAGCTGGTCTGAGAGGATGATCAGCCACACTGGGACTGAGACACGGC |
| | | CCAGACTCCTACGGGAGGCAGCAGTGGGGAATATTGGACAATGGGCGCA |
| | | AGCCTGATCCAGCCATGCCGCGTGAGTGATGAAGGCCTTAGGGTTGTAAA |
| | | GCTCTTTTGTCCGGGACGATAATGACGGTACCGGAAGAATAAGCCCCGGC |
| | | TAACTTCGTGCCAGCAGCCGCGGTAATACGAAGGGGGCTAGCGTTGCTCG |
| | | GAATCACTGGGCGTAAAGGGCGCGTAGGCGGCCATTCAAGTCGGGGGTGA |
| | | AAGCCTGTGGCTCAACCACAGAATTGCCTTCGATACTGTTTGGCTTGAGTT |
| | | TGGTAGAGGTTGGTGGAACTGCGAGTGTAGAGGTGAAATTCGTAGATATT |
| | | CGCAAGAACACCAGTGGCGAAGGCGGCCAACTGGACCAATACTGACGCT |
| | | GAGGCGCGAAAGCGTGGGGAGCAAACAGGATTAGATACCCTGGTAGTCC |
| | | ACGCCGTAAACGATGAATGCTAGCTGTTGGGGTGCTTGCACCTCAGTAGC |
| | | GCAGCTAACGCTTTAAGCATTCCGCCTGGGGAGTACGGTCGCAAGATTAA |
| | | AACTCAAAGGAATTGACGGGGGCCCGCACAAGCGGTGGAGCATGTGGTTT |
| | | AATTCGAAGCAACGCGCAGAACCTTACCATCCCTTGACATGTCGTGCCATC |
| | | CGGAGAGATCCGGGGTTCCCTTCGGGGACGCGAACACAGGTGCTGCATGG |
| | | CTGTCGTCAGCTCGTGTCGTGAGATGTTGGGTTAAGTCCCGCAACGAGCGC |
| | | AACCCACGTCCTTAGTTGCCATCATTTAGTTGGGCACTCTAGGGAGACTGC |
| | | CGGTGATAAGCCGCGAGGAAGGTGTGGATGACGTC |
| 55 | DP55 16S | TCGGAGAGTTTGATCCTGGCTCAGGATGAACGCTGGCGGCGTGCCTAATA |
| | rRNA | CATGCAAGTCGAGCGAACTGATTAGAAGCTTGCTTCTATGACGTTAGCGG |
| | | CGGACGGGTGAGTAACACGTGGGCAACCTGCCTGTAAGACTGGGATAACT |
| | | TCGGGAAACCGAAGCTAATACCGGATAGGATCTTCTCCTTCATGGGAGAT |
| | | GATTGAAAGATGGTTTCGGCTATCACTTACAGATGGGCCCGCGGTGCATT |
| | | AGCTAGTTGGTGAGGTAACGGCTCACCAAGGCAACGATGCATAGCCGACC |
| | | TGAGAGGGTGATCGGCCACACTGGGACTGAGACACGGCCCAGACTCCTAC |
| | | GGGAGGCAGCAGTAGGGAATCTTCCGCAATGGACGAAAGTCTGACGGAG |
| | | CAACGCCGCGTGAGTGATGAAGGCTTTCGGGTCGTAAAACTCTGTTGTTA |
| | | GGGAAGAACAAGTACAAGAGTAACTGCTTGTACCTTGACGGTACCTAACC |
| | | AGAAAGCCACGGCTAACTACGTGCCAGCAGCCGCGGTAATACGTAGGTGG |
| | | CAAGCGTTATCCGGAATTATTGGGCGTAAAGCGCGCGCAGGCGGTTTCTT |
| | | AAGTCTGATGTGAAAGCCCACGGCTCAACCGTGGAGGGTCATTGGAAACT |
| | | GGGGAACTTGAGTGCAGAAGAGAAAAGCGGAATTCCACGTGTAGCGGTG |
| | | AAATGCGTAGAGATGTGGAGGAACACCAGTGGCGAAGGCGGCTTTTTGGT |
| | | CTGTAACTGACGCTGAGGCGCGAAAGCGTGGGGAGCAAACAGGATTAGA |
| | | TACCCTGGTAGTCCACGCCGTAAACGATGAGTGCTAAGTGTTAGAGGGTT |
| | | TCCGCCCTTTAGTGCTGCAGCTAACGCATTAAGCACTCCGCCTGGGGAGTA |
| | | CGGTCGCAAGACTGAAACTCAAAGGAATTGACGGGGGCCCGCACAAGCG |
| | | GTGGAGCATGTGGTTTAATTCGAAGCAACGCGAAGAACCTTACCAGGTCT |
| | | TGACATCCTCTGACAACTCTAGAGATAGAGCGTTCCCCTTCGGGGGACAG |
| | | AGTGACAGGTGGTGCATGGTTGTCGTCAGCTCGTGTCGTGAGATGTTGGGT |
| | | TAAGTCCCGCAACGAGCGCAACCCTTGATCTTAGTTGCCAGCATTTAGTTG |
| | | GGCACTCTAAGGTGACTGCCGGTGACAAACCGGAGGAAGGTGGGGATGA |
| | | CGTCAAATCATCATGCCCCTTATGACCTGGGCTACACACGTGCTACAATGG |
| | | ATGGTACAAAGGGCTGCAAGACCGCGAGGTCAAGCCAATCCCATAAAACC |
| | | ATTCTCAGTTCGGATTGTAGGCTGCAACTCGCCTACATGAAGCTGGAATCG |
| | | CTAGTAATCGCGGATCAGCATGCT |
| 56 | DP56 16S | ATTGGAGAGTTTGATCCTGGCTCAGGACGAACGCTGGCGGCGTGCCTAAT |
| | rRNA | ACATGCAAGTCGAGCGGACCTGATGGAGTGCTTGCACTCCTGATGGTTAG |
| | | CGGCGGACGGGTGAGTAACACGTAGGCAACCTGCCCTCAAGACTGGGATA |
| | | ACTACCGGAAACGGTAGCTAATACCGGATAATTTATTTCACAGCATTGTG |
| | | GAATAATGAAAGACGGAGCAATCTGTCACTTGGGGATGGGCCTGCGGCGC |
| | | ATTAGCTAGTTGGTGGGGTAACGGCTCACCAAGGCGACGATGCGTAGCCG |
| | | ACCTGAGAGGGTGAACGGCCACACTGGGACTGAGACACGGCCCAGACTCC |
| | | TACGGGAGGCAGCAGTAGGGAATCTTCCGCAATGGGCGAAAGCCTGACG |
| | | GAGCAACGCCGCGTGAGTGATGAAGGTTTTCGGATCGTAAAGCTCTGTTG |
| | | CCAAGGAAGAACGTCTTCTAGAGTAACTGCTAGGAGAGTGACGGTACTTG |
| | | AGAAGAAAGCCCCGGCTAACTACGTGCCAGCAGCCGCGGTAATACGTAGG |
| | | GGGCAAGCGTTGTCCGGAATTATTGGGCGTAAAGCGCGCGCAGGCGGTTC |
| | | TTTAAGTCTGGTGTTTAAACCCGAGGCTCAACTTCGGGTCGCACTGGAAAC |
| | | TGGGGAACTTGAGTGCAGAAGAGGAGAGTGGAATTCCACGTGTAGCGGTG |
| | | AAATGCGTAGATATGTGGAGGAACACCAGTGGCGAAGGCGACTCTCTGGG |
| | | CTGTAACTGACGCTGAGGCGCGAAAGCGTGGGGAGCAAACAGGATTAGA |
| | | TACCCTGGTAGTCCACGCCGTAAACGATGAATGCTAGGTGTTAGGGGTTTC |
| | | GATACCCTTGGTGCCGAAGTTAACACATTAAGCATTCCGCCTGGGGAGTA |
| | | CGGTCGCAAGACTGAAACTCAAAGGAATTGACGGGGACCCGCACAAGCA |
| | | GTGGAGTATGTGGTTTAATTCGAAGCAACGCGAAGAACCTTACCAAGTCT |
| | | TGACATCCCTCTGAATCCTCTAGAGATAGAGGCGGCCTTCGGGACAGAGG |
| | | TGACAGGTGGTGCATGGTTGTCGTCAGCTCGTGTCGTGAGATGTTGGGTTA |
| | | AGTCCCGCAACGAGCGCAACCCTTGATTTTAGTTGCCAGCACATCATGGTG |
| | | GGCACTCTAGAATGACTGCCGGTGACAAACCGGAGGAAGGCGGGGATGA |
| | | CGTCAAATCATCATGCCCCTTATGACTTGGGCTACACACGTACTACAATGG |
| | | CTGGTACAACGGGAAGCGAAGCCGCGAGGTGGAGCCAATCCTATAAAAG |
| | | CCAGTCTCAGTTCGGATTGCAGGCTGCAACTCGCCTGCATGAAGTCGGAA |
| | | TTGCTAGTAATCGCGGATCAGCATGCCGCGGTGAATACGTTCCCGGGTCTT |
| | | GTACACACCGCCCGTCACACCACGAGAGTTTACAACACCCGAAGTCGGTG |
| | | GGGTAACCCGCAAGGGAGCCAGCCGCCGAAGGTGGGGTAGATGATTGGG |
| | | GTGAAGTCGTAACAAGGTAGCCGTATCGGAAGGTGCGGCTGGATCACCTC |
| | | CTTT |
| 57 | DP57 16S | ATTGGAGAGTTTGATCCTGGCTCAGGATGAACGCTGGCGGCGTGCCTAAT |
| | rRNA | ACATGCAAGTCGAGCGAATGGATTAAGAGCTTGCTCTTATGAAGTTAGCG |
| | | GCGGACGGGTGAGTAACACGTGGGTAACCTGCCCATAAGACTGGGATAAC |
| | | TCCGGGAAACCGGGGCTAATACCGGATAACATTTTGCACCGCATGGTGCG |
| | | AAATTCAAAGGCGGCTTCGGCTGTCACTTATGGATGGACCCGCGTCGCATT |
| | | AGCTAGTTGGTGAGGTAACGGCTCACCAAGGCAACGATGCGTAGCCGACC |
| | | TGAGAGGGTGATCGGCCACACTGGGACTGAGACACGGCCCAGACTCCTAC |
| | | GGGAGGCAGCAGTAGGGAATCTTCCGCAATGGACGAAAGTCTGACGGAG |
| | | CAACGCCGCGTGAGTGATGAAGGCTTTCGGGTCGTAAAACTCTGTTGTTA |
| | | GGGAAGAACAAGTGCTAGTTGAATAAGCTGGCACCTTGACGGTACCTAAC |
| | | CAGAAAGCCACGGCTAACTACGTGCCAGCAGCCGCGGTAATACGTAGGTG |
| | | GCAAGCGTTATCCGGAATTATTGGGCGTAAAGCGCGCGCAGGTGGTTTCT |
| | | TAAGTCTGATGTGAAAGCCCACGGCTCAACCGTGGAGGGTCATTGGAAAC |
| | | TGGGAGACTTGAGTGCAGAAGAGGAAAGTGGAATTCCATGTGTAGCGGTG |
| | | AAATGCGTAGAGATATGGAGGAACACCAGTGGCGAAGGCGACTTTCTGGT |
| | | CTGTAACTGACACTGAGGCGCGAAAGCGTGGGGAGCAAACAGGATTAGA |
| | | TACCCTGGTAGTCCACGCCGTAAACGATGAGTGCTAAGTGTTAGAGGGTT |
| | | TCCGCCCTTTAGTGCTGAAGTTAACGCATTAAGCACTCCGCCTGGGGAGTA |
| | | CGGCCGCAAGGCTGAAACTCAAAGGAATTGACGGGGGCCCGCACAAGCG |
| | | GTGGAGCATGTGGTTTAATTCGAAGCAACGCGAAGAACCTTACCAGGTCT |
| | | TGACATCCTCTGACAACCCTAGAGATAGGGCTTCCCCTTCGGGGGCAGAG |
| | | TGACAGGTGGTGCATGGTTGTCGTCAGCTCGTGTCGTGAGATGTTGGGTTA |
| | | AGTCCCGCAACGAGCGCAACCCTTGATCTTAGTTGCCATCATTAAGTTGGG |
| | | CACTCTAAGGTGACTGCCGGTGACAAACCGGAGGAAGGTGGGGATGACGT |
| | | CAAATCATCATGCCCCTTATGACCTGGGCTACACACGTGCTACAATGGAC |
| | | GGTACAAAGAGCTGCAAGACCGCGAGGTGGAGCTAATCTCATAAAACCGT |
| | | TCTCAGTTCGGATTGTAGGCTGCAACTCGCCTACATGAAGCTGGAATCGCT |
| | | AGTAATCGCGGATCAGCATGCCGCGGTGAATACGTTCCCGGGCCTTGTAC |
| | | ACACCGCCCGTCACACCACGAGAGTTTGTAACACCCGAAGTCGGTGGGGT |
| | | AACCTTTTTGGAGCCAGCCGCCTAAGGTGGGACAGATGATTGGGGTGAAG |
| | | TCGTAACAAGGTAGCCGTATCGGAAGGTGCGGCTGGATCACCTCCTTT |
| 58 | DP58 16S | AATGACGGTACCTGAAGAATAAGCACCGGCTAACTACGTGCCAGCAGCCG |
| | rRNA | CGGTAATACGTAGGGTGCAAGCGTTAATCGGAATTACTGGGCGTAAAGCG |
| | | TGCGCAGGCGGTTTTGTAAGTCTGATGTGAAATCCCCGGGCTCAACCTGG |
| | | GAATTGCATTGGAGACTGCAAGGCTAGAATCTGGCAGAGGGGGGTAGAAT |
| | | TCCACGTGTAGCAGTGAAATGCGTAGATATGTGGAGGAACACCGATGGCG |
| | | AAGGCAGCCCCCTGGGTCAAGATTGACGCTCATGCACGAAAGCGTGGGGA |
| | | GCAAACAGGATTAGATACCCTGGTAGTCCACGCCCTAAACGATGTCTACT |
| | | AGTTGTCGGGTCTTAATTGACTTGGTAACGCAGCTAACGCGTGAAGTAGA |
| | | CCGCCTGGGGAGTACGGTCGCAAGATTAAAACTCAAAGGAATTGACGGGG |
| | | ACCCGCACAAGCGGTGGATGATGTGGATTAATTCGATGCAACGCGAAAAA |
| | | CCTTACCTACCCTTGACATGGCTGGAATCCTCGAGAGATTGGGGAGTGCTC |
| | | GAAAGAGAACCAGTACACAGGTGCTGCATGGCTGTCGTCAGCTCGTGTCG |
| | | TGAGATGTTGGGTTAAGTCCCGCAACGAGCGCAACCCTTGTCATTAGTTGC |
| | | TACGAAAGGGCACTCTAATGAGACTGCCGGTGACAAACCGGAGGAAGGT |
| | | GGGGATGACGTCAAGTCCTCATGGCCCTTATGGGTAGGGCTTCACACGTC |
| | | ATACAATGGTACATACAGAGCGCCGCCAACCCGCGAGGGGGAGCTAATCG |
| | | CAGAAAGTGTATCGTAGTCCGGATTGTAGTCTGCAACTCGACTGCATGAA |
| | | GTTGGAATCGCTAGTAATCGCGGATCAGCATGTCGCGGTGAATACGTTCC |
| | | CGGGTCTTGTACACACCGCCCGTCACACCATGGGAGCGGGTTTTACCAGA |
| | | AGTAGGTAGCTTAACCGTAAGGAGGGCGCTTACCACGGTAGGATTCGTGA |
| | | CTGGGGTGAAGTCGTAACAAGGTAGCCGTATCGGAAGGTGCGGCTGGATC |
| | | ACCTCCTTT |
| 59 | DP59 16S | TTGAAGAGTTTGATCATGGCTCAGATTGAACGCTGGCGGCAGGCCTAACA |
| | rRNA | CATGCAAGTCGAACGGTAACAGGAAGCAGCTTGCTGCTTTGCTGACGAGT |
| | | GGCGGACGGGTGAGTAATGTCTGGGAAACTGCCTGATGGAGGGGGATAA |
| | | CTACTGGAAACGGTAGCTAATACCGCATAACGTCGCAAGACCAAAGAGGG |
| | | GGACCTTCGGGCCTCTTGCCATCAGATGTGCCCAGATGGGATTAGCTAGTA |
| | | GGTGGGGTAACGGCTCACCTAGGCGACGATCCCTAGCTGGTCTGAGAGGA |
| | | TGACCAGCCACACTGGAACTGAGACACGGTCCAGACTCCTACGGGAGGCA |
| | | GCAGTGGGGAATATTGCACAATGGGCGCAAGCCTGATGCAGCCATGCCGC |
| | | GTGTATGAAGAAGGCCTTCGGGTTGTAAAGTACTTTCAGCGGGGAGGAAG |
| | | GCGATGCGGTTAATAACCGCGTCGATTGACGTTACCCGCAGAAGAAGCAC |
| | | CGGCTAACTCCGTGCCAGCAGCCGCGGTAATACGGAGGGTGCAAGCGTTA |
| | | ATCGGAATTACTGGGCGTAAAGCGCACGCAGGCGGTCTGTCAAGTCGGAT |
| | | GTGAAATCCCCGGGCTCAACCTGGGAACTGCATCCGAAACTGGCAGGCTT |
| | | GAGTCTCGTAGAGGGGGGTAGAATTCCAGGTGTAGCGGTGAAATGCGTAG |
| | | AGATCTGGAGGAATACCGGTGGCGAAGGCGGCCCCCTGGACGAAGACTG |
| | | ACGCTCAGGTGCGAAAGCGTGGGGAGCAAACAGGATTAGATACCCTGGTA |
| | | GTCCACGCCGTAAACGATGTCGACTTGGAGGTTGTGCCCTTGAGGCGTGG |
| | | CTTCCGGAGCTAACGCGTTAAGTCGACCGCCTGGGGAGTACGGCCGCAAG |
| | | GTTAAAACTCAAATGAATTGACGGGGGCCCGCACAAGCGGTGGAGCATGT |
| | | GGTTTAATTCGATGCAACGCGAAGAACCTTACCTGGTCTTGACATCCACAG |
| | | AACTTGGCAGAGATGCCTTGGTGCCTTCGGGAACTGTGAGACAGGTGCTG |
| | | CATGGCTGTCGTCAGCTCGTGTTGTGAAATGTTGGGTTAAGTCCCGCAACG |
| | | AGCGCAACCCTTATCCTTTGTTGCCAGCGGTTAGGCCGGGAACTCAAAGG |
| | | AGACTGCCAGTGATAAACTGGAGGAAGGTGGGGATGACGTCAAGTCATCA |
| | | TGGCCCTTACGACCAGGGCTACACACGTGCTACAATGGCGCATACAAAGA |
| | | GAAGCGATCTCGCGAGAGCCAGCGGACCTCATAAAGTGCGTCGTAGTCCG |
| | | GATTGGAGTCTGCAACTCGACTCCATGAAGTCGGAATCGCTAGTAATCGT |
| | | GAATCAGAATGTCACGGTGAATACGTTCCCGGGCCTTGTACACACCGCCC |
| | | GTCACACCATGGGAGTGGGTTGCAAAAGAAGTAGGTAGCTTAACCTTCGG |
| | | GAGGGCGCTTACCACTTTGTGATTCATGACTGGGGTGAAGTCGTAACAAG |
| | | GTAACCGTAGGGGAACCTGCGGTTGGATCACCTCCTT |
| 60 | DP60 16S | TCGGAGAGTTTGATCCTGGCTCAGGACGAACGCTGGCGGCGTGCCTAATA |
| | rRNA | CATGCAAGTCGAGCGAATCGATGGGAGCTTGCTCCCTGAGATTAGCGGCG |
| | | GACGGGTGAGTAACACGTGGGCAACCTGCCTATAAGACTGGGATAACTTC |
| | | GGGAAACCGGAGCTAATACCGGATACGTTCTTTTCTCGCATGAGAGAAGA |
| | | TGGAAAGACGGTTTTGCTGTCACTTATAGATGGGCCCGCGGCGCATTAGCT |
| | | AGTTGGTGAGGTAATGGCTCACCAAGGCGACGATGCGTAGCCGACCTGAG |
| | | AGGGTGATCGGCCACACTGGGACTGAGACACGGCCCAGACTCCTACGGGA |
| | | GGCAGCAGTAGGGAATCTTCCGCAATGGACGAAAGTCTGACGGAGCAAC |
| | | GCCGCGTGAACGAAGAAGGCCTTCGGGTCGTAAAGTTCTGTTGTTAGGGA |
| | | AGAACAAGTACCAGAGTAACTGCTGGTACCTTGACGGTACCTAACCAGAA |
| | | AGCCACGGCTAACTACGTGCCAGCAGCCGCGGTAATACGTAGGTGGCAAG |
| | | CGTTGTCCGGAATTATTGGGCGTAAAGCGCGCGCAGGTGGTTCCTTAAGTC |
| | | TGATGTGAAAGCCCACGGCTCAACCGTGGAGGGTCATTGGAAACTGGGGA |
| | | ACTTGAGTGCAGAAGAGGAAAGTGGAATTCCAAGTGTAGCGGTGAAATGC |
| | | GTAGAGATTTGGAGGAACACCAGTGGCGAAGGCGACTTTCTGGTCTGTAA |
| | | CTGACACTGAGGCGCGAAAGCGTGGGGAGCAAACAGGATTAGATACCCT |
| | | GGTAGTCCACGCCGTAAACGATGAGTGCTAAGTGTTAGAGGGTTTCCGCC |
| | | CTTTAGTGCTGCAGCTAACGCATTAAGCACTCCGCCTGGGGAGTACGGCC |
| | | GCAAGGCTGAAACTCAAAGGAATTGACGGGGGCCCGCACAAGCGGTGGA |
| | | GCATGTGGTTTAATTCGAAGCAACGCGAAGAACCTTACCAGGTCTTGACA |
| | | TCCTCTGACAACCCTAGAGATAGGGCGTTCCCCTTCGGGGGACAGAGTGA |
| | | CAGGTGGTGCATGGTTGTCGTCAGCTCGTGTCGTGAGATGTTGGGTTAAGT |
| | | CCCGCAACGAGCGCAACCCTTGATCTTAGTTGCCAGCATTCAGTTGGGCAC |
| | | TCTAAGGTGACTGCCGGTGACAAACCGGAGGAAGGTGGGGATGACGTCA |
| | | AATCATCATGCCCCTTATGACCTGGGCTACACACGTGCTACAATGGATGGT |
| | | ACAAAGGGCTGCAAACCTGCGAAGGTAAGCGAATCCCATAAAGCCATTCT |
| | | CAGTTCGGATTGTAGGCTGCAACTCGCCTACATGAAGCCGGAATCGCTAG |
| | | TAATCGCGGATCAGCATGCCGCGGTGAATACGTTCCCGGGCCTTGTACAC |
| | | ACCGCCCGTCACACCACGAGAGTTTGTAACACCCGAAGTCGGTGAGGTAA |
| | | CCTTTATGGAGCCAGCCGCCTAAGGTGGGACAGATGATTGGGGTGAAGTC |
| | | GTAACAAGGTAGCCGTATCGGAAGGTGCGGCTGGATCACCTCCTTT |
| 61 | DP61 16S | GGAAGGCGGTCTGTCAAGTCGGATGTGAAATCCCCGGGCTCAACCTGGGA |
| | rRNA | ACTGCATTCGAAACTGGCAGGCTAGAGTCTTGTAGAGGGGGGTAGAATTC |
| | | CAGGTGTAGCGGTGAAATGCGTAGAGATCTGGAGGAATACCGGTGGCGA |
| | | AGGCGGCCCCCTGGACAAAGACTGACGCTCAGGTGCGAAAGCGTGGGGA |
| | | GCAAACAGGATTAGATACCCTGGTAGTCCACGCCGTAAACGATGTCGACT |
| | | TGGAGGTTGTTCCCTTGAGGAGTGGCTTCCGGAGCTAACGCGTTAAGTCG |
| | | ACCGCCTGGGGAGTACGGCCGCAAGGTTAAAACTCAAATGAATTGACGGG |
| | | GGCCCGCACAAGCGGTGGAGCATGTGGTTTAATTCGATGCAACGCGAAGA |
| | | ACCTTACCTACTCTTGACATCCACGGAATTTAGCAGAGATGCTTTAGTGCC |
| | | TTCGGGAACCGTGAGACAGGTGCTGCATGGCTGTCGTCAGCTCGTGTTGTG |
| | | AAATGTTGGGTTAAGTCCCGCAACGAGCGCAACCCTTATCCTTTGTTGCCA |
| | | GCGGTCCGGCCGGGAACTCAAAGGAGACTGCCAGTGATAAACTGGAGGA |
| | | AGGTGGGGATGACGTCAAGTCATCATGGCCCTTACGAGTAGGGCTACACA |
| | | CGTGCTACAATGGCGCATACAAAGAGAAGCGACCTCGCGAGAGCAAGCG |
| | | GACCTCATAAAGTGCGTCGTAGTCCGGATCGGAGTCTGCAACTCGACTCC |
| | | GTGAAGTCGGAATCGCTAGTAATCGTAGATCAGAATGCTACGGTGAATAC |
| | | GTTCCCGGGCCTTGTACACACCGCCCGTCACACCATGGGAGTGGGTTGCA |
| | | AAAGAAGTAGGTAGCTTAACCTTCGGGAGGGCGCTTACCACTTTGTGATT |
| | | CATGACTGGGGTGAAGTCGTAACAAGGTAACCGTAGGGGAACCTGCGGTT |
| | | GGATCACCTCCTT |
| 62 | DP62 16S | TGGCTCAGATTGAACGCTGGCGGCAGGCCTAACACATGCAAGTCGAACGG |
| | rRNA | TAGCACAGAGGAGCTTGCTCCTTGGGTGACGAGTGGCGGACGGGTGAGTA |
| | | ATGTCTGGGAAACTGCCCGATGGAGGGGGATAACTACTGGAAACGGTAGC |
| | | TAATACCGCATAACGTCTTCGGACCAAAGTGGGGGACCTTCGGGCCTCAC |
| | | ACCATCGGATGTGCCCAGATGGGATTAGCTAGTAGGTGGGGTAATGGCTC |
| | | ACCTAGGCGACGATCCCTAGCTGGTCTGAGAGGATGACCAGCCACACTGG |
| | | AACTGAGACACGGTCCAGACTCCTACGGGAGGCAGCAGTGGGGAATATTG |
| | | CACAATGGGCGCAAGCCTGATGCAGCCATGCCGCGTGTATGAAGAAGGCC |
| | | TTCGGGTTGTAAAGTACTTTCAGTGGGGAGGAAGGCGTTAAGGTTAATAA |
| | | CCTTGGCGATTGACGTTACCCGCAGAAGAAGCACCGGCTAACTCCGTGCC |
| | | AGCAGCCGCGGTAATACGGAGGGTGCAAGCGTTAATCGGAATTACTGGGC |
| | | GTAAAGCGCACGCAGGCGGTCTGTCAAGTCGGATGTGAAATCCCCGGGCT |
| | | CAACCTGGGAACTGCATTCGAAACTGGCAGGCTAGAGTCTTGTAGAGGGG |
| | | GGTAGAATTCCAGGTGTAGCGGTGAAATGCGTAGAGATCTGGAGGAATAC |
| | | CGGTGGCGAAGGCGGCCCCCTGGACAAAGACTGACGCTCAGGTGCGAAA |
| | | GCGTGGGGAGCAAACAGGATTAGATACCCTGGTAGTCCACGCCGTAAACG |
| | | ATGTCGACTTGGAGGTTGTTCCCTTGAGGAGTGGCTTCCGGAGCTAACGCG |
| | | TTAAGTCGACCGCCTGGGGAGTACGG |
| 63 | DP63 16S | TGAAGAGTTTGATCATGGCTCAGATTGAACGCTGGCGGCAGGCCTAACAC |
| | rRNA | ATGCAAGTCGAGCGGTAGAGAGAAGCTTGCTTCTCTTGAGAGCGGCGGAC |
| | | GGGTGAGTAATGCCTAGGAATCTGCCTGGTAGTGGGGGATAACGTTCGGA |
| | | AACGGACGCTAATACCGCATACGTCCTACGGGAGAAAGCAGGGGACCTTC |
| | | GGGCCTTGCGCTATCAGATGAGCCTAGGTCGGATTAGCTAGTTGGTGAGG |
| | | TAATGGCTCACCAAGGCGACGATCCGTAACTGGTCTGAGAGGATGATCAG |
| | | TCACACTGGAACTGAGACACGGTCCAGACTCCTACGGGAGGCAGCAGTGG |
| | | GGAATATTGGACAATGGGCGAAAGCCTGATCCAGCCATGCCGCGTGTGTG |
| | | AAGAAGGTCTTCGGATTGTAAAGCACTTTAAGTTGGGAGGAAGGGTTGTA |
| | | GATTAATACTCTGCAATTTTGACGTTACCGACAGAATAAGCACCGGCTAA |
| | | CTCTGTGCCAGCAGCCGCGGTAATACAGAGGGTGCAAGCGTTAATCGGAA |
| | | TTACTGGGCGTAAAGCGCGCGTAGGTGGTTTGTTAAGTTGGATGTGAAAT |
| | | CCCCGGGCTCAACCTGGGAACTGCATTCAAAACTGACTGACTAGAGTATG |
| | | GTAGAGGGTGGTGGAATTTCCTGTGTAGCGGTGAAATGCGTAGATATAGG |
| | | AAGGAACACCAGTGGCGAAGGCGACCACCTGGACTAATACTGACACTGA |
| | | GGTGCGAAAGCGTGGGGAGCAAACAGGATTAGATACCCTGGTAGTCCACG |
| | | CCGTAAACGATGTCAACTAGCCGTTGGAAGCCTTGAGCTTTTAGTGGCGC |
| | | AGCTAACGCATTAAGTTGACCGCCTGGGGAGTACGGCCGCAAGGTTAAAA |
| | | CTCAAATGAATTGACGGGGGCCCGCACAAGCGGTGGAGCATGTGGTTTAA |
| | | TTCGAAGCAACGCGAAGAACCTTACCAGGCCTTGACATCCAATGAACTTT |
| | | CTAGAGATAGATTGGTGCCTTCGGGAACATTGAGACAGGTGCTGCATGGC |
| | | TGTCGTCAGCTCGTGTCGTGAGATGTTGGGTTAAGTCCCGTAACGAGCGCA |
| | | ACCCTTGTTCTTAGTTACCAGCACGTTATGGTGGGCACTCTAAGGAGACTG |
| | | CCGGTGACAAACCGGAGGAAGGTGGGGATGACGTCAAGTCATCATGGCCC |
| | | TTACGGCCTGGGCTACACACGTGCTACAATGGTCGGTACAGAGGGTTGCC |
| | | AAGCCGCGAGGTGGAGCTAATCCCATAAAACCGATCGTAGTCCGGATCGC |
| | | AGTCTGCAACTCGACTGCGTGAAGTCGGAATCGCTAGTAATCGCGAATCA |
| | | GAATGTCGCGGTGAATACGTTCCCGGGCCTTGTACACACCGCCCGTCACA |
| | | CCATGGGAGTGGGTTGCACCAGAAGTAGCTAGTCTAACCTTCGGGAGGAC |
| | | GGTTACCACGGTGTGATTCATGACTGGGGTGAAGTCGTAACAAGGTAGCC |
| | | GTAGGGGAACCTGCGGCTGGATCACCTCCTT |
| 64 | DP64 ITS | GCTCGAGTTCTTGTTTAGATCTTTTACAATAATGTGTATCTTTACTGAAGAT |
| | sequence | GTGCGCTTAATTGCGCTGCTTCTTTAGAGTGTCGCAGTGAAAGTAGTCTTG |
| | | CTTGAATCTCAGTCAACGCTACACACATTGGAGTTTTTTTACTTTAATTTAA |
| | | TTCTTTCTGCTTTGAATCGAAAGGTTCAAGGCAAAAAACAAACACAAACA |
| | | ATTTTATTTTATTATAATTTTTTAAACTAAACCAAAATTCCTAACGGAAATT |
| | | TTAAAATAATTTAAAACTTTCAACAACGGATCTCTTGGTTCTCGCATCGAT |
| | | GAAGAACGTAGCGAATTGCGATAAGTAATGTGAATTGCAGATACTCGTGA |
| | | ATCATTGAATTTTTGAACGCACATTGCGCCCTTGAGCATTCTCAGGGGCAT |
| | | GCCTGTTTGAGCGTCATTTCCTTCTCAAAAGATAATTTATTATTTTTTGGTT |
| | | GTGGGCGATACTCAGGGTTAGCTTGAAATTGGAGACTGTTTCAGTCTTTTT |
| | | TAATTCAACACTTAGCTTCTTTGGAGACGCTGTTCTCGCTGTGATGTATTTA |
| | | TGGATTTATTCGTTTTACTTTACAAGGGAAATGGTAACGTACCTTAGGCAA |
| | | AGGGTTGCTTTTAATATTCATCAAGTTTGACCTCAAATCAGGTAGGATTAC |
| | | CCGCTGAACTTAAGCATATCAATAAGCGGAGGAAAAGAAACCAACTGGG |
| | | ATTACCTTAGTAACGGCGAGTGAAGCGGTAAAAGCTCAAATTTGAAATCT |
| | | GGTACTTTCAGTGCCCGAGTTGTAATTTGTAGAATTTGTCTTTGATTAGGT |
| | | CCTTGTCTATGTTCCTTGGNANCAGGACGTCATAGAGGGTGAGAATCCCGT |
| | | TTGGCGAGGATACCTTTTCTCTGTAAGACTTTTTCGAANANTCGAGTTGTT |
| | | TGGGAATGCAGCTCAAAGTGGGTGGTAAANTTCCATCTAAAGCTAAATNT |
| | | TGGCGAGAGACCGATAGCGAACNAGTACAGTGATGGAAAGATGAAAAAG |
| | | AANTTTN |
| 65 | DP65 16S | ATTGGAGAGTTTGATCCTGGCTCAGGATGAACGCTGGCGGCGTGCCTAAT |
| | rRNA | ACATGCAAGTCGAGCGAATGGATTAAGAGCTTGCTCTTATGAAGTTAGCG |
| | | GCGGACGGGTGAGTAACACGTGGGTAACCTGCCCATAAGACTGGGATAAC |
| | | TCCGGGAAACCGGGGCTAATACCGGATAACATTTTGAACTGCATGGTTCG |
| | | AAATTGAAAGGCGGCTTCGGCTGTCACTTATGGATGGACCCGCGTCGCAT |
| | | TAGCTAGTTGGTGAGGTAACGGCTCACCAAGGCAACGATGCGTAGCCGAC |
| | | CTGAGAGGGTGATCGGCCACACTGGGACTGAGACACGGCCCAGACTCCTA |
| | | CGGGAGGCAGCAGTAGGGAATCTTCCGCAATGGACGAAAGTCTGACGGA |
| | | GCAACGCCGCGTGAGTGATGAAGGCTTTCGGGTCGTAAAACTCTGTTGTT |
| | | AGGGAAGAACAAGTGCTAGTTGAATAAGCTGGCACCTTGACGGTACCTAA |
| | | CCAGAAAGCCACGGCTAACTACGTGCCAGCAGCCGCGGTAATACGTAGGT |
| | | GGCAAGCGTTATCCGGAATTATTGGGCGTAAAGCGCGCGCAGGTGGTTTC |
| | | TTAAGTCTGATGTGAAAGCCCACGGCTCAACCGTGGAGGGTCATTGGAAA |
| | | CTGGGAGACTTGAGTGCAGAAGAGGAAAGTGGAATTCCATGTGTAGCGGT |
| | | GAAATGCGTAGAGATATGGAGGAACACCAGTGGCGAAGGCGACTTTCTGG |
| | | TCTGTAACTGACACTGAGGCGCGAAAGCGTGGGGAGCAAACAGGATTAG |
| | | ATACCCTGGTAGTCCACGCCGTAAACGATGAGTGCTAAGTGTTAGAGGGT |
| | | TTCCGCCCTTTAGTGCTGAAGTTAACGCATTAAGCACTCCGCCTGGGGAGT |
| | | ACGGCCGCAAGGCTGAAACTCAAAGGAATTGACGGGGGCCCGCACAAGC |
| | | GGTGGAGCATGTGGTTTAATTCGAAGCAACGCGAAGAACCTTACCAGGTC |
| | | TTGACATCCTCTGAAAACCCTAGAGATAGGGCTTCTCCTTCGGGAGCAGA |
| | | GTGACAGGTGGTGCATGGTTGTCGTCAGCTCGTGTCGTGAGATGTTGGGTT |
| | | AAGTCCCGCAACGAGCGCAACCCTTGATCTTAGTTGCCATCATTAAGTTGG |
| | | GCACTCTAAGGTGACTGCCGGTGACAAACCGGAGGAAGGTGGGGATGAC |
| | | GTCAAATCATCATGCCCCTTATGACCTGGGCTACACACGTGCTACAATGGA |
| | | CGGTACAAAGAGCTGCAAGACCGCGAGGTGGAGCTAATCTCATAAAACCG |
| | | TTCTCAGTTCGGATTGTAGGCTGCAACTCGCCTACATGAAGCTGGAATCGC |
| | | TAGTAATCGCGGATCAGCATGCCGCGGTGAATACGTTCCCGGGCCTTGTA |
| | | CACACCGCCCGTCACACCACGAGAGTTTGTAACACCCGAAGTCGGTGGGG |
| | | TAACCTTTTTGGAGCCAGCCGCCTAAGGTGGGACAGATGATTGGGGTGAA |
| | | GTCGTAACAAGGTAGCCGTATCGGAAGGTGCGGCTGGATCACCTCCTTT |
| 66 | DP66 ITS | GATTTTTTGGGGTTGCTTCGAACTTGCAGACAGAGTGTCGAGACTTGTGAG |
| | sequence | CCTGCGCTTAATTGCGCGGCCTAGAGTCGAGTGCTTGTTATTGGCTGCGAG |
| | | GGACGAGTGCCTTTTGAAAAAATCCATTACACACTGTGAAGATTTTTTTTC |
| | | ATACATTTTACTTCTTTGGGGCTTTCGAGCTCCAAAGGCTATAAACACAAA |
| | | CCAAACTTTTTTTTTTATTATTTGTTAATCAAGAAATTTTCTTATTGAAATT |
| | | AAATATTTTAAAACTTTCAACAACGGATCTCTTGGTTCTCGCATCGATGAA |
| | | GAACGTAGCGAATTGCGATAAGTAATGTGAATTGCAGATTCTCGTGAATC |
| | | ATTGAATTTTTGAACGCACATTGCGCCCTCTGGTATTCCAGGGGGCATGCC |
| | | TGTTTGAGCGTCATTTCCTTCTCAAAATCTCGATTTTGGTTGTGAGTGATAC |
| | | TCTGTTACAGGGTTAACTTGAAAGTGCTATTGCCCTAGCTACTCTTTTTTTT |
| | | ACTTGCTAAGAAAAAGATTTTTGGATAATTTCAATGTATTTAGGTATTTAT |
| | | ACCGACTTTCATTGGATGCTGAGAGTCTTGTCTAAGCGCTTTTGTGAGATT |
| | | GAGCAGAAGGGATTAACAGTATTCATAAAGTTTGACCTCAAATCAGGTAG |
| | | GATTACCCGCTGAACTTAAGCATATCAATAAGCGGAGGAAAAGAAACCAA |
| | | CCGGGATTGCCTCAGTAACGGCGAGTGAAGCGGCAAAAGCTCAAATTTGA |
| | | AATCTGGCACTTTCAGTGTCCGAGTTGTAATTTGTAGAAGTAGTTTTGGGA |
| | | CTGGTCCTTATCTATGTTTCTTGGAACAGGACGTCATAGAGGGTGAGANCC |
| | | CGTATGATGAGGCCCCCAGTCCTTTGTAAAACGCTNCGAAGAGTCGAGTT |
| | | GTTTGGGAATGCAGCTCTAAGTGGGINGNAATTNNTCTAAAGCTAAATNN |
| | | NNNNNANACNNTNGCGANAGTACNGTGATGNNGATGANNACTTTGAAAN |
| | | ANANTGAAAAGTACGTGAA |
| 137 | DP72 16S | TTCGGAGAGTTTGATCCTGGCTCAGGACGAACGCTGGCGGCGTGCCTAAT |
| | rRNA | ACATGCAAGTCGAGCGGACAGAAGGGAGCTTGCTCCCGGATGTTAGCGGC |
| | | GGACGGGTGAGTAACACGTGGGTAACCTGCCTGTAAGACTGGGATAACTC |
| | | CGGGAAACCGGAGCTAATACCGGATAGTTCCTTGAACCGCATGGTTCAAG |
| | | GATGAAAGACGGTTTCGGCTGTCACTTACAGATGGACCCGCGGCGCATTA |
| | | GCTAGTTGGTGGGGTAATGGCTCACCAAGGCGACGATGCGTAGCCGACCT |
| | | GAGAGGGTGATCGGCCACACTGGGACTGAGACACGGCCCAGACTCCTACG |
| | | GGAGGCAGCAGTAGGGAATCTTCCGCAATGGACGAAAGTCTGACGGAGC |
| | | AACGCCGCGTGAGTGATGAAGGTTTTCGGATCGTAAAGCTCTGTTGTTAG |
| | | GGAAGAACAAGTGCGAGAGTAACTGCTCGCACCTTGACGGTACCTAACCA |
| | | GAAAGCCACGGCTAACTACGTGCCAGCAGCCGCGGTAATACGTAGGTGGC |
| | | AAGCGTTGTCCGGAATTATTGGGCGTAAAGGGCTCGCAGGCGGTTTCTTA |
| | | AGTCTGATGTGAAAGCCCCCGGCTCAACCGGGGAGGGTCATTGGAAACTG |
| | | GGAAACTTGAGTGCAGAAGAGGAGAGTGGAATTCCACGTGTAGCGGTGA |
| | | AATGCGTAGAGATGTGGAGGAACACCAGTGGCGAAGGCGACTCTCTGGTC |
| | | TGTAACTGACGCTGAGGAGCGAAAGCGTGGGGAGCGAACAGGATTAGAT |
| | | ACCCTGGTAGTCCACGCCGTAAACGATGAGTGCTAAGTGTTAGGGGGTTT |
| | | CCGCCCCTTAGTGCTGCAGCTAACGCATTAAGCACTCCGCCTGGGGAGTA |
| | | CGGTCGCAAGACTGAAACTCAAAGGAATTGACGGGGGCCCGCACAAGCG |
| | | GTGGAGCATGTGGTTTAATTCGAAGCAACGCGAAGAACCTTACCAGGTCT |
| | | TGACATCCTCTGACAACCCTAGAGATAGGGCTTTCCCTTCGGGGACAGAG |
| | | TGACAGGTGGTGCATGGTTGTCGTCAGCTCGTGTCGTGAGATGTTGGGTTA |
| | | AGTCCCGCAACGAGCGCAACCCTTGATCTTAGTTGCCAGCATTTAGTTGGG |
| | | CACTCTAAGGTGACTGCCGGTGACAAACCGGAGGAAGGTGGGGATGACGT |
| | | CAAATCATCATGCCCCTTATGACCTGGGCTACACACGTGCTACAATGGAC |
| | | AGAACAAAGGGCTGCGAGACCGCAAGGTTTAGCCAATCCCATAAATCTGT |
| | | TCTCAGTTCGGATCGCAGTCTGCAACTCGACTGCGTGAAGCTGGAATCGCT |
| | | AGTAATCGCGGATCAGCATGCCGCGGTGAATACGTTCCCGGGCCTTGTAC |
| | | ACACCGCCCGTCACACCACGAGAGTTTGCAACACCCGAAGTCGGTGAGGT |
| | | AACCTTTATGGAGCCAGCCGCCGAAGGTGGGGCAGATGATTGGGGTGAAG |
| | | TCGTAACAAGGTAGCCGTATCGGAAGGTGCGGCTGGATCACCTCCTTT |
| 138 | DP73 16S | AACGGAGAGTTTGATCCTGGCTCAGGACGAACGCTGGCGGCGTGCCTAAT |
| | rRNA | ACATGCAAGTCGAGCGGACAGAAGGGAGCTTGCTCCCGGACGTTAGCGGC |
| | | GGACGGGTGAGTAACACGTGGGCAACCTGCCCCTTAGACTGGGATAACTC |
| | | CGGGAAACCGGAGCTAATACCGGATAATCCCTTTCTCCACCTGGAGAGAG |
| | | GGTGAAAGATGGCTTCGGCTATCACTAAGGGATGGGCCCGCGGCGCATTA |
| | | GCTAGTTGGTAAGGTAACGGCTTACCAAGGCGACGATGCGTAGCCGACCT |
| | | GAGAGGGTGATCGGCCACACTGGGACTGAGACACGGCCCAGACTCCTACG |
| | | GGAGGCAGCAGTAGGGAATCTTCCGCAATGGACGAAAGTCTGACGGAGC |
| | | AACGCCGCGTGAGTGAGGAAGGCCTTCGGGTCGTAAAGCTCTGTTGTGAG |
| | | GGAAGAAGCGGTGCCGTTCGAATAGGGCGGTACCTTGACGGTACCTCACC |
| | | AGAAAGCCACGGCTAACTACGTGCCAGCAGCCGCGGTAATACGTAGGTGG |
| | | CAAGCGTTGTCCGGAATTATTGGGCGTAAAGCGCGCGCAGGCGGCTTCTT |
| | | AAGTCTGATGTGAAATCTCGGGGCTCAACCCCGAGCGGCCATTGGAAACT |
| | | GGGGAGCTTGAGTGCAGAAGAGGAGAGTGGAATTCCACGTGTAGCGGTG |
| | | AAATGCGTAGAGATGTGGAGGAACACCAGTGGCGAAGGCGACTCTCTGGT |
| | | CTGTAACTGACGCTGAGGCGCGAAAGCGTGGGGAGCAAACAGGATTAGA |
| | | TACCCTGGTAGTCCACGCCGTAAACGATGAGTGCTAGGTGTTAG |
| 139 | DP74 16S | GCCTAATACATGCAAGTCGTGCGGACCTTTTAAAAGCTTGCTTTTAAAAGG |
| | rRNA | TTAGCGGCGAACGGGTGAGTAACACGTGGGCAACCTGCCTGTAAGATCGG |
| | | GATAATGCCGGGAAACCGGGGCTAATACCGGATAGTTTTTTCCTCCGCAT |
| | | GGAGGAAAAAGGAAAGACGGCTTCGGCTGTCACTTACAGATGGGCCCGC |
| | | GGCGCATTAGCTTGTTGGTGGGGTAACGGCTCACCAAGGCAACGATGCGT |
| | | AGCCGACCTGAGAGGGTGATCGGCCACATTGGGACTGAGACACGGCCCAA |
| | | ACTCCTACGGGAGGCAGCAGTAGGGAATCTTCCGCAATGGACGAAAGTCT |
| | | GACGGAGCAACGCCGCGTGAGTGAAGAAGGCCTTCGGGTCGTAAAACTCT |
| | | GTTGCCGGGGAAGAACAAGTGCCGTTCGAACAGGGCGGCGCCTTGACGGT |
| | | ACCCGGCCAGAAAGCCACGGCTAACTACGTGCCAGCAGCCGCGGTAATAC |
| | | GTAGGTGGCAAGCGTTGTCCGGAATTATTGGGCGTAAAGCGCGCGCAGGC |
| | | GGCTTCTTAAGTCTGATGTGAAATCTTGCGGCTCAACCGCAAGCGGTCATT |
| | | GGAAACTGGGAGGCTTGAGTGCAGAAGAGGAGAGTGGAATTCCACGTGT |
| | | AGCGGTGAAATGCGTAGAGATGTGGAGGAACACCAGTGGCGAAGGCGGC |
| | | TCTCTGGTCTGTAACTGACGCTGAGGCGCGAAAGCGTGGGGAGCAAACAG |
| | | GATTAGATACCCTGGTAGTCCACGCCGTAAACGATGAGTGCTAAGTGTTA |
| | | GAGGGTTTCCGCCCTTTAGTGCTGCAGCTAACGCATTAAGCACTCCGCCTG |
| | | GGGAGTACGGCCGCAAGGCTGAAACTCAAAGGAATTGACGGGGGCCCGC |
| | | ACAAGCGGTGGAGCATGTGGTTTAATTCGAAGCAACGCGAAGAACCTTAC |
| | | CAGGTCTTGACATCCTCTGACCTCCCTGGAGACAGGGCCTTCCCCTTCGGG |
| | | GGACAGAGTGACAGGTGGTGCATGGTTGTCGTCAGCTCGTGTCGTGAGAT |
| | | GTTGGGTTAAGTCCCGCAACGAGCGCAACCCTTGACCTTAGTTGCCAGCAT |
| | | TCAG |
| 140 | DP75 16S | TGAAGAGTTTGATCATGGCTCAGATTGAACGCTGGCGGCAGGCCTAACAC |
| | rRNA | ATGCAAGTCGAGCGGTAGAGAGAAGCTTGCTTCTCTTGAGAGCGGCGGAC |
| | | GGGTGAGTAATGCCTAGGAATCTGCCTGGTAGTGGGGGATAACGTTCGGA |
| | | AACGGACGCTAATACCGCATACGTCCTACGGGAGAAAGCAGGGGACCTTC |
| | | GGGCCTTGCGCTATCAGATGAGCCTAGGTCGGATTAGCTAGTTGGTGAGG |
| | | TAATGGCTCACCAAGGCGACGATCCGTAACTGGTCTGAGAGGATGATCAG |
| | | TCACACTGGAACTGAGACACGGTCCAGACTCCTACGGGAGGCAGCAGTGG |
| | | GGAATATTGGACAATGGGCGAAAGCCTGATCCAGCCATGCCGCGTGTGTG |
| | | AAGAAGGTCTTCGGATTGTAAAGCACTTTAAGTTGGGAGGAAGGGTTGTA |
| | | GATTAATACTCTGCAATTTTGACGTTACCGACAGAATAAGCACCGGCTAA |
| | | CTCTGTGCCAGCAGCCGCGGTAATACAGAGGGTGCAAGCGTTAATCGGAA |
| | | TTACTGGGCGTAAAGCGCGCGTAGGTGGTTCGTTAAGTTGGATGTGAAAG |
| | | CCCCGGGCTCAACCTGGGAACTGCATTCAAAACTGACGAGCTAGAGTATG |
| | | GTAGAGGGTGGTGGAATTTCCTGTGTAGCGGTGAAATGCGTAGATATAGG |
| | | AAGGAACACCAGTGGCGAAGGCGACCACCTGGACTGATACTGACACTGA |
| | | GGTGCGAAAGCGTGGGGAGCAAACAGGATTAGATACCCTGGTAGTCCACG |
| | | CCGTAAACGATGTCAACTAGCCGTTGGAATCCTTGAGATTTTAGTGGCGCA |
| | | GCTAACGCATTAAGTTGACCGCCTGGGGAGTACGGCCGCAAGGTTAAAAC |
| | | TCAAATGAATTGACGGGGGCCCGCACAAGCGGTGGAGCATGTGGTTTAAT |
| | | TCGAAGCAACGCGAAGAACCTTACCAGGCCTTGACATCCAATGAACTTTC |
| | | CAGAGATGGATGGGTGCCTTCGGGAACATTGAGACAGGTGCTGCATGGCT |
| | | GTCGTCAGCTCGTGTCGTGAGATGTTGGGTTAAGTCCCGTAACGAGCGCA |
| | | ACCCTTGTCCTTAGTTACCAGCACGTTATGGTGGGCACTCTAAGGAGACTG |
| | | CCGGTGACAAACCGGAGGAAGGTGGGGATGACGTCAAGTCATCATGGCCC |
| | | TTACGGCCTGGGCTACACACGTGCTACAATGGTCGGTACAAAGGGTTGCC |
| | | AAGCCGCGAGGTGGAGCTAATCCCATAAAACCGATCGTAGTCCGGATCGC |
| | | AGTCTGCAACTCGACTGCGTGAAGTCGGAATCGCTAGTAATCGCGAATCA |
| | | GAATGTCGCGGTGAATACGTTCCCGGGCCTTGTACACACCGCCCGTCACA |
| | | CCATGGGAGTGGGTTGCACCAGAACGGGAGGACGGTTACCACGGTGTGAT |
| | | TCATGACTGGGGTGAAGTCGTAACAAGGTAGCCGTAGGGGAACCTGCGGC |
| | | TGGATCACCTCCTT |
| 141 | DP76 16S | CTTGAGAGTTTGATCCTGGCTCAGAACGAACGCTGGCGGCAGGCTTAACA |
| | rRNA | CATGCAAGTCGAGCGCCCCGCAAGGGGAGCGGCAGACGGGTGAGTAACG |
| | | CGTGGGAATCTACCTTTTGCTACGGAACAACAGTTGGAAACGACTGCTAA |
| | | TACCGTATGTGCCCTTCGGGGGAAAGATTTATCGGCAAAGGATGAGCCCG |
| | | CGTTGGATTAGCTAGTTGGTGAGGTAAAGGCTCACCAAGGCGACGATCCA |
| | | TAGCTGGTCTGAGAGGATGATCAGCCACACTGGGACTGAGACACGGCCCA |
| | | GACTCCTACGGGAGGCAGCAGTGGGGAATATTGGACAATGGGCGCAAGC |
| | | CTGATCCAGCCATGCCGCGTGAGTGATGAAGGCCCTAGGGTTGTAAAGCT |
| | | CTTTCACCGGTGAAGATAATGACGGTAACCGGAGAAGAAGCCCCGGCTAA |
| | | CTTCGTGCCAGCAGCCGCGGTAATACGAAGGGGGCTAGCGTTGTTCGGAT |
| | | TTACTGGGCGTAAAGCGCACGTAGGCGGATTTTTAAGTCAGGGGTGAAAT |
| | | CCCGGGGCTCAACCCCGGAACTGCCTTTGATACTGGAAGTCTTGAGTATG |
| | | GTAGAGGTGAGTGGAATTCCGAGTGTAGAGGTGAAATTCGTAGATATTCG |
| | | GAGGAACACCAGTGGCGAAGGCGGCTCACTGGACCATTACTGACGCTGAG |
| | | GTGCGAAAGCGTGGGGAGCAAACAGGATTAGATACCCTGGTAGTCCACGC |
| | | CGTAAACGATGAATGTTAGCCGTCGGGGGGTTTACCTTTCGGTGGCGCAG |
| | | CTAACGCATTAAACATTCCGCCTGGGGAGTACGGTCGCAAGATTAAAACT |
| | | CAAAGGAATTGACGGGGGCCCGCACAAGCGGTGGAGCATGTGGTTTAATT |
| | | CGAAGCAACGCGCAGAACCTTACCAGCCCTTGACATACCGGTCGCGGACA |
| | | CAGAGATGTGTCTTTCAGTTCGGCTGGACCGGATACAGGTGCTGCATGGCT |
| | | GTCGTCAGCTCGTGTCGTGAGATGTTGGGTTAAGTCCCGCAACGAGCGCA |
| | | ACCCTCGCCTTTAGTTGCCAGCATTTAGTTGGGCACTCTAAAGGGACTGCC |
| | | AGTGATAAGCTGGAGGAAGGTGGGGATGACGTCAAGTCCTCATGGCCCTT |
| | | ACGGGCTGGGCTACACACGTGCTACAATGGTGGTGACAGTGGGCAGCAAG |
| | | CACGCGAGTGTGAGCTAATCTCCAAAAGCCATCTCAGTTCGGATTGCACTC |
| | | TGCAACTCGAGTGCATGAAGTTGGAATCGCTAGTAATCGCGGATCAGCAT |
| | | GCCGCGGTGAATACGTTCCCGGGCCTTGTACACACCGCCCGTCACACCAT |
| | | GGGAGTTGGTTTTACCCGAAGGCACTGTGCTAACCGCAAGGAGGCAGGTG |
| | | ACCACGGTAGGGTCAGCGACTGGGGTGAAGTCGTAACAAGGTAGCCGTAG |
| | | GGGAACCTGCGGCTGGATCACCTCCTTT |
| 142 | DP77 16S | TCGGAGAGTTTGATCCTGGCTCAGGATGAACGCTGGCGGCGTGCCTAATA |
| | rRNA | CATGCAAGTCGAGCGAACTGATTAGAAGCTTGCTTCTATGACGTTAGCGG |
| | | CGGACGGGTGAGTAACACGTGGGCAACCTGCCTGTAAGACTGGGATAACT |
| | | TCGGGAAACCGAAGCTAATACCGGATAGGATCTTCTCCTTCATGGGAGAT |
| | | GATTGAAAGATGGTTTCGGCTATCACTTACAGATGGGCCCGCGGTGCATT |
| | | AGCTAGTTGGTGAGGTAACGGCTCACCAAGGCAACGATGCATAGCCGACC |
| | | TGAGAGGGTGATCGGCCACACTGGGACTGAGACACGGCCCAGACTCCTAC |
| | | GGGAGGCAGCAGTAGGGAATCTTCCGCAATGGACGAAAGTCTGACGGAG |
| | | CAACGCCGCGTGAGTGATGAAGGCTTTCGGGTCGTAAAACTCTGTTGTTA |
| | | GGGAAGAACAAGTACAAGAGTAACTGCTTGTACCTTGACGGTACCTAACC |
| | | AGAAAGCCACGGCTAACTACGTGCCAGCAGCCGCGGTAATACGTAGGTGG |
| | | CAAGCGTTATCCGGAATTATTGGGCGTAAAGCGCGCGCAGGCGGTTTCTT |
| | | AAGTCTGATGTGAAAGCCCACGGCTCAACCGTGGAGGGTCATTGGAAACT |
| | | GGGGAACTTGAGTGCAGAAGAGAAAAGCGGAATTCCACGTGTAGCGGTG |
| | | AAATGCGTAGAGATGTGGAGGAACACCAGTGGCGAAGGCGGCTTTTTGGT |
| | | CTGTAACTGACGCTGAGGCGCGAAAGCGTGGGGAGCAAACAGGATTAGA |
| | | TACCCTGGTAGTCCACGCCGTAAACGATGAGTGCTAAGTGTTAGAGGGTT |
| | | TCCGCCCTTTAGTGCTGCAGCTAACGCATTAAGCACTCCGCCTGGGGAGTA |
| | | CGGTCGCAAGACTGAAACTCAAAGGAATTGACGGGGGCCCGCACAAGCG |
| | | GTGGAGCATGTGGTTTAATTCGAAGCAACGCGAAGAACCTTACCAGGTCT |
| | | TGACATCCTCTGACAACTCTAGAGATAGAGCGTTCCCCTTCGGGGGACAG |
| | | AGTGACAGGTGGTGCATGGTTGTCGTCAGCTCGTGTCGTGAGATGTTGGGT |
| | | TAAGTCCCGCAACGAGCGCAACCCTTGATCTTAGTTGCCAGCATTCAGTTG |
| | | GGCACTCTAAGGTGACTGCCGGTGACAAACCGGAGGAAGGTGGGGATGA |
| | | CGTCAAATCATCATGCCCCTTATGACCTGGGCTACACACGTGCTACAATGG |
| | | ATGGTACAAAGGGCTGCAAGACCGCGAGGTCAAGCCAATCCCATAAAACC |
| | | ATTCTCAGTTCGGATTGTAGGCTGCAACTCGCCTACATGAAGCTGGAATCG |
| | | CTAGTAATCGCGGATCAGCATGCCGCGGTGAATACGTTCCCGGGCCTTGT |
| | | ACACACCGCCCGTCACACCACGAGAGTTTGTAACACCCGAAGTCGGTGGA |
| | | GTAACCGTAAGGAGCTAGCCGCCTAAGGTGGGACAGATGATTGGGGTGAA |
| | | GTCGTAACAAGGTAGCCGTATCGGAAGGTGCGGCTGGATCACCTCCTTT |
| 143 | DP78 16S | TTGAAGAGTTTGATCATGGCTCAGATTGAACGCTGGCGGCAGGCCTAACA |
| | rRNA | CATGCAAGTCGAACGGTAGCACAGAGAGCTTGCTCTTGGGTGACGAGTGG |
| | | CGGACGGGTGAGTAATGTCTGGGAAACTGCCCGATGGAGGGGGATAACTA |
| | | CTGGAAACGGTAGCTAATACCGCATAACGTCTTCGGACCAAAGTGGGGGA |
| | | CCTTCGGGCCTCACACCATCGGATGTGCCCAGATGGGATTAGCTAGTAGG |
| | | TGGGGTAATGGCTCACCTAGGCGACGATCCCTAGCTGGTCTGAGAGGATG |
| | | ACCAGCCACACTGGAACTGAGACACGGTCCAGACTCCTACGGGAGGCAGC |
| | | AGTGGGGAATATTGCACAATGGGCGCAAGCCTGATGCAGCCATGCCGCGT |
| | | GTATGAAGAAGGCCTTCGGGTTGTAAAGTACTTTCAGTGGGGAGGAAGGC |
| | | GATGAAGTTAATAGCTTCGTCGATTGACGTTACCCGCAGAAGAAGCACCG |
| | | GCTAACTCCGTGCCAGCAGCCGCGGTAATACGGAGGGTGCAAGCGTTAAT |
| | | CGGAATTACTGGGCGTAAAGCGCACGCAGGCGGTCTGTCAAGTCGGATGT |
| | | GAAATCCCCGGGCTCAACCTGGGAACTGCATTCGAAACTGGCAGGCTAGA |
| | | GTCTTGTAGAGGGGGGTAGAATTCCAGGTGTAGCGGTGAAATGCGTAGAG |
| | | ATCTGGAGGAATACCGGTGGCGAAGGCGGCCCCCTGGACAAAGACTGAC |
| | | GCTCAGGTGCGAAAGCGTGGGGAGCAAACAGGATTAGATACCCTGGTAGT |
| | | CCACGCCGTAAACGATGTCGACTTGGAGGTTGTGCCCTTGAGGCGTGGCTT |
| | | CCGGAGCTAACGCGTTAAGTCGACCGCCTGGGGAGTACGGCCGCAAGGTT |
| | | AAAACTCAAATGAATTGACGGGGGCCCGCACAAGCGGTGGAGCATGTGGT |
| | | TTAATTCGATGCAACGCGAAGAACCTTACCTGGCCTTGACATCCACGGAA |
| | | TTCGGCAGAGATGCCTTAGTGCCTTCGGGAACCGTGAGACAGGTGCTGCA |
| | | TGGCTGTCGTCAGCTCGTGTTGTGAAATGTTGGGTTAAGTCCCGCAACGAG |
| | | CGCAACCCTTATCCTTTGTTGCCAGCGAGTAATGTCGGGAACTCAAAGGA |
| | | GACTGCCGGTGATAAACCGGAGGAAGGTGGGGATGACGTCAAGTCATCAT |
| | | GGCCCTTACGGCCAGGGCTACACACGTGCTACAATGGCGCATACAAAGAG |
| | | AAGCGACCTCGCGAGAGCAAGCGGACCTCATAAAGTGCGTCGTAGTCCGG |
| | | ATCGGAGTCTGCAACTCGACTCCGTGAAGTCGGAATCGCTAGTAATCGTA |
| | | GATCAGAATGCTACGGTGAATACGTTCCCGGGCCTTGTACACACCGCCCG |
| | | TCACACCATGGGAGTGGGTTGCAAAAGAAGTAGGTAGCTTAACCTTCGGG |
| | | AGGGCGCTTACCACTTTGTGATTCATGACTGGGGTGAAGTCGTAACAAGG |
| | | TAACCGTAGGGGAACCTGCGGTTGGATCACCTCCTT |
| 144 | DP79 16S | TGAAGAGTTTGATCATGGCTCAGATTGAACGCTGGCGGCAGGCCTAACAC |
| | rRNA | ATGCAAGTCGAGCGGTAGAGAGAAGCTTGCTTCTCTTGAGAGCGGCGGAC |
| | | GGGTGAGTAATACCTAGGAATCTGCCTGATAGTGGGGGATAACGTTCGGA |
| | | AACGGACGCTAATACCGCATACGTCCTACGGGAGAAAGCAGGGGACCTTC |
| | | GGGCCTTGCGCTATCAGATGAGCCTAGGTCGGATTAGCTAGTTGGTGAGG |
| | | TAATGGCTCACCAAGGCTACGATCCGTAACTGGTCTGAGAGGATGATCAG |
| | | TCACACTGGAACTGAGACACGGTCCAGACTCCTACGGGAGGCAGCAGTGG |
| | | GGAATATTGGACAATGGGCGAAAGCCTGATCCAGCCATGCCGCGTGTGTG |
| | | AAGAAGGTCTTCGGATTGTAAAGCACTTTAAGTTGGGAGGAAGGGCAGTT |
| | | ACCTAATACGTGACTGTCTTGACGTTACCGACAGAATAAGCACCGGCTAA |
| | | CTCTGTGCCAGCAGCCGCGGTAATACAGAGGGTGCAAGCGTTAATCGGAA |
| | | TTACTGGGCGTAAAGCGCGCGTAGGTGGTTTGTTAAGTTGAATGTGAAAT |
| | | CCCCGGGCTCAACCTGGGAACTGCATCCAAAACTGGCAAGCTAGAGTATG |
| | | GTAGAGGGTAGTGGAATTTCCTGTGTAGCGGTGAAATGCGTAGATATAGG |
| | | AAGGAACACCAGTGGCGAAGGCGACTACCTGGACTGATACTGACACTGAG |
| | | GTGCGAAAGCGTGGGGAGCAAACAGGATTAGATACCCTGGTAGTCCACGC |
| | | CGTAAACGATGTCAACTAGCCGTTGGGAGTCTTGAACTCTTAGTGGCGCA |
| | | GCTAACGCATTAAGTTGACCGCCTGGGGAGTACGGCCGCAAGGTTAAAAC |
| | | TCAAATGAATTGACGGGGGCCCGCACAAGCGGTGGAGCATGTGGTTTAAT |
| | | TCGAAGCAACGCGAAGAACCTTACCAGGCCTTGACATCCAATGAACTTTC |
| | | TAGAGATAGATTGGTGCCTTCGGGAACATTGAGACAGGTGCTGCATGGCT |
| | | GTCGTCAGCTCGTGTCGTGAGATGTTGGGTTAAGTCCCGTAACGAGCGCA |
| | | ACCCTTGTCCTTAGTTACCAGCACGTAATGGTGGGCACTCTAAGGAGACTG |
| | | CCGGTGACAAACCGGAGGAAGGTGGGGATGACGTCAAGTCATCATGGCCC |
| | | TTACGGCCTGGGCTACACACGTGCTACAATGGTCGGTACAAAGGGTTGCC |
| | | AAGCCGCGAGGTGGAGCTAATCCCATAAAACCGATCGTAGTCCGGATCGC |
| | | AGTCTGCAACTCGACTGCGTGAAGTCGGAATCGCTAGTAATCGTGAATCA |
| | | GAATGTCACGGTGAATACGTTCCCGGGCCTTGTACACACCGCCCGTCACA |
| | | CCATGGGAGTGGGTTGCACCAGAAGTAGCTAGTCTAACCTTCGGGAGGAC |
| | | GGTTACCACGGTGTGATTCATGACTGGGGTGAAGTCGTAACAAGGTAGCC |
| | | GTAGGGGAACCTGCGGCTGGATCACCTCCTT |
| 145 | DP80 16S | CTTGAGAGTTTGATCCTGGCTCAGAGCGAACGCTGGCGGCAGGCTTAACA |
| | rRNA | CATGCAAGTCGAGCGGGCACCTTCGGGTGTCAGCGGCAGACGGGTGAGTA |
| | | ACACGTGGGAACGTACCCTTCGGTTCGGAATAACGCTGGGAAACTAGCGC |
| | | TAATACCGGATACGCCCTTTTGGGGAAAGGTTTACTGCCGAAGGATCGGC |
| | | CCGCGTCTGATTAGCTAGTTGGTGGGGTAACGGCCTACCAAGGCGACGAT |
| | | CAGTAGCTGGTCTGAGAGGATGATCAGCCACACTGGGACTGAGACACGGC |
| | | CCAGACTCCTACGGGAGGCAGCAGTGGGGAATATTGGACAATGGGCGCA |
| | | AGCCTGATCCAGCCATGCCGCGTGAGTGATGAAGGCCTTAGGGTTGTAAA |
| | | GCTCTTTTGTCCGGGACGATAATGACGGTACCGGAAGAATAAGCCCCGGC |
| | | TAACTTCGTGCCAGCAGCCGCGGTAATACGAAGGGGGCTAGCGTTGCTCG |
| | | GAATCACTGGGCGTAAAGGGCGCGTAGGCGGCCATTCAAGTCGGGGGTGA |
| | | AAGCCTGTGGCTCAACCACAGAATTGCCTTCGATACTGTTTGGCTTGAGTT |
| | | TGGTAGAGGTTGGTGGAACTGCGAGTGTAGAGGTGAAATTCGTAGATATT |
| | | CGCAAGAACACCAGTGGCGAAGGCGGCCAACTGGACCAATACTGACGCT |
| | | GAGGCGCGAAAGCGTGGGGAGCAAACAGGATTAGATACCCTGGTAGTCC |
| | | ACGCCGTAAACGATGAATGCTAGCTGTTGGGGTGCTTGCACCTCAGTAGC |
| | | GCAGCTAACGCTTTAAGCATTCCGCCTGGGGAGTACGGTCGCAAGATTAA |
| | | AACTCAAAGGAATTGACGGGGGCCCGCACAAGCGGTGGAGCATGTGGTTT |
| | | AATTCGAAGCAACGCGCAGAACCTTACCATCCCTTGACATGTCGTGCCATC |
| | | CGGAGAGATCCGGGGTTCCCTTCGGGGACGCGAACACAGGTGCTGCATGG |
| | | CTGTCGTCAGCTCGTGTCGTGAGATGTTGGGTTAAGTCCCGCAACGAGCGC |
| | | AACCCACGTCCTTAGTTGCCATCATTTAGTTGGGCACTCTAGGGAGACTGC |
| | | CGGTGATAAGCCGCGAGGAAGGTGTGGATGACGTC |
| 146 | DP81 16S | AACGGAGAGTTTGATCCTGGCTCAGGACGAACGCTGGCGGCGTGCCTAAT |
| | rRNA | ACATGCAAGTCGAGCGGACAGAAGGGAGCTTGCTCCCGGACGTTAGCGGC |
| | | GGACGGGTGAGTAACACGTGGGCAACCTGCCCCTTAGACTGGGATAACTC |
| | | CGGGAAACCGGAGCTAATACCGGATAATCCCTTTCTCCACCTGGAGAGAG |
| | | GGTGAAAGATGGCTTCGGCTATCACTAGGGGATGGGCCCGCGGCGCATTA |
| | | GCTAGTTGGTAAGGTAACGGCTTACCAAGGCGACGATGCGTAGCCGACCT |
| | | GAGAGGGTGATCGGCCACACTGGGACTGAGACACGGCCCAGACTCCTACG |
| | | GGAGGCAGCAGTAGGGAATCTTCCGCAATGGACGAAAGTCTGACGGAGC |
| | | AACGCCGCGTGAGTGAGGAAGGCTTTCGGGTCGTAAAGCTCTGTTGTGAG |
| | | GGAAGAAGCGGTACCGTTCGAATAGGGCGGTACCTTGACGGTACCTCACC |
| | | AGAAAGCCACGGCTAACTACGTGCCAGCAGCCGCGGTAATACGTAGGTGG |
| | | CAAGCGTTGTCCGGAATTATTGGGCGTAAAGCGCGCGCAGGCGGCTTCTT |
| | | AAGTCTGATGTGAAATCTCGGGGCTCAACCCCGAGCGGCCATTGGAAACT |
| | | GGGGAGCTTGAGTGCAGAAGAGGAGAGTGGAATTCCACGTGTAGCGGTG |
| | | AAATGCGTAGAGATGTGGAGGAACACCAGTGGCGAAGGCGACTCTCTGGT |
| | | CTGTAACTGACGCTGAGGCGCGAAAGCGTGGGGAGCAAACAGGATTAGA |
| | | TACCCTGGTAGTCCACGCCGTAAACGATGAGTGCTAGGTGTTAGGGGTTTC |
| | | GATGCCCGTAGTGCCGAAGTTAACACATTAAGCACTCCGCCTGGGGAGTA |
| | | CGGCCGCAAGGCTGAAACTCAAAGGAATTGACGGGGACCCGCACAAGCA |
| | | GTGGAGCATGTGGTTTAATTCGAAGCAACGCGAAGAACCTTACCAGGTCT |
| | | TGACATCCTTTGACCACCCAAGAGATTGGGCTTCCCCTTCGGGGGCAAAGT |
| | | GACAGGTGGTGCATGGTTGTCGTCAGCTCGTGTCGTGAGATGTTGGGTTAA |
| | | GTCCCGCAACGAGCGCAACCCTTGATCTTAGTTGCCAGCATTGAGTTGGGC |
| | | ACTCTAAGGTGACTGCCGGTGACAAACCGGAGGAAGGTGGGGATGACGTC |
| | | AAATCATCATGCCCCTTATGACCTGGGCTACACACGTGCTACAATGGATG |
| | | GTACAAAGGGCAGCGAAACCGCGAGGTGAAGCCAATCCCATAAAGCCAT |
| | | TCTCAGTTCGGATTGCAGGCTGCAACTCGCCTGCATGAAGCCGGAATTGCT |
| | | AGTAATCGCGGATCAGCATGCCGCGGTGAATACGTTCCCGGGTCTTGTAC |
| | | ACACCGCCCGTCACACCACGAGAGTTTGTAACACCCGAAGTCGGTGAGGC |
| | | AACCTTTTGGAGCCAGCCGCCTAAGGTGGGACAAATGATTGGGGTGAAGT |
| | | CGTAACAAGGTAGCCGTATCGGAAGGTGCGGCTGGATCACCTCCTTT |
| 147 | DP82 16S | AACGGAGAGTTTGATCCTGGCTCAGGACGAACGCTGGCGGCGTGCCTAAT |
| | rRNA | ACATGCAAGTCGAGCGGACAGAAGGGAGCTTGCTCCCGGACGTTAGCGGC |
| | | GGACGGGTGAGTAACACGTGGGCAACCTGCCCCTTAGACTGGGATAACTC |
| | | CGGGAAACCGGAGCTAATACCGGATAATCCCTTTCTCCACCTGGAGAGAG |
| | | GGTGAAAGATGGCTTCGGCTATCACTAAGGGATGGGCCCGCGGCGCATTA |
| | | GCTAGTTGGTAAGGTAACGGCTTACCAAGGCAACGATGCGTAGCCGACCT |
| | | GAGAGGGTGATCGGCCACACTGGGACTGAGACACGGCCCAGACTCCTACG |
| | | GGAGGCAGCAGTAGGGAATCTTCCGCAATGGACGAAAGTCTGACGGAGC |
| | | AACGCCGCGTGAGTGAGGAAGGCCTTCGGGTCGTAAAGCTCTGTTGTGAG |
| | | GGAAGAAGCGGTACCGTTCGAATAGGGCGGTACCTTGACGGTACCTCACC |
| | | AGAAAGCCACGGCTAACTACGTGCCAGCAGCCGCGGTAATACGTAGGTGG |
| | | CAAGCGTTGTCCGGAATTATTGGGCGTAAAGCGCGCGCAGGCGGCTTCTT |
| | | AAGTCTGATGTGAAATCTCGGGGCTCAACCCCGAGCGGCCATTGGAAACT |
| | | GGGGAGCTTGAGTGCAGAAGAGGAGAGTGGAATTCCACGTGTAGCGGTG |
| | | AAATGCGTAGAGATGTGGAGGAACACCAGTGGCGAAGGCGACTCTCTGGT |
| | | CTGTAACTGACGCTGAGGCGCGAAAGCGTGGGGAGCAAACAGGATTAGA |
| | | TACCCTGGTAGTCCACGCCGTAAACGATGAGTGCTAGGTGTTAGGGGTTTC |
| | | GATGCCCGTAGTGCCGAAGTTAACACATTAAGCACTCCGCCTGGGGAGTA |
| | | CGGCCGCAAGGCTGAAACTCAAAGGAATTGACGGGGACCCGCACAAGCA |
| | | GTGGAGCATGTGGTTTAATTCGAAGCAACGCGAAGAACCTTACCAGGTCT |
| | | TGACATCCTTTGACCACCCAAGAGATTGGGCTTCCCCTTCGGGGGCAAAGT |
| | | GACAGGTGGTGCATGGTTGTCGTCAGCTCGTGTCGTGAGATGTTGGGTTAA |
| | | GTCCCGCAACGAGCGCAACCCTTGATCTTAGTTGCCAGCATTCAGTTGGGC |
| | | ACTCTAAGGTGACTGCCGGTGACAAACCGGAGGAAGGTGGGGATGACGTC |
| | | AAATCATCATGCCCCTTATGACCTGGGCTACACACGTGCTACAATGGATG |
| | | GTACAAAGGGCAGCGAAACCGCGAGGTGAAGCCAATCCCATAAAGCCAT |
| | | TCTCAGTTCGGATTGCAGGCTGCAACTCGCCTGCATGAAGCCGGAATTGCT |
| | | AGTAATCGCGGATCAGCATGCCGCGGTGAATACGTTCCCGGGTCTTGTAC |
| | | ACACCGCCCGTCACACCACGAGAGTTTGTAACACCCGAAGTCGGTGAGGC |
| | | AACCTTTTGGAGCCAGCCGCCTAAGGTGGGACAAATGATTGGGGTGAAGT |
| | | CGTAACAAGGTAGCCGTATCGGAAGGTGCGGCTGGATCACCTCCTTT |
| 148 | DP83 16S | ACGGAGAGTTTGATCCTGGCTCAGGACGAACGCTGGCGGCGTGCCTAATA |
| | rRNA | CATGCAAGTCGAGCGGAGTTTCAAGAAGCTTGCTTTTTGAAACTTAGCGG |
| | | CGGACGGGTGAGTAACACGTGGGCAACCTGCCCCTTAGACTGGGATAACT |
| | | CCGGGAAACCGGAGCTAATACCGGATAATCCCTTTCTCCACCTGGAGAGA |
| | | GGGTGAAAGATGGCTTCGGCTATCACTAAGGGATGGGCCCGCGGCGCATT |
| | | AGCTAGTTGGTAAGGTAACGGCTTACCAAGGCAACGATGCGTAGCCGACC |
| | | TGAGAGGGTGATCGGCCACACTGGGACTGAGACACGGCCCAGACTCCTAC |
| | | GGGAGGCAGCAGTAGGGAATCTTCCGCAATGGACGAAAGTCTGACGGAG |
| | | CAACGCCGCGTGAGTGAGGAAGGCCTTCGGGTCGTAAAGCTCTGTTGTGA |
| | | GGGAAGAAGCGGTACCGTTCGAATAGGGCGGTACCTTGACGGTACCTCAC |
| | | CAGAAAGCCACGGCTAACTACGTGCCAGCAGCCGCGGTAATACGTAGGTG |
| | | GCAAGCGTTGTCCGGAATTATTGGGCGTAAAGCGCGCGCAGGCGGCTTCT |
| | | TAAGTCTGATGTGAAATCTCGGGGCTCAACCCCGAGCGGCCATTGGAAAC |
| | | TGGGGAGCTTGAGTGCAGAAGAGGAGAGTGGAATTCCACGTGTAGCGGTG |
| | | AAATGCGTAGAGATGTGGAGGAACACCAGTGGCGAAGGCGACTCTCTGGT |
| | | CTGTAACTGACGCTGAGGCGCGAAAGCGTGGGGAGCAAACAGGATTAGA |
| | | TACCCTGGTAGTCCACGCCGTAAACGATGAGTGCTAGGTGTTAGGGGTTTC |
| | | GATGCCCGTAGTGCCGAAGTTAACACATTAAGCACTCCGCCTGGGGAGTA |
| | | CGGCCGCAAGGCTGAAACTCAAAGGAATTGACGGGGACCCGCACAAGCA |
| | | GTGGAGCATGTGGTTTAATTCGAAGCAACGCGAAGAACCTTACCAGGTCT |
| | | TGACATCCTTTGACCACCCAAGAGATTGGGCTTCCCCTTCGGGGGCAAAGT |
| | | GACAGGTGGTGCATGGTTGTCGTCAGCTCGTGTCGTGAGATGTTGGGTTAA |
| | | GTCCCGCAACGAGCGCAACCCTTGATCTTAGTTGCCAGCATTCAGTTGGGC |
| | | ACTCTAAGGTGACTGCCGGTGACAAACCGGAGGAAGGTGGGGATGACGTC |
| | | AAATCATCATGCCCCTTATGACCTGGGCTACACACGTGCTACAATGGATG |
| | | GTACAAAGGGCAGCGAAGCCGCGAGGTGAAGCCAATCCCATAAAGCCAT |
| | | TCTCAGTTCGGATTGCAGGCTGCAACTCGCCTGCATGAAGCCGGAATTGCT |
| | | AGTAATCGCGGATCAGCATGCCGCGGTGAATACGTTCCCGGGTCTTGTAC |
| | | ACACCGCCCGTCACACCACGAGAGTTTGTAACACCCGAAGTCGGTGAGGC |
| | | AACCTTTTGGAGCCAGCCGCCTAAGGTGGGACAAATGATTGGGGTGAAGT |
| | | CGTAACAAGGTAGCCGTATCGGAAGGTGCGGCTGGATCACCTCCTTT |
| 149 | DP84 16S | TACGGAGAGTTTGATCCTGGCTCAGGATGAACGCTGGCGGCGTGCTTAAC |
| | rRNA | ACATGCAAGTCGAACGGTGAAGCCAAGCTTGCTTGGTGGATCAGTGGCGA |
| | | ACGGGTGAGTAACACGTGAGCAACCTGCCCTGGACTCTGGGATAAGCGCT |
| | | GGAAACGGCGTCTAATACTGGATATGAGCTCTCATCGCATGGTGGGGGTT |
| | | GGAAAGATTTTTTGGTCTGGGATGGGCTCGCGGCCTATCAGCTTGTTGGTG |
| | | AGGTAATGGCTCACCAAGGCGTCGACGGGTAGCCGGCCTGAGAGGGTGAC |
| | | CGGCCACACTGGGACTGAGACACGGCCCAGACTCCTACGGGAGGCAGCA |
| | | GTGGGGAATATTGCACAATGGGCGAAAGCCTGATGCAGCAACGCCGCGTG |
| | | AGGGATGACGGCCTTCGGGTTGTAAACCTCTTTTAGCAGGGAAGAAGCGA |
| | | AAGTGACGGTACCTGCAGAAAAAGCGCCGGCTAACTACGTGCCAGCAGCC |
| | | GCGGTAATACGTAGGGCGCAAGCGTTATCCGGAATTATTGGGCGTAAAGA |
| | | GCTCGTAGGCGGTTTGTCGCGTCTGCTGTGAAATCCCGAGGCTCAACCTCG |
| | | GGCCTGCAGTGGGTACGGGCAGACTAGAGTGCGGTAGGGGAGATTGGAA |
| | | TTCCTGGTGTAGCGGTGGAATGCGCAGATATCAGGAGGAACACCGATGGC |
| | | GAAGGCAGATCTCTGGGCCGTAACTGACGCTGAGGAGCGAAAGGGTGGG |
| | | GAGCAAACAGGCTTAGATACCCTGGTAGTCCACCCCGTAAACGTTGGGAA |
| | | CTAGTTGTGGGGACCATTCCACGGTTTCCGTGACGCAGCTAACGCATTAAG |
| | | TTCCCCGCCTGGGGAGTACGGCCGCAAGGCTAAAACTCAAAGGAATTGAC |
| | | GGGGACCCGCACAAGCGGCGGAGCATGCGGATTAATTCGATGCAACGCG |
| | | AAGAACCTTACCAAGGCTTGACATACACCAGAACGGGCCAGAAATGGTCA |
| | | ACTCTTTGGACACTGGTGAACAGGTGGTGCATGGTTGTCGTCAGCTCGTGT |
| | | CGTGAGATGTTGGGTTAAGTCCCGCAACGAGCGCAACCCTCGTTCTATGTT |
| | | GCCAGCACGTAATGGTGGGAACTCATGGGATACTGCCGGGGTCAACTCGG |
| | | AGGAAGGTGGGGATGACGTCAAATCATCATGCCCCTTATGTCTTGGGCTTC |
| | | ACGCATGCTACAATGGCCGGTACAAAGGGCTGCAATACCGTGAGGTGGAG |
| | | CGAATCCCAAAAAGCCGGTCCCAGTTCGGATTGAGGTCTGCAACTCGACC |
| | | TCATGAAGTCGGAGTCGCTAGTAATCGCAGATCAGCAACGCTGCGGTGAA |
| | | TACGTTCCCGGGTCTTGTACACACCGCCCGTCAAGTCATGAAAGGAGCCG |
| | | TCGAAGGTGGGATCGGTAATTAGGACTAAGTCGTAACAAGGTAGCCGTAC |
| | | CGGAAGGTGCGGCTGGATCACCTCCTTT |
| 150 | DP85 16S | TGCAGTCGTACGCTTCTTTTTCCNCCGGAGCTTGCTCCACCGGAAAAAGAG |
| | rRNA | GAGTGGCGAACGGGTGAGTAACACGTGGGTAACCTGCCCATCAGAAGGG |
| | | GATAACACTTGGAAACAGGTGCTAATACCGTATAACAATCGAAACCGCAT |
| | | GGTTTTGATTTGAAAGGCGCTTTCGGGTGTCGCTGATGGATGGACCCGCGG |
| | | TGCATTAGCTAGTTGGTGAGGTAACGGCTCACCAAGGCCACGATGCATAG |
| | | CCGACCTGAGAGGGTGATCGGCCACATTGGGACTGAGACACGGCCCAAAC |
| | | TCCTACGGGAGGCAGCAGTAGGGAATCTTCGGCAATGGACGAAAGTCTGA |
| | | CCGAGCAACGCCGCGTGAGTGAAGAAGGTTTTCGGATCGTAAAACTCTGT |
| | | TGTTAGAGAAGAACAAGGATGAGAGTAACTGTTCATCCCTTGACGGTATC |
| | | TAACCAGAAAGCCACGGCTAACTACGTGCCAGCAGCCGCGGTAATACGTA |
| | | GGTGGCAAGCGTTGTCCGGATTTATTGGGCGTAAAGCGAGCGCAGGCGGT |
| | | TTCTTAAGTCTGATGTGAAAGCCCCCGGCTCAACCGGGGAGGGTCATTGG |
| | | AAACTGGGAGACTTGAGTGCAGAAGAGGAGAGTGGAATTCCATGTGTAGC |
| | | GGTGAAATGCGTAGATATATGGAGGAACACCAGTGGCGAAGGCGGCTCTC |
| | | TGGTCTGTAACTGACGCTGNNCTCGAAAGCGTGGGGAGCAAACAGGATTA |
| | | GATACCCTGGTAGTCCACGCCGTAAACGATGAGTGCTAAGTGTTGGAGGG |
| | | TTTCCGCCCTTCAGTGCTGCAGCTAACGCATTAAGCACTCCGCCTGGGGAG |
| | | TACGACCGCAAGGTTGAAACTCAAGGAATTGACGGGGGCCCGCACAGCG |
| | | GTGGAGCATGNNGNTTANNGANCACGCGANANNTACNNNCTNACATCNTT |
| | | GACNCTCTANAGATAGAGCTTCCCTTCGGGGCAAGTGACNG |
| 151 | DP86 16S | CGATGCGTAGCCGACCTGAGAGGGTGATCGGCCACACTGGGACTGAGACA |
| | rRNA | CGGCCCAGACTCCTACGGGAGGCAGCAGTAGGGAATCTTCCGCAATGGAC |
| | | GAAAGTCTGACGGAGCAACGCCGCGTGAGTGATGAAGGTTTTCGGATCGT |
| | | AAAGCTCTGTTGTTAGGGAAGAACAAGTGCCGTTCAAATAGGGCGGCACC |
| | | TTGACGGTACCTAACCAGAAAGCCACGGCTAACTACGTGCCAGCAGCCGC |
| | | GGTAATACGTAGGTGGCAAGCGTTGTCCGGAATTATTGGGCGTAAAGGGC |
| | | TCGCAGGCGGTTTCTTAAGTCTGATGTGAAAGCCCCCGGCTCAACCGGGG |
| | | AGGGTCATTGGAAACTGGGGAACTTGAGTGCAGAAGAGGAGAGTGGAAT |
| | | TCCACGTGTAGCGGTGAAATGCGTAGAGATGTGGAGGAACACCAGTGGCG |
| | | AAGGCGACTCTCTGGTCTGTAACTGACGCTGAGGAGCGAAAGCGTGGGGA |
| | | GCGAACAGGATTAGATACCCTGGTAGTCCACGCCGTAAACGATGAGTGCT |
| | | AAGTGTTAGGGGGTTTCCGCCCCTTAGTGCTGCAGCTAACGCATTAAGCAC |
| | | TCCGCCTGGGGAGTACGGTCGCAAGACTGAAACTCAAAGGAATTGACGGG |
| | | GGCCCGCACAAGCGGTGGAGCATGTGGTTTAATTCGAAGCAACGCGAAGA |
| | | ACCTTACCAGGTCTTGACATCCTCTGACAATCCTAGAGATAGGACGTCCCC |
| | | TTCGGGGGCAGAGTGACAGGTGGTGCATGGTTGTCGTCAGCTCGTGTCGT |
| | | GAGATGTTGGGTTAAGTCCCGCAACGAGCGCAACCCTTGATCTTAGTTGCC |
| | | AGCATTCAGTTGGGTGTTCTTTGAAAACT |
| 152 | DP87 16S | TTTGAGAGTTTGATCCTGGCTCAGGACGAACGCTGGCGGCGTGCCTAATA |
| | rRNA | CATGCAAGTCGAACGAACTCTGGTATTGATTGGTGCTTGCATCATGATTTA |
| | | CATTTGAGTGAGTGGCGAACTGGTGAGTAACACGTGGGAAACCTGCCCAG |
| | | AAGCGGGGGATAACACCTGGAAACAGATGCTAATACCGCATAACAACTTG |
| | | GACCGCATGGTCCGAGCTTGAAAGATGGCTTCGGCTATCACTTTTGGATGG |
| | | TCCCGCGGCGTATTAGCTAGATGGTGGGGTAACGGCTCACCATGGCAATG |
| | | ATACGTAGCCGACCTGAGAGGGTAATCGGCCACATTGGGACTGAGACACG |
| | | GCCCAAACTCCTACGGGAGGCAGCAGTAGGGAATCTTCCACAATGGACGA |
| | | AAGTCTGATGGAGCAACGCCGCGTGAGTGAAGAAGGGTTTCGGCTCGTAA |
| | | AACTCTGTTGTTAAAGAAGAACATATCTGAGAGTAACTGTTCAGGTATTG |
| | | ACGGTATTTAACCAGAAAGCCACGGCTAACTACGTGCCAGCAGCCGCGGT |
| | | AATACGTAGGTGGCAAGCGTTGTCCGGATTTATTGGGCGTAAAGCGAGCG |
| | | CAGGCGGTTTTTTAAGTCTGATGTGAAAGCCTTCGGCTCAACCGAAGAAG |
| | | TGCATCGGAAACTGGGAAACTTGAGTGCAGAAGAGGACAGTGGAACTCC |
| | | ATGTGTAGCGGTGAAATGCGTAGATATATGGAAGAACACCAGTGGCGAAG |
| | | GCGGCTGTCTGGTCTGTAACTGACGCTGAGGCTCGAAAGTATGGGTAGCA |
| | | AACAGGATTAGATACCCTGGTAGTCCATACCGTAAACGATGAATGCTAAG |
| | | TGTTGGAGGGTTTCCGCCCTTCAGTGCTGCAGCTAACGCATTAAGCATTCC |
| | | GCCTGGGGAGTACGGCCGCAAGGCTGAAACTCAAAGGAATTGACGGGGG |
| | | CCCGCACAAGCGGTGGAGCATGTGGTTTAATTCGAAGCTACGCGAAGAAC |
| | | CTTACCAGGTCTTGACATACTATGCAAATCTAAGAGATTAGACGTTCCCTT |
| | | CGGGGACATGGATACAGGTGGTGCATGGTTGTCGTCAGCTCGTGTCGTGA |
| | | GATGTTGGGTTAAGTCCCGCAACGAGCGCAACCCTTATTATCAGTTGCCAG |
| | | CATTAAGTTGGGCACTCTGGTGAGACTGCCGGTGACAAACCGGAGGAAGG |
| | | TGGGGATGACGTCAAATCATCATGCCCCTTATGACCTGGGCTACACACGT |
| | | GCTACAATGGATGGTACAACGAGTTGCGAACTCGCGAGAGTAAGCTAATC |
| | | TCTTAAAGCCATTCTCAGTTCGGATTGTAGGCTGCAACTCGCCTACATGAA |
| | | GTCGGAATCGCTAGTAATCGCGGATCAGCATGCCGCGGTGAATACGTTCC |
| | | CGGGCCTTGTACACACCGCCCGTCACACCATGAGAGTTTGTAACACCCAA |
| | | AGTCGGTGGGGTAACCTTTTAGGAACCAGCCGCCTAAGGTGGGACAGATG |
| | | ATTAGGGTGAAGTCGTAACAAGGTAGCCGTAGGAGAACCTGCGGCTGGAT |
| | | CACCTCCTT |
| 153 | DP88 16S | TAGTGGGTTTGATCCTGGCTCAGGACGAACGCTGGCGGCGTGCCTAATAC |
| | rRNA | ATGCAAGTCGAGCGGACAGATGGGAGCTTGCTCCCTGATGTTAGCGGCGG |
| | | ACGGGTGAGTAACACGTGGGTAACCTGCCTGTAAGACTGGGATAACTCCG |
| | | GGAAACCGGGGCTAATACCGGATGGTTGTCTGAACCGCATGGTTCAGACA |
| | | TAAAAGGTGGCTTCGGCTACCACTTACAGATGGACCCGCGGCGCATTAGC |
| | | TAGTTGGTGAGGTAACGGCTCACCAAGGCGACGATGCGTAGCCGACCTGA |
| | | GAGGGTGATCGGCCACACTGGGACTGAGACACGGCCCAGACTCCTACGGG |
| | | AGGCAGCAGTAGGGAATCTTCCGCAATGGACGAAAGTCTGACGGAGCAA |
| | | CGCCGCGTGAGTGATGAAGGTTTTCGGATCGTAAAGCTCTGTTGTTAGGG |
| | | AAGAACAAGTGCCGTTCAAATAGGGCGGCACCTTGACGGTACCTAACCAG |
| | | AAAGCCACGGCTAACTACGTGCCAGCAGCCGCGGTAATACGTAGGTGGCA |
| | | AGCGTTGTCCGGAATTATTGGGCGTAAAGGGCTCGCAGGCGGTTTCTTAA |
| | | GTCTGATGTGAAAGCCCCCGGCTCAACCGGGGAGGGTCATTGGAAACTGG |
| | | GGAACTTGAGTGCAGAAGAGGAGAGTGGAATTCCACGTGTAGCGGTGAA |
| | | ATGCGTAGAGATGTGGAGGAACACCAGTGGCGAAGGCGACTCTCTGGTCT |
| | | GTAACTGACGCTGAGGAGCGAAAGCGTGGGGAGCGAACAGGATTAGATA |
| | | CCCTGGTAGTCCACGCCGTAAACGATGAGTGCTAAGTGTTAGGGGGTTTC |
| | | CGCCCCTTAGTGCTGCAGCTAACGCATTAAGCACTCCGCCTGGGGAGTAC |
| | | GGTCGCAAGACTGAAACTCAAAGGAATTGACGGGGGCCCGCACAAGCGG |
| | | TGGAGCATGTGGTTTAATTCGAAGCAACGCGAAGAACCTTACCAGGTCTT |
| | | GACATCCTCTGACAATCCTAGAGATAGGACGTCCCCTTCGGGGGCAGAGT |
| | | GACAGGTGGTGCATGGTTGTCGTCAGCTCGTGTCGTGAGATGTTGGGTTAA |
| | | GTCCCGCAACGAGCGCAACCCTTGATCTTAGTTGCCAGCATTCAGTTGGGC |
| | | ACTCTAAGGTGACTGCCGGTGACAAACCGGAGGAAGGTGGGGATGACGTC |
| | | AAATCATCATGCCCCTTATGACCTGGGCTACACACGTGCTACAATGGACA |
| | | GAACAAAGGGCAGCGAAACCGCGAGGTTAAGCCAATCCCACAAATCTGTT |
| | | CTCAGTTCGGATCGCAGTCTGCAACTCGACTGCGTGAAGCTGGAATCGCT |
| | | AGTAATCGCGGATCAGCATGCCGCGGTGAATACGTTCCCGGGCCTTGTAC |
| | | ACACCGCCCGTCACACCACGAGAGTTTGTAACACCCGAAGTCGGTGAGGT |
| | | AACCTTTATGGAGCCAGCCGCCGAAGGTGGGACAGATGATTGGGGTGAAG |
| | | TCGTAACAAGGTAGCCGTATCGGAAGGTGCGGCTGGATCACCTCCTTT |
| 154 | DP89 16S | GTAACGGCTCACCAAGGCAACGATGCGTAGCCGACCTGAGAGGGTGATCG |
| | rRNA | GCCACACTGGGACTGAGACACGGCCCAGACTCCTACGGGAGGCAGCAGTA |
| | | GGGAATCTTCCGCAATGGACGAAAGTCTGACGGAGCAACGCCGCGTGAGT |
| | | GATGAAGGTTTTCGGATCGTAAAGCTCTGTTGTTAGGGAAGAACAAGTAC |
| | | CGTTCGAATAGGGCGGTACCTTGACGGTACCTAACCAGAAAGCCACGGCT |
| | | AACTACGTGCCAGCAGCCGCGGTAATACGTAGGTGGCAAGCGTTGTCCGG |
| | | AATTATTGGGCGTAAAGGGCTCGCAGGCGGTTTCTTAAGTCTGATGTGAA |
| | | AGCCCCCGGCTCAACCGGGGAGGGTCATTGGAAACTGGGGAACTTGAGTG |
| | | CAGAAGAGGAGAGTGGAATTCCACGTGTAGCGGTGAAATGCGTAGAGAT |
| | | GTGGAGGAACACCAGTGGCGAAGGCGACTCTCTGGTCTGTAACTGACGCT |
| | | GAGGAGCGAAAGCGTGGGGAGCGAACAGGATTAGATACCCTGGTAGTCC |
| | | ACGCCGTAAACGATGAGTGCTAAGTGTTAGGGGGTTTCCGCCCCTTAGTG |
| | | CTGCAGCTAACGCATTAAGCACTCCGCCTGGGGAGTACGGTCGCAAGACT |
| | | GAAACTCAAAGGAATTGACGGGGGCCCGCACAAGCGGTGGAGCATGTGG |
| | | TTTAATTCGAAGCAACGCGAAGAACCTTACCAGGTCTTGACATCCTCTGAC |
| | | AATCCTAGAGATAGGACGTCCCCTTCGGGGGCAGAGTGACAGGTGGTGCA |
| | | TGGTTGTCGTCAGCTCGTGTCGTGAGATGTTGGGTTAAGTCCCGCAACGAG |
| | | CGCAACCCTTGATCTTAGTTGCCAGCATTCAGTTGGGCACTCTAAGGTGAC |
| | | TGCCGGTGACAAACCGGAGGAAGGTGGGGATGACGTCAAATCATCATGCC |
| | | CCTTATGACCTGGGCTACACACGTGCTACAATGGACAGAACAAAGGGCAG |
| | | CGAAACCGCGAGGTTAAGCCAATCCCACAAATCTGTTCTCAGTTCGGATC |
| | | GCAGTCTGCAACTCGACTGCGTGAAGCTGGAATCGCTAGTAATCGCGGAT |
| | | CAGCATGCCGCGGTGAATACGTTCCCGGGCCTTGTACACACCGCCCGTCA |
| | | CACCACGAGAGTTTGTAACACCCGAAGTCGGTGAGGTAACCTTTTAGGAG |
| | | CCAGCCGCCGAAGGTGGGACAGATGATTGGGGTGAAGTCGTAACAAGGT |
| | | AGCCGTATCGGAAGGTGCGGCTGGATCACCTCCTTT |
| 155 | DP90 16S | TTTGAGAGTTTGATCCTGGCTCAGGACGAACGCTGGCGGCGTGCCTAATA |
| | rRNA | CATGCAAGTCGAACGAACTCTGGTATTGATTGGTGCTTGCATCATGATTTA |
| | | CATTTGAGTGAGTGGCGAACTGGTGAGTAACACGTGGGAAACCTGCCCAG |
| | | AAGCGGGGGATAACACCTGGAAACAGATGCTAATACCGCATAACAACTTG |
| | | GACCGCATGGTCCGAGCTTGAAAGATGGCTTCGGCTATCACTTTTGGATGG |
| | | TCCCGCGGCGTATTAGCTAGATGGTGGGGTAACGGCTCACCATGGCAATG |
| | | ATACGTAGCCGACCTGAGAGGGTAATCGGCCACATTGGGACTGAGACACG |
| | | GCCCAAACTCCTACGGGAGGCAGCAGTAGGGAATCTTCCACAATGGACGA |
| | | AAGTCTGATGGAGCAACGCCGCGTGAGTGAAGAAGGGTTTCGGCTCGTAA |
| | | AACTCTGTTGTTAAAGAAGAACATATCTGAGAGTAACTGTTCAGGTATTG |
| | | ACGGTATTTAACCAGAAAGCCACGGCTAACTACGTGCCAGCAGCCGCGGT |
| | | AATACGTAGGTGGCAAGCGTTGTCCGGATTTATTGGGCGTAAAGCGAGCG |
| | | CAGGCGGTTTTTTAAGTCTGATGTGAAAGCCTTCGGCTCAACCGAAGAAG |
| | | TGCATCGGAAACTGGGAAACTTGAGTGCAGAAGAGGACAGTGGAACTCC |
| | | ATGTGTAGCGGTGAAATGCGTAGATATATGGAAGAACACCAGTGGCGAAG |
| | | GCGGCTGTCTGGTCTGTAACTGACGCTGAGGCTCGAAAGTATGGGTAGCA |
| | | AACAGGATTAGATACCCTGGTAGTCCATACCGTAAACGATGAATGCTAAG |
| | | TGTTGGAGGGTTTCCGCCCTTCAGTGCTGCAGCTAACGCATTAAGCATTCC |
| | | GCCTGGGGAGTACGGCCGCAAGGCTGAAACTCAAAGGAATTGACGGGGG |
| | | CCCGCACAAGCGGTGGAGCATGTGGTTTAATTCGAAGCTACGCGAAGAAC |
| | | CTTACCAGGTCTTGACATACTATGCAAATCTAAGAGATTAGACGTTCCCTT |
| | | CGGGGACATGGATACAGGTGGTGCATGGTTGTCGTCAGCTCGTGTCGTGA |
| | | GATGTTGGGTTAAGTCCCGCAACGAGCGCAACCCTTATTATCAGTTGCCAG |
| | | CATTAAGTTGGGCACTCTGGTGAGACTGCCGGTGACAAACCGGAGGAAGG |
| | | TGGGGATGACGTCAAATCATCATGCCCCTTATGACCTGGGCTACACACGT |
| | | GCTACAATGGATGGTACAACGAGTTGCGAACTCGCGAGAGTAAGCTAATC |
| | | TCTTAAAGCCATTCTCAGTTCGGATTGTAGGCTGCAACTCGCCTACATGAA |
| | | GTCGGAATCGCTAGTAATCGCGGATCAGCATGCCGCGGTGAATACGTTCC |
| | | CGGGCCTTGTACACACCGCCCGTCACACCATGAGAGTTTGTAACACCCAA |
| | | AGTCGGTGGGGTAACCTTTTAGGAACCAGCCGCCTAAGGTGGGACAGATG |
| | | ATTAGGGTGAAGTCGTAACAAGGTAGCCGTAGGAGAACCTGCGGCTGGAT |
| | | CACCTCCTT |
| 156 | DP92 16S | CGATGCGTAGCCGACCTGAGAGGGTGATCGGCCACACTGGGACTGAGACA |
| | rRNA | CGGCCCAGACTCCTACGGGAGGCAGCAGTAGGGAATCTTCCGCAATGGAC |
| | | GAAAGTCTGACGGAGCAACGCCGCGTGAGTGATGAAGGTTTTCGGATCGT |
| | | AAAGCTCTGTTGTTAGGGAAGAACAAGTACCGTTCGAATAGGGCGGTACC |
| | | TTGACGGTACCTAACCAGAAAGCCACGGCTAACTACGTGCCAGCAGCCGC |
| | | GGTAATACGTAGGTGGCAAGCGTTGTCCGGAATTATTGGGCGTAAAGGGC |
| | | TCGCAGGCGGTTTCTTAAGTCTGATGTGAAAGCCCCCGGCTCAACCGGGG |
| | | AGGGTCATTGGAAACTGGGGAACTTGAGTGCAGAAGAGGAGAGTGGAAT |
| | | TCCACGTGTAGCGGTGAAATGCGTAGAGATGTGGAGGAACACCAGTGGCG |
| | | AAGGCGACTCTCTGGTCTGTAACTGACGCTGAGGAGCGAAAGCGTGGGGA |
| | | GCGAACAGGATTAGATACCCTGGTAGTCCACGCCGTAAACGATGAGTGCT |
| | | AAGTGTTAGGGGGTTTCCGCCCCTTAGTGCTGCAGCTAACGCATTAAGCAC |
| | | TCCGCCTGGGGAGTACGGTCGCAAGACTGAAACTCAAAGGAATTGACGGG |
| | | GGCCCGCACAAGCGGTGGAGCATGTGGTTTAATTCGAAGCAACGCGAAGA |
| | | ACCTTACCAGGTCTTGACATCCTCTGACAATCCTAGAGATAGGACGTCCCC |
| | | TTCGGGGGCAGAGTGACAGGTGGTGCATGGTTGTCGTCAGCTCGTGTCGT |
| | | GAGATGTTGGGTTAAGTCCCGCAACGAGCGCAACCCTTGATCTTAGTTGCC |
| | | AGCATTCAGTTGGGCACTCTAAGGTGACTGCCGGTGACAAACCGGAGGAA |
| | | GGTGGGGATGACGTCAAATCATCATGCCCCTTATGACCTGGGCTACACAC |
| | | GTGCTACAATGGACAGAACAAAGGGCAGCGAAACCGCGAGGTTAAGCCA |
| | | ATCCCACAAATCTGTTCTCAGTTCGGATCGCAGTCTGCAACTCGACTGCGT |
| | | GAAGCTGGAATCGCTAGTAATCGCGGATCAGCATGCCGCGGTGAATACGT |
| | | TCCCGGGCCTTGTACACACCGCCCGTCACACCACGAGAGTTTGTAACACCC |
| | | GAAGTCGGTGAGGTAACCTTTTAGGAGCCAGCCGCCGAAGGTGGGACAGA |
| | | TGATTGGGGTGAAGTCGTAACAAGGTAGCCGTATCGGAAGGTGCGGCTGG |
| | | ATCACCTCCTTT |
| 157 | DP93 16S | ATTGAGAGTTTGATCCTGGCTCAGGATGAACGCTGGCGGCGTGCCTAATA |
| | rRNA | CATGCAAGTCGAACGCACAGCGAAAGGTGCTTGCACCTTTCAAGTGAGTG |
| | | GCGAACGGGTGAGTAACACGTGGACAACCTGCCTCAAGGCTGGGGATAAC |
| | | ATTTGGAAACAGATGCTAATACCGAATAAAACTTAGTGTCGCATGACAAA |
| | | AAGTTAAAAGGCGCTTCGGCGTCACCTAGAGATGGATCCGCGGTGCATTA |
| | | GTTAGTTGGTGGGGTAAAGGCCTACCAAGACAATGATGCATAGCCGAGTT |
| | | GAGAGACTGATCGGCCACATTGGGACTGAGACACGGCCCAAACTCCTACG |
| | | GGAGGCTGCAGTAGGGAATCTTCCACAATGGGCGAAAGCCTGATGGAGCA |
| | | ACGCCGCGTGTGTGATGAAGGCTTTCGGGTCGTAAAGCACTGTTGTATGG |
| | | GAAGAACAGCTAGAATAGGAAATGATTTTAGTTTGACGGTACCATACCAG |
| | | AAAGGGACGGCTAAATACGTGCCAGCAGCCGCGGTAATACGTATGTCCCG |
| | | AGCGTTATCCGGATTTATTGGGCGTAAAGCGAGCGCAGACGGTTTATTAA |
| | | GTCTGATGTGAAAGCCCGGAGCTCAACTCCGGAATGGCATTGGAAACTGG |
| | | TTAACTTGAGTGCAGTAGAGGTAAGTGGAACTCCATGTGTAGCGGTGGAA |
| | | TGCGTAGATATATGGAAGAACACCAGTGGCGAAGGCGGCTTACTGGACTG |
| | | CAACTGACGTTGAGGCTCGAAAGTGTGGGTAGCAAACAGGATTAGATACC |
| | | CTGGTAGTCCACACCGTAAACGATGAACACTAGGTGTTAGGAGGTTTCCG |
| | | CCTCTTAGTGCCGAAGCTAACGCATTAAGTGTTCCGCCTGGGGAGTACGA |
| | | CCGCAAGGTTGAAACTCAAAGGAATTGACGGGGACCCGCACAAGCGGTG |
| | | GAGCATGTGGTTTAATTCGAAGCAACGCGAAGAACCTTACCAGGTCTTGA |
| | | CATCCTTTGAAGCTTTTAGAGATAGAAGTGTTCTCTTCGGAGACAAAGTGA |
| | | CAGGTGGTGCATGGTCGTCGTCAGCTCGTGTCGTGAGATGTTGGGTTAAGT |
| | | CCCGCAACGAGCGCAACCCTTATTGTTAGTTGCCAGCATTCAGATGGGCA |
| | | CTCTAGCGAGACTGCCGGTGACAAACCGGAGGAAGGCGGGGACGACGTC |
| | | AGATCATCATGCCCCTTATGACCTGGGCTACACACGTGCTACAATGGCGTA |
| | | TACAACGAGTTGCCAACCCGCGAGGGTGAGCTAATCTCTTAAAGTACGTC |
| | | TCAGTTCGGATTGTAGTCTGCAACTCGACTACATGAAGTCGGAATCGCTAG |
| | | TAATCGCGGATCAGCACGCCGCGGTGAATACGTTCCCGGGTCTTGTACAC |
| | | ACCGCCCGTCACACCATGGGAGTTTGTAATGCCCAAAGCCGGTGGCCTAA |
| | | CCTTTTAGGAAGGAGCCGTCTAAGGCAGGACAGATGACTGGGGTGAAGTC |
| | | GTAACAAGGTAGCCGTAGGAGAACCTGCGGCTGGATCACCTCCTTT |
| 158 | DP94 16S | ATCTGCCCAGAAGCAGGGGATAACACTTGGAAACAGGTGCTAATACCGTA |
| | rRNA | TAACAACAAAATCCGCATGGATTTTGTTTGAAAGGTGGCTTCGGCTATCAC |
| | | TTCTGGATGATCCCGCGGCGTATTAGTTAGTTGGTGAGGTAAAGGCCCACC |
| | | AAGACGATGATACGTAGCCGACCTGAGAGGGTAATCGGCCACATTGGGAC |
| | | TGAGACACGGCCCAAACTCCTACGGGAGGCAGCAGTAGGGAATCTTCCAC |
| | | AATGGACGAAAGTCTGATGGAGCAATGCCGCGTGAGTGAAGAAGGGTTTC |
| | | GGCTCGTAAAACTCTGTTGTTAAAGAAGAACACCTTTGAGAGTAACTGTTC |
| | | AAGGGTTGACGGTATTTAACCAGAAAGCCACGGCTAACTACGTGCCAGCA |
| | | GCCGCGGTAATACGTAGGTGGCAAGCGTTGTCCGGATTTATTGGGCGTAA |
| | | AGCGAGCGCAGGCGGTTTTTTAAGTCTGATGTGAAAGCCTTCGGCTTAACC |
| | | GGAGAAGTGCATCGGAAACTGGGAGACTTGAGTGCAGAAGAGGACAGTG |
| | | GAACTCCATGTGTAGCGGTGGAATGCGTAGATATATGGAAGAACACCAGT |
| | | GGCGAAGGCGGCTGTCTAGTCTGTAACTGACGCTGAGGCTCGAAAGCATG |
| | | GGTAGCGAACAGGATTAGATACCCTGGTAGTCCATGCCGTAAACGATGAG |
| | | TGCTAAGTGTTGGAGGGTTTCCGCCCTTCAGTGCTGCAGCTAACGCATTAA |
| | | GCACTCCGCCTGGGGAGTACGACCGCAAGGTTGAAACTCAAAGGAATTGA |
| | | CGGGGGCCCGCACAAGCGGTGGAGCATGTGGTTTAATTCGAAGCTACGCG |
| | | AAGAACCTTACCAGGTCTTGACATCTTCTGCCAATCTTAGAGATAAGACGT |
| | | TCCCTTCGGGGACAGAATGACAGGTGGTGCATGGTTGTCGTCAGCTCGTGT |
| | | CGTGAGATGTTGGGTTAAGTCCCGCAACGAGCGCAACCCTTATTATCAGTT |
| | | GCCAGCATTCAGTTGGGCACTCTGGTGAGACTGCCGGTGACAAACCGGAG |
| | | GAAGGTGGGGATGACGTCAAATCATCATGCCCCTTATGACCTGGGCTACA |
| | | CACGTGCTACAATGGACGGTACAACGAGTTGCGAAGTCGTGAGGCTAAGC |
| | | TAATCTCTTAAAGCCGTTCTCAGTTCGGATTGTAGGCTGCAACTCGCCTAC |
| | | ATGAAGTTGGAATCGCTAGTAATCGCGGATCAGCATGCCGCGGTGAATAC |
| | | GTTCCCGGGCCTTGTACACACCGCCCGTCACACCATGAGAGTTTGTAACAC |
| | | CCAAAGCCGGTGAGATAACCTTCGGGAGTCAGCCGTCTAAGGTGGGACAG |
| | | ATGATTAGGGTGAAGTCGTAACAAGGTAGCCGTAGGAGAACCTGCGGCTG |
| | | GATCACCTCCTT |
| 159 | DP95 16S | TGCTAATACCGCATAGATCCAAGAACCGCATGGTTCTTGGCTGAAAGATG |
| | rRNA | GCGTAAGCTATCGCTTTTGGATGGACCCGCGGCGTATTAGCTAGTTGGTGA |
| | | GGTAATGGCTCACCAAGGCGATGATACGTAGCCGAACTGAGAGGTTGATC |
| | | GGCCACATTGGGACTGAGACACGGCCCAAACTCCTACGGGAGGCAGCAGT |
| | | AGGGAATCTTCCACAATGGACGCAAGTCTGATGGAGCAACGCCGCGTGAG |
| | | TGAAGAAGGCTTTCGGGTCGTAAAACTCTGTTGTTGGAGAAGAATGGTCG |
| | | GCAGAGTAACTGTTGTCGGCGTGACGGTATCCAACCAGAAAGCCACGGCT |
| | | AACTACGTGCCAGCAGCCGCGGTAATACGTAGGTGGCAAGCGTTATCCGG |
| | | ATTTATTGGGCGTAAAGCGAGCGCAGGCGGTTTTTTAAGTCTGATGTGAA |
| | | AGCCCTCGGCTTAACCGAGGAAGCGCATCGGAAACTGGGAAACTTGAGTG |
| | | CAGAAGAGGACAGTGGAACTCCATGTGTAGCGGTGAAATGCGTAGATATA |
| | | TGGAAGAACACCAGTGGCGAAGGCGGCTGTCTGGTCTGTAACTGACGCTG |
| | | AGGCTCGAAAGCATGGGTAGCGAACAGGATTAGATACCCTGGTAGTCCAT |
| | | GCCGTAAACGATGAATGCTAGGTGTTGGAGGGTTTCCGCCCTTCAGTGCC |
| | | GCAGCTAACGCATTAAGCATTCCGCCTGGGGAGTACGACCGCAAGGTTGA |
| | | AACTCAAAGGAATTGACGGGGGCCCGCACAAGCGGTGGAGCATGTGGTTT |
| | | AATTCGAAGCAACGCGAAGAACCTTACCAGGTCTTGACATCTTTTGATCAC |
| | | CTGAGAGATCAGGTTTCCCCTTCGGGGGCAAAATGACAGGTGGTGCATGG |
| | | TTGTCGTCAGCTCGTGTCGTGAGATGTTGGGTTAAGTCCCGCAACGAGCGC |
| | | AACCCTTATGACTAGTTGCCAGCATTTAGTTGGGCACTCTAGTAAGACTGC |
| | | CGGTGACAAACCGGAGGAAGGTGGGGATGACGTCAAATCATCATGCCCCT |
| | | TATGACCTGGGCTACACACGTGCTACAATGGATGGTACAACGAGTTGCGA |
| | | GACCGCGAGGTCAAGCTAATCTCTTAAAGCCATTCTCAGTTCGGACTGTAG |
| | | GCTGCAACTCGCCTACACGAAGTCGGAATCGCTAGTAATCGCGGATCAGC |
| | | ACGCCGCGGTGAATACGTTCCCGGGCCTTGTACACACCGCCCGTCACACC |
| | | ATGAGAGTTTGTAACACCCGAAGCCGGTGGCGTAACCCTTTTAGGGAGCG |
| | | AGCCGTCTAAGGTGGGACAAATGATTAGGGTGAAGTCGTAACAAGGTAGC |
| | | CGTAGGAGAACCTGCGGCTGGATCACCTCCTTT |
| 160 | DP96 16S | ACACGGCCCAAACTCCTACGGGAGGCAGCAGTAGGGAATCTTCCACAATG |
| | rRNA | GACGCAAGTCTGATGGAGCAACGCCGCGTGAGTGAAGAAGGCTTTCGGGT |
| | | CGTAAAACTCTGTTGTTGGAGAAGAATGGTCGGCAGAGTAACTGTTGTCG |
| | | GCGTGACGGTATCCAACCAGAAAGCCACGGCTAACTACGTGCCAGCAGCC |
| | | GCGGTAATACGTAGGTGGCAAGCGTTATCCGGATTTATTGGGCGTAAAGC |
| | | GAGCGCAGGCGGTTTTTTAAGTCTGATGTGAAAGCCCTCGGCTTAACCGA |
| | | GGAAGCGCATCGGAAACTGGGAAACTTGAGTGCAGAAGAGGACAGTGGA |
| | | ACTCCATGTGTAGCGGTGAAATGCGTAGATATATGGAAGAACACCAGTGG |
| | | CGAAGGCGGCTGTCTGGTCTGTAACTGACGCTGAGGCTCGAAAGCATGGG |
| | | TAGCGAACAGGATTAGATACCCTGGTAGTCCATGCCGTAAACGATGAATG |
| | | CTAGGTGTTGGAGGGTTTCCGCCCTTCAGTGCCGCAGCTAACGCATTAAGC |
| | | ATTCCGCCTGGGGAGTACGACCGCAAGGTTGAAACTCAAAGGAATTGACG |
| | | GGGGCCCGCACAAGCGGTGGAGCATGTGGTTTAATTCGAAGCAACGCGAA |
| | | GAACCTTACCAGGTCTTGACATCTTTTGATCACCTGAGAGATCAGGTTTCC |
| | | CCTTCGGGGGCAAAATGACAGGTGGTGCATGGTTGTCGTCAGCTCGTGTC |
| | | GTGAGATGTTGGGTTAAGTCCCGCAACGAGCGCAACCCTTATGACTAGTT |
| | | GCCAGCATTTAGTTGGGCACTCTAGTAAGACTGCCGGTGACAAACCGGAG |
| | | GAAGGTGGGGATGACGTCAAATCATCATGCCCCTTATGACCTGGGCTACA |
| | | CACGTGCTACAATGGATGGTACAACGAGTTGCGAGACCGCGAGGTCAAGC |
| | | TAATCTCTTAAAGCCATTCTCAGTTCGGACTGTAGGCTGCAACTCGCCTAC |
| | | ACGAAGTCGGAATCGCTAGTAATCGCGGATCAGCACGCCGCGGTGAATAC |
| | | GTTCCCGGGCCTTGTACACACCGCCCGTCACACCATGAGAGTTTGTAACAC |
| | | CCGAAGCCGGTGGCGTAACCCTTTTAGGGAGCGAGCCGTCTAAGGTGGGA |
| | | CAAATGATTAGGGTGAAGTCGTAACAAGGTAGCCGTAGGAGAACCTGCGG |
| | | CTGGATCACCTCCTTT |
| 161 | DP97 16S | AATGAGAGTTTGATCCTGGCTCAGGACGAACGCTGGCGGCGTGCCTAATA |
| | rRNA | CATGCAAGTCGAGCGATGATTAAAGATAGCTTGCTATTTTTATGAAGAGC |
| | | GGCGAACGGGTGAGTAACGCGTGGGAAATCTGCCGAGTAGCGGGGGACA |
| | | ACGTTTGGAAACGAACGCTAATACCGCATAACAATGAGAATCGCATGATT |
| | | CTTATTTAAAAGAAGCAATTGCTTCACTACTTGATGATCCCGCGTTGTATT |
| | | AGCTAGTTGGTAGTGTAAAGGACTACCAAGGCGATGATACATAGCCGACC |
| | | TGAGAGGGTGATCGGCCACACTGGGACTGAGACACGGCCCAGACTCCTAC |
| | | GGGAGGCAGCAGTAGGGAATCTTCGGCAATGGGGGCAACCCTGACCGAG |
| | | CAACGCCGCGTGAGTGAAGAAGGTTTTCGGATCGTAAAACTCTGTTGTTA |
| | | GAGAAGAACGTTAAGTAGAGTGGAAAATTACTTAAGTGACGGTATCTAAC |
| | | CAGAAAGGGACGGCTAACTACGTGCCAGCAGCCGCGGTAATACGTAGGTC |
| | | CCAAGCGTTGTCCGGATTTATTGGGCGTAAAGCGAGCGCAGGTGGTTTCTT |
| | | AAGTCTGATGTAAAAGGCAGTGGCTCAACCATTGTGTGCATTGGAAACTG |
| | | GGAGACTTGAGTGCAGGAGAGGAGAGTGGAATTCCATGTGTAGCGGTGA |
| | | AATGCGTAGATATATGGAGGAACACCGGAGGCGAAAGCGGCTCTCTGGCC |
| | | TGTAACTGACACTGAGGCTCGAAAGCGTGGGGAGCAAACAGGATTAGATA |
| | | CCCTGGTAGTCCACGCCGTAAACGATGAGTGCTAGCTGTAGGGAGCTATA |
| | | AGTTCTCTGTAGCGCAGCTAACGCATTAAGCACTCCGCCTGGGGAGTACG |
| | | ACCGCAAGGTTGAAACTCAAAGGAATTGACGGGGGCCCGCACAAGCGGT |
| | | GGAGCATGTGGTTTAATTCGAAGCAACGCGAAGAACCTTACCAGGTCTTG |
| | | ACATACTCGTGATATCCTTAGAGATAAGGAGTTCCTTCGGGACACGGGAT |
| | | ACAGGTGGTGCATGGTTGTCGTCAGCTCGTGTCGTGAGATGTTGGGTTAAG |
| | | TCCCGCAACGAGCGCAACCCTTATTACTAGTTGCCATCATTAAGTTGGGCA |
| | | CTCTAGTGAGACTGCCGGTGATAAACCGGAGGAAGGTGGGGATGACGTCA |
| | | AATCATCATGCCCCTTATGACCTGGGCTACACACGTGCTACAATGGATGGT |
| | | ACAACGAGTCGCCAACCCGCGAGGGTGCGCTAATCTCTTAAAACCATTCT |
| | | CAGTTCGGATTGCAGGCTGCAACTCGCCTGCATGAAGTCGGAATCGCTAG |
| | | TAATCGCGGATCAGCACGCCGCGGTGAATACGTTCCCGGGCCTTGTACAC |
| | | ACCGCCCGTCACACCACGGAAGTTGGGAGTACCCAAAGTAGGTTGCCTAA |
| | | CCGCAAGGAGGGCGCTTCCTAAGGTAAGACCGATGACTGGGGTGAAGTCG |
| | | TAACAAGGTAGCCGTATCGGAAGGTGCGGCTGGATCACCTCCTTT |
| 162 | DP98 16S | AATGAGAGTTTGATCCTGGCTCAGGACGAACGCTGGCGGCGTGCCTAATA |
| | rRNA | CATGCAAGTCGAGCGATGATTAAAGATAGCTTGCTATTTTTATGAAGAGC |
| | | GGCGAACGGGTGAGTAACGCGTGGGAAATCTGCCGAGTAGCGGGGGACA |
| | | ACGTTTGGAAACGAACGCTAATACCGCATAACAATGAGAATCGCATGATT |
| | | CTTATTTAAAAGAAGCAATTGCTTCACTACTTGATGATCCCGCGTTGTATT |
| | | AGCTAGTTGGTAGTGTAAAGGACTACCAAGGCGATGATACATAGCCGACC |
| | | TGAGAGGGTGATCGGCCACACTGGGACTGAGACACGGCCCAGACTCCTAC |
| | | GGGAGGCAGCAGTAGGGAATCTTCGGCAATGGGGGCAACCCTGACCGAG |
| | | CAACGCCGCGTGAGTGAAGAAGGTTTTCGGATCGTAAAACTCTGTTGTTA |
| | | GAGAAGAACGTTAAGTAGAGTGGAAAATTACTTAAGTGACGGTATCTAAC |
| | | CAGAAAGGGACGGCTAACTACGTGCCAGCAGCCGCGGTAATACGTAGGTC |
| | | CCAAGCGTTGTCCGGATTTATTGGGCGTAAAGCGAGCGCAGGTGGTTTCTT |
| | | AAGTCTGATGTAAAAGGCAGTGGCTCAACCATTGTGTGCATTGGAAACTG |
| | | GGAGACTTGAGTGCAGGAGAGGAGAGTGGAATTCCATGTGTAGCGGTGA |
| | | AATGCGTAGATATATGGAGGAACACCGGAGGCGAAAGCGGCTCTCTGGCC |
| | | TGTAACTGACACTGAGGCTCGAAAGCGTGGGGAGCAAACAGGATTAGATA |
| | | CCCTGGTAGTCCACGCCGTAAACGATGAGTGCTAGCTGTAGGGAGCTATA |
| | | AGTTCTCTGTAGCGCAGCTAACGCATTAAGCACTCCGCCTGGGGAGTACG |
| | | ACCGCAAGGTTGAAACTCAAAGGAATTGACGGGGGCCCGCACAAGCGGT |
| | | GGAGCATGTGGTTTAATTCGAAGCAACGCGAAGAACCTTACCAGGTCTTG |
| | | ACATACTCGTGATATCCTTAGAGATAAGGAGTTCCTTCGGGACACGGGAT |
| | | ACAGGTGGTGCATGGTTGTCGTCAGCTCGTGTCGTGAGATGTTGGGTTAAG |
| | | TCCCGCAACGAGCGCAACCCTTATTACTAGTTGCCATCATTAAGTTGGGCA |
| | | CTCTAGTGAGACTGCCGGTGATAAACCGGAGGAAGGTGGGGATGACGTCA |
| | | AATCATCATGCCCCTTATGACCTGGGCTACACACGTGCTACAATGGATGGT |
| | | ACAACGAGTCGCCAACCCGCGAGGGTGCGCTAATCTCTTAAAACCATTCT |
| | | CAGTTCGGATTGCAGGCTGCAACTCGCCTGCATGAAGTCGGAATCGCTAG |
| | | TAATCGCGGATCAGCACGCCGCGGTGAATACGTTCCCGGGCCTTGTACAC |
| | | ACCGCCCGTCACACCACGGAAGTTGGGAGTACCCAAAGTAGGTTGCCTAA |
| | | CCGCAAGGAGGGCGCTTCCTAAGGTAAGACCGATGACTGGGGTGAAGTCG |
| | | TAACAAGGTAGCCGTATCGGAAGGTGCGGCTGGATCACCTCCTTT |
| 163 | DP100 16S | TTTGAGAGTTTGATCCTGGCTCAGGACGAACGCTGGCGGCGTGCCTAATA |
| | rRNA | CATGCAAGTCGAACGAACTCTGGTATTGATTGGTGCTTGCATCATGATTTA |
| | | CATTTGAGTGAGTGGCGAACTGGTGAGTAACACGTGGGAAACCTGCCCAG |
| | | AAGCGGGGGATAACACCTGGAAACAGATGCTAATACCGCATAACAACTTG |
| | | GACCGCATGGTCCGAGCTTGAAAGATGGCTTCGGCTATCACTTTTGGATGG |
| | | TCCCGCGGCGTATTAGCTAGATGGTGGGGTAACGGCTCACCATGGCAATG |
| | | ATACGTAGCCGACCTGAGAGGGTAATCGGCCACATTGGGACTGAGACACG |
| | | GCCCAAACTCCTACGGGAGGCAGCAGTAGGGAATCTTCCACAATGGACGA |
| | | AAGTCTGATGGAGCAACGCCGCGTGAGTGAAGAAGGGTTTCGGCTCGTAA |
| | | AACTCTGTTGTTAAAGAAGAACATATCTGAGAGTAACTGTTCAGGTATTG |
| | | ACGGTATTTAACCAGAAAGCCACGGCTAACTACGTGCCAGCAGCCGCGGT |
| | | AATACGTAGGTGGCAAGCGTTGTCCGGATTTATTGGGCGTAAAGCGAGCG |
| | | CAGGCGGTTTTTTAAGTCTGATGTGAAAGCCTTCGGCTCAACCGAAGAAG |
| | | TGCATCGGAAACTGGGAAACTTGAGTGCAGAAGAGGACAGTGGAACTCC |
| | | ATGTGTAGCGGTGAAATGCGTAGATATATGGAAGAACACCAGTGGCGAAG |
| | | GCGGCTGTCTGGTCTGTAACTGACGCTGAGGCTCGAAAGTATGGGTAGCA |
| | | AACAGGATTAGATACCCTGGTAGTCCATACCGTAAACGATGAATGCTAAG |
| | | TGTTGGAGGGTTTCCGCCCTTCAGTGCTGCAGCTAACGCATTAAGCATTCC |
| | | GCCTGGGGAGTACGGCCGCAAGGCTGAAACTCAAAGGAATTGACGGGGG |
| | | CCCGCACAAGCGGTGGAGCATGTGGTTTAATTCGAAGCTACGCGAAGAAC |
| | | CTTACCAGGTCTTGACATACTATGCAAATCTAAGAGATTAGACGTTCCCTT |
| | | CGGGGACATGGATACAGGTGGTGCATGGTTGTCGTCAGCTCGTGTCGTGA |
| | | GATGTTGGGTTAAGTCCCGCAACGAGCGCAACCCTTATTATCAGTTGCCAG |
| | | CATTAAGTTGGGCACTCTGGTGAGACTGCCGGTGACAAACCGGAGGAAGG |
| | | TGGGGATGACGTCAAATCATCATGCCCCTTATGACCTGGGCTACACACGT |
| | | GCTACAATGG |
| 164 | DP101 16S | ATGAGAGTTTGATCTTGGCTCAGGATGAACGCTGGCGGCGTGCCTAATAC |
| | rRNA | ATGCAAGTCGAACGAACTTCCGTTAATTGATTATGACGTACTTGTACTGAT |
| | | TGAGATTTTAACACGAAGTGAGTGGCGAACGGGTGAGTAACACGTGGGTA |
| | | ACCTGCCCAGAAGTAGGGGATAACACCTGGAAACAGATGCTAATACCGTA |
| | | TAACAGAGAAAACCGCATGGTTTTCTTTTAAAAGATGGCTCTGCTATCACT |
| | | TCTGGATGGACCCGCGGCGTATTAGCTAGTTGGTGAGGCAAAGGCTCACC |
| | | AAGGCAGTGATACGTAGCCGACCTGAGAGGGTAATCGGCCACATTGGGAC |
| | | TGAGACACGGCCCAGACTCCTACGGGAGGCAGCAGTAGGGAATCTTCCAC |
| | | AATGGACGCAAGTCTGATGGAGCAACGCCGCGTGAGTGAAGAAGGGTTTC |
| | | GGCTCGTAAAGCTCTGTTGTTAAAGAAGAACGTGGGTAAGAGTAACTGTT |
| | | TACCCAGTGACGGTATTTAACCAGAAAGCCACGGCTAACTACGTGCCAGC |
| | | AGCCGCGGTAATACGTAGGTGGCAAGCGTTATCCGGATTTATTGGGCGTA |
| | | AAGCGAGCGCAGGCGGTCTTTTAAGTCTAATGTGAAAGCCTTCGGCTCAA |
| | | CCGAAGAAGTGCATTGGAAACTGGGAGACTTGAGTGCAGAAGAGGACAG |
| | | TGGAACTCCATGTGTAGCGGTGAAATGCGTAGATATATGGAAGAACACCA |
| | | GTGGCGAAGGCGGCTGTCTGGTCTGCAACTGACGCTGAGGCTCGAAAGCA |
| | | TGGGTAGCGAACAGGATTAGATACCCTGGTAGTCCATGCCGTAAACGATG |
| | | ATTACTAAGTGTTGGAGGGTTTCCGCCCTTCAGTGCTGCAGCTAACGCATT |
| | | AAGTAATCCGCCTGGGGAGTACGACCGCAAGGTTGAAACTCAAAAGAATT |
| | | GACGGGGGCCCGCACAAGCGGTGGAGCATGTGGTTTAATTCGAAGCTACG |
| | | CGAAGAACCTTACCAGGTCTTGACATCTTCTGACAGTCTAAGAGATTAGA |
| | | GGTTCCCTTCGGGGACAGAATGACAGGTGGTGCATGGTTGTCGTCAGCTC |
| | | GTGTCGTGAGATGTTGGGTTAAGTCCCGCAACGAGCGCAACCCTTATTACT |
| | | AGTTGCCAGCATTAAGTTGGGCACTCTAGTGAGACTGCCGGTGACAAACC |
| | | GGAGGAAGGTGGGGACGACGTCAAATCATCATGCCCCTTATGACCTGGGC |
| | | TACACACGTGCTACAATGGATGGTACAACGAGTCGCGAGACCGCGAGGTT |
| | | AAGCTAATCTCTTAAAACCATTCTCAGTTCGGACTGTAGGCTGCAACTCGC |
| | | CTACACGAAGTCGGAATCGCTAGTAATCGCGGATCAGCATGCCGCGGTGA |
| | | ATACGTTCCCGGGCCTTGTACACACCGCCCGTCACACCATGAGAGTTTGTA |
| | | AC |
| 165 | DP102 ITS | TCCGTAGGTGAACCTGCGGAAGGATCATTACTGTGATTTAGTACTACACTG |
| | sequence | CGTGAGCGGAACGAAAACAACAACACCTAAAATGTGGAATATAGCATAT |
| | | AGTCGACAAGAGAAATCTACGAAAAACAAACAAAACTTTCAACAACGGA |
| | | TCTCTTGGTTCTCGCATCGATGAAGAGCGCAGCGAAATGCGATACCTAGT |
| | | GTGAATTGCAGCCATCGTGAATCATCGAGTTCTTGAACGCACATTGCGCCC |
| | | CTCGGCATTCCGGGGGGCATGCCTGTTTGAGCGTCGTTTCCATCTTGCGCG |
| | | TGCGCAGAGTTGGGGGAGCGGAGCGGACGACGTGTAAAGAGCGTCGGAG |
| | | CTGCGACTCGCCTGAAAGGGAGCGAAGCTGGCCGAGCGAACTAGACTTTT |
| | | TTTCAGGGACGCTTGGCGGCCGAGAGCGAGTGTTGCGAGACAACAAAAAG |
| | | CTCGACCTCAAATCAGGTAGGAATACCCGCTGAACTTAAGCATATCAATA |
| | | AGCGGAGGAAAAGAAACCAACAGGGATTGCCTCAGTAGCGGCGAGTGAA |
| | | GCGGCAAGAGCTCAGATTTGAAATCGTGCTTTGCGGCACGAGTTGTAGAT |
| | | TGCAGGTTGGAGTCTGTGTGGAAGGCGGTGTCCAAGTCCCTTGGAACAGG |
| | | GCGCCCAGGAGGGTGAGAGCCCCGTGGGATGCCGGCGGAAGCAGTGAGG |
| | | CCCTTCTGACGAGTCGAGTTGTTTGGGAATGCAGCTCCAAGCGGGTGGTA |
| | | AATTCCATCTAAGGCTAAATACTGGCGAGAGACCGATAGCGAACAAGTAC |
| | | TGTGAAGGAAAGATGAAAAGCACTTTGAAAAGAGAGTGAAACAGCACGT |
| | | GAAATTGTTGAAAGGGAAGGGTATTGCGCCCGACATGGGGATTGCGCACC |
| | | GCTGCCTCTCGTGGGCGGCGCTCTGGGCTTTCCCTGGGCCAGCATCGGTTC |
| | | TTGCTGCAGGAGAAGGGGTTCTGGAACGTGGCTCTTCGGAGTGTTATAGC |
| | | CAGGGCCAGATGCTGCGTGCGGGGACCGAGGACTGCGGCCGTGTAGGTCA |
| | | CGGATGCTGGCAGAACGGCGCAACACCGCCCGTCTTGAAACATGGACCAA |
| | | GGAGTCTAACGTCTATGCGAGTGTTTGGGTGTGAAACCCGTACGCGTAAT |
| | | GAAAGTGAACGTAGGTCGGACCCCCTGCCCTCGGGGAGGGGAGCACGATC |
| | | GACCGATCCCGATGTTTATCGGAAGGATTTGAGTAGGAGCATAGCTGTTG |
| | | GGACCCGAAAGATGGTGAACTATGCCTGAATAGGGTGAAGCCAGAGGAA |
| | | ACTCTGGTGGAGGCTCGTAGCGGTTCTGACGTGCAAATCGATCGTCGAATT |
| | | TGGGTATAGGGGCGAAAGACTAATCGAACCATCTAGTAGCTGGTTCCTGC |
| | | CGAAGTTTCCCTCAGGA |
| 166 | DP67 16S | TCGAGCGGACAGATGGGAGCTTGCTCCCTGATGTTAGCGGCGGACGGGTG |
| | rRNA | AGTAACACGTGGGTAACCTGCCTGTAAGACTGGGATAACTCCGGGAAACC |
| | | GGGGCTAATACCGGATGCTTGTTTGAACCGCATGGTTCAAACATAAAAGG |
| | | TGGCTTCGGCTACCACTTACAGATGGACCCGCGGCGCATTAGCTAGTTGGT |
| | | GAGGTAATGGCTCACCAAGGCAACGATGCGTAGCCGACCTGAGAGGGTG |
| | | ATCGGCCACACTGGGACTGAGACACGGCCCAGACTCCTACGGGAGGCAGC |
| | | AGTAGGGAATCTTCCGCAATGGACGAAAGTCTGACGGAGCAACGCCGCGT |
| | | GAGTGATGAAGGTTTTCGGATCGTAAAGCTCTGTTGTTAGGGAAGAACAA |
| | | GTGCCGTTCAAATAGGGCGGCACCTTGACGGTACCTAACCAGAAAGCCAC |
| | | GGCTAACTACGTGCCAGCAGCCGCGGTAATACGTAGGTGGCAAGCGTTGT |
| | | CCGGAATTATTGGGCGTAAAGGGCTCGCAGGCGGTTTCTTAAGTCTGATGT |
| | | GAAAGCCCCCGGCTCAACCGGGGAGGGTCATTGGAAACTGGGGAACTTGA |
| | | GTGCAGAAGAGGAGAGTGGAATTCCACGTGTAGCGGTGAAATGCGTAGA |
| | | GATGTGGAGGAACACCAGTGGCGAAGGCGACTCTCTGGTCTGTAACTGAC |
| | | GCTGAGGAGCGAAAGCGTGGGGAGCGAACAGGATTAGATACCCTGGTAG |
| | | TCCACGCCGTAAACGATGAGTGCTAAGTGTTAGGGGGTTTCCGCCCCTTAG |
| | | TGCTGCAGCTAACGCATTAAGCACTCCGCCTGGGGAGTACGGTCGCAAGA |
| | | CTGAAACTCAAAGGAATTGACGGGGGCCCGCACAAGCGGTGGAGCATGT |
| | | GGTTTAATTCGAAGCAACGCGAAGAACCTTACCAGGTCTTGACATCCTCTG |
| | | ACACCCTAGAGATAGGGCTTCCCTTCGGGG |
| 167 | DP68 16S | TGCAGTCGAGCGGACAGATGGGAGCTTGCTCCCTGATGTTAGCGGCGGAC |
| | rRNA | GGGTGAGTAACACGTGGGTAACCTGCCTGTAAGACTGGGATAACTCCGGG |
| | | AAACCGGGGCTAATACCGGATGCTTGTTTGAACCGCATGGTTCAAACATA |
| | | AAAGGTGGCTTCGGCTACCACTTACAGATGGACCCGCGGCGCATTAGCTA |
| | | GTTGGTGAGGTAATGGCTCACCAAGGCAACGATGCGTAGCCGACCTGAGA |
| | | GGGTGATCGGCCACACTGGGACTGAGACACGGCCCAGACTCCTACGGGAG |
| | | GCAGCAGTAGGGAATCTTCCGCAATGGACGAAAGTCTGACGGAGCAACGC |
| | | CGCGTGAGTGATGAAGGTTTTCGGATCGTAAAGCTCTGTTGTTAGGGAAG |
| | | AACAAGTGCCGTTCAAATAGGGCGGCACCTTGACGGTACCTAACCAGAAA |
| | | GCCACGGCTAACTACGTGCCAGCAGCCGCGGTAATACGTAGGTGGCAAGC |
| | | GTTGTCCGGAATTATTGGGCGTAAAGGGCTCGCAGGCGGTTTCTTAAGTCT |
| | | GATGTGAAAGCCCCCGGCTCAACCGGGGAGGGTCATTGGAAACTGGGGA |
| | | ACTTGAGTGCAGAAGAGGAGAGTGGAATTCCACGTGTAGCGGTGAAATGC |
| | | GTAGAGATGTGGAGGAACACCAGTGGCGAA |
| 168 | DP69 16S | TGCAGTCGAGCGGACAGATGGGAGCTTGCTCCCTGATGTTAGCGGCGGAC |
| | rRNA | GGGTGAGTAACACGTGGGTAACCTGCCTGTAAGACTGGGATAACTCCGGG |
| | | AAACCGGGGCTAATACCGGATGCTTGTTTGAACCGCATGGTTCAAACATA |
| | | AAAGGTGGCTTCGGCTACCACTTACAGATGGACCCGCGGCGCATTAGCTA |
| | | GTTGGTGAGGTAATGGCTCACCAAGGCAACGATGCGTAGCCGACCTGAGA |
| | | GGGTGATCGGCCACACTGGGACTGAGACACGGCCCAGACTCCTACGGGAG |
| | | GCAGCAGTAGGGAATCTTCCGCAATGGACGAAAGTCTGACGGAGCAACGC |
| | | CGCGTGAGTGATGAAGGTTTTCGGATCGTAAAGCTCTGTTGTTAGGGAAG |
| | | AACAAGTGCCGTTCAAATAGGGCGGCACCTTGACGGTACCTAACCAGAAA |
| | | GCCACGGCTAACTACGTGCCAGCAGCCGCGGTAATACGTAGGTGGCAAGC |
| | | GTTGTCCGGAATTATTGGGCGTAAAGGGCTCGCAGGCGGTTTCTTAAGTCT |
| | | GATGTGAAAGCCCCCGGCTCAACCGGGGAGGGTCATTGGAAACTGGGGA |
| | | ACTTGAGTGCAGAAGAGGAGAGTGGAATTCCACGTGTAGCGGTG |
| 169 | DP70 16S | TGCAAGTCGAGCGGACAGATGGGAGCTTGCTCCCTGATGTTAGCGGCGGA |
| | rRNA | CGGGTGAGTAACACGTGGGTAACCTGCCTGTAAGACTGGGATAACTCCGG |
| | | GAAACCGGGGCTAATACCGGATGGTTGTTTGAACCGCATGGTTCAAACAT |
| | | AAAAGGTGGCTTCGGCTACCACTTACAGATGGACCCGCGGCGCATTAGCT |
| | | AGTTGGTGAGGTAACGGCTCACCAAGGCAACGATGCGTAGCCGACCTGAG |
| | | AGGGTGATCGGCCACACTGGGACTGAGACACGGCCCAGACTCCTACGGGA |
| | | GGCAGCAGTAGGGAATCTTCCGCAATGGACGAAAGTCTGACGGAGCAAC |
| | | GCCGCGTGAGTGATGAAGGTTTTCGGATCGTAAAGCTCTGTTGTTAGGGA |
| | | AGAACAAGTACCGTTCGAATAGGGCGGTACCTTGACGGTACCTAACCAGA |
| | | AAGCCACGGCTAACTACGTGCCAGCAGCCGCGGTAATACGTAGGTGGCAA |
| | | GCGTTGTCCGGAATTATTGGGCGTAAAGGGCTCGCAGGCGGTTTCTTAAGT |
| | | CTGATGTGAAAGCCCCCGGCTCAACCGGGGAGGGTCATTGGAAACTGGGG |
| | | AACTTGAGTGCAGAAGAGGAGAGTGGAATTCCACGTGTAGCGGTGAAATG |
| | | CGTAGAGATGTGGAGGAACACCAGTGGCGAAGGCGACTCTCTGGTCTGTA |
| | | ACTGACGCTGANGAGCGAAAGCGTGGGGAGCGAACAGGATTAGATACCC |
| | | TGGTAGTCCACGCCGTAAACGATGAGTGCTAAGTGTTA |
| 170 | DP71 16S | TTACTTGGAGTCCGAACTCTCACTTTTTAACCCTGTGCATCTGTTAATTGGA |
| | rRNA | ATAGTAGCTCTTCGGAGTGAACCACCATTCACTTATAAAACACAAAGTCT |
| | | ATGAATGTATACAAATTTATAACAAAACAAAACTTTCAACAACGGATCTC |
| | | TTGGCTCTCGCATCGATGAAGAACGCAGCGAAATGCGATACGTAATGTGA |
| | | ATTGCAGAATTCAGTGAATCATCGAATCTTTGAACGCACCTTGCGCTCCTT |
| | | GGTATTCCGAGGAGCATGCCTGTTTGAGTGTCATGAAATCTTCAACCCACC |
| | | TCTTTCTTAGTGAATCTGGTGGTGCTTGGTTTCTGAGCGCTGCTCTGCTTCG |
| | | GCTTAGCTCGTTCGTAATGCATTAGCATCCGCAACCGAACTTCGGATTGAC |
| | | TTGGCGTAATAGACTATTCGCTGAGGATTCTAGTTTACTAGAGCCGAGTTG |
| | | GGTTAAAGGAAGCTCCTAATCCTAAAGTCTATTTTTTGATTAGATCTCAAA |
| | | TCAGGTAGGACTACCCGCTGAACTTAAGCATATCAATAAGCGGAGGAAAA |
| | | GAAACTAACAAGGATTCCCCTAGTAGCGGCGAGCGAAGCGGGAAGAGCT |
| | | CAAATTTATAATCTGGCACCTTCGGTGTCCGAGTTGTAATCTCTAGAAGTG |
| | | TTTTCCGCGTTGGACCGCACACAAGTCTGTTGGAATACAGCGGCATAGTG |
| | | GTGAAACCCCCGTATATGGTGCGGACGCCCAGCGCTTTGTGATACACTTTC |
| | | AATGAGTCGAGTTGTTTGGGAATGCAGCTCAAATTGGGTGGTAAATTCCA |
| | | TCTAAAGCTAAATATTGGCGAGAGACCGATAGCGAACAAGTACCGTGAGG |
| | | GAAAGATGAAAAGCACTTTGGAAAGAGAGTTAACAGTACGTGAAATTGTT |
| | | GGAA |
| 171 | DP21 18S | GGGGGCATCAGTATTCAGTTGTCAGAGGTGAAATTCTTGGATTTACTGAA |
| | rRNA | GACTAACTACTGCGAAAGCATTTGCCAAGGACGTTTTCATTAATCAAGAA |
| | | CGAAAGTTAGGGGATCGAAGATGATCAGATACCGTCGTAGTCTTAACCAT |
| | | AAACTATGCCGACTAGGGATCGGGTGTTGTTCTTTTTTTGACGCACTCGGC |
| | | ACCTTACGAGAAATCAAAGTCTTTGGGTTCTGGGGGGAGTATGGTCGCAA |
| | | GGCTGAAACTTAAAGGAATTGACGGAAGGGCACCACCAGGAGTGGAGCC |
| | | TGCGGCTTAATTTGACTCAACACGGGGAAACTCACCAGGTCCAGACACAA |
| | | TAAGGATTGACAGATTGAGAGCTCTTTCTTGATTTTGTGGGTGGTGGTGCA |
| | | TGGCCGTTCTTAGTTGGTGGAGTGATTTGTCTGCTTAATTGCGATAACGAA |
| | | CGAGACCTTAACCTACTAAATAGTGCTGCTAGCTTTTGCTGGTATAGTCAC |
| | | TTCTTAGAGGGACTATCGATTTCAAGTCGATGGAAGTTTGAGGCAATAAC |
| | | AGGTCTGTGATGCCCTTAGACGTTCTGGGCCGCACGCGCGCTACACTGAC |
| | | GGAGCCAGCGAGTTCTAACCTTGGCCGAGAGGTCTGGGTAATCTTGTGAA |
| | | ACTCCGTCGTGCTGGGGATAGAGCATTGTAATTATTGCTCTTCAACGAGGA |
| | | ATTCCTAGTAAGCGCAAGTCATCAGCTTGCGTTGATTACGTCCCTGCCCTT |
| | | TGTACACACCGCCCGTCGCTACTACCGATTGAATGGCTTAGTGAGGCTTCC |
| | | GGATTGGTTTAAAGAAGGGGGCAACTCCATCTTGGAACCGAAAAGCTAGT |
| | | CAAACTTGGTCATTTAGAGGAAGTAAAAGTCGTAACAAGGTTTCCGTAGG |
| | | TGAACCTGCGGAAGGATCATT |
| 172 | DP99 16S | GATTTGAAGAGCTTGCTCAGATATGACGATGGACATTGCAAAGAGTGGCG |
| | rRNA | AACGGGTGAGTAACACGTGGGAAACCTACCTCTTAGCAGGGGATAACATT |
| | | TGGAAACAGATGCTAATACCGTATAACAATAGCAACCGCATGGTTGCTAC |
| | | TTAAAAGATGGTTCTGCTATCACTAAGAGATGGTCCCGCGGTGCATTAGTT |
| | | AGTTGGTGAGGTAATGGCTCACCAAGACGATGATGCATAGCCGAGTTGAG |
| | | AGACTGATCGGCCACAATGGGACTGAGACACGGCCCATACTCCTACGGGA |
| | | GGCAGCAGTAGGGAATCTTCCACAATGGGCGAAAGCCTGATGGAGCAAC |
| | | GCCGCGTGTGTGATGAAGGGTTTCGGCTCGTAAAACACTGTTGTAAGAGA |
| | | AGAATGACATTGAGAGTAACTGTTCAATGTGTGACGGTATCTTACCAGAA |
| | | AGGAACGGCTAAATACGTGCCAGCAGCCGCGGTAATACGTATGTTCCAAG |
| | | CGTTATCCGGATTTATTGGGCGTAAAGCGAGCGCAGACGGTTATTTAAGTC |
| | | TGAAGTGAAAGCCCTCAGCTCAACTGAGGAATTGCTTTGGAAACTGGATG |
| | | ACTTGAGTGCAGTAGAGG |
| | DP3 | AACGCACAGCGAAAGGTGCTTGCACCTTTCAAGTGAGTGGCGAACGGGTG |
| | Reisolate | AGTAACACGTGGACAACCTGCCTCAAGGCTGGGGATAACATTTGGAAACA |
| | #1 | GATGCTAATACCGAATAAAACTTAGTGTCGCATGACAAAAAGTTAAAAGGC |
| | | GCTTCGGCGTCACCTAGAGATGGATCCGCGGTGCATTAGTTAGTTGGTGGG |
| | | GTAAAGGCCTACCAAGACAATGATGCATAGCCGAGTTGAGAGACTGATCG |
| | | GCCACATTGGGACTGAGACACGGCCCAAACTCCTACGGGAGGCTGCAGTA |
| | | GGGAATCTTCCACAATGGGCGAAAGCCTGATGGAGCAACGCCGCGTGTGT |
| | | GATGAAGGCTTTCGGGTCGTAAAGCACTGTTGTATGGGAAGAACAGCTAG |
| | | AATAGGAAATGATTTTAGTTTGACGGTACCATACCAGAAAGGGACGGCTAA |
| | | ATACGTGCCAGCAGCCGCGGTAATACGTATGTCCCGAGCGTTATCCGGATT |
| | | TATTGGGCGTAAAGCGAGCGCAGACGGTTTATTAAGTCTGATGTGAAAGCC |
| | | CGGAGCTCAACTCCGGAATGGCATTGGAAACTGGTTAACTTGAGTGCAGTA |
| | | GAGGTAAGTGGAACTCCATGTGTAGCGGTGGAATGCGTAGATATATGGAA |
| | | GAACACC |
| | DP3 | ATTGAGAGTTTGATCCTGGCTCAGGATGAACGCTGGCGGCGTGCCTAATAC |
| | Reisolate | ATGCAAGTCGAACGCACAGCGAAAGGTGCTTGCACCTTTCAAGTGAGTGGC |
| | #2 | GAACGGGTGAGTAACACGTGGACAACCTGCCTCAAGGCTGGGGATAACAT |
| | | TTGGAAACAGATGCTAATACCGAATAAAACTCAGTGTCGCATGACACAAAG |
| | | TTAAAAGGCGCTTTGGCGTCACCTAGAGATGGATCCGCGGTGCATTAGTTA |
| | | GTTGGTGGGGTAAAGGCCTACCAAGACAATGATGCATAGCCGAGTTGAGA |
| | | GACTGATCGGCCACATTGGGACTGAGACACGGCCCAAACTCCTACGGGAG |
| | | GCTGCAGTAGGGAATCTTCCACAATGGGCGAAAGCCTGATGGAGCAACGC |
| | | CGCGTGTGTGATGAAGGCTTTCGGGTCGTAAAGCACTGTTGTACGGGAAG |
| | | AACAGCTAGAATAGGGAATGATTTTAGTTTGACGGTACCATACCAGAAAGG |
| | | GACGGCTAAATACGTGCCAGCAGCCGCGGTAATACGTATGTCCCGAGCGTT |
| | | ATCCGGATTTATTGGGCGTAAAGCGAGCGCAGACGGTTGATTAAGTCTGAT |
| | | GTGAAAGCCCGGAGCTCAACTCCGGAATGGCATTGGAAACTGGTTAACTTG |
| | | AGTGCAGTAGAGGTAAGTGGAACTCCATGTGTAGCGGTGGAATGCGTAGA |
| | | TATATGGAAGAACACCAGTGGCGAAGGCGGCTTACTGGACTGTAACTGAC |
| | | GTTGAGGCTCGAAAGTGTGGGTAGCAAACAGGATTAGATACCCTGGTAGT |
| | | CCACACCGTAAACGATGAACACTAGGTGTTAGGAGGTTTCCGCCTCTTAGT |
| | | GCCGAAGCTAACGCATTAAGTGTTCCGCCTGGGGAGTACGACCGCAAGGTT |
| | | GAAACTCAAAGGAATTGACGGGGACCCGCACAAGCGGTGGAGCATGTGGT |
| | | TTAATTCGAAGCAACGCGAAGAACCTTACCAGGTCTTGACATCCTTTGAAGC |
| | | TTTTAGAGATAGAAGTGTTCTCTTCGGAGACAAAGTGACAGGTGGTGCATG |
| | | GTCGTCGTCAGCTCGTGTCGTGAGATGTTGGGTTAAGTCCCGCAACGAGCG |
| | | CAACCCTTATTGTTAGTTGCCAGCATTCAGATGGGCACTCTAGCGAGACTGC |
| | | CGGTGACAAACCGGAGGAAGGCGGGGACGACGTCAGATCATCATGCCCCT |
| | | TATGACCTGGGCTACACACGTGCTACAATGGCGTATACAACGAGTTGCCAA |
| | | CCCGCGAGGGTGAGCTAATCTCTTAAAGTACGTCTCAGTTCGGATTGTAGT |
| | | CTGCAACTCGACTACATGAAGTCGGAATCGCTAGTAATCGCGGATCAGCAC |
| | | GCCGCGGTGAATACGTTCCCGGGTCTTGTACACACCGCCCGTCACACCATG |
| | | GGAGTTTGTAATGCCCAAAGCCGGTGGCCTAACCTTTTAGGAAGGAGCCGT |
| | | CTAAGGCAGGACAGATGACTGGGGTGAAGTCGTAACAAGGTAGCCGTAGG |
| | | AGAACCTGCGGCTGGATCACCTCCTTT |
| | DP3 | GCAGTCGAACGCACAGCGAAAGGTGCTTGCACCTTTCAAGTGAGTGGCGA |
| | Reisolate | ACGGGTGAGTAACACGTGGACAACCTGCCTCAAGGCTGGGGATAACATTT |
| | #3 | GGAAACAGATGCTAATACCGAATAAAACTCAGTGTCGCATGACACAAAGTT |
| | | AAAAGGCGCTTTGGCGTCACCTAGAGATGGATCCGCGGTGCATTAGTTAGT |
| | | TGGTGGGGTAAAGGCCTACCAAGACAATGATGCATAGCCGAGTTGAGAGA |
| | | CTGATCGGCCACATTGGGACTGAGACACGGCCCAAACTCCTACGGGAGGCT |
| | | GCAGTAGGGAATCTTCCACAATGGGCGAAAGCCTGATGGAGCAACGCCGC |
| | | GTGTGTGATGAAGGCTTTCGGGTCGTAAAGCACTGTTGTACGGGAAGAAC |
| | | AGCTAGAATAGGGAATGATTTTAGTTTGACGGTACCATACCAGAAAGGGAC |
| | | GGCTAAATACGTGCCAGCAGCCGCGGTAATACGTATGTCCCGAGCGTTATC |
| | | CGGATTTATTGGGCGTAAAGCGAGCGCAGACGGTTGATTAAGTCTGATGTG |
| | | AAAGCCCGGAGCTCAACTCCGGAATGGCATTGGAAACTGGTTAACTTGAGT |
| | | GCAGTAGAGGTAAGTGGAACTCCATGTGTAGCGGTGGAATGCG |
| | DP3 | GTCGAACGCACAGCGAAAGGTGCTTGCACCTTTCAAGTGAGTGGCGAACG |
| | Reisolate | GGTGAGTAACACGTGGACAACCTGCCTCAAGGCTGGGGATAACATTTGGA |
| | #4 | AACAGATGCTAATACCGAATAAAACTCAGTGTCGCATGACACAAAGTTAAA |
| | | AGGCGCTTTGGCGTCACCTAGAGATGGATCCGCGGTGCATTAGTTAGTTGG |
| | | TGGGGTAAAGGCCTACCAAGACAATGATGCATAGCCGAGTTGAGAGACTG |
| | | ATCGGCCACATTGGGACTGAGACACGGCCCAAACTCCTACGGGAGGCTGC |
| | | AGTAGGGAATCTTCCACAATGGGCGAAAGCCTGATGGAGCAACGCCGCGT |
| | | GTGTGATGAAGGCTTTCGGGTCGTAAAGCACTGTTGTACGGGAAGAACAG |
| | | CTAGAATAGGGAATGATTTTAGTTTGACGGTACCATACCAGAAAGGGACGG |
| | | CTAAATACGTGCCAGCAGCCGCGGTAATACGTATGTCCCGAGCGTTATCCG |
| | | GATTTATTGGGCGTAAAGCGAGCGCAGACGGTTGATTAAGTCTGATGTGAA |
| | | AGCCCGGAGCTCAACTCCGGAATGGCATTGGAAACTGGTTAACTTGAGTGC |
| | | AGTAGAGGTAAGTGGAACTCCATGTGTAGCGGTGGAATGCGTAGATATAT |
| | | GGAAGAACACCAGTGGCGAAGGCGGCTTACTGGACTGTAAC |
| | DP9 | ATGAGAGTTTGATCTTGGCTCAGGATGAACGCTGGCGGCGTGCCTAATACA |
| | Reisolate | TGCAAGTCGAACGAACTTCCGTTAATTGATTATGACGTACTTGTACTGATTG |
| | #1 | AGATTTTAACACGAAGTGAGTGGCGAACGGGTGAGTAACACGTGGGTAAC |
| | | CTGCCCAGAAGTAGGGGATAACACCTGGAAACAGATGCTAATACCGTATAA |
| | | CAGAGAAAACCGCATGGTTTTCTTTTAAAAGATGGCTCTGCTATCACTTCTG |
| | | GATGGACCCGCGGCGTATTAGCTAGTTGGTGAGGCAAAGGCTCACCAAGG |
| | | CAGTGATACGTAGCCGACCTGAGAGGGTAATCGGCCACATTGGGACTGAG |
| | | ACACGGCCCAGACTCCTACGGGAGGCAGCAGTAGGGAATCTTCCACAATG |
| | | GACGCAAGTCTGATGGAGCAACGCCGCGTGAGTGAAGAAGGGTTTCGGCT |
| | | CGTAAAGCTCTGTTGTTAAAGAAGAACGTGGGTAAGAGTAACTGTTTACCC |
| | | AGTGACGGTATTTAACCAGAAAGCCACGGCTAACTACGTGCCAGCAGCCGC |
| | | GGTAATACGTAGGTGGCAAGCGTTATCCGGATTTATTGGGCGTAAAGCGA |
| | | GCGCAGGCGGTCTTTTAAGTCTAATGTGAAAGCCTTCGGCTCAACCGAAGA |
| | | AGTGCATTGGAAACTGGGAGACTTGAGTGCAGAAGAGGACAGTGGAACTC |
| | | CATGTGTAGCGGTGAAATGCGTAGATATATGGAAGAACACCAGTGGCGAA |
| | | GGCGGCTGTCTGGTCTGCAACTGACGCTGAGGCTCGAAAGCATGGGTAGC |
| | | GAACAGGATTAGATACCCTGGTAGTCCATGCCGTAAACGATGATTACTAAG |
| | | TGTTGGAGGGTTTCCGCCCTTCAGTGCTGCAGCTAACGCATTAAGTAATCCG |
| | | CCTGGGGAGTACGACCGCAAGGTTGAAACTCAAAAGAATTGACGGGGGCC |
| | | CGCACAAGCGGTGGAGCATGTGGTTTAATTCGAAGCTACGCGAAGAACCTT |
| | | ACCAGGTCTTGACATCTTCTGACAGTCTAAGAGATTAGAGGTTCCCTTCGGG |
| | | GACAGAATGACAGGTGGTGCATGGTTGTCGTCAGCTCGTGTCGTGAGATGT |
| | | TGGGTTAAGTCCCGCAACGAGCGCAACCCTTATTACTAGTTGCCAGCATTAA |
| | | GTTGGGCACTCTAGTGAGACTGCCGGTGACAAACCGGAGGAAGGTGGGGA |
| | | CGACGTCAAATCATCATGCCCCTTATGACCTGGGCTACACACGTGCTACAAT |
| | | GGATGGTACAACGAGTCGCGAGACCGCGAGGTTAAGCTAATCTCTTAAAAC |
| | | CATTCTCAGTTCGGACTGTAGGCTGCAACTCGCCTACACGAAGTCGGAATC |
| | | GCTAGTAATCGCGGATCAGCATGCCGCGGTGAATACGTTCCCGGGCCTTGT |
| | | ACACACCGCCCGTCACACCATGAGAGTTTGTAAC |
| | DP9 | TGCAGTCGAACGAACTTCCGTTAATTGATTATGACGTACTTGTACTGATTGA |
| | Reisolate | GATTTTAACACGAAGTGAGTGGCGAACGGGTGAGTAACACGTGGGTAACC |
| | #2 | TGCCCAGAAGTAGGGGATAACACCTGGAAACAGATGCTAATACCGTATAAC |
| | | AGAGAAAACCGCATGGTTTTCTTTTAAAAGATGGCTCTGCTATCACTTCTGG |
| | | ATGGACCCGCGGCGTATTAGCTAGTTGGTGAGGCAAAGGCTCACCAAGGC |
| | | AGTGATACGTAGCCGACCTGAGAGGGTAATCGGCCACATTGGGACTGAGA |
| | | CACGGCCCAGACTCCTACGGGAGGCAGCAGTAGGGAATCTTCCACAATGG |
| | | ACGCAAGTCTGATGGAGCAACGCCGCGTGAGTGAAGAAGGGTTTCGGCTC |
| | | GTAAAGCTCTGTTGTTAAAGAAGAACGTGGGTAAGAGTAACTGTTTACCCA |
| | | GTGACGGTATTTAACCAGAAAGCCACGGCTAACTACGTGCCAGCAGCCGCG |
| | | GTAATACGTAGGTGGCAAGCGTTATCCGGATTTATTGGGCGTAAAGCGAGC |
| | | GCAGGCGGTCTTTTAAGTCTAATGTGAAAGCCTTCGGCTCAACCGAAGAAG |
| | | TGCATTGGAAACTGGGAGACTTGAGTGCAGAAGAGGACAGTGGAACTCCA |
| | | TGTGTAGCGGTGAAATGCGTAGATATATGGAAGAACACCAGTGGCGAAGG |
| | | CGGCTGTCTGGTCTGCAACTGACGCTGAGGCT |
| | DP9 | AGTCGAACGAACTTCCGTTAATTGATTATGACGTACTTGTACTGATTGAGAT |
| | Reisolate | TTTAACACGAAGTGAGTGGCGAACGGGTGAGTAACACGTGGGTAACCTGC |
| | #3 | CCAGAAGTAGGGGATAACACCTGGAAACAGATGCTAATACCGTATAACAG |
| | | AGAAAACCGCATGGTTTTCTTTTAAAAGATGGCTCTGCTATCACTTCTGGAT |
| | | GGACCCGCGGCGTATTAGCTAGTTGGTGAGGCAAAGGCTCACCAAGGCAG |
| | | TGATACGTAGCCGACCTGAGAGGGTAATCGGCCACATTGGGACTGAGACA |
| | | CGGCCCAGACTCCTACGGGAGGCAGCAGTAGGGAATCTTCCACAATGGAC |
| | | GCAAGTCTGATGGAGCAACGCCGCGTGAGTGAAGAAGGGTTTCGGCTCGT |
| | | AAAGCTCTGTTGTTAAAGAAGAACGTGGGTAAGAGTAACTGTTTACCCAGT |
| | | GACGGTATTTAACCAGAAAGCCACGGCTAACTACGTGCCAGCAGCCGCGGT |
| | | AATACGTAGGTGGCAAGCGTTATCCGGATTTATTGGGCGTAAAGCGAGCG |
| | | CAGGCGGTCTTTTAAGTCTAATGTGAAAGCCTTCGGCTCAACCGAAGAAGT |
| | | GCATTGGAAACTGGGAGACTTGAGTGCAGAAGAGGACAGTGGAACTCCAT |
| | | GTGTAGCGGTGAAATGCGTAGATATATGGAAGAACACCAGTGGCGAAG |
| | DP9 | TCGAACGAACTTCCGTTAATTGATTATGACGTACTTGTACTGATTGAGATTT |
| | Reisolate | TAACACGAAGTGAGTGGCGAACGGGTGAGTAACACGTGGGTAACCTGCCC |
| | #4 | AGAAGTAGGGGATAACACCTGGAAACAGATGCTAATACCGTATAACAGAG |
| | | AAAACCGCATGGTTTTCTTTTAAAAGATGGCTCTGCTATCACTTCTGGATGG |
| | | ACCCGCGGCGTATTAGCTAGTTGGTGAGGCAAAGGCTCACCAAGGCAGTG |
| | | ATACGTAGCCGACCTGAGAGGGTAATCGGCCACATTGGGACTGAGACACG |
| | | GCCCAGACTCCTACGGGAGGCAGCAGTAGGGAATCTTCCACAATGGACGC |
| | | AAGTCTGATGGAGCAACGCCGCGTGAGTGAAGAAGGGTTTCGGCTCGTAA |
| | | AGCTCTGTTGTTAAAGAAGAACGTGGGTAAGAGTAACTGTTTACCCAGTGA |
| | | CGGTATTTAACCAGAAAGCCACGGCTAACTACGTGCCAGCAGCCGCGGTAA |
| | | TACGTAGGTGGCAAGCGTTATCCGGATTTATTGGGCGTAAAGCGAGCGCA |
| | | GGCGGTCTTTTAAGTCTAATGTGAAAGCCTTCGGCTCAACCGAAGAAGTGC |
| | | ATTGGAAACTGGGAGACTTGAGTGCAGAAGAGGACAGTGGAACTCCATGT |
| | | GTAGCGGTGAAATGCG |
| | DP9 | TGCAGTCGAACGCATTTCCGTTAAAAGAATCAGAAGTGCTTGCACGGAAGA |
| | Reisolate | TGATTTTAACAATGAAATGAGTGGCGAACGGGTGAGTAACACGTGGGTAA |
| | #5 | CCTGCCCAGAAGAGGGGGATAACACTTGGAAACAGGTGCTAATACCGCAT |
| | | AATAAAGAAAACCGCATGGTTTTCCTTTAAAAGATGGTTTCGGCTATCACTT |
| | | CTGGATGGACCCGCGGCGTATTAGCTAGTTGGTAAGGTAAAGGCTTACCAA |
| | | GGCAGTGATACGTAGCCGACCTGAGAGGGTAATCGGCCACATTGGGACTG |
| | | AGACACGGCCCAGACTCCTACGGGAGGCAGCAGTAGGGAATCTTCCACAA |
| | | TGGACGAAAGTCTGATGGAGCAACGCCGCGTGAGTGAAGAAGGGTTTCGG |
| | | CTCGTAAAACTCTGTTGTTAAAGAAGAACGTGGGTGAGAGTAACTGTTCAC |
| | | CCAGTGACGGTATTTAACCAGAAAGCCACGGCTAACTACGTGCCAGCAGCC |
| | | GCGGTAATACGTAGGTGGCAAGCGTTATCCGGATTTATTGGGCGTAAAGC |
| | | GAGCGCAGGCGGTCTTTTAAGTCTAATGTGAAAGCCTTCGGCTCAACCGAA |
| | | GAAGTGCATTGGAAACTGGGAGACTTGAGTGCAGAAGAGGACAGTGGAA |
| | | CTCCATGTGTAGCGGTGAAATGC |
| | DP9 | AGTCGAACGAACTCTGGTATTGATTGGTGCTTGCATCATGATTTACATTTGA |
| | Reisolate | GTGAGTGGCGAACTGGTGAGTAACACGTGGGAAACCTGCCCAGAAGCGGG |
| | #6 | GGATAACACCTGGAAACAGATGCTAATACCGCATAACAACTTGGACCGCAT |
| | | GGTCCGAGTTTGAAAGATGGCTTCGGCTATCACTTTTGGATGGTCCCGCGG |
| | | CGTATTAGCTAGATGGTGGGGTAACGGCTCACCATGGCAATGATACGTAGC |
| | | CGACCTGAGAGGGTAATCGGCCACATTGGGACTGAGACACGGCCCAAACT |
| | | CCTACGGGAGGCAGCAGTAGGGAATCTTCCACAATGGACGAAAGTCTGAT |
| | | GGAGCAACGCCGCGTGAGTGAAGAAGGGTTTCGGCTCGTAAAACTCTGTT |
| | | GTTAAAGAAGAACATATCTGAGAGTAACTGTTCAGGTATTGACGGTATTTA |
| | | ACCAGAAAGCCACGGCTAACTACGTGCCAGCAGCCGCGGTAATACGTAGG |
| | | TGGCAAGCGTTGTCCGGATTTATTGGGCGTAAAGCGAGCGCAGGCGGTTTT |
| | | TTAAGTCTGATGTGAAAGCCTTCGGCTCAACCGAAGAAGTGCATCGGAAAC |
| | | TGGGAAACTTGAGTGCAGAAGAGGACAGTGGAACTCCATGTGTAGCGGTG |
| | DP53 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #1 | GGTGAGTAATACCTAGGAATCTGCCTGATAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCTACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGCAGTTACCT |
| | | AATACGTGATTGTCTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGAATGTGAAATCCCCGG |
| | | GCTCAACCTGGGAACTGCATCCAAAACTGGCAAGCTAGAGTATGGTAGAG |
| | | GGTAGTGGAATTTCCTGTGTAGCGGTGAAATGCGTAGATATAGGAAGGAA |
| | | CACCAGTGGCGAAGGCGACTACCTGGACTGATACTGACACTGAGGTGCGA |
| | | AAGCGTGGGGAGCAAACAGGATTAGATACCCTGGTAGTCCACGCCGTAAA |
| | | CGATGTCAACTAGCCGTTGGGAGTCTTGAACTCTTAGTGGCGCAGCTAACG |
| | | CATTAAGTTGACCGCCTGGGGAGTACGGCCGCAAGGTTAAAACTCAAATGA |
| | | ATTGACGGGGGCCCGCACAAGCGGTGGAGCATGTGGTTTAATTCGAAGCA |
| | | ACGCGAAGAACCTTACCAGGCCTTGACATCCAATGAACTTTCTAGAGATAG |
| | | ATTGGTGCCTTCGGGAACATTGAGACAGGTGCTGCATGGCTGTCGTCAGCT |
| | | CGTGTCGTGAGATGTTGGGTTAAGTCCCGTAACGAGCGCAACCCTTGTCCT |
| | | TAGTTACCAGCACGTAATGGTGGGCACTCTAAGGAGACTGCCGGTGACAAA |
| | | CCGGAGGAAGGTGGGGATGACGTCAAGTCATCATGGCCCTTACGGCCTGG |
| | | GCTACACACGTGCTACAATGGTCGGTACAAAGGGTTGCCAAGCCGCGAGG |
| | | TGGAGCTAATCCCATAAAACCGATCGTAGTCCGGATCGCAGTCTGCAACTC |
| | | GACTGCGTGAAGTCGGAATCGCTAGTAATCGTGAATCAGAATGTCACGGTG |
| | | AATACGTTCCCGGGCCTTGTACACACCGCCCGTCACACCATGGGAGTGGGT |
| | | TGCACCAGAAGTAGCTAGTCTAACCTTCGGGAGGACGGTTACCACGGTGTG |
| | | ATTCATGACTGGGGTGAAGTCGTAACAAGGTAGCCGTAGGGGAACCTGCG |
| | | GCTGGATCACCTCCTT |
| | DP53 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #2 | GGTGAGTAATACCTAGGAATCTGCCTGATAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCTACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGTATTAACCT |
| | | AATACGTTAGTACTTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGAATGTGAAATCCCCGG |
| | | GCTCAACCTGGGAACTGCATCCAAAACTGGCAAGCTAGAGTATGGTAGAG |
| | | GGTAGTGGAATTTCCTGTGTAGCGGTGAAATGCGTAGATATAGGAAGGAA |
| | | CACCAGTGGCGAAGGCGACTACCTGGACTGATACTGACACTGAGGTGCGA |
| | | AAGCGTGGGGAGCAAACAGGATTAGATACCCTGGTAGTCCACGCCGTAAA |
| | | CGATGTCAACTAGCCGTTGGGAGTCTTGAACTCTTAGTGGCGCAGCTAACG |
| | | CATTAAGTTGACCGCCTGGGGAGTACGGCCGCAAGGTTAAAACTCAAATGA |
| | | ATTGACGGGGGCCCGCACAAGCGGTGGAGCATGTGGTTTAATTCGAAGCA |
| | | ACGCGAAGAACCTTACCAGGCCTTGACATCCAATGAACTTTCTAGAGATAG |
| | | ATTGGTGCCTTCGGGAACATTGAGACAGGTGCTGCATGGCTGTCGTCAGCT |
| | | CGTGTCGTGAGATGTTGGGTTAAGTCCCGTAACGAGCGCAACCCTTGTCCT |
| | | TAGTTACCAGCACATAATGGTGGGCACTCTAAGGAGACTGCCGGTGACAAA |
| | | CCGGAGGAAGGTGGGGATGACGTCAAGTCATCATGGCCCTTACGGCCTGG |
| | | GCTACACACGTGCTACAATGGTCGGTACAAAGGGTTGCCAAGCCGCGAGG |
| | | TGGAGCTAATCCCATAAAACCGATCGTAGTCCGGATCGCAGTCTGCAACTC |
| | | GACTGCGTGAAGTCGGAATCGCTAGTAATCGTGAATCAGAATGTCACGGTG |
| | | AATACGTTCCCGGGCCTTGTACACACCGCCCGTCACACCATGGGAGTGGGT |
| | | TGCACCAGAAGTAGCTAGTCTAACCTTCGGGAGGACGGTTACCACGGTGTG |
| | | ATTCATGACTGGGGTGAAGTCGTAACAAGGTAGCCGTAGGGGAACCTGCG |
| | | GCTGGATCACCTCCTT |
| | DP53 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #3 | GGTGAGTAATACCTAGGAATCTGCCTGATAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCTACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGCAGTTACCT |
| | | AATACGTGATTGTCTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGAATGTGAAATCCCCGG |
| | | GCTCAACCTGGGAACTGCATCCAAAACTGGCAAGCTAGAGTATGGTAGAG |
| | | GGTAGTGGAATTTCCTGTGTAGCGGTGAAATGCGTAGATATAGGAAGGAA |
| | | CACCAGTGGCGAAGGCGACTACCTGGACTGATACTGACACTGAGGTGCGA |
| | | AAGCGTGGGGAGCAAACAGGATTAGATACCCTGGTAGTCCACGCCGTAAA |
| | | CGATGTCAACTAGCCGTTGGGAGTCTTGAACTCTTAGTGGCGCAGCTAACG |
| | | CATTAAGTTGACCGCCTGGGGAGTACGGCCGCAAGGTTAAAACTCAAATGA |
| | | ATTGACGGGGGCCCGCACAAGCGGTGGAGCATGTGGTTTAATTCGAAGCA |
| | | ACGCGAAGAACCTTACCAGGCCTTGACATCCAATGAACTTTCTAGAGATAG |
| | | ATTGGTGCCTTCGGGAACATTGAGACAGGTGCTGCATGGCTGTCGTCAGCT |
| | | CGTGTCGTGAGATGTTGGGTTAAGTCCCGTAACGAGCGCAACCCTTGTCCT |
| | | TAGTTACCAGCACGTAATGGTGGGCACTCTAAGGAGACTGCCGGTGACAAA |
| | | CCGGAGGAAGGTGGGGATGACGTCAAGTCATCATGGCCCTTACGGCCTGG |
| | | GCTACACACGTGCTACAATGGTCGGTACAAAGGGTTGCCAAGCCGCGAGG |
| | | TGGAGCTAATCCCATAAAACCGATCGTAGTCCGGATCGCAGTCTGCAACTC |
| | | GACTGCGTGAAGTCGGAATCGCTAGTAATCGTGAATCAGAATGTCACGGTG |
| | | AATACGTTCCCGGGCCTTGTACACACCGCCCGTCACACCATG |
| | DP53 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #4 | GGTGAGTAATACCTAGGAATCTGCCTGATAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCTACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGCATTAACCT |
| | | AATACGTTGGTGTCTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGAATGTGAAATCCCCGG |
| | | GCTCAACCTGGGAACTGCATCCAAAACTGGCAAGCTAGAGTATGGTAGAG |
| | | GGTAGTGGAATTTCCTGTGTAGCGGTGAAATGCGTAGATATAGGAAGGAA |
| | | CACCAGTGGCGAAGGCGACTACCTGGACTGATACTGACACTGAGGTGCGA |
| | | AAGCGTGGGGAGCAAACAGGATTAGATACCCTGGTAGTCCACGCCGTAAA |
| | | CGATGTCAACTAGCCGTTGGGAGTCTTGAACTCTTAGTGGCGCAGCTAACG |
| | | CATTAAGTTGACCGCCTGGGGAGTACGGCCGCAAGGTTAAAACTCAAATGA |
| | | ATTGACGGGGGCCCGCACAAGCGGTGGAGCATGTGGTTTAATTCGAAGCA |
| | | ACGCGAAGAACCTTACCAGGCCTTGACATCCAATGAACTTTCTAGAGATAG |
| | | ATTGGTGCCTTCGGGAACATTGAGACAGGTGCTGCATGGCTGTCGTCAGCT |
| | | CGTGTCGTGAGATGTTGGGTTAAGTCCCGTAACGAGCGCAACCCTTGTCCT |
| | | TAGTTACCAGCACGTAATGGTGGGCACTCTAAGGAGACTGCCGGTGACAAA |
| | | CCGGAGGAAGGTGGGGATGACGTCAAGTCATCATGGCCCTTACGGCCTGG |
| | | GCTACACACGTGCTACAATGGTCGGTACAAAGGGTTGCCAAGCCGCGAGG |
| | | TGGAGCTAATCCCATAAAACCGATCGTAGTCCGGATCGCAGTCTGCAACTC |
| | | GACTGCGTGAAGTCGGAATCGCTAGTAATCGTGAATCAGAATGTCACGGTG |
| | | AATACGTTCCCGGGCCTTGTACACACCGCCCGTCACACCATGGGAGTGGGT |
| | | TGCACCAGAAGTAGCTAGTCTAACCCTCGGGAGGACGGTTACCACGGTGTG |
| | | ATTCATGACTGGGGTGAAGTCGTAACAAGGTAGCCGTAGGGGAACCTGCG |
| | | GCTGGATCACCTCCTT |
| | DP53 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #5 | GGTGAGTAATACCTAGGAATCTGCCTGATAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCTACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGCAGTTACCT |
| | | AATACGTGACTGTCTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGAATGTGAAATCCCCGG |
| | | GCTCAACCTGGGAACTGCATCCAAAACTGGCAAGCTAGAGTATGGTAGAG |
| | | GGTAGTGGAATTTCCTGTGTAGCGGTGAAATGCGTAGATATAGGAAGGAA |
| | | CACCAGTGGCGAAGGCGACTACCTGGACTGATACTGACACTGAGGTGCGA |
| | | AAGCGTGGGGAGCAAACAGGATTAGATACCCTGGTAGTCCACGCCGTAAA |
| | | CGATGTCAACTAGCCGTTGGGAGTCTTGAACTCTTAGTGGCGCAGCTAACG |
| | | CATTAAGTTGACCGCCTGGGGAGTACGGCCGCAAGGTTAAAACTCAAATGA |
| | | ATTGACGGGGGCCCGCACAAGCGGTGGAGCATGTGGTTTAATTCGAAGCA |
| | | ACGCGAAGAACCTTACCAGGCCTTGACATCCAATGAACTTTCCAGAGATGG |
| | | ATTGGTGCCTTCGGGAACATTGAGACAGGTGCTGCATGGCTGTCGTCAGCT |
| | | CGTGTCGTGAGATGTTGGGTTAAGTCCCGTAACGAGCGCAACCCTTGTCCT |
| | | TAGTTACCAGCACGTAATGGTGGGCACTCTAAGGAGACTGCCGGTGACAAA |
| | | CCGGAGGAAGGTGGGGATGACGTCAAGTCATCATGGCCCTTACGGCCTGG |
| | | GCTACACACGTGCTACAATGGTCGGTACAAAGGGTTGCCAAGCCGCGAGG |
| | | TGGAGCTAATCCCATAAAACCGATCGTAGTCCGGATCGCAGTCTGCAACTC |
| | | GACTGCGTGAAGTCGGAATCGCTAGTAATCGTGAATCAGAATGTCACGGTG |
| | | AATACGTTCCCGGGCCTTGTACACACCGCCCGTCACACCATGGGAGTGGGT |
| | | TGCACCAGAAGTAGCTAGTCTAACCTTCGGGAGGACGGTTACCACGGTGTG |
| | | ATTCATGACTGGGGTGAAGT |
| | DP53 | TGAATCAAGCAATTCGTGTGGGTGCTTGTGGAGTCAGACTGATAGTCAACA |
| | Reisolate | AGATTATCAGCATCACAAGTTACTCCGCCGGACGGGTGAGTAATACCTAGG |
| | #6 | AATCTGCCTGATAGTGGGGGATAACGTTCGGAAACGGACGCTAATACCGCA |
| | | TACGTCCTACGGGAGAAAGCAGGGGACCTTCGGGCCTTGCGCTATCAGAT |
| | | GAGCCTAGGTCGGATTAGCTAGTTGGTGAGGTAATGGCTCACCAAGGCTAC |
| | | GATCCGTAACTGGTCTGAGAGGATGATCAGTCACACTGGAACTGAGACACG |
| | | GTCCAGACTCCTACGGGAGGCAGCAGTGGGGAATATTGGACAATGGGCGA |
| | | AAGCCTGATCCAGCCATGCCGCGTGTGTGAAGAAGGTCTTCGGATTGTAAA |
| | | GCACTTTAAGTTGGGAGGAAGGGCATTAACCTAATACGTTAGTGTCTTGAC |
| | | GTTACCGACAGAATAAGCACCGGCTAACTCTGTGCCAGCAGCCGCGGTAAT |
| | | ACAGAGGGTGCAAGCGTTAATCGGAATTACTGGGCGTAAAGCGCGCGTAG |
| | | GTGGTTTGTTAAGTTGAATGTGAAATCCCCGGGCTCAACCTGGGAACTGCA |
| | | TCCAAAACTGGCAAGCTAGAGTATGGTAGAGGGTAGTGGAATTTCCTGTGT |
| | | AGCGGTGAAATGCGTAGATATAGGAAGGAACACCAGTGGCGAAGGCGACT |
| | | ACCTGGACTGATACTGACACTGAGGTGCGAAAGCGTGGGGAGCAAACAGG |
| | | ATTAGATACCCTGGTAGTCCACGCCGTAAACGATGTCAACTAGCCGTTGGG |
| | | AGTCTTGAACTCTTAGTGGCGCAGCTAACGCATTAAGTTGACCGCCTGGGG |
| | | AGTACGGCCGCAAGGTTAAAACTCAAATGAATTGACGGGGGCCCGCACAA |
| | | GCGGTGGAGCATGTGGTTTAATTCGAAGCAACGCGAAGAACCTTACCAGG |
| | | CCTTGACATCCAATGAACTTTCTAGAGATAGATTGGTGCCTTCGGGAACATT |
| | | GAGACAGGTGCTGCATGGCTGTCGTCAGCTCGTGTCGTGAGATGTTGGGTT |
| | | AAGTCCCGTAACGAGCGCAACCCTTGTCCTTAGTTACCAGCACGTAATGGT |
| | | GGGCACTCTAAGGAGACTGCCGGTGACAAACCGGAGGAAGGTGGGGATG |
| | | ACGTCAAGTCATCATGGCCCTTACGGCCTGGGCTACACACGTGCTACAATG |
| | | GTCGGTACAAAGGGTTGCCAAGCCGCGAGGTGGAGCTAATCCCATAAAAC |
| | | CGATCGTAGTCCGGATCGCAGTCTGCAACTCGACTGCGTGAAGTCGGAATC |
| | | GCTAGTAATCGTGAATCAGAATGTCACGGTGAATACGTTCCCGGGCCTTGT |
| | | ACACACCGCCCGTCACACCATGGGAGTGGGTTGCACCAGAAGTAGCTAGTC |
| | | TAACCTTCGGGAGGACGGTTACCACGGTGTGATTCATGACTGGGGTGAAGT |
| | | CGTAACAAGGTAGCCGTAGGGGAACCTGCGGCTGGATCACCTCCTT |
| | DP1 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #1 | GGTGAGTAATGCCTAGGAATCTGCCTGGTAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCGACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGTTGTAGATT |
| | | AATACTCTGCAATTTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGGATGTGAAATCCCCGG |
| | | GCTCAACCTGGGAACTGCATTCAAAACTGACTGACTAGAGTATGGTAGAGG |
| | | GTGGTGGAATTTCCTGTGTAGCGGTGAAATGCGTAGATATAGGAAGGAAC |
| | | ACCAGTGGCGAAGGCGACCACCTGGACTAATACTGACACTGAGGTGCGAA |
| | | AGCGTGGGGAGCAAACAGGATTAGATACCCTGGTAGTCCACGCCGTAAAC |
| | | GATGTCAACTAGCCGTTGGAAGCCTTGAGCTTTTAGTGGCGCAGCTAACGC |
| | | ATTAAGTTGACCGCCTGGGGAGTACGGCCGCAAGGTTAAAACTCAAATGAA |
| | | TTGACGGGGGCCCGCACAAGCGGTGGAGCATGTGGTTTAATTCGAAGCAA |
| | | CGCGAAGAACCTTACCAGGCCTTGACATCCAATGAACTTTCTAGAGATAGA |
| | | TTGGTGCCTTCGGGAACATTGAGACAGGTGCTGCATGGCTGTCGTCAGCTC |
| | | GTGTCGTGAGATGTTGGGTTAAGTCCCGTAACGAGCGCAACCCTTGTTCTT |
| | | AGTTACCAGCACGTTATGGTGGGCACTCTAAGGAGACTGCCGGTGACAAAC |
| | | CGGAGGAAGGTGGGGATGACGTCAAGTCATCATGGCCCTTACGGCCTGGG |
| | | CTACACACGTGCTACAATGGTCGGTACAGAGGGTTGCCAAGCCGCGAGGT |
| | | GGAGCTAATCCCATAAAACCGATCGTAGTCCGGATCGCAGTCTGCAACTCG |
| | | ACTGCGTGAAGTCGGAATCGCTAGTAATCGCGAATCAGAATGTCGCGGTGA |
| | | ATACGTTCCCGGGCCTTGTACACACCGCCCGTCACACCATGGGAGTGGGTT |
| | | GCACCAGAAGTAGCTAGTCTAACCTTCGGGAGGACGGTTACCACGGTGTGA |
| | | TTCATGACTGGGGTGAAGTCGTAACAAGGTAGCCGTAGGGGAACCTGCGG |
| | | CTGGATCACCTCCTT |
| | DP1 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #2 | GGTGAGTAATGCCTAGGAATCTGCCTGGTAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCGACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGTTGTAGATT |
| | | AATACTCTGCAATTTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGGATGTGAAATCCCCGG |
| | | GCTCAACCTGGGAACTGCATTCAAAACTGACTGACTAGAGTATGGTAGAGG |
| | | GTGGTGGAATTTCCTGTGTAGCGGTGAAATGCGTAGATATAGGAAGGAAC |
| | | ACCAGTGGCGAAGGCGACCACCTGGACTAATACTGACACTGAGGTGCGAA |
| | | AGCGTGGGGAGCAAACAGGATTAGATACCCTGGTAGTCCACGCCGTAAAC |
| | | GATGTCAACTAGCCGTTGGAAGCCTTGAGCTTTTAGTGGCGCAGCTAACGC |
| | | ATTAAGTTGACCGCCTGGGGAGTACGGCCGCAAGGTTAAAACTCAAATGAA |
| | | TTGACGGGGGCCCGCACAAGCGGTGGAGCATGTGGTTTAATTCGAAGCAA |
| | | CGCGAAGAACCTTACCAGGCCTTGACATCCAATGAACTTTCTAGAGATAGA |
| | | TTGGTGCCTTCGGGAACATTGAGACAGGTGCTGCATGGCTGTCGTCAGCTC |
| | | GTGTCGTGAGATGTTGGGTTAAGTCCCGTAACGAGCGCAACCCTTGTTCTT |
| | | AGTTACCAGCACGTTATGGTGGGCACTCTAAGGAGACTGCCGGTGACAAAC |
| | | CGGAGGAAGGTGGGGATGACGTCAAGTCATCATGGCCCTTACGGCCTGGG |
| | | CTACACACGTGCTACAATGGTCGGTACAGAGGGTTGCCAAGCCGCGAGGT |
| | | GGAGCTAATCCCATAAAACCGATCGTAGTCCGGATCGCAGTCTGCAACTCG |
| | | ACTGCGTGAAGTCGGAATCGCTAGTAATCGCGAATCAGAATGTCGCGGTGA |
| | | ATACGTTCCCGGGCCTTGTACACACCGCCCGTCACACCATGGGAGTGGGTT |
| | | GCACCAGAAGTAGCTAGTCTAACCTTCGGGAGGACGGTTACCACGGTGTGA |
| | | TTCATGACTGGGGTGAAGTCGTAACAAGGTAGCCGTAGGGGAACCTGCGG |
| | | CTGGATCACCTCCTT |
| | DP1 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #3 | GGTGAGTAATGCCTAGGAATCTGCCTGGTAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCGACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGTTGTAGATT |
| | | AATACTCTGCAATTTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGGATGTGAAATCCCCGG |
| | | GCTCAACCTGGGAACTGCATTCAAAACTGACTGACTAGAGTATGGTAGAGG |
| | | GTGGTGGAATTTCCTGTGTAGCGGTGAAATGCGTAGATATAGGAAGGAAC |
| | | ACCAGTGGCGAAGGCGACCACCTGGACTAATACTGACACTGAGGTGCGAA |
| | | AGCGTGGGGAGCAAACAGGATTAGATACCCTGGTAGTCCACGCCGTAAAC |
| | | GATGTCAACTAGCCGTTGGAAGCCTTGAGCTTTTAGTGGCGCAGCTAACGC |
| | | ATTAAGTTGACCGCCTGGGGAGTACGGCCGCAAGGTTAAAACTCAAATGAA |
| | | TTGACGGGGGCCCGCACAAGCGGTGGAGCATGTGGTTTAATTCGAAGCAA |
| | | CGCGAAGAACCTTACCAGGCCTTGACATCCAATGAACTTTCTAGAGATAGA |
| | | TTGGTGCCTTCGGGAACATTGAGACAGGTGCTGCATGGCTGTCGTCAGCTC |
| | | GTGTCGTGAGATGTTGGGTTAAGTCCCGTAACGAGCGCAACCCTTGTCCTT |
| | | AGTTACCAGCACGTTATGGTGGGCACTCTAAGGAGACTGCCGGTGACAAAC |
| | | CGGAGGAAGGTGGGGATGACGTCAAGTCATCATGGCCCTTACGGCCTGGG |
| | | CTACACACGTGCTACAATGGTCGGTACAGAGGGTTGCCAAGCCGCGAGGT |
| | | GGAGCTAATCCCATAAAACCGATCGTAGTCCGGATCGCAGTCTGCAACTCG |
| | | ACTGCGTGAAGTCGGAATCGCTAGTAATCGCGAATCAGAATGTCGCGGTGA |
| | | ATACGTTCCCGGGCCTTGTACACACCGCCCGTCACACCATGGGAGTGGGTT |
| | | GCACCAGAAGTAGCTAGTCTAACCTTCGGGAGGACGGTTACCACGGTGTGA |
| | | TTCATGACTGGGGTGAAGTCGTAACAAGGTAGCCGTAGGGGAACCTGCGG |
| | | CTGGATCACCTCCTT |
| | DP1 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #4 | GGTGAGTAATGCCTAGGAATCTGCCTGGTAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCGACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGTTGTAGATT |
| | | AATACTCTGCAATTTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGGATGTGAAATCCCCGG |
| | | GCTCAACCTGGGAACTGCATTCAAAACTGACTGACTAGAGTATGGTAGAGG |
| | | GTGGTGGAATTTCCTGTGTAGCGGTGAAATGCGTAGATATAGGAAGGAAC |
| | | ACCAGTGGCGAAGGCGACCACCTGGACTAATACTGACACTGAGGTGCGAA |
| | | AGCGTGGGGAGCAAACAGGATTAGATACCCTGGTAGTCCACGCCGTAAAC |
| | | GATGTCAACTAGCCGTTGGAAGCCTTGAGCTTTTAGTGGCGCAGCTAACGC |
| | | ATTAAGTTGACCGCCTGGGGAGTACGGCCGCAAGGTTAAAACTCAAATGAA |
| | | TTGACGGGGGCCCGCACAAGCGGTGGAGCATGTGGTTTAATTCGAAGCAA |
| | | CGCGAAGAACCTTACCAGGCCTTGACATCCAATGAACTTTCTAGAGATAGA |
| | | TTGGTGCCTTCGGGAACATTGAGACAGGTGCTGCATGGCTGTCGTCAGCTC |
| | | GTGTCGTGAGATGTTGGGTTAAGTCCCGTAACGAGCGCAACCCTTGTCCTT |
| | | AGTTACCAGCACGTTATGGTGGGCACTCTAAGGAGACTGCCGGTGACAAAC |
| | | CGGAGGAAGGTGGGGATGACGTCAAGTCATCATGGCCCTTACGGCCTGGG |
| | | CTACACACGTGCTACAATGGTCGGTACAGAGGGTTGCCAAGCCGCGAGGT |
| | | GGAGCTAATCCCATAAAACCGATCGTAGTCCGGATCGCAGTCTGCAACTCG |
| | | ACTGCGTGAAGTCGGAATCGCTAGTAATCGCGAATCAGAATGTCGCGGTGA |
| | | ATACGTTCCCGGGCCTTGTACACACCGCCCGTCACACCATGGGAGTG |
| | DP1 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #5 | GGTGAGTAATGCCTAGGAATCTGCCTGGTAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCGACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGTTGTAGATT |
| | | AATACTCTGCAATTTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGGATGTGAAATCCCCGG |
| | | GCTCAACCTGGGAACTGCATTCAAAACTGACTGACTAGAGTATGGTAGAGG |
| | | GTGGTGGAATTTCCTGTGTAGCGGTGAAATGCGTAGATATAGGAAGGAAC |
| | | ACCAGTGGCGAAGGCGACCACCTGGACTAATACTGACACTGAGGTGCGAA |
| | | AGCGTGGGGAGCAAACAGGATTAGATACCCTGGTAGTCCACGCCGTAAAC |
| | | GATGTCAACTAGCCGTTGGAAGCCTTGAGCTTTTAGTGGCGCAGCTAACGC |
| | | ATTAAGTTGACCGCCTGGGGAGTACGGCCGCAAGGTTAAAACTCAAATGAA |
| | | TTGACGGGGGCCCGCACAAGCGGTGGAGCATGTGGTTTAATTCGAAGCAA |
| | | CGCGAAGAACCTTACCAGGCCTTGACATCCAATGAACTTTCTAGAGATAGA |
| | | TTGGTGCCTTCGGGAACATTGAGACAGGTGCTGCATGGCTGTCGTCAGCTC |
| | | GTGTCGTGAGATGTTGGGTTAAGTCCCGTAACGAGCGCAACCCTTGTTCTT |
| | | AGTTACCAGCACGTTATGGTGGGCACTCTAAGGAGACTGCCGGTGACAAAC |
| | | CGGAGGAAGGTGGGGATGACGTCAAGTCATCATGGCCCTTACGGCCTGGG |
| | | CTACACACGTGCTACAATGGTCGGTACAGAGGGTTGCCAAGCCGCGAGGT |
| | | GGAGCTAATCCCATAAAACCGATCGTAGTCCGGATCGCAGTCTGCAACTCG |
| | | ACTGCGTGAAGTCGGAATCGCTAGTAATCGCGAATCAGAATGTCGCGGTGA |
| | | ATACGTTCCCGGGCCTTGTACACACCGCCCGTCACACCATGGGAGTGGGTT |
| | | GCACCAGAAGTAGCTAGTCTAACCTTCGGGAGGACGGTTACCACGGTGTGA |
| | | TTCATGACTGGGGTGAAGTCGTAACAAGGTAGCCGTAGGGGAACCTGCGG |
| | | CTGGATCACCTCCTT |
| | DP1 | TGAAGAGTTTGATCATGGCTCAGATTGAACGCTGGCGGCAGGCCTAACACA |
| | Reisolate | TGCAAGTCGAGCGGTAGAGAGAAGCTTGCTTCTCTTGAGAGCGGCGGACG |
| | #6 | GGTGAGTAATGCCTAGGAATCTGCCTGGTAGTGGGGGATAACGTTCGGAA |
| | | ACGGACGCTAATACCGCATACGTCCTACGGGAGAAAGCAGGGGACCTTCG |
| | | GGCCTTGCGCTATCAGATGAGCCTAGGTCGGATTAGCTAGTTGGTGAGGTA |
| | | ATGGCTCACCAAGGCGACGATCCGTAACTGGTCTGAGAGGATGATCAGTCA |
| | | CACTGGAACTGAGACACGGTCCAGACTCCTACGGGAGGCAGCAGTGGGGA |
| | | ATATTGGACAATGGGCGAAAGCCTGATCCAGCCATGCCGCGTGTGTGAAG |
| | | AAGGTCTTCGGATTGTAAAGCACTTTAAGTTGGGAGGAAGGGTTGTAGATT |
| | | AATACTCTGCAATTTTGACGTTACCGACAGAATAAGCACCGGCTAACTCTGT |
| | | GCCAGCAGCCGCGGTAATACAGAGGGTGCAAGCGTTAATCGGAATTACTG |
| | | GGCGTAAAGCGCGCGTAGGTGGTTTGTTAAGTTGGATGTGAAATCCCCGG |
| | | GCTCAACCTGGGAACTGCATTCAAAACTGACTGACTAGAGTATGGTAGAGG |
| | | GTGGTGGAATTTCCTGTGTAGCGGTGAAATGCGTAGATATAGGAAGGAAC |
| | | ACCAGTGGCGAAGGCGACCACCTGGACTAATACTGACACTGAGGTGCGAA |
| | | AGCGTGGGGAGCAAACAGGATTAGATACCCTGGTAGTCCACGCCGTAAAC |
| | | GATGTCAACTAGCCGTTGGAAGCCTTGAGCTTTTAGTGGCGCAGCTAACGC |
| | | ATTAAGTTGACCGCCTGGGGAGTACGGCCGCAAGGTTAAAACTCAAATGAA |
| | | TTGACGGGGGCCCGCACAAGCGGTGGAGCATGTGGTTTAATTCGAAGCAA |
| | | CGCGAAGAACCTTACCAGGCCTTGACATCCAATGAACTTTCTAGAGATAGA |
| | | TTGGTGCCTTCGGGAACATTGAGACAGGTGCTGCATGGCTGTCGTCAGCTC |
| | | GTGTCGTGAGATGTTGGGTTAAGTCCCGTAACGAGCGCAACCCTTGTTCTT |
| | | AGTTACCAGCACGTTATGGTGGGCACTCTAAGGAGACTGCCGGTGACAAAC |
| | | CGGAGGAAGGTGGGGATGACGTCAAGTCATCATGGCCCTTACGGCCTGGG |
| | | CTACACACGTGCTACAATGGTCGGTACAGAGGGTTGCCAAGCCGCGAGGT |
| | | GGAGCTAATCCCATAAAACCGATCGTAGTCCGGATCGCAGTCTGCAACTCG |
| | | ACTGCGTGAAGTCGGAATCGCTAGTAATCGCGAATCAGAATGTCGCGGTGA |
| | | ATACGTTCCCGGGCCTTGTACACACCGCCCGTCACACCATGGGAGTGGGTT |
| | | GCACCAGAAGTAGCTAGTCTAACCTTCGGGAGGACGGTTACCACGGTGTGA |
| | | TTCATGACTGGGGTGAAGTCGTAACAAGGTAGCCGTAGGGGAACCTGCGG |
| | | CTGGATCACCTCCTT |
| | DP22 | TTGAAGAGTTTGATCATGGCTCAGATTGAACGCTGGCGGCAGGCCTAACAC |
| | Reisolate | ATGCAAGTCGAGCGGCAGCGGGAAGTAGCTTGCTACTTTGCCGGCGAGCG |
| | #1 | GCGGACGGGTGAGTAATGTCTGGGAAACTGCCTGATGGAGGGGGATAACT |
| | | ACTGGAAACGGTAGCTAATACCGCATGACCTCGCAAGAGCAAAGTGGGGG |
| | | ACCTTCGGGCCTCACGCCATCGGATGTGCCCAGATGGGATTAGCTAGTAGG |
| | | TGAGGTAATGGCTCACCTAGGCGACGATCCCTAGCTGGTCTGAGAGGATG |
| | | ACCAGCCACACTGGAACTGAGACACGGTCCAGACTCCTACGGGAGGCAGC |
| | | AGTGGGGAATATTGCACAATGGGCGCAAGCCTGATGCAGCCATGCCGCGT |
| | | GTGTGAAGAAGGCCTTAGGGTTGTAAAGCACTTTCAGCGAGGAGGAAGGG |
| | | TTCAGTGTTAATAGCACTGTGCATTGACGTTACTCGCAGAAGAAGCACCGG |
| | | CTAACTCCGTGCCAGCAGCCGCGGTAATACGGAGGGTGCAAGCGTTAATCG |
| | | GAATTACTGGGCGTAAAGCGCACGCAGGCGGTTTGTTAAGTCAGATGTGA |
| | | AATCCCCGAGCTTAACTTGGGAACTGCATTTGAAACTGGCAAGCTAGAGTC |
| | | TTGTAGAGGGGGGTAGAATTCCAGGTGTAGCGGTGAAATGCGTAGAGATC |
| | | TGGAGGAATACCGGTGGCGAAGGCGGCCCCCTGGACAAAGACTGACGCTC |
| | | AGGTGCGAAAGCGTGGGGAGCAAACAGGATTAGATACCCTGGTAGTCCAC |
| | | GCTGTAAACGATGTCGACTTGGAGGTTGTGCCCTTGAGGCGTGGCTTCCGG |
| | | AGCTAACGCGTTAAGTCGACCGCCTGGGGAGTACGGCCGCAAGGTTAAAA |
| | | CTCAAATGAATTGACGGGGGCCCGCACAAGCGGTGGAGCATGTGGTTTAA |
| | | TTCGATGCAACGCGAAGAACCTTACCTACTCTTGACATCCAGAGAATTCGCT |
| | | AGAGATAGCTTAGTGCCTTCGGGAACTCTGAGACAGGTGCTGCATGGCTGT |
| | | CGTCAGCTCGTGTTGTGAAATGTTGGGTTAAGTCCCGCAACGAGCGCAACC |
| | | CTTATCCTTTGTTGCCAGCACGTAATGGTGGGAACTCAAAGGAGACTGCCG |
| | | GTGATAAACCGGAGGAAGGTGGGGATGACGTCAAGTCATCATGGCCCTTA |
| | | CGAGTAGGGCTACACACGTGCTACAATGGCATATACAAAGAGAAGCGAAC |
| | | TCGCGAGAGCAAGCGGACCTCATAAAGTATGTCGTAGTCCGGATTGGAGTC |
| | | TGCAACTCGACTCCATGAAGTCGGAATCGCTAGTAATCGTAGATCAGAATG |
| | | CTACGGTGAATACGTTCCCGGGCCTTGTACACACCGCCCGTCACACCATGG |
| | | GAGTGGGTTGCAAAAGAAGTAGGTAGCTTAACCTTCGGGAGGGCGCTTAC |
| | | CACTTTGTGATTCATGACTGGGGTGAAGTCGTAACAAGGTAACCGTAGGGG |
| | | AACCTGCGGTTGGATCACCTCCTT |
| | DP22 | TGACGAGCGGCGGACGGGTGAGTAATGTCTGGGAAACTGCCTGATGGAG |
| | Reisolate | GGGGATAACTACTGGAAACGGTAGCTAATACCGCATGACGTCGCAAGACC |
| | #2 | AAAGTGGGGGACCTTCGGGCCTCACGCCATCGGATGTGCCCAGATGGGAT |
| | | TAGCTAGTAGGTGAGGTAATGGCTCACCTAGGCGACGATCCCTAGCTGGTC |
| | | TGAGAGGATGACCAGCCACACTGGAACTGAGACACGGTCCAGACTCCTAC |
| | | GGGAGGCAGCAGTGGGGAATATTGCACAATGGGCGCAAGCCTGATGCAGC |
| | | CATGCCGCGTGTGTGAAGAAGGCCTTAGGGTTGTAAAGCACTTTCAGCGAG |
| | | GAGGAAGGCGTTGCAGTTAATAGCTGCAACGATTGACGTTACTCGCAGAA |
| | | GAAGCACCGGCTAACTCCGTGCCAGCAGCCGCGGTAATACGGAGGGTGCA |
| | | AGCGTTAATCGGAATTACTGGGCGTAAAGCGCACGCAGGCGGTTTGTTAA |
| | | GTCAGATGTGAAATCCCCGAGCTTAACTTGGGAACTGCATTTGAAACTGGC |
| | | AAGCTAGAGTCTTGTAGAGGGGGGTAGAATTCCAGGTGTAGCGGTGAAAT |
| | | GCGTAGAGATCTGGAGGAATACCGGTGGCGAAGGCGGCCCCCTGGACAAA |
| | | GACTGACGCTCAGGTGCGAAAGCGTGGGGAGCAAACAGGATTAGATACCC |
| | | TGGTAGTCCACGCTGTAAACGATGTCGACTTGGAGGTTGTGCCCTTGAGGC |
| | | GTGGCTTCCGGAGCTAACGCGTTAAGTCGACCGCCTGGGGAGTACGGCCG |
| | | CAAGGTTAAAACTCAAATGAATTGACGGGGGCCCGCACAAGCGGTGGAGC |
| | | ATGTGGTTTAATTCGATGCAACGCGAAGAACCTTACCTACTCTTGACATCCA |
| | | GAGAATTCGCTAGAGATAGCTTAGTGCCTTCGGGAACTCTGAGACAGGTGC |
| | | TGCATGGCTGTCGTCAGCTCGTGTTGTGAAATGTTGGGTTAAGTCCCGCAA |
| | | CGAGCGCAACCCTTATCCTTTGTTGCCAGCACGTAATGGTGGGAACTCAAA |
| | | GGAGACTGCCGGTGATAAACCGGAGGAAGGTGGGGATGACGTCAAGTCAT |
| | | CATGGCCCTTACGAGTAGGGCTACACACGTGCTACAATGGCATATACAAAG |
| | | AGAAGCGAACTCGCGAGAGCAAGCGGACCTCATAAAGTATGTCGTAGTCC |
| | | GGATTGGAGTCTGCAACTCGACTCCATGAAGTCGGAATCGCTAGTAATCGT |
| | | AGATCAGAATGCTACGGTGAATACGTTCCCGGGCCTTGTACACACCGCCCG |
| | | TCACACCATGGGAGTGGGTTGCAAAAGAAGTAGGTAGCTTAACCTTCGGG |
| | | AGGGCGCTTACCACTTTGTGATTCATGACTGGGGTGAAGTCGTAACAAGGT |
| | | AACCGTAGGGGAACCTGCGGTTGGATCACCTCCTT |
| | DP22 | TTGAAGAGTTTGATCATGGCTCAGATTGAACGCTGGCGGCAGGCCTAACAC |
| | Reisolate | ATGCAAGTCGAGCGGTAGCACAGGAGAGCTTGCTCTCCGGGTGACGAGCG |
| | #3 | GCGGACGGGTGAGTAATGTCTGGGAAACTGCCTGATGGAGGGGGATAACT |
| | | ACTGGAAACGGTAGCTAATACCGCATGATGTCGCAAGACCAAAGTGGGGG |
| | | ACCTTCGGGCCTCACGCCATCGGATGTGCCCAGATGGGATTAGCTAGTAGG |
| | | TGAGGTAATGGCTCACCTAGGCGACGATCCCTAGCTGGTCTGAGAGGATG |
| | | ACCAGCCACACTGGAACTGAGACACGGTCCAGACTCCTACGGGAGGCAGC |
| | | AGTGGGGAATATTGCACAATGGGCGCAAGCCTGATGCAGCCATGCCGCGT |
| | | GTGTGAAGAAGGCCTTAGGGTTGTAAAGCACTTTCAGCGAGGAGGAAGGC |
| | | GTTGCAGTTAATAGCTGCAACGATTGACGTTACTCGCAGAAGAAGCACCGG |
| | | CTAACTCCGTGCCAGCAGCCGCGGTAATACGGAGGGTGCAAGCGTTAATCG |
| | | GAATTACTGGGCGTAAAGCGCACGCAGGCGGTTTGTTAAGTCAGATGTGA |
| | | AATCCCCGAGCTTAACTTGGGAACTGCATTTGAAACTGGCAAGCTAGAGTC |
| | | TTGTAGAGGGGGGTAGAATTCCAGGTGTAGCGGTGAAATGCGTAGAGATC |
| | | TGGAGGAATACCGGTGGCGAAGGCGGCCCCCTGGACAAAGACTGACGCTC |
| | | AGGTGCGAAAGCGTGGGGAGCAAACAGGATTAGATACCCTGGTAGTCCAC |
| | | GCTGTAAACGATGTCGACTTGGAGGTTGTGCCCTTGAGGCGTGGCTTCCGG |
| | | AGCTAACGCGTTAAGTCGACCGCCTGGGGAGTACGGCCGCAAGGTTAAAA |
| | | CTCAAATGAATTGACGGGGGCCCGCACAAGCGGTGGAGCATGTGGTTTAA |
| | | TTCGATGCAACGCGAAGAACCTTACCTACTCTTGACATCCAGAGAATTCGCT |
| | | AGAGATAGCTTAGTGCCTTCGGGAACTCTGAGACAGGTGCTGCATGGCTGT |
| | | CGTCAGCTCGTGTTGTGAAATGTTGGGTTAAGTCCCGCAACGAGCGCAACC |
| | | CTTATCCTTTGTTGCCAGCACGTAATGGTGGGAACTCAAAGGAGACTGCCG |
| | | GTGATAAACCGGAGGAAGGTGGGGATGACGTCAAGTCATCATGGCCCTTA |
| | | CGAGTAGGGCTACACACGTGCTACAATGGCATATACAAAGAGAAGCGAAC |
| | | TCGCGAGAGCAAGCGGACCTCATAAAGTATGTCGTAGTCCGGATTGGAGTC |
| | | TGCAACTCGACTCCATGAAGTCGGAATCGCTAGTAATCGTAGATCAGAATG |
| | | CTACGGTGAATACGTTCCCGGGCCTTGTA |
| | DP22 | CGAGCGGCGGACGGGTGAGTAATGTCTGGGAAACTGCCTGATGGAGGGG |
| | Reisolate | GATAACTACTGGAAACGGTAGCTAATACCGCATGACGTCGCAAGACCAAAG |
| | #4 | TGGGGGACCTTCGGGCCTCACGCCATCGGATGTGCCCAGATGGGATTAGCT |
| | | AGTAGGTGAGGTAATGGCTCACCTAGGCGACGATCCCTAGCTGGTCTGAG |
| | | AGGATGACCAGCCACACTGGAACTGAGACACGGTCCAGACTCCTACGGGA |
| | | GGCAGCAGTGGGGAATATTGCACAATGGGCGCAAGCCTGATGCAGCCATG |
| | | CCGCGTGTGTGAAGAAGGCCTTAGGGTTGTAAAGCACTTTCAGCGAGGAG |
| | | GAAGGCGTTGCAGTTAATAGCTGCAGCGATTGACGTTACTCGCAGAAGAA |
| | | GCACCGGCTAACTCCGTGCCAGCAGCCGCGGTAATACGGAGGGTGCAAGC |
| | | GTTAATCGGAATTACTGGGCGTAAAGCGCACGCAGGCGGTTTGTTAAGTCA |
| | | GATGTGAAATCCCCGAGCTTAACTTGGGAACTGCATTTGAAACTGGCAAGC |
| | | TAGAGTCTTGTAGAGGGGGGTAGAATTCCAGGTGTAGCGGTGAAATGCGT |
| | | AGAGATCTGGAGGAATACCGGTGGCGAAGGCGGCCCCCTGGACAAAGACT |
| | | GACGCTCAGGTGCGAAAGCGTGGGGAGCAAACAGGATTAGATACCCTGGT |
| | | AGTCCACGCTGTAAACGATGTCGACTTGGAGGTTGTGCCCTTGAGGCGTGG |
| | | CTTCCGGAGCTAACGCGTTAAGTCGACCGCCTGGGGAGTACGGCCGCAAG |
| | | GTTAAAACTCAAATGAATTGACGGGGGCCCGCACAAGCGGTGGAGCATGT |
| | | GGTTTAATTCGATGCAACGCGAAGAACCTTACCTACTCTTGACATCCAGAGA |
| | | ATTCGCTAGAGATAGCTTAGTGCCTTCGGGAACTCTGAGACAGGTGCTGCA |
| | | TGGCTGTCGTCAGCTCGTGTTGTGAAATGTTGGGTTAAGTCCCGCAACGAG |
| | | CGCAACCCTTATCCTTTGTTGCCAGCACGTAATGGTGGGAACTCAAAGGAG |
| | | ACTGCCGGTGATAAACCGGAGGAAGGTGGGGATGACGTCAAGTCATCATG |
| | | GCCCTTACGAGTAGGGCTACACACGTGCTACAATGGCATATACAAAGAGAA |
| | | GCGAACTCGCGAGAGCAAGCGGACCTCATAAAGTATGTCGTAGTCCGGATT |
| | | GGAGTCTGCAACTCGACTCCATGAAGTCGGAATCGCTAGTAATCGTAGATC |
| | | AGAATGCTACGGTGAATACGTTCCCGGGCCTTGTACACACCGCCCGTCACA |
| | | CCATGGGAGTGGGTTGCAAAAGAAGTAGGTAGCTTAACCTTCGGGAGGGC |
| | | GCTTACCACTTTGTGATTCATGACTGGGGTGAAGTCGTAACAAGGTAACCG |
| | | TAGGGGAACCTGCGGTTGGATCACCTCCTT |
| | DP22 | GTAATGTCTGGGAAACTGCCTGATGGAGGGGGATAACTACTGGAAACGGT |
| | Reisolate | AGCTAATACCGCATGATGTCGCAAGACCAAAGTGGGGGACCTTCGGGCCTC |
| | #5 | ACGCCATCGGATGTGCCCAGATGGGATTAGCTAGTAGGTGAGGTAATGGC |
| | | TCACCTAGGCGACGATCCCTAGCTGGTCTGAGAGGATGACCAGCCACACTG |
| | | GAACTGAGACACGGTCCAGACTCCTACGGGAGGCAGCAGTGGGGAATATT |
| | | GCACAATGGGCGCAAGCCTGATGCAGCCATGCCGCGTGTGTGAAGAAGGC |
| | | CTTAGGGTTGTAAAGCACTTTCAGCGAGGAGGAAGGCGTTGCAGTTAATA |
| | | GCTGCAACGATTGACGTTACTCGCAGAAGAAGCACCGGCTAACTCCGTGCC |
| | | AGCAGCCGCGGTAATACGGAGGGTGCAAGCGTTAATCGGAATTACTGGGC |
| | | GTAAAGCGCACGCAGGCGGTTTGTTAAGTCAGATGTGAAATCCCCGAGCTT |
| | | AACTTGGGAACTGCATTTGAAACTGGCAAGCTAGAGTCTTGTAGAGGGGG |
| | | GTAGAATTCCAGGTGTAGCGGTGAAATGCGTAGAGATCTGGAGGAATACC |
| | | GGTGGCGAAGGCGGCCCCCTGGACAAAGACTGACGCTCAGGTGCGAAAGC |
| | | GTGGGGAGCAAACAGGATTAGATACCCTGGTAGTCCACGCTGTAAACGAT |
| | | GTCGACTTGGAGGTTGTGCCCTTGAGGCGTGGCTTCCGGAGCTAACGCGTT |
| | | AAGTCGACCGCCTGGGGAGTACGGCCGCAAGGTTAAAACTCAAATGAATT |
| | | GACGGGGGCCCGCACAAGCGGTGGAGCATGTGGTTTAATTCGATGCAACG |
| | | CGAAGAACCTTACCTACTCTTGACATCCAGAGAATTCGCTAGAGATAGCTTA |
| | | GTGCCTTCGGGAACTCTGAGACAGGTGCTGCATGGCTGTCGTCAGCTCGTG |
| | | TTGTGAAATGTTGGGTTAAGTCCCGCAACGAGCGCAACCCTTATCCTTTGTT |
| | | GCCAGCACGTAATGGTGGGAACTCAAAGGAGACTGCCGGTGATAAACCGG |
| | | AGGAAGGTGGGGATGACGTCAAGTCATCATGGCCCTTACGAGTAGGGCTA |
| | | CACACGTGCTACAATGGCATATACAAAGAGAAGCGAACTCGCGAGAGCAA |
| | | GCGGACCTCATAAAGTATGTCGTAGTCCGGATTGGAGTCTGCAACTCGACT |
| | | CCATGAAGTCGGAATCGCTAGTAATCGTAGATCAGAATGCTACGGTGAATA |
| | | CGTTCCCGGGCCTTGTACACACCGCCCGTCACACCATGGGAGTGGGTTGCA |
| | | AAAGAAGTAGGTAGCTTAACCTTCGGGAGGGCGCTTACCACTTTGTGATTC |
| | | ATGACTGGGGTGAAGTCGTAACAAGGTAACCGTAGGGGAACCTGCGGTTG |
| | | GATCACCTCCTT |
| | DP22 | TTGAAGAGTTTGATCATGGCTCAGATTGAACGCTGGCGGCAGGCCTAACAC |
| | Reisolate | ATGCAAGTCGAGCGGTAGCACAGGAGAGCTTGCTCTCCGGGTGACGAGCG |
| | #6 | GCGGACGGGTGAGTAATGTCTGGGAAACTGCCTGATGGAGGGGGATAACT |
| | | ACTGGAAACGGTAGCTAATACCGCATGACGTCGCAAGACCAAAGTGGGGG |
| | | ACCTTCGGGCCTCACGCCATCGGATGTGCCCAGATGGGATTAGCTAGTAGG |
| | | TGAGGTAATGGCTCACCTAGGCGACGATCCCTAGCTGGTCTGAGAGGATG |
| | | ACCAGCCACACTGGAACTGAGACACGGTCCAGACTCCTACGGGAGGCAGC |
| | | AGTGGGGAATATTGCACAATGGGCGCAAGCCTGATGCAGCCATGCCGCGT |
| | | GTGTGAAGAAGGCCTTAGGGTTGTAAAGCACTTTCAGCGAGGAGGAAGGC |
| | | GTTGCAGTTAATAGCTGCAGCGATTGACGTTACTCGCAGAAGAAGCACCGG |
| | | CTAACTCCGTGCCAGCAGCCGCGGTAATACGGAGGGTGCAAGCGTTAATCG |
| | | GAATTACTGGGCGTAAAGCGCACGCAGGCGGTTTGTTAAGTCAGATGTGA |
| | | AATCCCCGAGCTTAACTTGGGAACTGCATTTGAAACTGGCAAGCTAGAGTC |
| | | TTGTAGAGGGGGGTAGAATTCCAGGTGTAGCGGTGAAATGCGTAGAGATC |
| | | TGGAGGAATACCGGTGGCGAAGGCGGCCCCCTGGACAAAGACTGACGCTC |
| | | AGGTGCGAAAGCGTGGGGAGCAAACAGGATTAGATACCCTGGTAGTCCAC |
| | | GCTGTAAACGATGTCGACTTGGAGGTTGTGCCCTTGAGGCGTGGCTTCCGG |
| | | AGCTAACGCGTTAAGTCGACCGCCTGGGGAGTACGGCCGCAAGGTTAAAA |
| | | CTCAAATGAATTGACGGGGGCCCGCACAAGCGGTGGAGCATGTGGTTTAA |
| | | TTCGATGCAACGCGAAGAACCTTACCTACTCTTGACATCCAGAGAATTCGCT |
| | | AGAGATAGCTTAGTGCCTTCGGGAACTCTGAGACAGGTGCTGCATGGCTGT |
| | | CGTCAGCTCGTGTTGTGAAATGTTGGGTTAAGTCCCGCAACGAGCGCAACC |
| | | CTTATCCTTTGTTGCCAGCACGTAATGGTGGGAACTCAAAGGAGACTGCCG |
| | | GTGATAAACCGGAGGAAGGTGGGGATGACGTCAAGTCATCATGGCCCTTA |
| | | CGAGTAGGGCTACACACGTGCTACAATGGCATATACAAAGAGAAGCGAAC |
| | | TCGCGAGAGCAAGCGGACCTCATAAAGTATGTCGTAGTCCGGATTGGAGTC |
| | | TGCAACTCGACTCCATGAAGTCGGAATCGCTAGTAATCGTAGATCAGAATG |
| | | CTACGGTGAATACGTTCCCGGGCCTTGTACACACCGCCCGTCACACCATGG |
| | | GAGTGGGTTGCAAAAGAAGTAGGTAGCTTAACCTTCGGGAGGGCGCTTAC |
| | | CACTTTGTGATTCATGACTGGGGTGAAGTCGTAACAAGGTAACCGTAGGGG |
| | | AACCTGCGGTTGGATCACCTCCTT |

### Example 4: Computation of Microbial Entity Average Nucleotide Identity (ANI)

We applied a whole-genome based method, the average nucleotide identity (ANI), to estimate the genetic relatedness among bacterial genomes and profile hundreds of microbial species at a higher resolution taxonomic level (i.e., species- and strain-level classification). ANI is based on the average of the nucleotide identity of all orthologous genes shared between a genome pair. Genomes of the same species present ANI values above 95% and of the same genus values above 80% (Jain et al. 2018).

Taxonomic annotation of the strains combined into DMAs using ANI and the NCBI RefSeq database indicated that these microbes represent species not present in the database and most likely are new bacterial species (Table G). Multiple independent isolates were obtained for all of DP1, DP3, DP9, DP22, and DP53, suggesting that it is well within the level of ordinary skill of one in the art to isolate these species following the teachings of this specification (16S sequence alignment identity of the multiple isolates is shown in Table G.1). The successful isolation of these species can be determined by 16S sequence comparison to the reference sequences of these species provided in Table F. In other embodiments, a person of ordinary skill can determine that substitutions for these novel species may be made using either or both of the most closely matching species set out in Table G, either by 16S or ANI sequence comparison. Further it is within the level of ordinary skill to distinguish operable from inoperable substitutions by assembling a substituted DMA and assaying for any one of the activities set forth, e.g., in any one of the working examples provided in this specification.

TABLE G
Predictive power of Average Nucleotide Identity (ANI) analysis.
ANI analysis demonstrates that the overall genome sequence
of the microbial entities isolated from plants and described
herein as compared to reference strains is different enough
in many cases to qualify as a different species.

| | | 16S | | |
|---|---|---|---|---|
| | | rRNA | | |
| | | gene | Closest Reference | ANI |
| ID | NCBI match | (%) | genome at NCBI | (%) |
| DP3 | Leuconostoc | 99 | Leuconostoc | 91.77 |
| | mesenteroides | | pseudomesenteroides | |
| | (NR_074957.1.) | | (JDVA01000001.1.) | |
| DP9 | Pediococcus | 99 | Pediococcus pentosauceus | 99.6 |
| | pentosauceus | | (NC_022780.1.) | |
| | (NR_042058.1.) | | | |
| DP53 | Pseudomonas | 99 | Pseudomonas psychrophile | 86.82 |
| | helleri | | (NZ_LT629795.1.) | |
| | (NR_148763.1.) | | | |
| DP1 | Pseudomonas | 99 | Pseudomonas antarctica | 94.48 |
| | fluorescens | | (NZ_CP015600.1.) | |
| | (NR_115715.1.) | | | |
| DP22 | Rahnella aquatilis | 98 | Rahnella sp. | 88.31 |
| | (NR_025337.1) | | (NC_015061.1.) | |

TABLE G.1
Sequence Identity of Additional Isolates

| | | | 16S rRNA gene |
|---|---|---|---|
| ID | Reisolate # | Strain Name | (% Identity) |
| DP3 | 1 | Leuconostoc | 100 |
| | 2 | mesenteroides | 99.55 |
| | 3 | | 99.07 |
| | 4 | | 99 |
| DP9 | 1 | Pediococcus | 100 |
| | 2 | pentosauceus | 100 |
| | 3 | | 100 |
| | 4 | | 100 |
| | 5 | | 100 |
| | 6 | | 97.38 |
| DP53 | 1 | Pseudomonas | 100 |
| | 2 | fragi | 99.36 |
| | 3 | | 100 |
| | 4 | | 99.64 |
| | 5 | | 99.79 |
| | 6 | | 99.62 |
| DP1 | 1 | Pseudomonas | 98.89 |
| | 2 | fluorescens | 98.89 |
| | 3 | | 98.96 |
| | 4 | | 98.87 |
| | 5 | | 100 |
| | 6 | | 98.89 |
| DP22 | 1 | Rahnella sp. | 98.38 |
| | 2 | | 99.93 |
| | 3 | | 99.86 |
| | 4 | | 100 |
| | 5 | | 99.86 |
| | 6 | | 100 |

Alignment with respect to original isolate

### Example 5: Methods of Plant Inoculation

Seed Disinfection by Chlorine Gas: Seeds can be surface-disinfected prior to inoculation by a modified the technique described for Arabidopsis seeds (Lindsey et al. “Standardized Method for High-throughput Sterilization of Arabidopsis Seeds.” 2017. Jove). Seeds are placed within sterile containers and placed within an airtight jar inside of a chemical fume hood. A 250 ml bottle containing 200 ml bleach is added to the jar. 4 ml of 12N HCl is added to the bottle to generate the chlorine gas. The jar is sealed, and the seeds are incubated in the gas for 2-3 hrs before being ventilated inside the fume hood and then removed and kept in sterile containers.

Seed Treatment: A complex, fungal, or bacterial endophyte is inoculated onto seeds as a liquid or powder using a range of formulations including the following components: sodium alginate and/or methyl cellulose as stickers, talc and flowability polymers. Seeds are air dried after treatment and planted according to common practice for each crop type.

Seed Inoculation: Debaryomyces hansenii DP5, Pichia kudriavzevii DP102, Pseudomonas fluorescens DP1, Lactobacillus plantarum DP100, Lactobacillus brevis DP94, Lactococcus garvieae DP97, Lactobacillus paracasei DP95 and Leuconostoc mesenteroides DP93 are grown in appropriate medium, aerobically or anaerobically, at 30° C. or 37° C. depending on the strain. Strains are selected based on their known use as commercial probiotics, their safe use in human health and nutrition, and having originated from plant tissues. The strains are sourced from the samples as described in Example 1 based on predicted beneficial functionalities as described in Example 2. A fundamental feature for the selection of the strains and their testing as seed coatings is that the colonization should not result in yield drag as is the case in some agricultural products, but instead to serve as a plant growth promoting treatment. This results in a duality of product benefit for facilitating farming, improving yield by providing some type of stress resilience to the crop, and providing improved nutrition by the consumption of fresh plant products enriched in probiotic flora. This microbial benefit goes above the observed increased colonization and microbial diversity observed in organic products compared to the conventional equivalent product treated with agrochemicals.

Another important practice in vegetable farming are seed coatings with agrochemicals or microbes such as is the case with Rhizobium for legumes. In embodiments where a seed coat polymer was used (Ashland Seed Coating Polymer: Agrimer VA 6W, product number 847943), it is diluted 1:5 in sterile water and vortexed to mix. Cultures were diluted to the appropriate concentrations in either water or polymer solution to achieve 1×10⁵-1×10⁷ CFU/seed inoculum. Dilution calculations are based on either OD600 measurements or direct enumeration via Quantom TXTM (Logos biosystems). DMA preparations are generated by combining two or more microbes in a single treatment (See Table H) for a description of each DMA). Mock treatments are generated by adding an equivalent amount of sterile culture medium to the water or polymer solution to replace microbes. Seeds are incubated in sterile tubes containing the diluted microbes and water or polymer for 20 minutes, after which time they are removed and potted.

TABLE H
Strain composition for tested DMAs.

| DMA # | DP Composition | Genus | Species |
|---|---|---|---|
| DMA #1 | DP1 | Pseudomonas | fluorescens |
| | DP102 | Pichia | kudriavzevii |
| | DP100 | Lactobacillus | plantarum |
| | DP93 | Leuconostoc | mesenteroides |
| | DP94 | Lactobacillus | brevis |
| DMA #2 | DP102 | Pichia | kudriavzevii |
| | DP100 | Lactobacillus | plantarum |
| | DP93 | Leuconostoc | mesenteroides |
| | DP94 | Lactobacillus | brevis |
| DMA #3 | DP93 | Leuconostoc | mesenteroides |
| | DP5 | Debaromyces | hansenii |
| DMA #4 | DP94 | Lactobacillus | brevis |
| | DP5 | Debaromyces | hansenii |
| DMA #5 | DP100 | Lactobacillus | plantarum |
| | DP102 | Pichia | kudriavzevii |
| DMA #6 | DP95 | Lactobacillus | paracasei |
| | DP102 | Pichia | kudriavzevii |

Osmopriming and Hydropriming: A complex, fungal, or bacterial endophyte is inoculated onto seeds during the osmopriming (soaking in polyethylene glycol solution to create a range of osmotic potentials) and/or hydropriming (soaking in de-chlorinated water) process. Osmoprimed seeds are soaked in a polyethylene glycol solution containing a bacterial and/or fungal endophyte for one to eight days and then air dried for one to two days. Hydroprimed seeds are soaked in water for one to eight days containing a bacterial and/or fungal endophyte and maintained under constant aeration to maintain a suitable dissolved oxygen content of the suspension until removal and air drying for one to two days. Talc and or flowability polymer are added during the drying process.

Foliar Application: A complex, fungal, or bacterial endophyte is inoculated onto aboveground plant tissue (leaves and stems) as a liquid suspension in dechlorinated water containing adjuvants, sticker-spreaders and UV protectants. The suspension is sprayed onto crops with a boom or other appropriate sprayer.

Soil Inoculation: A complex, fungal, or bacterial endophyte is inoculated onto soils in the form of a liquid suspension either; pre-planting as a soil drench, during planting as an in furrow application, or during crop growth as a side-dress. A fungal or bacterial endophyte is mixed directly into a fertigation system via drip tape, center pivot or other appropriate irrigation system.

Hydroponic and Aeroponic Inoculation: A complex, fungal, or bacterial endophyte is inoculated into a hydroponic or acroponic system either as a powder or liquid suspension applied directly to the rockwool substrate or applied to the circulating or sprayed nutrient solution.

Vector-Mediated Inoculation: A complex, fungal, or bacterial endophyte is introduced in power form in a mixture containing talc or other bulking agent to the entrance of a bechive (in the case of bee-mediation) or near the nest of another pollinator (in the case of other insects or birds. The pollinators pick up the powder when exiting the hive and deposit the inoculum directly to the crop's flowers during the pollination process.

Root Wash: The exterior surface of a plant's roots are contacted with a liquid inoculant formulation containing a purified bacterial population, a purified fungal population, a purified complex endophyte population, or a mixture of any of the preceding. The plant's roots are briefly passed through standing liquid microbial formulation or liquid formulation is liberally sprayed over the roots, resulting in both physical removal of soil and microbial debris from the plant roots, as well as inoculation with microbes in the formulation.

Seedling Soak: The exterior surfaces of a seedling are contacted with a liquid inoculant formulation containing a purified bacterial population, a purified fungal population, or a mixture of any of the preceding. The entire seedling is immersed in standing liquid microbial formulation for at least 30 seconds, resulting in both physical removal of soil and microbial debris from the plant roots, as well as inoculation of all plant surfaces with microbes in the formulation. Alternatively, the seedling can be germinated from seed in or transplanted into media soaked with the microbe(s) of interest and then allowed to grow in the media, resulting in soaking of the plantlet in microbial formulation for much greater time totaling as much as days or weeks. Endophytic microbes likely need time to colonize and enter the plant, as they explore the plant surface for cracks or wounds to enter, so the longer the soak, the more likely the microbes will successfully be installed in the plant.

Wound Inoculation: The wounded surface of a plant is contacted with a liquid or solid inoculant formulation containing a purified bacterial population, a purified fungal population, or a mixture of any of the preceding. Plant surfaces are designed to block entry of microbes into the endosphere, since pathogens attempting to infect plants in this way. In order to introduce beneficial endophytic microbes to plant endospheres, a way to access the interior of the plant is needed, which we can do by opening a passage by wounding. This wound takes a number of forms, including pruned roots, pruned branches, puncture wounds in the stem breaching the bark and cortex, puncture wounds in the tap root, puncture wounds in leaves, and puncture wounds seed allowing entry past the seed coat. Wounds are made using needles, hammer and nails, knives, drills, etc. Into the wound are then contacted with the microbial inoculant as liquid, as powder, inside gelatin capsules, in a pressurized capsule injection system, in a pressurized reservoir and tubing injection system, allowing entry and colonization by microbes into the endosphere. Alternatively, the entire wounded plant is soaked or washed in the microbial inoculant for at least 30 seconds, giving more microbes a chance to enter the wound, as well as inoculating other plant surfaces with microbes in the formulation—for example pruning seedling roots and soaking them in inoculant before transplanting is a very effective way to introduce endophytes into the plant.

Injection: Microbes are injected into a plant in order to successfully install them in the endosphere. Plant surfaces are designed to block entry of microbes into the endosphere, since pathogens attempting to infect plants in this way. In order to introduce beneficial endophytic microbes to endospheres, a way is needed to access the interior of the plant which we can do by puncturing the plant surface with a need and injecting microbes into the inside of the plant. Different parts of the plant are inoculated this way including the main stem or trunk, branches, tap roots, seminal roots, buttress roots, and even leaves. The injection is made with a hypodermic needle, a drilled hole injector, or a specialized injection system. Through the puncture wound the microbial inoculant as liquid, as powder, inside gelatin capsules, in a pressurized capsule injection system, in a pressurized reservoir and tubing injection system, is applied, allowing entry and colonization by microbes into the endosphere.

### Example 6: Measuring Colonization of Plants with DMA Microbes

Culturing to Confirm Colonization of Plant by Bacteria: The presence of complex endophytes in whole plants or plant elements, such as seeds, roots, leaves, or other parts, is detected by isolating microbes from plant or plant element homogenates (optionally surface-sterilized) on antibiotic-free media and identifying visually by colony morphotype and molecular methods described herein. Representative colony morphotypes are also used in colony PCR and sequencing for isolate identification via ribosomal gene sequence analysis as described herein. These trials are repeated twice per experiment, with 5 biological samples per treatment.

Culture-Independent Methods to Confirm Colonization of the Plant or Seeds by Complex Endophytes: The presence of complex endophytes on or within plants or seeds is determined by using quantitative PCR (qPCR). Internal colonization by the complex endophyte is demonstrated by using surface-sterilized plant tissue (including seed) to extract total DNA, and isolate-specific fluorescent MGB probes and amplification primers are used in a qPCR reaction. An increase in the product targeted by the reporter probe at each PCR cycle therefore causes a proportional increase in fluorescence due to the breakdown of the probe and release of the reporter. Fluorescence is measured by a quantitative PCR instrument and compared to a standard curve to estimate the number of fungal or bacterial cells within the plant.

The design of both species-specific amplification primers and isolate-specific fluorescent probes are well known in the art. Plant tissues (seeds, stems, leaves, flowers, etc.) are pre-rinsed and surface sterilized using the methods described herein: Total DNA is extracted using methods known in the art, for example using commercially available Plant-DNA extraction kits, or the following method. 1) Tissue is placed in a cold-resistant container and 10-50 mL of liquid nitrogen is applied. Tissues are then macerated to a powder. 2) Genomic DNA is extracted from each tissue preparation, following a chloroform: isoamyl alcohol 24:1 protocol (Sambrook, Joseph, Edward F. Fritsch, and Thomas Maniatis. Molecular cloning. Vol. 2. New York: Cold spring harbor laboratory press, 1989.). Quantitative PCR is performed essentially as described by Gao, Zhan, et al. Journal of clinical microbiology 48.10 (2010): 3575-3581 with primers and probe(s) specific to the desired isolate using a quantitative PCR instrument, and a standard curve is constructed by using serial dilutions of cloned PCR products corresponding to the specie-specific PCR amplicon produced by the amplification primers. Data are analyzed using instructions from the quantitative PCR instrument's manufacturer software. As an alternative to qPCR, Terminal Restriction Fragment Length Polymorphism, (TRFLP) can be performed, essentially as described in Johnston-Monje D, Raizada M N (2011) PLOS ONE 6 (6): c20396. Group specific, fluorescently labeled primers are used to amplify a subset of the microbial population, for example bacteria and fungi. This fluorescently labeled PCR product is cut by a restriction enzyme chosen for heterogeneous distribution in the PCR product population. The enzyme cut mixture of fluorescently labeled and unlabeled DNA fragments is then submitted for sequence analysis on a Sanger sequence platform such as the Applied Biosystems 3730 DNA Analyzer. Immunological Methods to Detect Complex Endophytes in Seeds and Vegetative Tissues. A polyclonal antibody is raised against specific the host fungus or bacterium via standard methods. Enzyme-linked immunosorbent assay (ELISA) and immunogold labeling is also conducted via standard methods, briefly outlined below.

Immunofluorescence microscopy procedures involve the use of semi-thin sections of seed or seedling or adult plant tissues transferred to glass objective slides and incubated with blocking buffer (20 mM Tris (hydroxymethyl)-aminomethane hydrochloride (TBS) plus 2% bovine serum albumin, pH 7.4) for 30 min at room temperature. Sections are first coated for 30 min with a solution of primary antibodies and then with a solution of secondary antibodies (goat anti-rabbit antibodies) coupled with fluorescein isothiocyanate (FITC) for 30 min at room temperature. Samples are then kept in the dark to eliminate breakdown of the light-sensitive FITC. After two 5-min washings with sterile potassium phosphate buffer (PB) (pH 7.0) and one with double-distilled water, sections are sealed with mounting buffer (100 mL 0.1 M sodium phosphate buffer (pH 7.6) plus 50 mL double-distilled glycerine) and observed under a light microscope equipped with ultraviolet light and a FITC Texas-red filter.

Ultrathin (50-to 70-nm) sections for TEM microscopy are collected on pioloform-coated nickel grids and are labeled with 15-nm gold-labeled goat anti-rabbit antibody. After being washed, the slides are incubated for 1 h in a 1:50 dilution of 5-nm gold-labeled goat anti-rabbit antibody in IGL buffer. The gold labeling is then visualized for light microscopy using a BioCell silver enhancement kit. Toluidine bluc (0.01%) is used to lightly counterstain the gold-labeled sections. In parallel with the sections used for immunogold silver enhancement, serial sections are collected on uncoated slides and stained with 1% toluidine bluc. The sections for light microscopy are viewed under an optical microscope, and the ultrathin sections are viewed by TEM.

PCR Detection of Strains: PCR probes for bacterial and fungal strains are designed using species-specific genes reported for each strain. In summary, Primer3 v 0.4.4 (bioinfo.ut.ee/primer3-0.4.0/) is used to calculate the annealing temperature and primers were constructed in the Genewiz user interface. Table I lists the specific genes, primer sequences and conditions for each probe. The PCR reaction was optimized in a final volume of 25 μL as follows: 12.5 μL of GoTaq Colorless Master Mix (Promega M7132), 2.0 μL of 10 μM Forward Primer, 2.0 μL of 10 UM Reverse Primer, 7.5 μL of molecular grade water (depending on the amount of DNA template), and 1 μL of DNA template. Genomic DNA is normalized to 2 ng/μL DNA. For plant DNA extractions, the DNEAsy Plant Pro Kit (Qiagen) was used, and PCRs were performed with 5 μL of DNA template. PCR is carried out on a thermal cycler (Eppendorf Nexus Gradient Model No. 6331) and the PCR conditions and programs are mentioned in Table I. PCR products are analyzed on a 2% agarose E-Gel (Invitrogen, USA) and visualized by UV transilluminator.

TABLE I
PCR assays to detect applied microbes onto crops.

| | | Product | | | |
|---|---|---|---|---|---|
| | | size | PCR | | |
| Species | Gene | (bp) | conditions | Primer | Sequence |
| L. plantarum | LPXTG-motif | 724 | 94° C. for | Forward | TTCGTCGGGA |
| | | | 10 min | | AGTGATGGTG |
| | | | 94° C. for | | |
| | | | 30 s | | |
| | | | 60° C. for | | |
| | | | 30 s | | |
| | | | 72° C. for | Reverse | CTTGGTCGTG |
| | | | 30 s | | GCATCAGTCT |
| | | | (35 | | |
| | | | cycles) | | |
| | | | 72° C. for | | |
| | | | 5 min | | |
| L. brevis | 16S-23S ribosomal | 558 | 94ºC for | Forward | TATGCCCATT |
| | RNA intergenic | | 5 min | | GACCGCAAGG |
| | spacer region | | 94° C. for | | |
| | | | 1 min | | |
| | | | 62ºC for | | |
| | | | 30 s | | |
| | | | 72° C. for | Reverse | AGCAAGCTTC |
| | | | 1 min | | CTGGTTTGGG |
| | | | (35 | | |
| | | | cycles) | | |
| | | | 72° C. for | | |
| | | | 5 min | | |
| Leuconostoc | metK: S- | 1,158 | 94° C. for | Forward | ATGGCAAAGT |
| mesenteroides | adenosylmethionine | | 2 min | | ATTTCACATC |
| | synthase | | 94° C. for | | GC |
| | | | 1 min | | |
| | | | 49° C. for | Reverse | TTAAAGTAAG |
| | | | 1 min | | TTTTTGATTT |
| | | | 72° C. for | | CTTTCACCTT |
| | | | 1 min | | |
| | | | (35 | | |
| | | | cycles) | | |
| | | | 72° C. for | | |
| | | | 10 min | | |
| Pichia | Saps: Secreted | 1,159 | 95° C. for | Forward | GGCGTTGTCC |
| kudriavzevii | Aspartic | | 5 mim | | ATCCAATG |
| | Proteinase | | 95° C. for | | |
| | | | 30 s | | |
| | | | 60° C. for | | |
| | | | 30 s | | |
| | | | 72° C. for | Reverse | CAGGAGAATT |
| | | | 30 s | | GCTGTTCCC |
| | | | (35 | | |
| | | | cycles) | | |
| | | | 72° C. for | | |
| | | | 8 min | | |

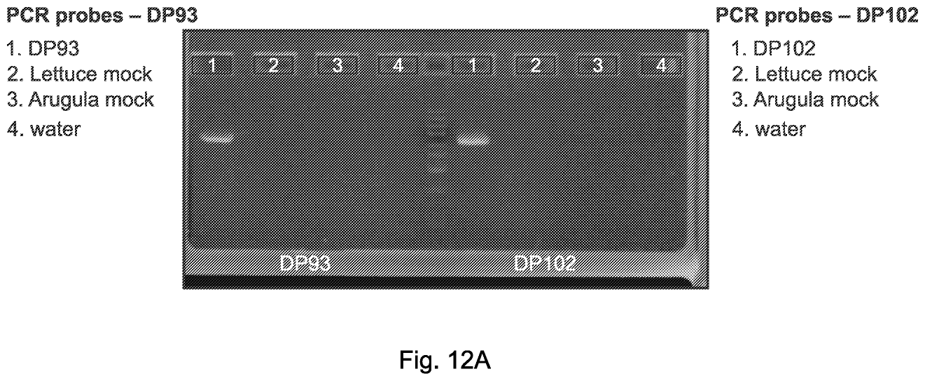

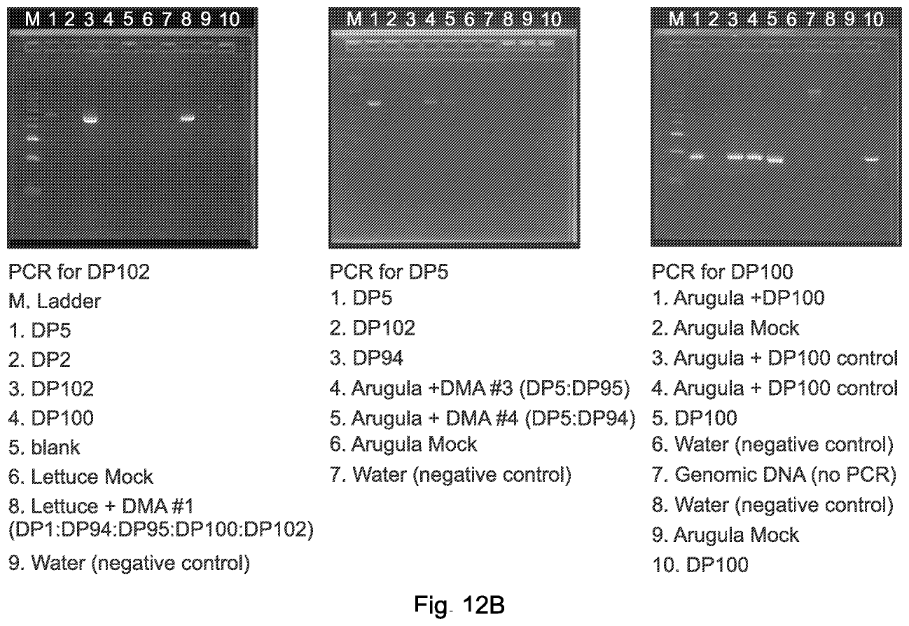

FIG. 12 provides images of PCR detection of microbes on plants using species-specific primers. FIG. 12A shows PCR assay Controls. Primers were tested against microbial genomic DNA (positive control) and each mock-treated plant type to verify primer specificity. FIG. 12B shows PCR assays for exemplary microbes tested. Primers were tested against genomic DNA from the microbe of interest and other microbes to verify specificity. On the left gel, bands are visible in the DP102 control well and the DMA #1 lettuce well. DMA #1 contains DP102. For the center gel, bands are seen with DP5 positive control and the arugula samples with DMA #3 and DMA #4 treatment, both of which contain DP5. The gel on the right demonstrates that DP100 is detected from arugula treated with DP100 as well as the positive controls. The use of PCR probes for specific strains allows to detect colonization in the plant tissues and to confirm counts based on colony forming units.

Quantitation of Microbial Colonization of Plants: Bacteria and fungi are enumerated from plants by Colony Forming Units (CFU) plating and counting. Plants are harvested, roots are removed, and the plant mass is measured. The plant is then either sectioned and reweighed or ground whole with a mortar and pestle with added PBS until complete maceration and liquefaction is achieved. A series of 10-fold dilutions are made in PBS and each dilution was plated in triplicate onto non-selective medium such as tryptic soy agar (TSA), and medium selective for the microbe of interest such as De Man, Rogosa and Sharpe agar (MRS) or potato dextrose agar containing chlorotetracycline (PDA+CTET) aerobically or anaerobically, at 30° C. or 37° C. depending on the strain-specific requirements.

The microbiological detection of strains using colony forming units allows to quantitate colonization with respect to the absolute number of cells applied to the seed or plant tissue. In addition, the colony features as well as taxonomic confirmation of the colonies resulted in a very effective way to measure colonization in treated plants and compare to mock plants where no microbes are applied. There is a very low or absent background for the target microorganism when plated in MRS agar media and incubated at 37° C. anaerobically given that most of the plant-associated microbiota grows at a lower temperature, acrobically and prefers other media formulations such as tryptic soy agar. Likewise, the use of PDA with antibiotics targeting lactic acid bacteria and others selects for the yeast applied (DP5 and DP102).

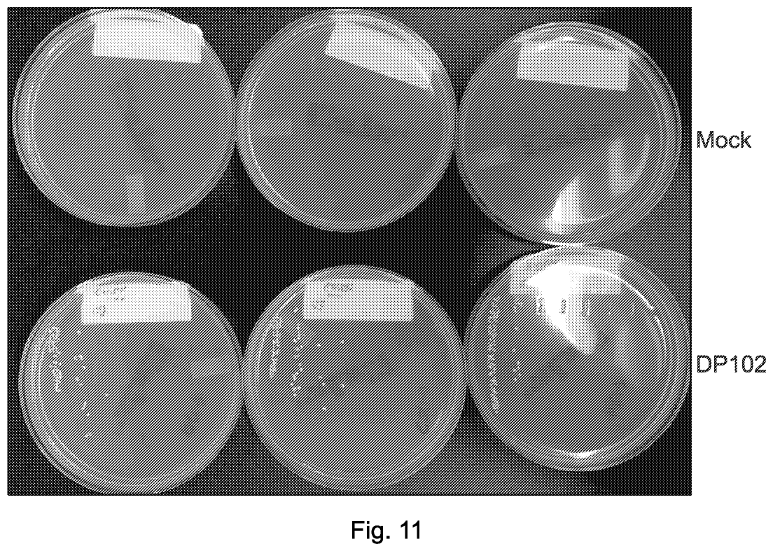

FIG. 11 depicts an example of dilution plating technique for colonization. DP102 inoculated plants (bottom) and mock treatment control (top) were diluted and plated on PDA containing chlorotetracycline. An aliquot of 5 μL for each 10-fold dilution was applied to a plate an held vertically to distribute the liquid along its length.

### Example 7: Beneficial Effects of Polymer Coating Seeds

Seed coating is widely used as a means of delivery for agriculture products. Here, seed coating was examined as a means to protect the seedling from environmental stress and to enhance colonization of seeds by the probiotic strains of interest. An oxygen permeable vinyl polymer with high adhesivity and evidence of improved rhizobia survival was selected for these experiments (Ashland Seed Coating Polymer: Agrimer VA 6W, product number 847943).

Little Gem (Johnny's Seeds Product No. 4120G.11), Black Seeded Simpson (Ferry Morse Product No. 2498), and Outredgeous (Johnny's Seeds Product No. 2208G.26), lettuce seeds and arugula (Johnny's Selected Seeds Cat no. 385.11) were disinfected and inoculated with L. plantarum DP100 L. brevis DP94, Leuconostoc mesenteroides DP93, DMA #1 or mock control with or without polymer. Seeds were planted in sterilized 36 mm peat pellets and placed within a Jiffy Seed Starting Greenhouse Kit (Ferry Morse) to germinate and grow. After 17-20 days growth, the seedlings were harvested, weighed, and colonization was assessed.

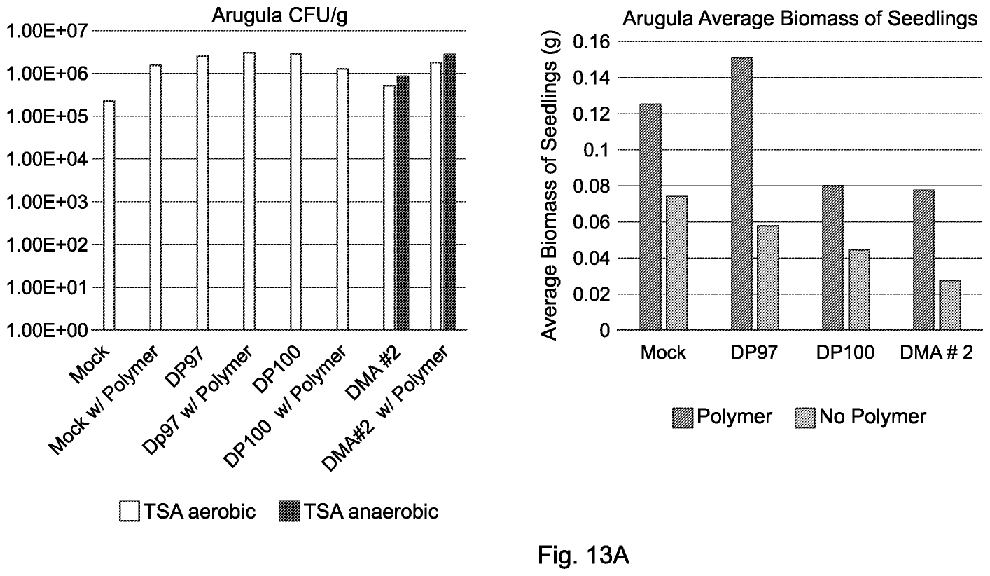

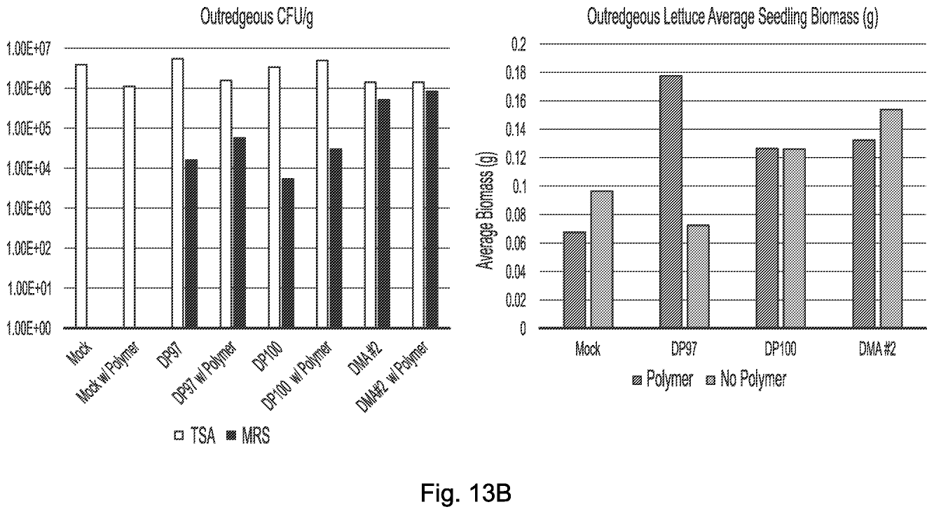

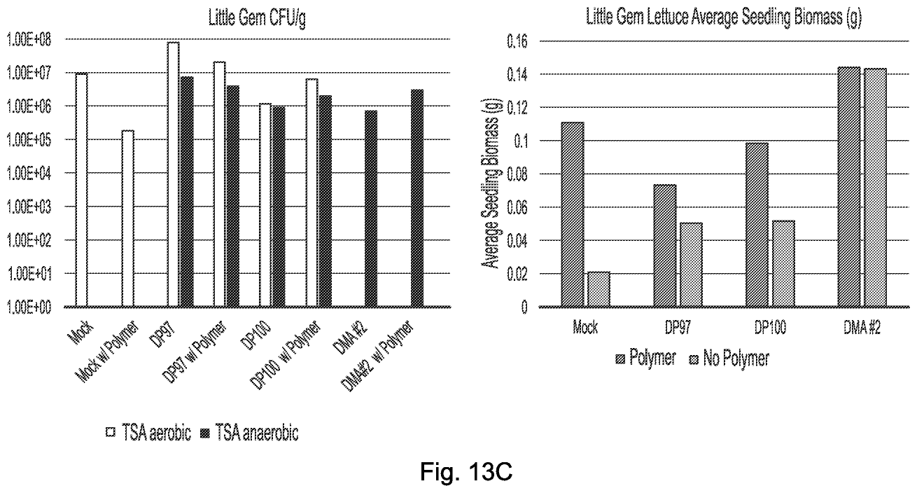

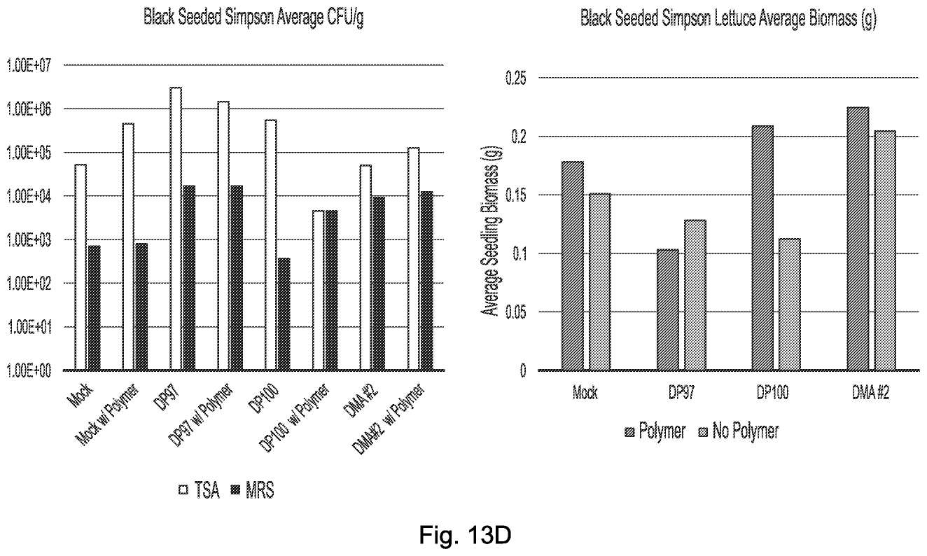

In general, colonization of the seedlings with the microbes tested was equal or greater with the addition of polymer. Most plants showed a 2-10-fold improvement in colonization when polymer was used in seed coating. In particular, DP100 exhibited the greatest benefit from polymer coating across multiple plant types. Average plant biomass and total microbial growth as measured by colony counts using TSA were improved with the addition of polymer whether or not microbes were added, suggesting that the polymer alone confers some growth advantage and when combined with the microbial treatment this advantage is amplified. For Outredgeous lettuce and arugula, the combination of DP97 and polymer conferred a greater biomass yield than the polymer alone. The same effect was seen with DMA #2 and the Little Gem and Black Seeded Simpson lettuce, suggesting some synergy between the polymer, specific plants, and specific microbes or DMAs. The selection of the best combinations will result in significantly higher agricultural yields as in the case of arugula and DP97, Outredgeous lettuce and DP97, Little Gem lettuce and DMA #2, and Black Seeded Simpson and DMA #2. It is clear there is a high degree of specificity in the crop, polymer and inoculant combination. Results are shown in FIG. 13.

FIG. 13. Demonstration of the effects of seed polymer coating in combination with microbe inoculation.

FIG. 13A Shows the effects of microbial inoculation and polymer coating on the colonization and biomass of arugula seedlings. The left graph demonstrates the level of colonization of these plants with each treatment. TSA incubated aerobically will grow microbes including endophytes natively present. Anaerobic incubation of TSA medium is selective for the microbes of interest as they are facultative anaerobes. Arugula was only colonized by DMA #2 by the end of the experiment, suggesting this DMA was capable of propagating on the plants under these conditions. The graph on the right shows the average biomass of the harvested plants. Note the strong biomass benefit seen with inoculation of polymer and DP97.

FIG. 13B Shows the effects of microbial inoculation and polymer coating on the colonization and biomass of Outredgeous lettuce seedlings. The left graph demonstrates the level of colonization of these plants with each treatment. TSA incubated aerobically will grow microbes including endophytes natively present. MRS medium incubated anaerobically is selective for the microbes of interest as they are facultative anaerobes. Outredgeous lettuce was successfully colonized by all microbial treatments, suggesting these microbes were all capable of propagating on the plants under these conditions. The graph on the right shows the average biomass of the harvested plants. Note the strong biomass benefit seen with inoculation of polymer and DP97.

FIG. 13C Shows the effects of microbial inoculation and polymer coating on the colonization and biomass of Little Gem lettuce seedlings. The left graph demonstrates the level of colonization of these plants with each treatment. TSA incubated aerobically will grow microbes including endophytes natively present. Anaerobic incubation of TSA medium is selective for the microbes of interest as they are facultative anaerobes. Little Gem lettuce was successfully colonized by all microbial treatments, suggesting these microbes were all capable of propagating on the plants under these conditions. The graph on the right shows the average biomass of the harvested plants. DMA #2 demonstrated the greatest benefit to biomass in these experiments regardless of polymer coating, indicating a specific synergy between this plant and DMA.

FIG. 13D Shows the effects of microbial inoculation and polymer coating on the colonization and biomass of Black Seeded Simpson lettuce seedlings. The left graph demonstrates the level of colonization of these plants with each treatment. TSA incubated aerobically will grow microbes including endophytes natively present. MRS medium incubated anaerobically is more selective for the microbes inoculated as they are facultative anaerobes. Outredgeous lettuce was successfully colonized by all microbial treatments, suggesting these microbes were all capable of propagating on the plants under these conditions. Of note, the mock treatment also had growth on the MRS plates which may indicate that the natural colonizers of these seeds include anaerobes. Upon inspection, the colonies observed in this treatment did not correspond to a background population of the inoculated microbes. The colonies were smaller and different in appearance than the inoculated strains. Only DP100 colonization showed a benefit with polymer coating for this type of lettuce. The right graph shows the average biomass of the harvested plants. Note the strong biomass benefit seen with inoculation of polymer and DP100. DMA #2, which contains DP100 also confers the same biomass benefit.

### Example 8: Plant Colonization by Single Species and DMAs

To determine whether we could successfully colonize a variety of plant types by inoculation of seeds and identify what level of inoculum was ideal for maximal colonization, we performed a series of experiments growing plants from seeds in sterile environments (enclosed boxes with a gel-based medium). The plants were cultivated in this manner for one to three weeks which is long enough for a large variety of plants to reach the seedling stage.

For these experiments, Little Gem lettuce (Johnny's Seeds Product No. 4120G.11), Black Seeded Simpson lettuce (Ferry Morse Product No. 2498), and Outredgeous lettuce (Johnny's Seeds Product No. 2208G.26), arugula (Johnny's Selected Seeds Product No. 385.11), tomato, var. sweetie (Ferry Morse Product No. 1505), red cabbage (Johnny's Seeds Product No. 2230M.30), Red Arrow Radish (Johnny's Seeds Product No. 3111M.30), arugula for microgreens (Johnny's Seeds Product No. 385.30), Bright Green Curly Kale (Johnny's Seeds Product No. 4085M.30), Daikon Radish (Johnny's Seeds Product No. 2155 MG.30), Broccoli (Johnny's Seeds Product No. 2290M.30), and Early Wonder Tall Top Bect (Johnny's Seeds Product No. 123M.30) seeds were disinfected by chlorine gas.

Seeds were inoculated in polymer with D. hansenii DP5, P. kudriavzevii DP102, P. fluorescens DP1, L. plantarum DP100, L. brevis DP94, L. garvieae DP97, L. paracasei DP95, Leuconostoc mesenteroides DP93, combinations thereof (DMAs), or mock control at concentrations ranging from 1×10³-1×10⁷ CFU per seed. Four to eight seeds per treatment were planted in autoclaved Magenta GA-7-3 Plant Culture boxes (Sigma Aldrich Catalog Number V8505) with sterile Murashige and Skoog basal medium (Sigma Aldrich M5519-50L) with 0.1% concentration of Phytagel (Sigma Aldrich P8169-250G). After 7-24 days growth the seedlings were harvested, photographed, and colonization was measured.

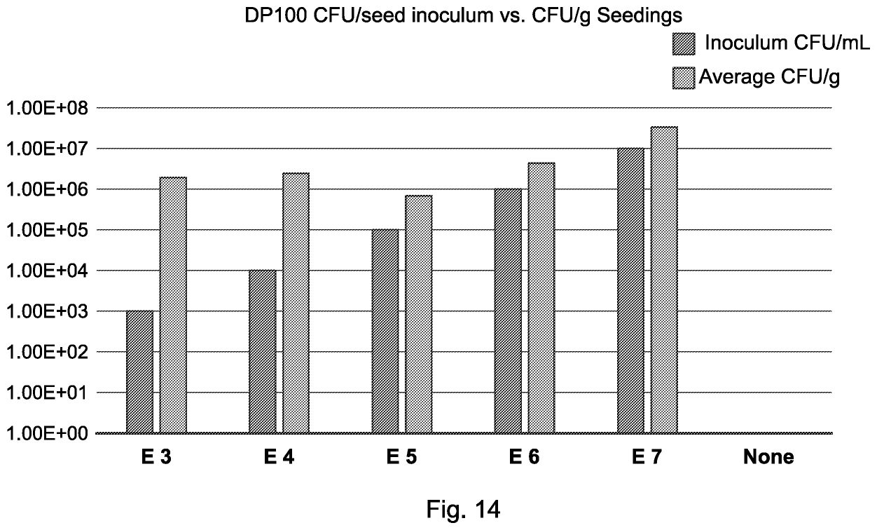

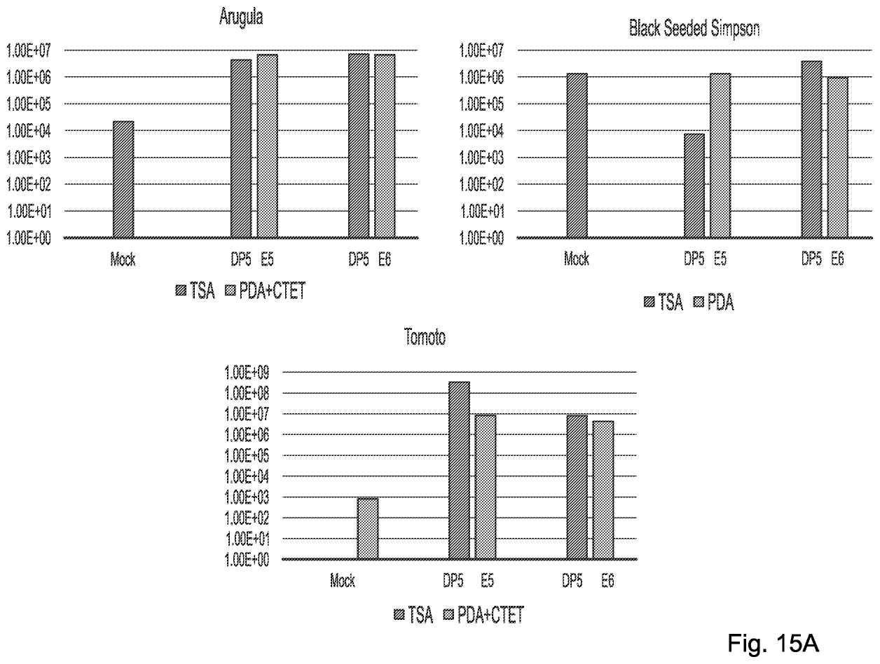

We performed an initial inoculum titration experiment to measure the dose response using a single strain (DP100) and seed type, arugula (FIG. 14). Increasing concentrations of seed inocula were tested with the highest 1×10⁷ (E7) CFU/seed achieving the highest colonization at 1×10⁸ CFU per gram. Interestingly, low microbial titers (E3-E5) CFU/seed resulted in colonization levels of approximately 1×10⁶ CFU per gram, indicating replication of the microbe on the plants at a very high growth rate.

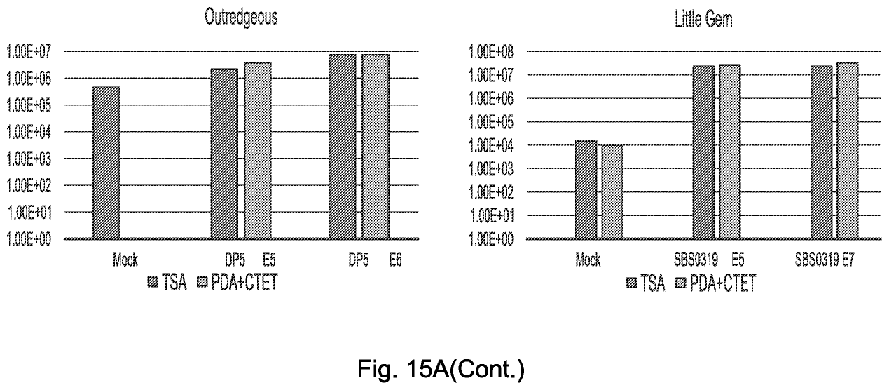

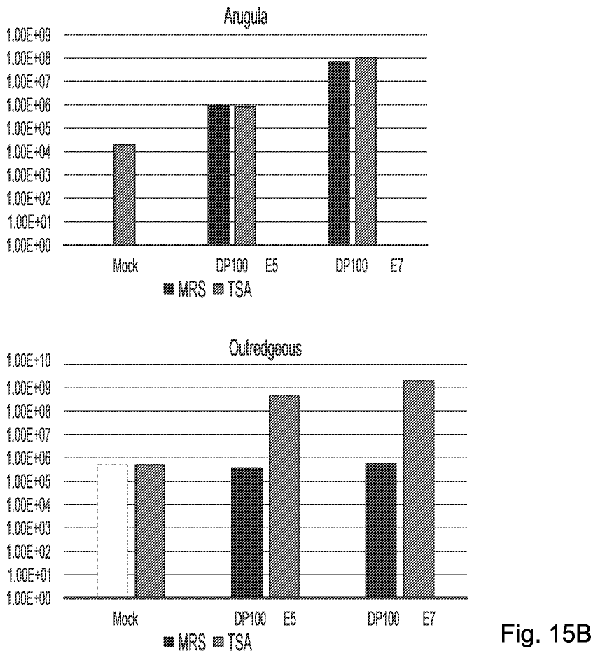

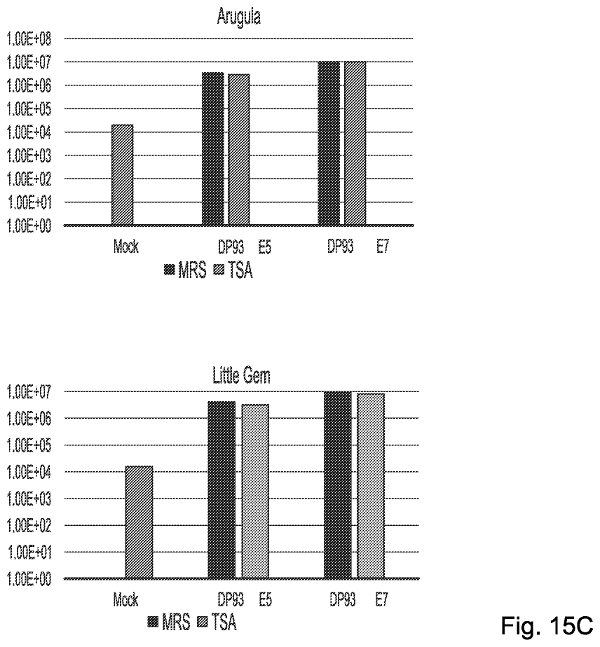

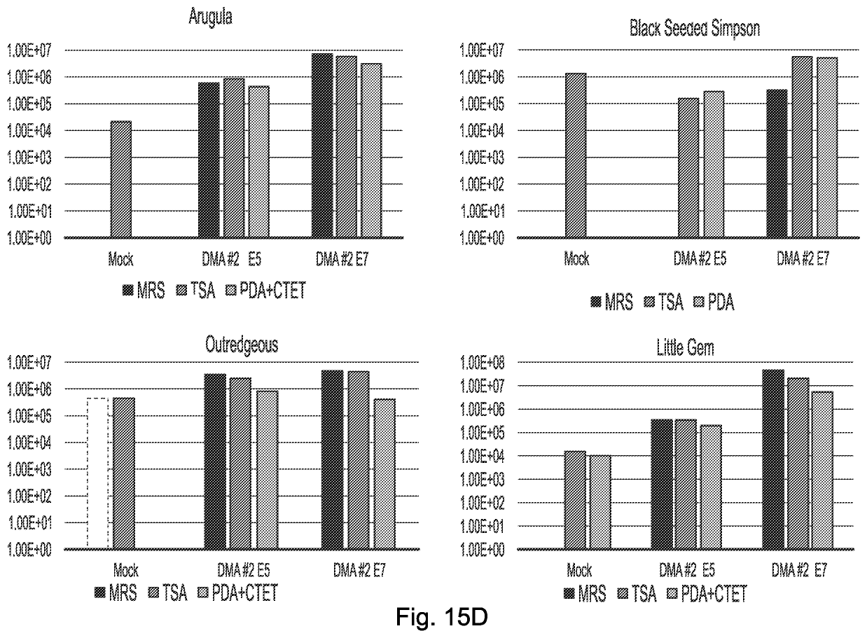

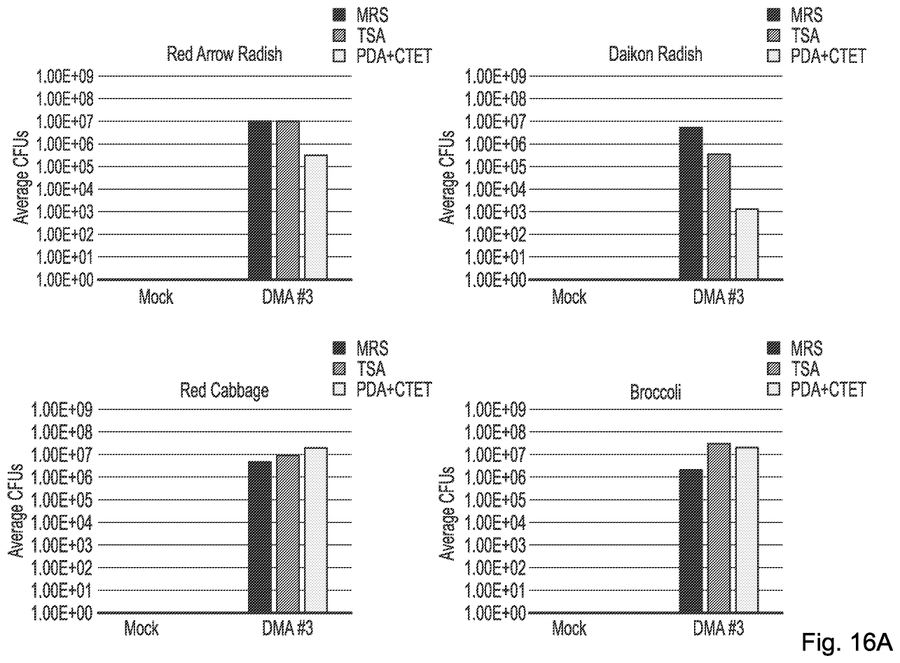

We further investigated titrations of microbes and their response to colonization using several crops and four single strain or DMA combinations. We compared inocula of 1×10⁵ versus 1×10⁷ CFU/seed for bacterial and DMA preparations and 1×10⁵ versus 1×10⁶ CFU/seed for yeast preparations. Overall, the vast majority of plant types were successfully colonized with the single microbes or DMAs used. Colonization was robust, equaling or exceeding the initial inoculum of the seeds, indicating propagation of the microbes or DMAs on the plants (FIG. 15). DP5 colonized all plant types tested and achieved a high titer regardless of inoculum size. DP100 showed a stepwise increase in colonization with increased inoculum on arugula but achieved lower titers on Outredgeous lettuce, highlighting the specific relationship between arugula and this strain. DMA #2, which is comprised of three lactic acid bacteria and a yeast, exhibited increased colonization with increased inoculum for arugula and Little Gem lettuce. This DMA achieved high titers regardless of inoculum size on Outredgeous lettuce whereas colonization was poor for the lactic acid bacterial portion on Black Seeded Simpson lettuce, again demonstrating specificity between colonizers and plants.

Finally, as microgreens have become a popular, nutrient-rich source of plant intake, we examined colonization of these faster-to-market plants. We selected several varieties of microgreens, including arugula, kale, radishes, and broccoli and inoculated them with DMAs consisting of one bacterial strain (1×10⁷ CFU/seed) and one yeast (1×10⁶ CFU/seed). The combination of bacteria and yeast reflects their synergistic interactions to promote growth in the plant, protect against abiotic and biotic stresses such as fungal pathogens during farming, and also their potential to be synergistic in the gastrointestinal tract of an animal consuming the fresh crop. For example, by the enhanced production of short chain fatty acids that have anti-inflammatory effects in the human host. These greens were harvested 7-10 days after planting, in line with harvest times for conventionally grown microgreens.

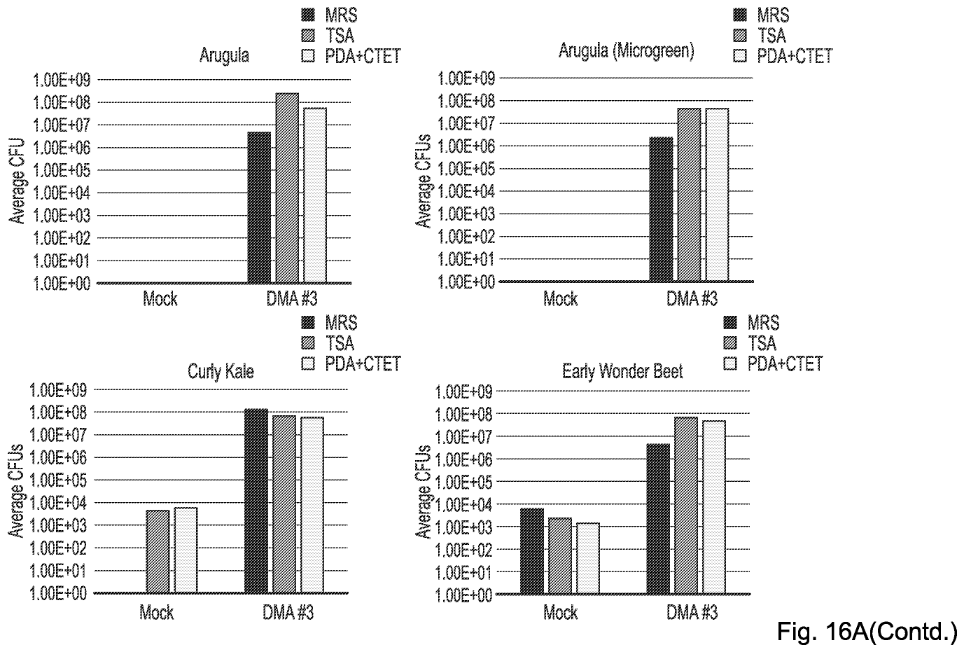

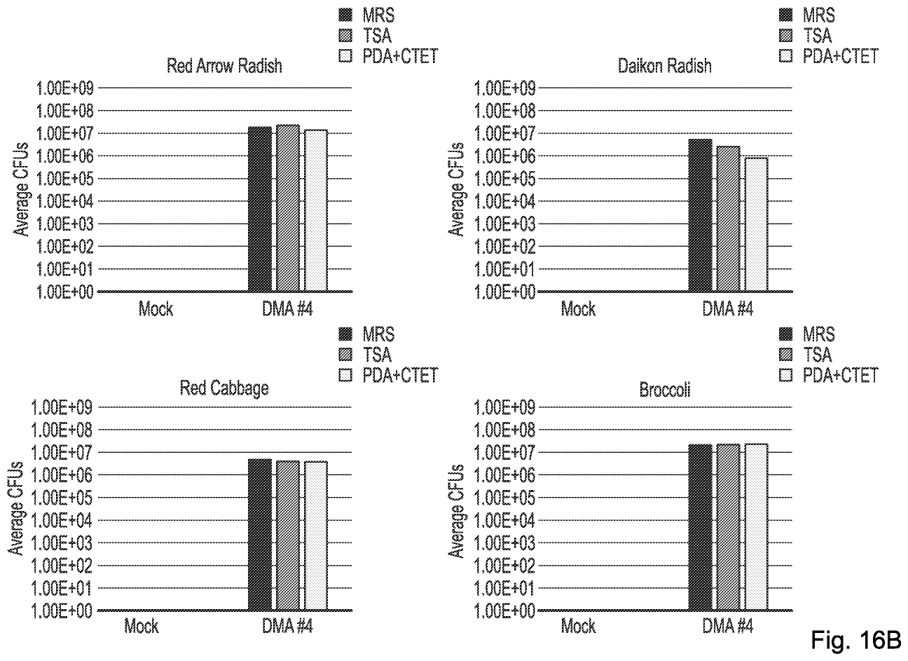

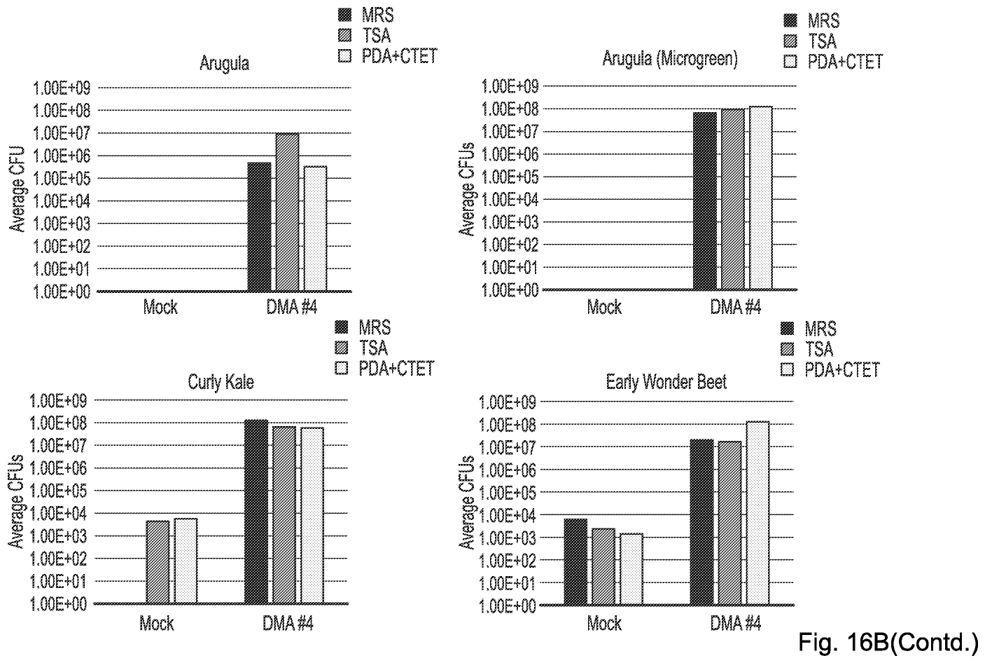

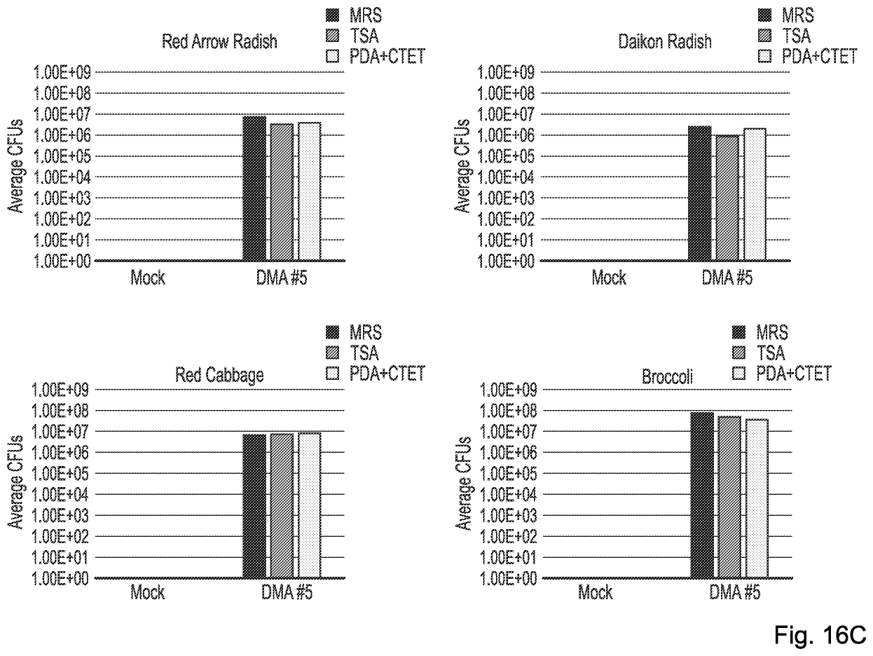

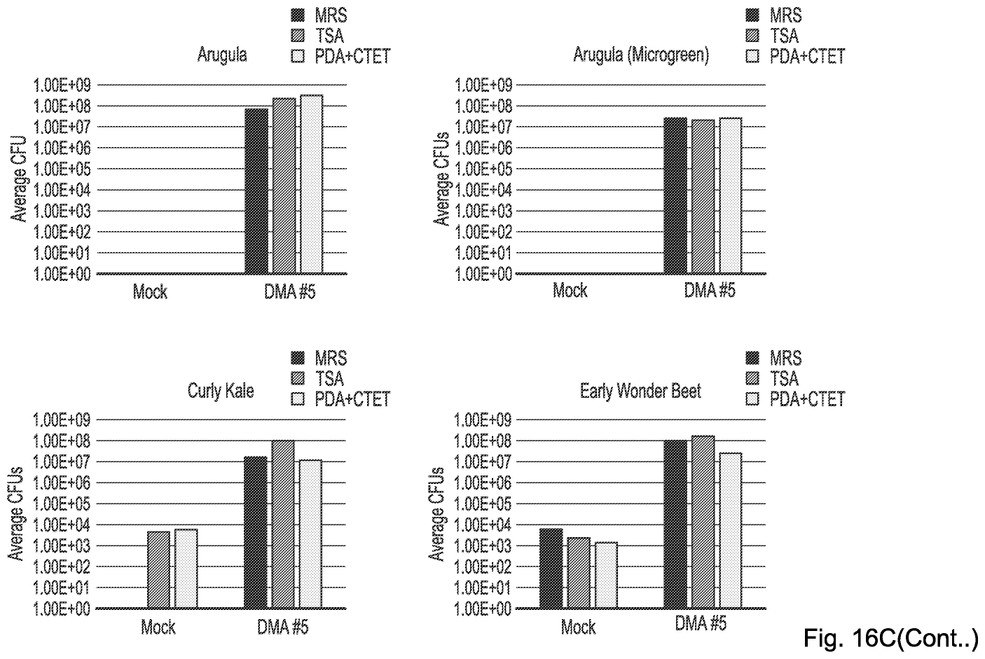

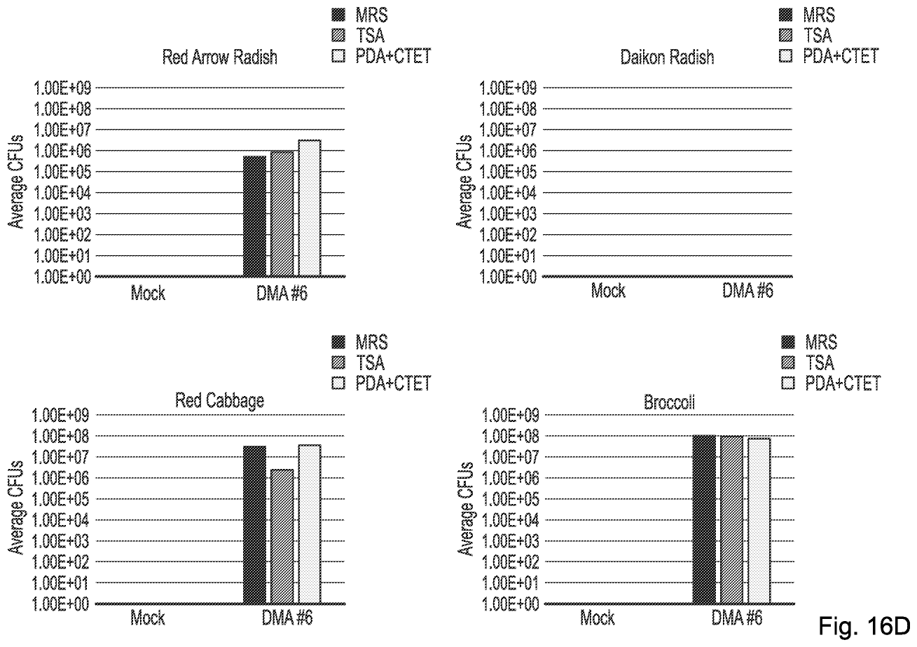

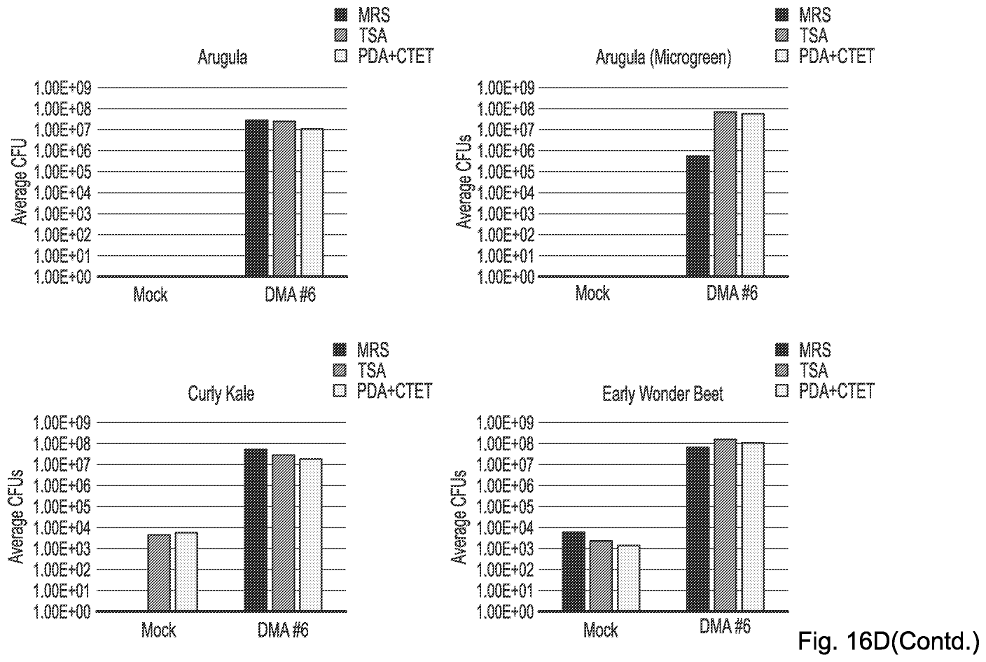

Colonization was robust across all DMAs and plant types tested with the exception of DMA #6 on Daikon Radish where no colonization was seen (FIG. 16). Despite a relatively high level of colonization throughout, microbial loads fluctuated as much as 1000-fold depending on the DMA and plant variety. These variances were specific to the DMA and plant combination, highlighting the importance of selecting the appropriate microbial inocula for a given plant and the impact of the selection in effective product development. Microbial loads at the 1×10⁷ to 1×10⁸ CFU/g level, as seen here, are equivalent to probiotic doses of commercial products sold as capsules and much higher than nutritive food products, for example yogurt, compared to the consumption of 10-100 grams of microgreens treated with the correct inocula.

FIG. 14 demonstrates the effect of increasing inoculum on plant colonization level. Arugula seeds were inoculated with DP100 at levels from 1×10³ up to 1×10⁷ CFU/seed (dark gray bars) and compared to the CFU/g microbial output on the resultant seedlings. Note the evident propagation of the microbe on the plants inoculated with low levels of microbe.

FIG. 15 shows the levels of colonization of seedlings with single microbes or DMAs on a variety of plant types after seed inoculation. Homogenized seedlings were diluted and plated on non-selective (TSA) medium and medium specific for lactic acid bacteria (MRS) and/or medium selective for yeast (PDA+CTET).

FIG. 15A. Colonization of seedlings with Debaryomyces hansenii DP5 expressed as average CFU per gram plant material. This microbe achieved a high titer on all plant types tested. The presence of growth on the mock treatment on non-selective medium indicates the presence of endophytic microbes.

FIG. 15B. Colonization of seedlings with Lactobacillus plantarum DP100 expressed as average CFU per gram plant material. This microbe achieved a high titer on arugula but not on Outredgeous lettuce. Growth of very small colonies, not resembling the strain of interest on MRS indicates the presence of endophytic microbes (white dotted bar).

FIG. 15C. Colonization of seedlings with Leuconostoc mesenteroides DP93 expressed as average CFU per gram plant material. This microbe achieved a high titer on arugula and Little Gem lettuce and only small increases were present with higher inoculum.

FIG. 15D. Colonization of seedlings with DMA #2 expressed as average CFU per gram plant material. This DMA achieved a high titer on arugula, Little Gem and Outredgeous lettuce. Of note, the bacterial component of the DMA (selected for on MRS) is impaired relative to the other groups for Black Seeded Simpson Lettuce. Growth of very small colonies, not resembling the strain of interest, on MRS for Outredgeous lettuce indicates the presence of endophytic microbes (white dotted bar).

FIG. 16. Colonization of seedlings with DMAs. Eight seed-types were inoculated with DMAs and colonization was examined. DMAs contained one lactic acid bacterium and one yeast and hence were plated on MRS (bacterial selection), TSA (all microbes), PDA with chlorotetracycline (yeast selection). Colonization is expressed as microbial CFUs per gram of plant material. Note the background colonization observed in the mock controls for Curly kale and Early Wonder beet. The microbes present here represent the naturally occurring endophytes of these plants different from the heterologous microbes added with the treatments.

FIG. 16A. Colonization of seedlings with DMA #3. High levels of colonization were achieved with this DMA on arugula and kale, but colonization was approximately 1000-fold lower on Daikon radish.

FIG. 16B. Colonization of seedlings with DMA #4. The microgreen variety of arugula, beet, and kale were all colonized strongly whereas the cabbage and conventional arugula were colonized less well.

FIG. 16C. Colonization of seedlings with DMA #5. Arugula, broccoli, and kale were all colonized to 1×10⁸ CFU, however, the Daikon radish only achieved a 100-fold lower level of colonization.

FIG. 16D. Colonization of seedlings with DMA #6. The microgreen variety of arugula, broccoli, beet, and kale all exhibited a high degree of colonization, while the Red Arrow radish was colonized to a lower level. The Daikon radish was not colonized.

### Example 9: Colonization and Plant Benefits Measured in Hydroponic Systems

Hydroponically grown plants represent an increasing share of agricultural crops as indoor farming provides a year-round production cycle and vertical farming offers an increased efficiency in land usage. In addition, these soil-less systems offer a great deal of control over the plant growth conditions but provide unique challenges for the agriculturalist and the plants themselves. In soil, many potentially pathogenic microbes are present which are often kept under control by the natural microbial symbionts of the plants. Pathogens may be less abundant in hydroponic systems, thus colonization by our microbes have the potential to be less beneficial to the plant. This reduces the need for agrochemicals such as fungicides that may be undesirable for the consumer. We aimed to determine if colonization could be achieved with hydroponically grown plants and if it could represent a benefit in the overall yield.

In order to examine whether colonization of plants could be successfully achieved in hydroponic systems, we selected three varieties of lettuce, Little Gem, Black Seeded Simpson, and Outredgeous, for colonization experiments. Surface-disinfected seeds were inoculated with DP100, DMA #1, or a mock condition. Inocula were combined with polymer prior to application to seeds. The seeds were then sent to Zea Biosciences (Walpole, MA), for growth aseptically using hydroponics. Twelve seedlings per condition were harvested 20 days later and plant colonization and weights were measured.

Colonization was achieved with at least one treatment for each type of lettuce, though to a lower level than the amount of inoculum used. The Black Seeded Simpson lettuce variety was exclusively colonized by DMA #1. The Little Gem lettuce was colonized by DP100. However, the Outredgeous lettuce was successfully colonized by both. Average and aggregate plant masses were unaffected by the colonization of the plants, indicating no detriment to growth.

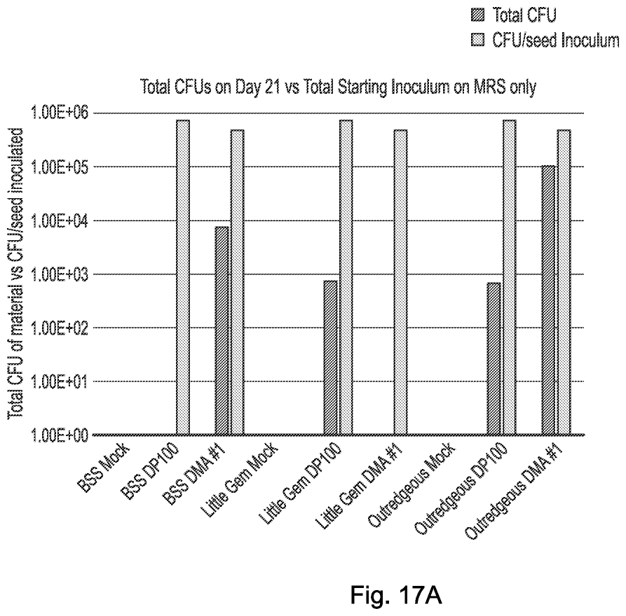

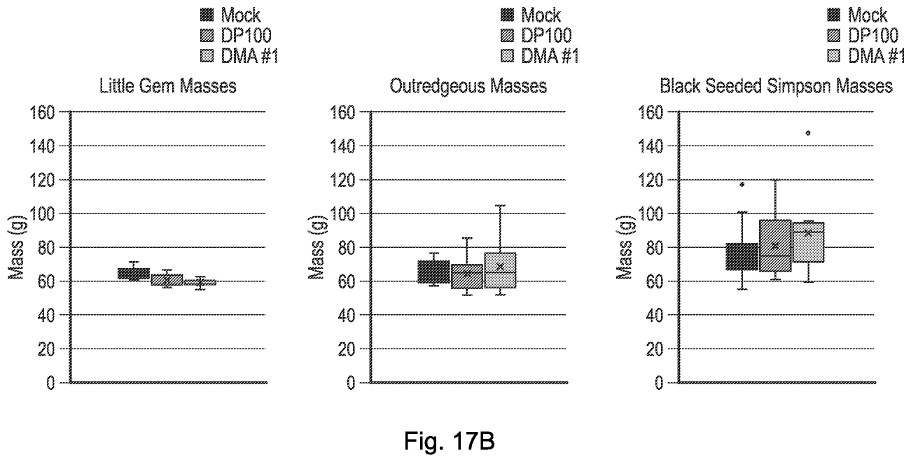

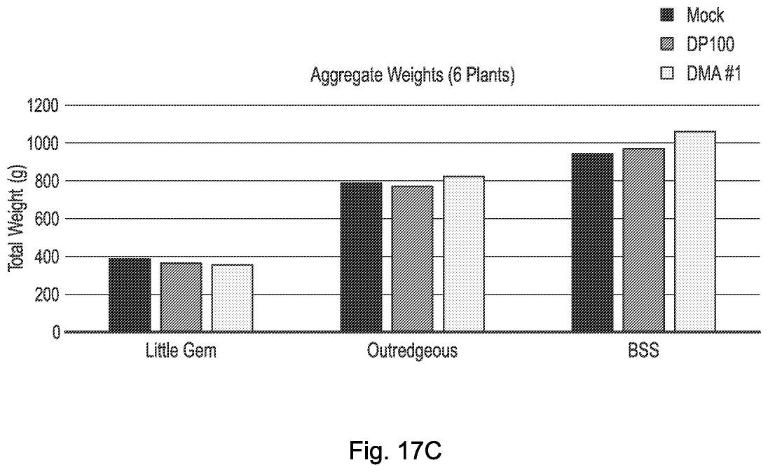

FIG. 17. Colonization and weights of hydroponically grown lettuces.

FIG. 17A. Shows the average colonization of per plant (dark grey bars) relative to the original seed inoculum (light gray bars). CFU plating was performed on MRS selective medium alone. Note the specificity between colonizer and lettuce type.

FIG. 17B. Box and whisker plots of lettuce plant masses. In general plant mass was unchanged by treatment type regardless of whether colonization was successful.

FIG. 17C. Histogram depicting aggregate plant masses. The total mass of 12 plants per treatment was measured. Differences in total yield can be seen between lettuce types but not within each group.

### Example 10: Colonization and Plant Benefits Measured in Peat-Grown Systems

Peat moss is a common substrate on which to germinate seeds before planting in home gardens and commercially. We researched whether colonization with our microbes improved tomato plant vigor. For this study, Tomato seeds, var sweetie ((Ferry Morse Product Number: 1505), were disinfected and inoculated with L. plantarum DP100, P. kudriavzevii DP102, L. garvieae DP97 or mock control and planted in sterilized peat pellets and SUPERthrive Sample, 50 mm Pellets (Ferry Morse Product No. J616ST) and placed in Jiffy professional tomato and vegetable seed starting greenhouses. After 29 days of growth in a greenhouse the plants were harvested, photographed, weighed, and microbial colonization was measured.

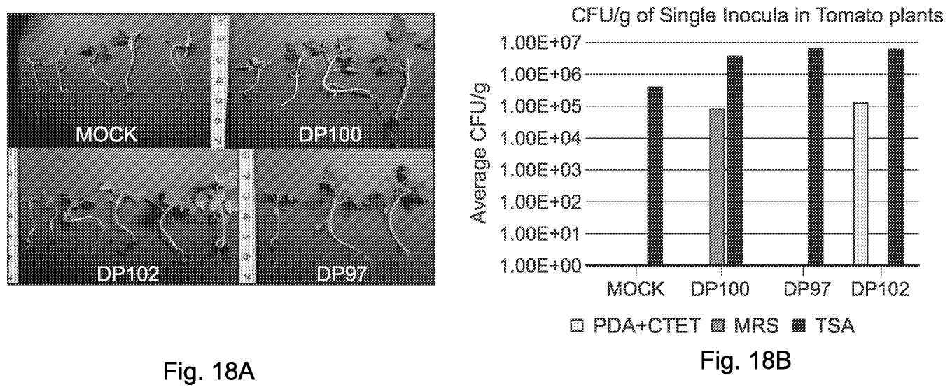

After approximately a month of growth on peat pellets, seeds treated with single microbes were larger (FIG. 18A). Colonization by endophytes was apparent for each treatment group, as seen on TSA plates (FIG. 18B). Total plant colonization was higher in the treatment groups. Despite a lack of successful colonization, DP97 improved plant size. This effect is consistent with benefits conferred by the microbe at earlier stages of growth by priming some of the hormone systems in the plant and by improving colonization by native beneficial microbes. DP100 and DP102 were observed at harvest, indicating successful colonization.

FIG. 18. Microbial preparation of seeds can enhance tomato plant growth. Tomato seeds were inoculated with three single microbe treatments or mock and grown on peat pellets for 29 days. Plants were harvested and photographed to demonstrate plant size (A). Colonization was measured on non-selective media (TSA) and media selective for DP100 and DP97 (MRS) and medium selective for DP102 (PDA+CTET) (B). DP100 and DP102 successfully colonized the tomato plants and DP97 did not. However, each treatment resulted in larger tomato plants. As it is the case in other agricultural systems tested, there is a high degree of specificity between crop and microbial inocula that will determine the best product efficacy. An early vigor increase can have a significant beneficial effect in total fruit yield.

### Example 11: Germination and Plant Benefits Under Abiotic Stress (Heat) Measured in Soil

During cultivation, plants encounter many abiotic stressors such as drought, heat, cold, mineral toxicity, and salinity as well as biotic stresses primarily driven by fungal plant pathogens. Certain plant-symbiotic microbes are known to ameliorate some of the effects of these abiotic stressors so we tested whether our probiotic microbes could provide a similar benefit to seeds and plants exposed to heat stress. In this combination crop-probiotic product a double benefit system is sought on which the crop is farmed under a more sustainable practice by improving water use efficiency or nutrients, and the resulting crop is more nutritious from the perspective of the edible beneficial microbiota provided. It was recently recognized that fresh fruits and vegetables consumed raw carry viable bacteria and fungi, some of which can pass through the GI tract. Some of the probiotics are adapted to colonize crops effectively and also to survive the acidity, anaerobiosis, and bile salts on the mammalian GI tract. This reflects their evolution and co-adaptation to alternating plant and animal hosts in their life cycle and therefore provides help during plant stress exposures.

Little Gem, Black Seeded Simpson, and Outredgeous, lettuce seeds were disinfected and inoculated in polymer with L. plantarum DP100, D. hansenii DP5, P. kudriavzevii DP102, L. brevis DP94, Leuconostoc mesenteroides DP93, DMA #2 or mock control and potted in soil sterilized by autoclaving in Pro-Hex trays (Ferry Morse). Eighteen seeds were planted per treatment group. Germination, defined as the appearance of a plant shoot, was measured and recorded over four weeks. During this time the plants encountered 4 days of excessive heat (above 38° C.) at irregular intervals. After 35 days, plants were harvested and weighed to determine aggregate weights.

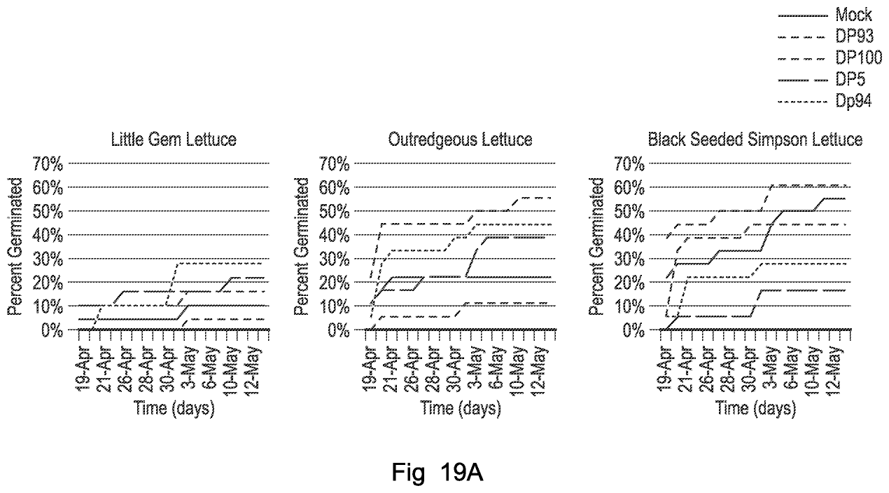

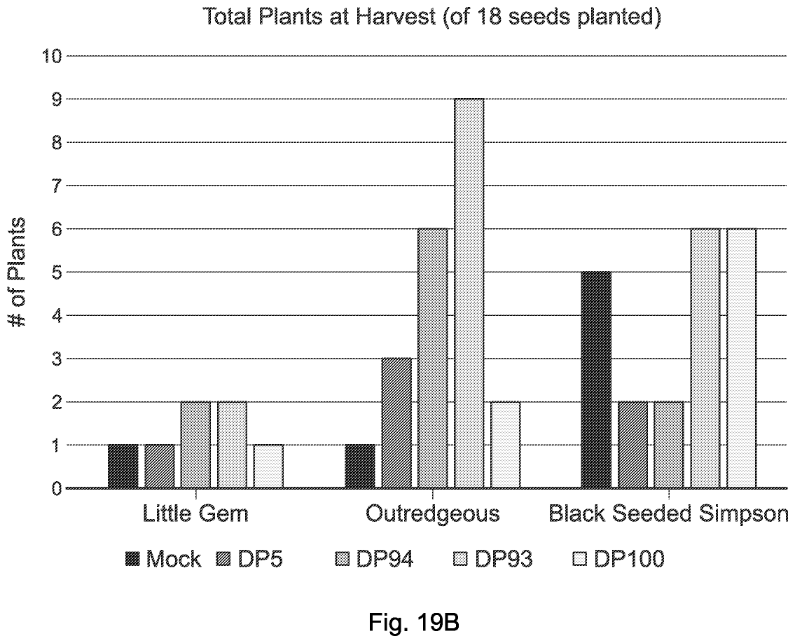

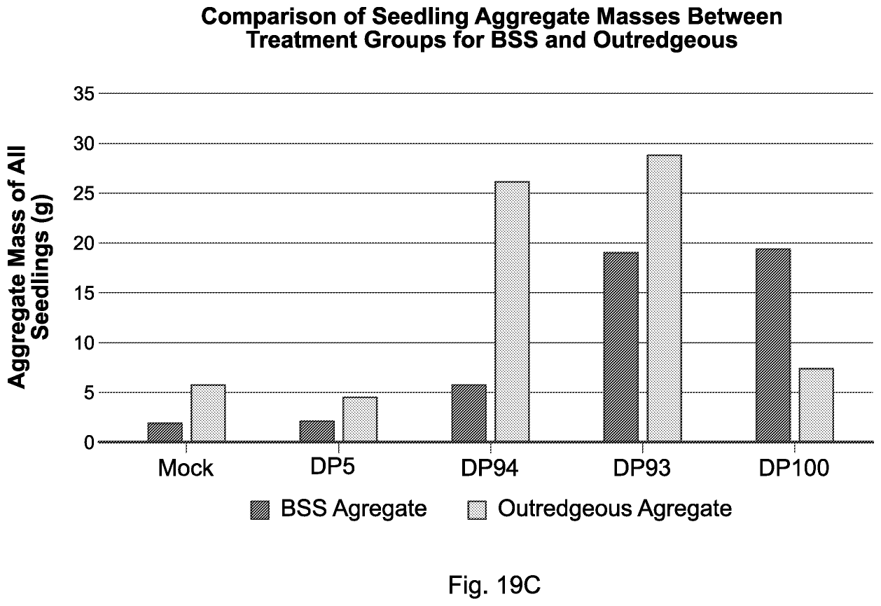

With each type of lettuce, one or more microbe treatments improved germination rates. Little Gem lettuce displayed the lowest heat-tolerance, with only a small percentage of plants germinating (FIG. 19A) and surviving (FIG. 19B), so aggregate weights were not measured due to small sample size. In spite of this, germination rates were improved for this lettuce with DP5, DP94, and DP93. Germination rates were lower than the mock control for DP100, indicating this microbe-plant pairing is not beneficial in this instance.

Outredgeous lettuce exhibited the greatest improvements in germination rates with microbe treatment under heat stress. Treatment with DP94 doubled the germination rate, while DP93 nearly tripled the number of germinated plants. Furthermore, plant survival was vastly improved all treatments except for DP100, indicating that microbial treatment is extremely beneficial in this context. Finally, aggregate plant masses were improved 5-6-fold with DP94 and DP93 seed treatment. This finding is important for agriculture in hot environments as revenue is generated from the number and total weight of plants harvested.

Black Seeded Simpson lettuce was the most heat-tolerant of the lettuce types tested (56% percent germination of the mock treatment group). Hence, the benefit of microbial treatment was diminished for this variety. Only DP100 demonstrated an improvement in germination under heat stress over the mock. This differs from the other two varieties where DP100 resulted in lower germination rates than the mock. Additionally, the aggregate weights of the DP100 lettuces were 10 times higher. This example highlights the specificity in relationship between plant and microbe. DP93 did not improve germination rates but did appear to improve plant aggregate weights (FIG. 18C), which may suggest that the benefits to the plant provided by this microbe are on growth rather than germination.

We also sought to examine the heat-tolerizing beneficial effects of our microbes on mature plants at harvest. Little Gem lettuce was selected because it displayed the least heat tolerance of the lettuces tested in the earlier study. To do this, Little Gem lettuce seeds were disinfected and inoculated in polymer with L. plantarum DP100, DMA #2 or mock control and given to Zea BioSciences to germinate in their hydroponic system. Three-week-old seedlings (five per treatment) were transplanted into conventional potting soil. The plants were grown for an additional 7 weeks in a greenhouse where they were exposed to 4 days of excessive heat (above 38° C.) at irregular intervals. After which, plants were harvested, photographed and weighed.

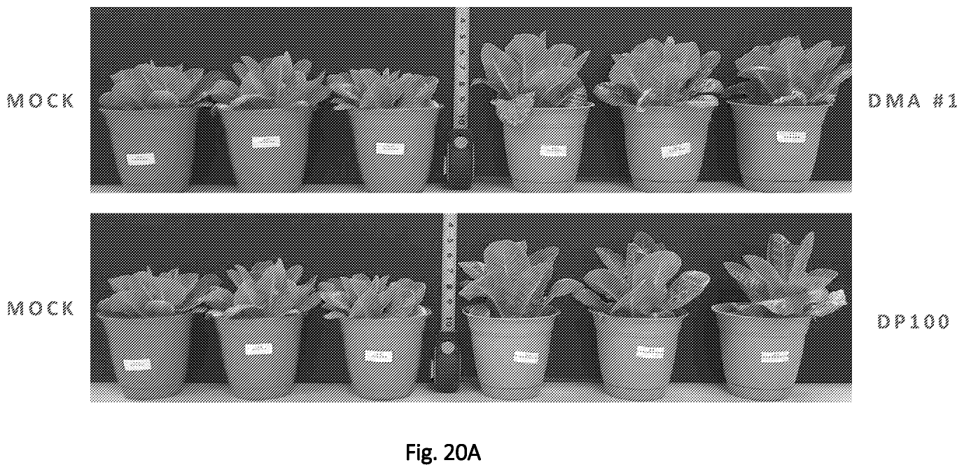

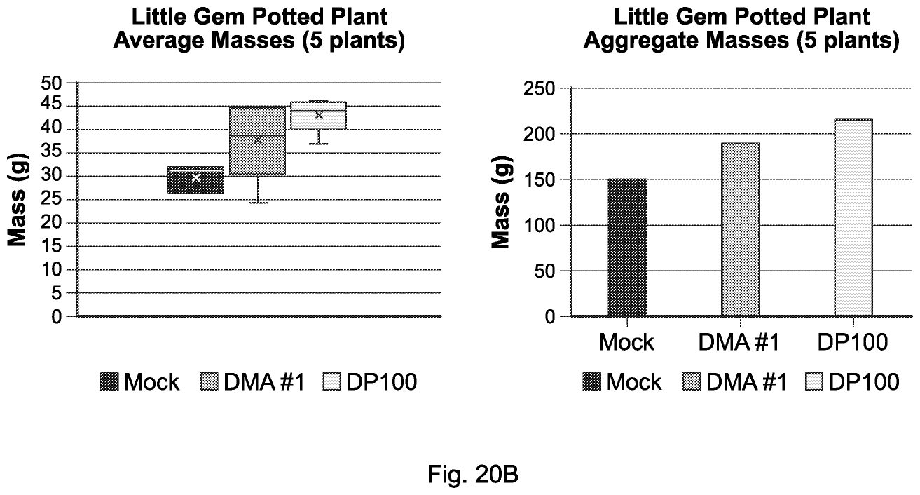

Single microbe or DMA treatment had a profound effect on the growth of the lettuce under heat stress. Plant vigor was improved with treatment of DMA #1 but further improvements were seen with single DP100 treatment. Importantly, aggregate plant weight was improved by 45% with DP100 treatment and 27% with DMA #1 treatment. Crop yield improvements of this sort would result in greater market values for farmers. FIG. 19A shows germination rates under heat stress. Germination rates for each lettuce variety are displayed as percent germination (of 18 seeds) over time. Not all microbe treatments improved germination rates over mock (black). The microbe that improved germination most was specific for each lettuce type. FIG. 19B depicts total plant survival under heat stress. Not all plants that germinated survived continued heat stress. This histogram indicates how many plants survived to the point of harvest. Treatment of Outredgeous lettuce seeds with DP93 and DP94 improved survival. FIG. 19C shows Pro-Hex aggregate weights under heat stress. The total weight of all Outredgeous and Black Seeded Simpson lettuce plants harvested at 35 days post planting. A combination of germination improvement and enhanced plant growth led to more biomass generated for lettuce treated with DP94, DP93, and DP100, when compared with mock and DP5 conditions. FIG. 20A depicts Little Gem seeds treated with microbes result in larger and more healthy plants when subjected to abiotic (heat) stress. Photographs of mature plants from mock-treated (left) and single microbe or DMA-treated seeds (right). A measuring tape reference is included for size in each photo. Note the larger plant sizes with probiotic microbe treatment. FIG. 20B depicts Little Gem potted plant masses grown with heat stress. Box and whisker plot of masses from five lettuce plants harvested (left) and a histogram of aggregate plant masses (right). With both measurements plant masses were improved with microbe or DMA inoculation.

### Example 12: Microbes can Colonize Plants and Humans as Part of their Life Cycle

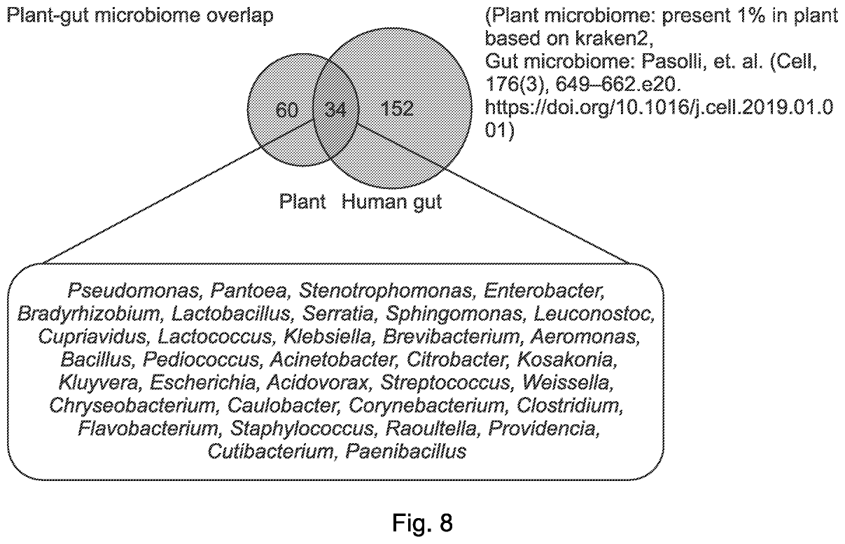

Lactic acid bacteria and other groups of bacteria colonize plant tissues on the surface or as endophytes inside tissues. Lactobacillus, Leuconostoc, and Lactococcus for example have been detected in fresh cabbage and then enriched in fermented products such as kimchi and considered probiotics providing health benefits to the human host. In addition to the plant host, they have been isolated from human stool or colonic biopsies indicating they can colonize or transit through the human gastrointestinal tract and therefore considered commensals for humans and safe to consume. A genomic survey of the samples listed in Table A revealed that there was a total of 94 bacterial genera and when compared to human stool meta studies there is an overlap of 34 genera found both in the plant host and in humans. The list with the overlapping genera in FIG. 8 contains the preferred candidates for nutriobiotics including multiple DP entries listed in Table E in addition to Lactobacillus, Leuconostoc and Lactococcus. In some embodiments, bacteria belonging to the genera listed in FIG. 8 can be developed into nutriobiotics.

### Example 13: Organic Farming Promotes a Higher Microbial Content and Diversity than Conventional Farming

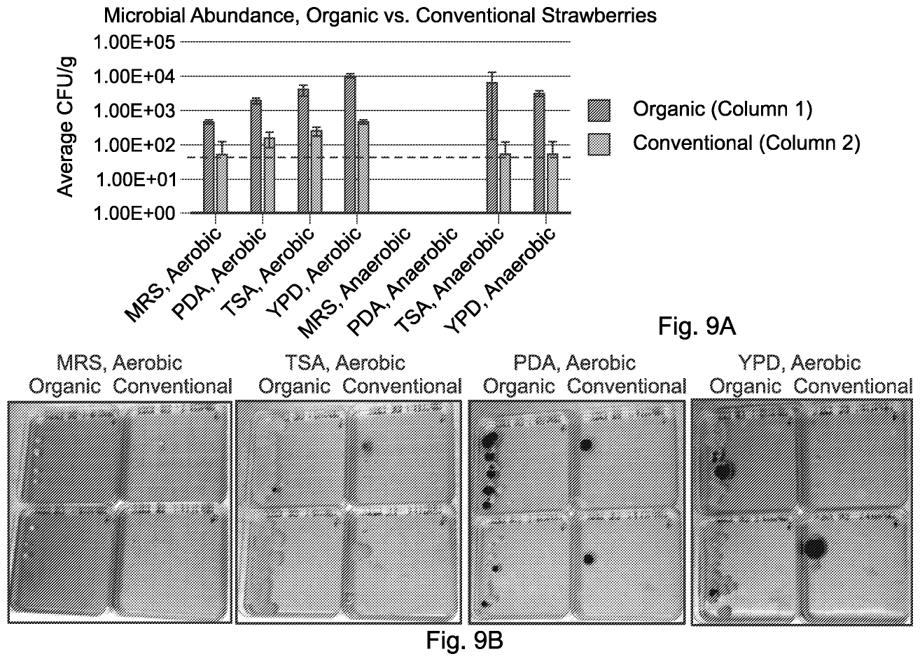

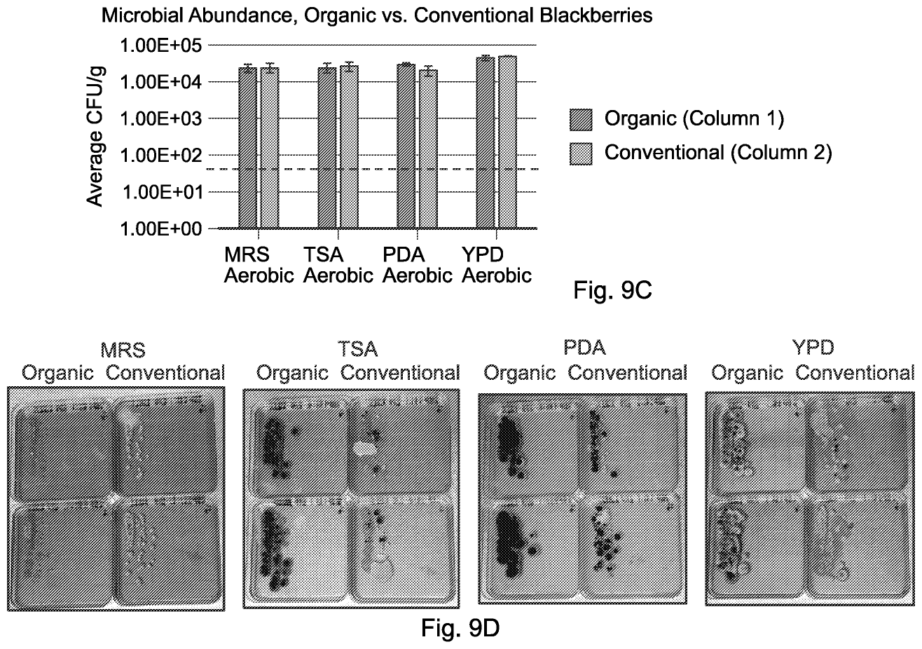

The use of agrochemicals since the green revolution has been aimed to increase crop yield by the use of chemical pesticides and herbicides with transgenic plant lines. This practice has a detrimental effect in the overall natural endogenous microbiota that decreases the product's nutritional value with a reduced content of beneficial microbes as the fungicides and pesticides are not specific to eliminate a target pathogen but affect also beneficial species. In FIG. 9 these differences are measured in strawberries and blackberries. For strawberries of the same variety farmed conventionally there is at least 10-fold decrease in the total microbial populations measured on several culture media (FIG. 9A) whereas not significant changes were observed in blackberries (FIG. 9B). In some embodiments, the use of nutriobiotics can provide a supplemental heterologous microbial load to restore some of the plant and human beneficial microbes.

### Example 14: Carbohydrate-Related Enzymes CAZymes in Nutiobiotics are Important for their Role in Plant and Human Host

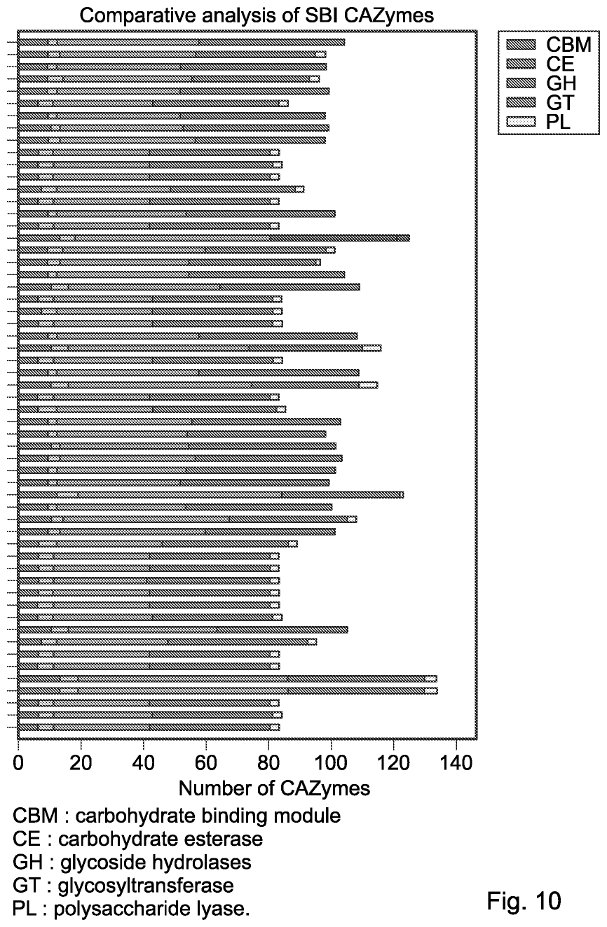

There are different enzyme families relevant in the role of bacteria with their beneficial role in crops and in humans. For example, glycosyl hydrolases (GH) cleave specific moieties on fungal cell walls that can serve as protectants against fungal pathogens protecting the crop from infections. Other families of GH break down components of plant fibers that microbes convert into anti-inflammatory short chain fatty acids in the colon to aid in the plant material complete digestion and production of fermentable substrates that can be beneficial and cross feed with other probiotic members in the gut. In FIG. 10 it is represented the abundance of the most relevant families of CAZymes and the feature of this as a nutriobiotic function.

All references, issued patents, and patent applications cited within the body of the instant specification are hereby incorporated by reference in their entirety, for all purposes.

### Example 15 Application of DMAs to Grapes

To apply probiotic DMAs to grapes, the first step is inoculating the grape plant with a selected DMA to create a culture of the beneficial microbes in the mature plant tissue. An inoculum of 1e10 CFU/ml is necessary to ensure a sufficient population of microbes exists to confer measurable benefit to the grape plant and the human that cats those grapes. Inoculation occurs using one of several available techniques. For grapes, available techniques include osmopriming/hydropriming seeds, foliar application on the flowering grape, soil inoculation where the vine is planted, vector-mediated inoculation using pollinators during grape flowering, wound inoculation though an incision in the grape vine, or injection into the grape vine/roots directly. Once inoculated, the plant's normal growth cycle proceeds as it otherwise would.

When the grape plant is inoculated using the osmopriming/hydropriming technique on the seed, shelf-life of the DMA is extended when a polymer seed coating is used. This coating is oxygen-permeable but protective, increasing the survival of the DMA from between coating and planting activities, thus increasing the overall efficacy of the DMA in delivering its benefits to the plant and human host.

The second step is measuring the colonization of the plant with the DMA microbes. At the grapes' maturity, colonization is measured using a quantitative PCR test collected from the grape tissue. The conditions required for this test vary based on the microbial species included in the DMA as specified in Table I. When the population of microbes in the sample reaches a threshold suitable for the grape plant [the threshold is determined by plant type], then colonization of the DMA is successful, and several benefits are observed, including increased plant stress resistance, increased plant health, increased probiotic content and enhanced flavor. These benefits are described in more detail below.

Increased plant stress resistance: survival of the treated grape plant under abiotic stress conditions during the plant growth cycle as compared to a control plant without the DMA (e.g., drought-resistant grapes)

Increased plant health: higher biomass of a treated grape plant as compared to a control plant without the DMA (e.g., increased yield of grapes)

Increased probiotic content: higher concentration in the treated grape plant of specific microbial species known to confer benefits on human health as compared to a control plant without the DMA (e.g., table grapes with increased levels of L. plantarum, known to improve symptoms of inflammatory bowel disease)

Enhanced flavor: improved taste and/or texture of the mature treated grape plant and its biproducts as compared to a control plant without the DMA (e.g., wine given a specific terroir because of grapes treated with the DMA)

### Example 16 Application of DMAs to Strawberries

Strawberries are inoculated with probiotic bacteria and fungi that colonize the fruit and propagate as the fruit develops increasing in numbers to reach an abundance between 1e4 CFU/g to 1e8 CFU/g. Inoculation can be done in several ways, but a preferred method is applying a spray with a suspension of 1e10 CFU/ml to the flower to pollinize the tissues that will become the fruit mesocarp and exocarp. Upon colonization, the probiotic DMA provides biological protection against fungal pathogens by two mechanisms: a competitive exclusion as they are occupying the space that the pathogen would colonize, and by the ability to produce inhibitory molecules against fungal pathogens. In one study, Chen et al 2020 demonstrated the co-application of Lactobacillus plantarum, a well-known human probiotic and Botrytis cinerea a strawberry pathogen that at a concentration of 1e9 CFU/ml the probiotic inhibits completely the fungal pathogen.

Once the fruit is harvested some pathogens can cause spoilage generating economic losses. Preservatives and anti-spoilage products are typically applied. Most of these are natural oils and other substances. DMA consisting of probiotic bacteria and fungi is applied to the surface of the fruit by mixing with an edible polymer such as alginate. A microbial suspension of 1e10 CFU/ml is sprayed on the fruit surface to offer a coating that prevents the colonization and establishment of fungal pathogens and other postharvest pests. This layer allows the DMA to colonize and attach to the fruit surface and maintain the viability and population levels applied or even to grow for one or two cell divisions depending on the storage temperature. In refrigeration growth is arrested but storage at ambient temperature, typically at 22° C. growth of the probiotics can occur on the fruit surface and inside the polymer coat.

The application of these methods results in a fruit enriched or fortified with human probiotics, reduction of the use of agrochemicals applied for the control of fungal pathogens and extension of the shelf life. The product quality is superior to that of conventional or organic farmed strawberries.

### Example 17 Application of DMAs to Fruits with Natural and Synthetic Polymers

Successful application of beneficial microbes to foliar surfaces and fruits that are exposed to environmental conditions such as sun, wind, and rain can prove challenging. Over the last few decades, the agricultural industry has incorporated application of non-toxic natural and synthetic polymers to improve the efficiency of pesticides, herbicides, and fertilizers (Sikder, A. et al., ACS Applied Polymer Materials 2021 3 (3), 1203-1217). Some of these polymers aid in adherence, disbursement, and water retention. It is reasonable to surmise that these polymers could also be used to aid in application of DMAs to fruit. The present example describes application DMA #5 to growing strawberry fruit using a variety of natural and synthetic polymers.

### Methods

DMA and Polymer Solution Preparation—For preparation of DMA #5 for growing fruit or flower inoculation, microbes were inoculated from cryostock preparations and grown overnight in appropriate media and conditions. Microbes were diluted to the desired concentration with water or pelleted by centrifugation and resuspended in water to remove spent media and metabolites. Alternatively, lyophilized microbes were produced by fermentation in a bioreactor, harvested by centrifugation, homogenized with an excipient blend of oligofructose, sucrose, and ascorbic acid or of inulin, trehalose, and ascorbic acid, then lyophilized. These microbe powders were enumerated and known concentrations were weighed aseptically and added to water. To these mixtures polymers were added (for example: xanthan gum (0.5%), Croda polymer Tween L-1010 (0.25%), or Croda polymer ATPlus UEP-100 (0.25%)). Lactose (0.1%) or cryobuffer 1 (CB1, 2%) may also be added. Mock control material, consisting of polymer and water, can also be prepared as a microbiological control.

Bolus Inoculation of Flowers—To apply a bolus, the pistil cluster located in the center of the flower was inoculated with the microbe and polymer solution. A micropipette was used to apply the DMA and allowed to dry. Flowers were homogenized to determine the CFU applied. After 17-25 days, mature fruits were harvested and homogenized to quantify the number of DMA microbes present on the fruit.

Spray Inoculation of Fruits and Flowers—DMA #5 in polymer solutions containing natural and synthetic polymers were applied to strawberry fruit and flowers via spray application. For these, the DMA and mock control solutions were prepared as described above and added to sterile plastic spray bottles. Each flower or fruit was sprayed with the solution, with spray volumes of 0.75 mL to 1.25 mL. The spray was allowed to dry before harvest at the TO timepoint for fruits and flowers. Fruit was also harvested at 48 hrs and 7 days. Strawberries from inoculated flowers were harvested at 17-25 days, when ripe.

Enumeration of Microbes on Strawberries and Flowers-To quantify the number of viable microbes present on each fruit or flower, dilution spot plating was performed. Each fruit or flower was harvested and weighed. The fruit or flowers were thoroughly homogenized with a sterile mortar and pestle. PBS was added to facilitate grinding and washing of the mortar. The homogenate was collected, and the total volume was recorded. The homogenate was serially diluted in PBS and 3 μl of each dilution was plated in triplicate onto selective or deMan Rogosa Sharpe (MRS) medium to selectively grow lactic acid bacteria, potato dextrose agar (PDA) with chlorotetracycline to selectively grow fungi, or tryptic soy agar (TSA) a non-selective medium. Plates were incubated (37° C. anaerobically on MRS or 30° C. aerobically for PDA or TSA) for 24-48 hrs before enumeration. The resultant CFU counts, weight, and volume measurements were used to calculate the number of microbes per gram of material and per fruit.

### Results

An example DMA, DMA #5, consisting of one bacterium, DP100 Lactobacillus plantarum, and one yeast, DP102 Pichia kudriavzevii, was used for each experiment. DP100 and DP102 were inoculated from cryostock preparations and grown overnight at 30° C. in deMan Rogosa Sharpe (MRS) and potato dextrose broth (PDB) liquid medium respectively. Microbes were diluted with water. Alternatively, lyophilized microbes were produced by fermentation in a bioreactor, harvested by centrifugation, homogenized with an excipient blend of oligofructose, sucrose, and ascorbic acid or of inulin, trehalose, and ascorbic acid, then lyophilized. These microbe powders were enumerated and known concentrations were weighed sterilely and added to water.

To these DMA-water mixtures, xanthan gum (0.5%), Croda polymer 1010 (0.25%), or Croda polymer ATPlus (0.25%) was added. Lactose (0.1%) or cryobuffer 1 (CB1, 2%) were also added where indicated. The concentration of each polymer used was determined by compatibility assay, where each of the DMA #5 microbes was grown in media with increasing concentrations of the polymer. The highest concentration at which no reduction in microbial growth was observed was selected. Mock control groups, consisting of polymer and water, were also prepared as a microbiological control.

The solutions were sprayed using sterilized sprayers onto growing flowers and strawberries. Strawberries were harvested 0, 2, and 7 days after inoculation, homogenized, and plated to determine the number of viable microbes present on the fruit.

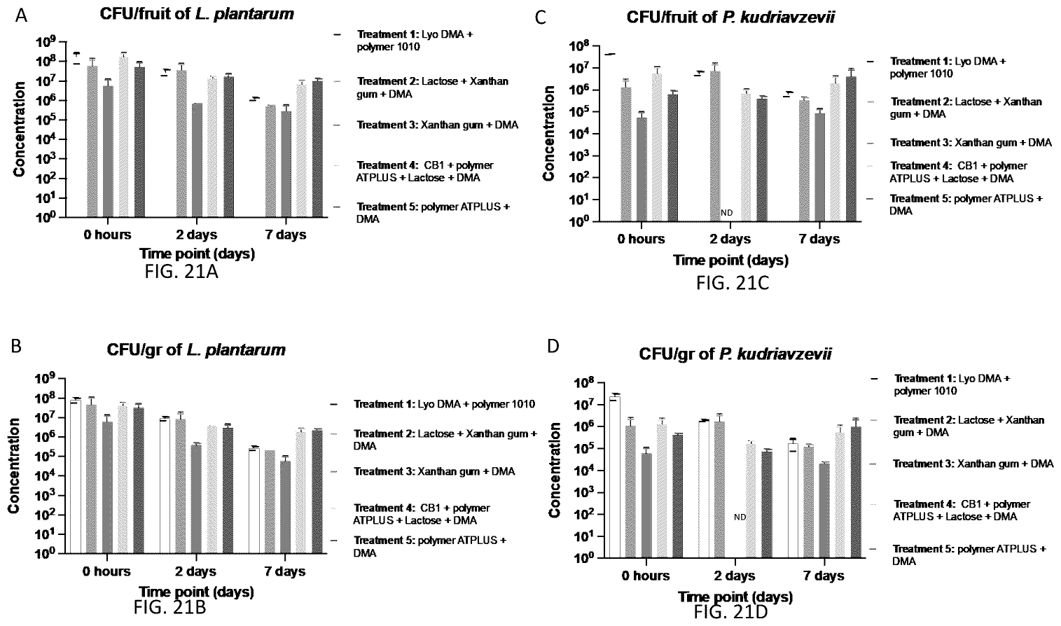

All treatment groups had recoverable yeast and bacteria at 0-, 2-, and 7-days post inoculation (FIG. 21). The CFUs for both the yeast and bacterium comprising DMA #5 decreased at two days and further at 7 days for each treatment group. Addition of lactose or CB1 to the polymers did not improve the DMA titers. Differences in the amount of loss in viable CFUs over 7 days for most groups usually varied from one to two logs (Table J). However, the treatment group with ATPlus polymer alone, demonstrated the highest total DMA survival rates at 7 days post application, with only a 73% decrease from TO.

These results confirm that methods described herein can be used for successful application of beneficial microbes to the surfaces and fruits.

TABLE J
Average CFU/fruit of achieved 7 days after application of a DMA with
different natural and synthetic polymers. Note that treatments 4 and 5
(ATPlus polymer) have the highest yields per fruit of the DMA.
DMA Average CFU/fruit (Lactobacillus plantarum & Pichia kudriazevzii)

| | 0 hours | 7 days |
|---|---|---|
| Treatment 1. Polymer 1010 (Lyophilized | 1.05E+08 | 9.15E+05 |
| DMA) | | |
| Treatment 2. Xanthan gum + lactose | 3.08E+07 | 4.18E+05 |
| Treatment 3. Xanthan gum | 2.94E+06 | 1.90E+05 |
| Treatment 4. ATPlus polymer + CB1 + lactose | 8.97E+07 | 4.51E+06 |
| Treatment 5. ATPlus polymer | 2.73E+07 | 7.25E+06 |

### Example 18: DMA Enrichment of Fruits by Direct Application of a Bolus to Flowers

Application of DMAs to ripening fruit may not always be possible in an agricultural setting due to environmental conditions, cultivation, or harvesting practices. Thus, probiotic enrichment may only be possible before the fruit appears. In the present example, a method of DMA enrichment of strawberries is achieved through application of microbes to the flower.

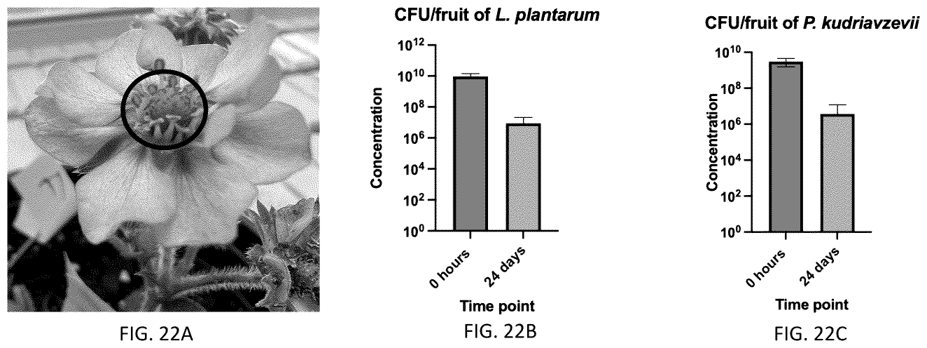

Briefly, lyophillized L. plantarum and P. kudriavzevii were resuspended in a 0.25% ATPlus polymer solution to a final concentration of 1×10¹¹ CFUs per mL. The inoculum solution was plated on selective media to determine CFU. The solution was applied to growing flowers via pipette application to the center, pistil containing region, (90 μl per flower, see FIG. 22A). Flowers were homogenized to determine the CFU applied. After 24 days, mature fruit was harvested and homogenized to quantify the number of DMA microbes present on the fruit. DMA-microbe identification was performed by 16S sequencing.

Successful colonization of mature fruit was achieved by application to flowers of a solution containing polymer and high concentrations of DMA microbes (FIG. 22). A two-log reduction in CFU was observed between flowers (0 days) and mature fruit (24 days), indicating that the DMA microbes are able to persist on the strawberry plants for an extended period.

### Example 19: DMA Enrichment of Fruits by Spray Application to Flowers

Application of DMAs to individual flowers could prove time-consuming and costly to the grower. Many farms use either mechanized or manual spray devices for application of fertilizers and pesticides. Application of DMAs in the field would be facilitated if existing spray equipment could be used. The present example describes application of DMAs to flowers by means of spraying.

Briefly, lyophillized L. plantarum and P. kudriavzevii were resuspended in a 0.25% ATPlus polymer solution. Mock control material, consisting of polymer and water, was also prepared as a microbiological control. The inoculum solution was plated on selective media to determine CFU. Flowers were homogenized to determine the CFU applied. The solution containing 1.2×10¹¹ CFU per mL DMA #5 was sprayed onto growing flowers (a volume of 1.1 mL per flower, see Table K). After 25 days, mature fruit was harvested and homogenized to quantify the number of DMA microbes present on the fruit. Verification of DMA microbes was performed by 16S rDNA sequencing.

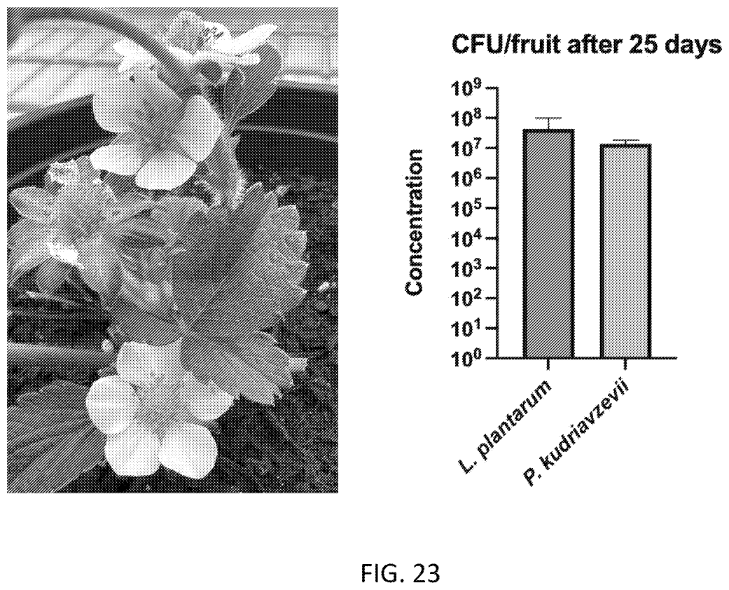

Successful colonization of mature fruit was achieved by spray application to flowers of a solution containing polymer and high concentrations of DMA microbes (Table K and FIG. 23). Strawberries had an average DMA concentration of 5.8×10⁷ CFUs per fruit 24 days after application. No growth was observed in the mock control groups.

TABLE K
Application of DMA via spray to flowers. Note that the final DMA
yield per fruit is similar to the bolus application method.

| | DMA Strain | | Average DMA |
|---|---|---|---|
| | Concentration | CFUs Sprayed | titer per Mature |
| | (per mL) | (1.1 mL) | Fruit |
| L. plantarum | 8.67E+10 | 9.54E+10 | 4.39E+07 |
| P. kudriavzevii | 3.94E+10 | 4.33E+10 | 1.40E+07 |

### Example 20: DMA Enrichment of Fruits and Flowers with Three Different Microbe Preparation Methods

In a small agricultural setting, culturing of microbes and preparation of DMAs for field application may prove challenging as specialized tools and equipment may be unavailable. Thus it is important to be able to provide convienient starting materials. Lyophilization is a common method used to preserve viable microbes and is an excellent alternative to microbial culture in the field, allowing for simple mixture preparation prior to application. Alternatively, large agricultural groups can reduce cost in by direct cultivation of the microbes rather than purchasing a lyophilized product. The present example describes methods to enrich fruit or flowers with DMA and polymer mixtures using lyophilized microbes, microbes cultured and washed of media and metabolites, and microbes directley diluted from liquid culture.

DP100 and DP102 were inoculated from cryostock preparations and grown overnight at 30° C. in deMan Rogosa Sharpe (MRS) and potato dextrose broth (PDB) liquid medium respectively. Microbes were diluted to a final concentration of 1×10¹¹ CFUs per mL with water or pelleted and resuspended in water to remove spent media and metabolites. Alternatively, lyophilized microbes were produced by fermentation in a bioreactor, harvested by centrifugation, homogenized with an excipient blend of oligofructose, sucrose, and ascorbic acid or of inulin, trehalose, and ascorbic acid, then lyophilized. These microbe powders were enumerated and known concentrations were weighed sterilely and added to water. To these DMA mixtures Croda polymer ATPlus (0.25%) was added. Mock control material, consisting of polymer and water, groups was also prepared. The solutions were sprayed onto growing flowers and strawberries. Strawberries were harvested 0, 2, and 7 days after inoculation, homogenized and plated to determine the number of viable microbes present on the fruit. Flowers were homogenized to determine the CFU applied. After 17-22 days, mature fruit was harvested and homogenized to quantify the number of DMA microbes present on the fruit. Verification of microbe ID was performed by 16S rDNA sequencing.

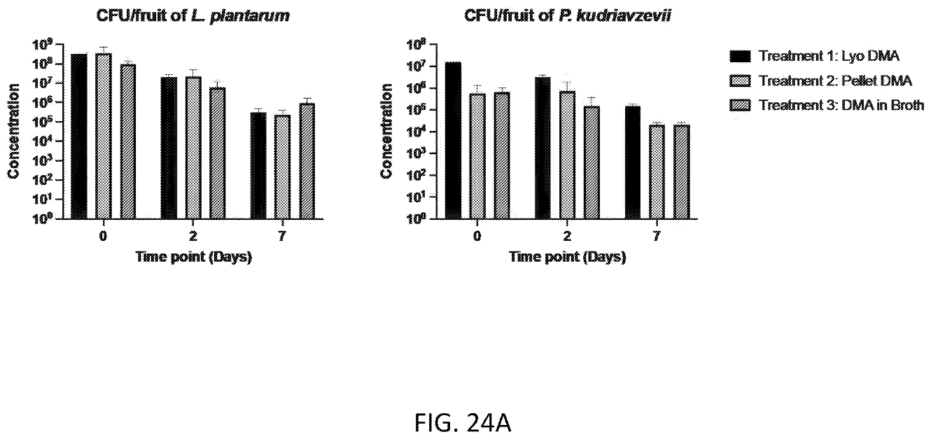

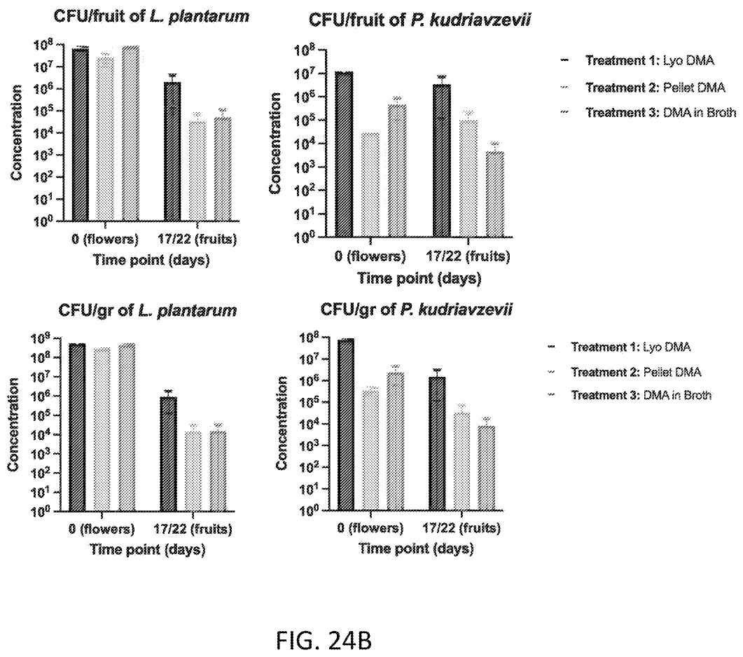

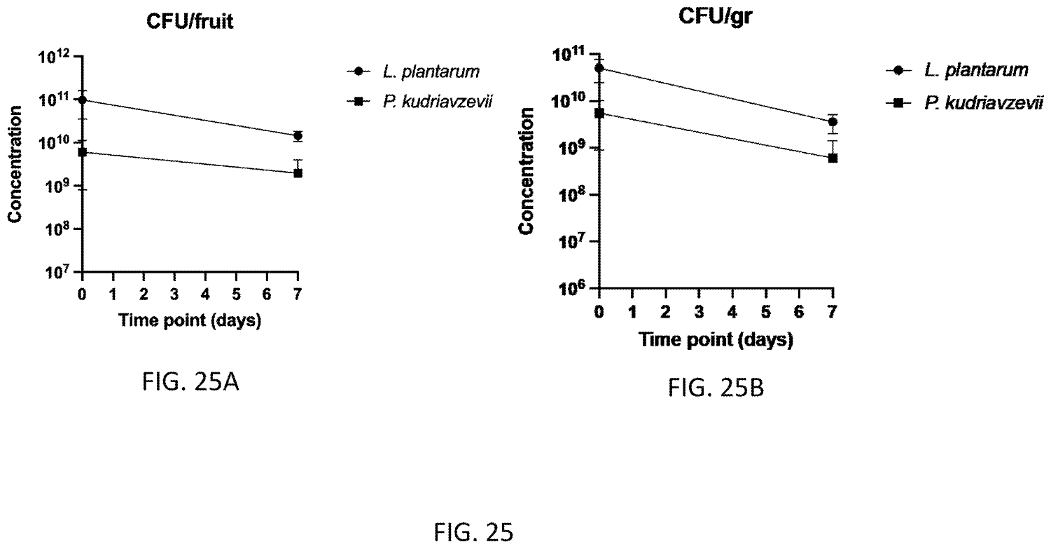

For strawberries, all DMA preparation methods resulted in colonization of fruits with viable microbes. No growth was detected for the mock control group. Each of the three bacterial preparation techniques resulted in similar starting titers on strawberries (FIG. 24A, left). These decreased similarly over the course of 7 days. The preparations of DP102 had differing concentrations initially, with the lyophilized yeast being the highest (FIG. 24A, right). Viable CFUs decreased proportionally for this organism with each preparation method, suggesting each is similarly suitable for fruit application.

DMA application to flowers, with colonization of the resultant fruit, was successful with each method of cultivation. No growth was detected for the mock control group. Bacterial CFUs from harvested flowers were similar across treatment groups (FIG. 24B, left) but differed on the resulting strawberries, with a greater that 10-fold increase in recoverable bacteria from the lyophilized starting material group. Initial yeast colonization levels were more variable (FIG. 24B, right). Likewise, the final yields on fruit varied, however, only the broth preparation showed a greater than one log decrease in CFUs between the first and second timepoint.

These results confirm that the methods described herein can successfully enrich fruit or flowers with DMA and polymer mixtures using lyophilized microbes, microbes cultured and washed of media and metabolites, and microbes directley diluted from liquid culture.

### Example 21 High Titers of DMA Microbes can be Achieved with High Titer Application to Fruits

Probiotic microbes are typically delivered in a capsule format at concentrations of 1×10⁹-1×10¹⁰ CFU per capsule (Wilkins, T. and Sequoia, J. Am Fam Physician. 2017; 96 (3): 170-178). However, many individuals either avoid swallowing pills or prefer to consume probiotic microbes via the consumption of fermented foods, which often deliver far fewer microbes per serving. The present example describes a method to enrich fruit with DMAs at titers equivalent to current probiotics by applying the a highly concentrated DMA to fruits in a polymer solution.

Briefly, lyophillized L. plantarum and P. kudriavzevii were resuspended in a 0.25% ATPlus polymer solution. Mock control material, consisting of polymer and water, groups was also prepared. The inoculum solution was plated on selective media to determine CFU. The solution was sprayed onto growing strawberries. Strawberries were harvested 0 and 7 days after inoculation, homogenized and plated to determine the number of viable microbes present on the fruit.

The DMA solution applied to the microbes contained yeast at a concentration above 1×10⁹ CFU/mL and above 1×10¹⁰ CFU/mL for the bacterium (Table L). Application of this solution resulted in a high titer of viable DMA microbes that persisted for 7 days with less than one order of magnitude lost for each DMA constituent (FIG. 26). No growth was detected for the mock control group. The final concentrations per fruit of each of the microbes in the DMA is equivalent to those present in probiotic capsules.

These results confirm that the methods described herien can succesfully enrich fruit with DMAs at titers equivalent to current probiotics by applying the a highly concentrated DMA to fruits in a polymer solution.

TABLE L
Application of high-concentration DMAs to strawberries increases
the resultant microbial titers. Note that the DMA titer achieved
per fruit after 7 days is similar to many probiotics.

| | DMA Inoculum | Average DMA | Average DMA |
|---|---|---|---|
| | Concentration | yield at 0 days | yield at 7 days |
| | (CFU/mL) | (CFU/fruit) | (CFU/fruit) |
| L. plantarum | 3.60E+10 | 9.77E+10 | 1.45E+10 |
| P. kudriavzevii | 2.00E+09 | 6.03E+09 | 1.97E+09 |

### Example 22: Enrichment of Exemplary DMAs in Fruits by Spray Application to Fruits and Flowers

Lyophilized DMAs, listed in Table M, were resuspended in a 0.25% ATPlus polymer solution. Mock control material, consisting of polymer and water, was also prepared as a microbiological control. The inoculum solution was plated on selective media to determine CFU. Flowers were homogenized to determine the CFU applied. The solution was sprayed onto growing flowers (a volume of 1.1 mL per flower). After 25 days, mature fruit was harvested and homogenized to quantify the number of DMA microbes present on the fruit. Verification of DMA microbes was performed by 16S sequencing. Successful colonization of mature fruit was achieved by spray application to flowers of a solution containing polymer and high concentrations of DMA microbes.

In addition, lyophillized DMAs, listed in Table M, were resuspended in a 0.25% ATPlus polymer solution. Mock control material, consisting of polymer and water, groups was also prepared. The inoculum solution was plated on selective media to determine CFU. The solution was sprayed onto growing strawberries. Strawberries were harvested 0 and 7 days after inoculation, homogenized, and plated to determine the number of viable microbes present on the fruit. Application of this solution resulted in a high titer of viable DMA microbes in the resulting fruit.

These results confirm that successful colonization of mature fruit can be achieved by spray application to flowers of a solution containing polymer (such as ATPLUS polymer) and high concentrations of DMA microbes.

TABLE M
Composition of Exemplary DMAs used for administration
to flowers and strawberries.

| Category | Strain | Genus | Species |
|---|---|---|---|
| Anaerobe | SBI4825 | Clostridium | sp. |
| | SBI4833 | Clostridioides | mangenotii |
| | SBI4259 | Weisella | cibaria |
| | SBI4260 | Lactobacillus | plantarum |
| Lactic | *SBS04254 | Lactobacillus | brevis |
| Acid | SBI4255 | Leuconostoc | mesenteroides |
| Bacteria | *SBI04881 | Lactobacillus | buchneri |
| | *SBS2335 | Pediococcus | pentosaceus |
| | *SBI04916 | Lactococcus | lactis |
| | SBI04913 | Lactobacillus | harbinensis |
| Bacteria | *SBI4877 | Bacillus | velezensis |
| (Other) | | | |
| Fungi | SBI4263 | Pichia | kudriavzevii |
| | SBI00303 | Meyerozyma | carribica |

*Indicates strain has Qualified Presumption of Safety status

### Example 23: Administration of DMA-Colonized Strawberries to Mice and Detection of DMAs in the Mouse Fecal Microbiota

Groups of 8-week-old male C57Bl/6J mice are group housed under standard vivarium conditions and fed standard rodent chow. Groups of mice (n=5/group) are randomized to receive dietary supplementation with strawberries colonized with a DMA comprising probiotic bacteria and fungi (10⁴-10⁹ CFU per g fruit). Control animals are supplemented with strawberries containing no added DMA. Strawberries are administered to groups of mice by gently crushing 10 g strawberries using a sterile mortar and pestle and placing the crushed fruit in a small petri dish, placed in the cage bedding. Strawberry supplements are replaced daily for 14 days, and fecal samples are collected from each mouse on days 0 (before strawberry supplementation), 1, 3, 5, 7, and 14. Strawberry supplementation is discontinued on day 15, and bedding is changed for all cages. Additional fecal samples are collected on days 1, 3, 5, 7, and 14 after discontinuing strawberry supplementation.

Mouse fecal samples are used to extract metagenomic DNA and subjected to whole-metagenome shotgun sequencing using the Illumina NovaSeq platform at a depth of 5 Gigabases per sample. The resulting sequence is then used to determine the impact of DMA administration via colonized strawberries on fecal microbial community composition and on the detection of DMA component strains using fragment recruitment plots. In addition, quantitative PCR assays are performed on fecal metagenomes using strain-specific primers to accurately quantify the kinetics of DMA component strains in the fecal microbiota over time.

These results confirm that administration of DMA-colonized berries described herein to mice are detected in mouse fecal microbiota, and the DMAs have successfully colonized the mouse gut.

### Example 24: Administration of DMA-Colonized Strawberries to Healthy Human Subjects and Detection of DMAs in the Fecal Microbiota

Groups of healthy human subjects (n=5/group) are randomized to receive dietary supplementation with strawberries colonized with a DMA comprising probiotic bacteria and fungi (10⁴-10⁹ CFU per g fruit) or supplementation with strawberries containing no added DMA. Healthy human subjects are instructed to consume 5 strawberries per day with a morning meal. Strawberries are administered daily for 14 days, and fecal samples are collected from each subject on days 0 (before strawberry supplementation), 1, 3, 5, 7, and 14. Strawberry supplementation is discontinued on day 15, and additional fecal samples are collected on days 1, 3, 5, 7, 14, 21, and 28 after discontinuing strawberry supplementation.

Fecal samples are used to extract metagenomic DNA and subjected to whole-metagenome shotgun sequencing using the Illumina NovaSeq platform at a depth of 5 Gigabases per sample. The resulting sequence is then used to determine the impact of DMA administration via colonized strawberries on fecal microbial community composition and on the detection of DMA component strains using fragment recruitment plots. In addition, quantitative PCR assays are performed on fecal metagenomes using strain-specific primers to accurately quantify the kinetics of DMA component strains in the fecal microbiota over time.

These results confirm that administration of DMA-colonized berries described herein to human subjects are detected in human fecal microbiota, and the DMAs have successfully colonized the gut of the human subject.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1.-73. (canceled)

74. A nutritive food product comprising at least a portion of an edible plant selected from the group consisting of: a vine crop, a leafy vegetable, a cucurbit, a root vegetable, and a perennial and annual bush, wherein the at least a portion of the edible plant comprises a diversified microbial ecology comprising at least one heterologous microbe.

75. The nutritive food product of claim 74, wherein the at least a portion of the edible plant comprises a part of the plant selected from: a berry, a root, and a leaf.

76. The nutritive food product of claim 74, wherein the at least one heterologous microbe comprises a bacterial species or a fungal species selected from Table B or Table E, or a combination thereof.

77. The nutritive food product of claim 74, wherein the at least one heterologous microbe comprises a nucleic acid sequence that has at least 97% identity to any one of the sequences shown in Table F.

78. The nutritive food product of claim 74, wherein the amount of the heterologous microbe comprises at least 1×10⁴ CFU/gram of the at least a portion of the edible plant.

79. The nutritive food product of claim 74, wherein the diversified microbial ecology comprising the at least one heterologous microbe benefits growth of the edible plant.

80. The nutritive food product of claim 74, wherein the diversified microbial ecology comprising the at least one heterologous microbe improves resistance of the edible plant to an abiotic stress selected from temperature and moisture level.

81. The nutritive food product of claim 74, wherein the diversified microbial ecology comprising the at least one heterologous microbe produces a heterologous metabolite or enhances the production of endogenous metabolites in a tissue of the edible plant.

82. The nutritive food product of claim 74, wherein the at least a portion of the edible plant comprises detectable amounts of the heterologous microbe.

83. A seed or seedling of an edible plant having deposited on an exterior surface of the seed or seedling a formulation comprising at least one heterologous microbe, wherein the at least one heterologous microbe is deposited on an exterior surface of the seed or seedling in an amount effective to colonize the plant, wherein the edible plant is selected from the group consisting of: a vine crop, a leafy vegetable, a cucurbit, a root vegetable, and a perennial and annual bush.

84. The seed or seedling of claim 83, wherein the at least one heterologous microbe comprises a bacterial species or a fungal species selected from Table B or Table E, or a combination thereof.

85. The seed or seedling of claim 83, wherein at least one heterologous microbe comprises a nucleic acid sequence that has at least 97% identity to any one of the sequences shown in Table F.

86. The seed or seeding of claim 83, wherein the formulation further comprises at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, a nutrient, and a polymeric and/or adhesive substance.

87. The seed or seedling of claim 86, wherein the polymeric substance comprises a vinyl pyrrolidone/vinyl acetate copolymer.

88. An edible plant having deposited on an exterior surface of a flower or fruit of the edible plant a formulation comprising at least one heterologous microbe, wherein the edible plant is selected from the group consisting of: a vine crop, a leafy vegetable, a cucurbit, a root vegetable, and a perennial and annual bush and wherein the at least one heterologous microbe is deposited on the exterior surface of the flower or fruit in an amount effective to colonize the edible plant.

89. The edible plant of claim 88, wherein the at least one heterologous microbe comprises a bacterial species or a fungal species selected from Table B or Table E, or a combination thereof.

90. The edible plant of claim 88, wherein at least one heterologous microbe comprises a nucleic acid sequence that has at least 97% identity to any one of the sequences shown in Table F.

91. The edible plant of claim 88, wherein the amount of the heterologous microbe effective to colonize the edible plant comprises at least 1×10⁴ CFU/gram of flower or fruit.

92. The edible plant of claim 88, further comprising at least one member selected from the group consisting of an agriculturally compatible carrier, a tackifier, a microbial stabilizer, a fungicide, an antibacterial agent, an herbicide, a nematicide, an insecticide, a plant growth regulator, a rodenticide, a humectant, plant penetration aid, a nutrient, and a polymeric and/or adhesive substance.

93. The edible plant of claim 92, wherein the polymeric substance comprises a vinyl pyrrolidone/vinyl acetate copolymer, an alkoxylated polyol ester, or a modified Tween 20 (polyoxyethylene/polyoxypropylene/sorbitan monolaurate) polymer.
